SEQUENCE LISTING (153 sequences)

SEQ ID NO: 1 - DNA, 5431 bp, Artificial Sequence
  Description of Artificial Sequence Synthetic polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccactgtgctggatatctgcagaattccaccacactggactagtggatccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 2 - DNA, 4479 bp, Artificial Sequence
  Description of Artificial Sequence Synthetic polynucleotide
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgacggtatcgataagcttgatatcgaattcacgtgggcccggtaccgtatactctagagcggccgcggatccagatctttttccctcgccaaaaattatggggacatcatgaagccccttgagcatctgacttctggctaataaaggaaatttatttcattgcaatagtgtgttggaattttttgtgtctctcactcggaaggacatatgggagggcaaatcatttaaaacatcagaatcagtatttggtttagagtttggcaacatatgccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccggggggggggggcgctgaggtctgcctcgtgaagaaggtgttgctgactcataccagggcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 3 - DNA, 7648 bp, Artificial Sequence
  Description of Artificial Sequence Synthetic polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccactgtgctggatatctgcagaattccaccacactggactagtggatccatgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcaaggatccatggctcgtgcggtcgggatcgacctcgggaccaccaactccgtcgtctcggttctggaaggtggcgacccggtcgtcgtcgccaactccgagggctccaggaccaccccgtcaattgtcgcgttcgcccgcaacggtgaggtgctggtcggccagcccgccaagaaccaggcagtgaccaacgtcgatcgcaccgtgcgctcggtcaagcgacacatgggcagcgactggtccatagagattgacggcaagaaatacaccgcgccggagatcagcgcccgcattctgatgaagctgaagcgcgacgccgaggcctacctcggtgaggacattaccgacgcggttatcacgacgcccgcctacttcaatgacgcccagcgtcaggccaccaaggacgccggccagatcgccggcctcaacgtgctgcggatcgtcaacgagccgaccgcggccgcgctggcctacggcctcgacaagggcgagaaggagcagcgaatcctggtcttcgacttgggtggtggcactttcgacgtttccctgctggagatcggcgagggtgtggttgaggtccgtgccacttcgggtgacaaccacctcggcggcgacgactgggaccagcgggtcgtcgattggctggtggacaagttcaagggcaccagcggcatcgatctgaccaaggacaagatggcgatgcagcggctgcgggaagccgccgagaaggcaaagatcgagctgagttcgagtcagtccacctcgatcaacctgccctacatcaccgtcgacgccgacaagaacccgttgttcttagacgagcagctgacccgcgcggagttccaacggatcactcaggacctgctggaccgcactcgcaagccgttccagtcggtgatcgctgacaccggcatttcggtgtcggagatcgatcacgttgtgctcgtgggtggttcgacccggatgcccgcggtgaccgatctggtcaaggaactcaccggcggcaaggaacccaacaagggcgtcaaccccgatgaggttgtcgcggtgggagccgctctgcaggccggcgtcctcaagggcgaggtgaaagacgttctgctgcttgatgttaccccgctgagcctgggtatcgagaccaagggcggggtgatgaccaggctcatcgagcgcaacaccacgatccccaccaagcggtcggagactttcaccaccgccgacgacaaccaaccgtcggtgcagatccaggtctatcagggggagcgtgagatcgccgcgcacaacaagttgctcgggtccttcgagctgaccggcatcccgccggcgccgcgggggattccgcagatcgaggtcactttcgacatcgacgccaacggcattgtgcacgtcaccgccaaggacaagggcaccggcaaggagaacacgatccgaatccaggaaggctcgggcctgtccaaggaagacattgaccgcatgatcaaggacgccgaagcgcacgccgaggaggatcgcaagcgtcgcgaggaggccgatgttcgtaatcaagccgagacattggtctaccagacggagaagttcgtcaaagaacagcgtgaggccgagggtggttcgaagttcgtaatcaagccgagacattggtctaccagacggagaagttcgtcaaagaacagcgtgaggccgagggtggttcgaaggtacctgaagacacgctgaacaaggttgatgccgcggtggcggaagcgaaggcggcacttggcggatcggatatttcggccatcaagtcggcgatggagaagctgggccaggagtcgcaggctctggggcaagcgatctacgaagcagctcaggctgcgtcacaggccactggcgctgcccaccccggctcggctgatgaaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaatgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatggctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 4 - DNA, 6221 bp, Artificial Sequence
  Description of Artificial Sequence Synthetic polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccactgtgctggatatctgcagaattcatgcgcctgcactttcccgagggcggcagcctggccgcgctgaccgcgcaccaggcttgccacctgccgctggagactttcacccgtcatcgccagccgcgcggctgggaacaactggagcagtgcggctatccggtgcagcggctggtcgccctctacctggcggcgcggctgtcgtggaaccaggtcgaccaggtgatccgcaacgccctggccagccccggcagcggcggcgacctgggcgaagcgatccgcgagcagccggagcaggcccgtctggccctgaccctggccgccgccgagagcgagcgcttcgtccggcagggcaccggcaacgacgaggccggcgcggccaacgccgacgtggtgagcctgacctgcccggtcgccgccggtgaatgcgcgggcccggcggacagcggcgacgccctgctggagcgcaactatcccactggcgcggagttcctcggcgacggcggcgacgtcagcttcagcacccgcggcacgcagaacgaattcatgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcaaggatccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaattgaatgtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 5 - DNA, 5970 bp, Artificial Sequence
  Description of Artificial Sequence Synthetic polynucleotide
gctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccagggcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattattaataggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgacatgctgctatccgtgccgctgctgctcggcctcctcggcctggccgtcgccgagcctgccgtctacttcaaggagcagtttctggacggggacgggtggacttcccgctggatcgaatccaaacacaagtcagattttggcaaattcgttctcagttccggcaagttctacggtgacgaggagaaagataaaggtttgcagacaagccaggatgcacgcttttatgctctgtcggccagtttcgagcctttcagcaacaaaggccagacgctggtggtgcagttcacggtgaaacatgagcagaacatcgactgtgggggcggctatgtgaagctgtttcctaatagtttggaccagacagacatgcacggagactcagaatacaacatcatgtttggtcccgacatctgtggccctggcaccaagaaggttcatgtcatcttcaactacaagggcaagaacgtgctgatcaacaaggacatccgttgcaaggatgatgagtttacacacctgtacacactgattgtgcggccagacaacacctatgaggtgaagattgacaacagccaggtggagtccggctccttggaagacgattgggacttcctgccacccaagaagataaaggatcctgatgcttcaaaaccggaagactgggatgagcgggccaagatcgatgatcccacagactccaagcctgaggactgggacaagcccgagcatatccctgaccctgatgctaagaagcccgaggactgggatgaagagatggacggagagtgggaacccccagtgattcagaaccctgagtacaagggtgagtggaagccccggcagatcgacaacccagattacaagggcacttggatccacccagaaattgacaaccccgagtattctcccgatcccagtatctatgcctatgataactttggcgtgctgggcctggacctctggcaggtcaagtctggcaccatctttgacaacttcctcatcaccaacgatgaggcatacgctgaggagtttggcaacgagacgtggggcgtaacaaaggcagcagagaaacaaatgaaggacaaacaggacgaggagcagaggcttaaggaggaggaagaagacaagaaacgcaaagaggaggaggaggcagaggacaaggaggatgatgaggacaaagatgaggatgaggaggatgaggaggacaaggaggaagatgaggaggaagatgtccccggccaggccaaggacgagctggaattcatgcatggagatacacctacattgcatgaatatatgttagatttgcaaccagagacaactgatctctacggttatgggcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccataaggatccagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccatag

SEQ ID NO: 6 - DNA, 1257 bp, Artificial Sequence
  Description of Artificial Sequence Synthetic polynucleotide
atgacctctcgccgctccgtgaagtcgggtccgcgggaggttccgcgcgatgagtacgaggatctgtactacaccccgtcttcaggtatggcgagtcccgatagtccgcctgacacctcccgccgtggcgccctacagacacgctcgcgccagaggggcgaggtccgtttcgtccagtacgacgagtcggattatgccctctacgggggctcgtcttccgaagacgacgaacacccggaggtcccccggacgcggcgtcccgtttccggggcggttttgtccggcccggggcctgcgcgggcgcctccgccacccgctgggtccggaggggccggacgcacacccaccaccgccccccgggccccccgaacccagcgggtggcgtctaaggcccccgcggccccggcggcggagaccacccgcggcaggaaatcggcccagccagaatccgccgcactcccagacgcccccgcgtcgacggcgccaacccgatccaagacacccgcgcaggggctggccagaaagctgcactttagcaccgcccccccaaaccccgacgcgccatggaccccccgggtggccggctttaacaagcgcgtcttctgcgccgcggtcgggcgcctggcggccatgcatgcccggatggcggctgtccagctctgggacatgtcgcgtccgcgcacagacgaagacctcaacgaactccttggcatcaccaccatccgcgtgacggtctgcgagggcaaaaacctgcttcagcgcgccaacgagttggtgaatccagacgtggtgcaggacgtcgacgcggccacggcgactcgagggcgttctgcggcgtcgcgccccaccgagcgacctcgagccccagcccgctccgcttctcgccccagacggcccgtcgagggtaccgagctcggatccatgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcaggataagcttaagtttaaaccgctgatcagcctcgactgtgccttctag

SEQ ID NO: 7 - DNA, 903 bp, Human herpesvirus
  CDS (1)..(903)
atgacctctcgccgctccgtgaagtcgggtccgcgggaggttccgcgcgatgagtacgaggatctgtactacaccccgtcttcaggtatggcgagtcccgatagtccgcctgacacctcccgccgtggcgccctacagacacgctcgcgccagaggggcgaggtccgtttcgtccagtacgacgagtcggattatgccctctacgggggctcgtcttccgaagacgacgaacacccggaggtcccccggacgcggcgtcccgtttccggggcggttttgtccggcccggggcctgcgcgggcgcctccgccacccgctgggtccggaggggccggacgcacacccaccaccgccccccgggccccccgaacccagcgggtggcgtctaaggcccccgcggccccggcggcggagaccacccgcggcaggaaatcggcccagccagaatccgccgcactcccagacgcccccgcgtcgacggcgccaacccgatccaagacacccgcgcaggggctggccagaaagctgcactttagcaccgcccccccaaaccccgacgcgccatggaccccccgggtggccggctttaacaagcgcgtcttctgcgccgcggtcgggcgcctggcggccatgcatgcccggatggcggctgtccagctctgggacatgtcgcgtccgcgcacagacgaagacctcaacgaactccttggcatcaccaccatccgcgtgacggtctgcgagggcaaaaacctgcttcagcgcgccaacgagttggtgaatccagacgtggtgcaggacgtcgacgcggccacggcgactcgagggcgttctgcggcgtcgcgccccaccgagcgacctcgagccccagcccgctccgcttctcgccccagacggcccgtcgag

SEQ ID NO: 8 - DNA, 297 bp, Human papillomavirus
  CDS (1)..(297)
atgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcaggataagctt

SEQ ID NO: 9 - protein, 99 aa, Human papillomavirus
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQDKL

SEQ ID NO: 10 - protein, 96 aa, Artificial Sequence
  Description of Artificial Sequence Synthetic polypeptide
MHGDTPTLHEYMLDLQPETTDLYGYEQDSSEEEDEIDGPAGQAEPDRAHYNIVTFCCKCDSTLRLCVQSTHVDIRTLEDLLMGTLGIVCPICSQKP

SEQ ID NO: 11 - DNA, 477 bp, Human papillomavirus
  CDS (1)..(474)
atgcaccaaaagagaactgcaatgtttcaggacccacaggagcgacccagaaagttaccacagttatgcacagagctgcaaacaactatacatgatataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtttgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctgtaa

SEQ ID NO: 12 - protein, 158 aa, Human papillomavirus
MHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

SEQ ID NO: 13 - protein, 149 aa, Human papillomavirus
MFQDPQERPRKLPQLCTELQTTIHDIICVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

SEQ ID NO: 14 - DNA, 1698 bp, Influenza A virus
atgaaggcaaacctactggtcctgttaagtgcacttgcagctgcagatgcagacacaatatgtataggctaccatgcgaacaattcaaccgacactgttgacacagtactcgagaagaatgtgacagtgacacactctgttaacctgctcgaagacagccacaacggaaaactatgtagattaaaaggaatagccccactacaattggggaaatgtaacatcgccggatggctcttgggaaacccagaatgcgacccactgcttccagtgagatcatggtcctacattgtagaaacaccaaactctgagaatggaatatgttatccaggagatttcatcgactatgaggagctgagggagcaattgagctcagtgtcatcattcgaaagattcgaaatatttcccaaagaaagctcatggcccaaccacaacacaaacggagtaacggcagcatgctcccatgaggggaaaagcagtttttacagaaatttgctatggctgacggagaaggagggctcatacccaaagctgaaaaattcttatgtgaacaaaaaagggaaagaagtccttgtactgtggggtattcatcacccgcctaacagtaaggaacaacagaatatctatcagaatgaaaatgcttatgtctctgtagtgacttcaaattataacaggagatttaccccggaaatagcagaaagacccaaagtaagagatcaagctgggaggatgaactattactggaccttgctaaaacccggagacacaataatatttgaggcaaatggaaatctaatagcaccaatgtatgctttcgcactgagtagaggctttgggtccggcatcatcacctcaaacgcatcaatgcatgagtgtaacacgaagtgtcaaacacccctgggagctataaacagcagtctcccttaccagaatatacacccagtcacaataggagagtgcccaaaatacgtcaggagtgccaaattgaggatggttacaggactaaggaacactccgtccattcaatccagaggtctatttggagccattgccggttttattgaagggggatggactggaatgatagatggatggtatggttatcatcatcagaatgaacagggatcaggctatgcagcggatcaaaaaagcacacaaaatgccattaacgggattacaaacaaggtgaacactgttatcgagaaaatgaacattcaattcacagctgtgggtaaagaattcaacaaattagaaaaaaggatggaaaatttaaataaaaaagttgatgatggatttctggacatttggacatataatgcagaattgttagttctactggaaaatgaaaggactctggatttccatgactcaaatgtgaagaatctgtatgagaaagtaaaaagccaattaaagaataatgccaaagaaatcggaaatggatgttttgagttctaccacaagtgtgacaatgaatgcatggaaagtgtaagaaatgggacttatgattatcccaaatattcagaagagtcaaagttgaacagggaaaaggtagatggagtgaaattggaatcaatggggatctatcagattctggcgatctactcaactgtcgccagttcactggtgcttttggtctccctgggggcaatcagtttctggatgtgttctaatggatctttgcagtgcagaatatgcatctga

SEQ ID NO: 15 - protein, 563 aa, Influenza A virus
MKANLLVLLSAAADADTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNIYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNTPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQILAIYSTVASSLVLLVSLGAISFWMCSNGSLQCRICI

SEQ ID NO: 16 - DNA, 501 bp, Artificial Sequence
  Description of Artificial Sequence Synthetic polynucleotide
atggcggcccccggcgcccggcggccgctgctcctgctgctgctggcaggccttgcacatggcgcctcagcactctttgaggatctaatcatgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgttaccttttgttgcaagtgtgactctacgcttcggttgtgcgtacaaagcacacacgtagacattcgtactttggaagacctgttaatgggcacactaggaattgtgtgccccatctgttctcaggatcttaacaacatgttgatccccattgctgtgggcggtgccctggcagggctggtcctcatcgtcctcattgcctacctcattggcaggaagaggagtcacgccggctatcagaccatctag

SEQ ID NO: 17 - protein, 166 aa, Artificial Sequence
  Description of Artificial Sequence Synthetic polypeptide
MAAPGARRPLLLLLLAGLAHGASALFEDLIMHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQDLNNMLIPIAVGGALAGLVLIVLIAYLIGRKRSHAGYQTI

SEQ ID NO: 18 - DNA, 5915 bp, Artificial Sequence
  Description of Artificial Sequence Synthetic polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccactgtgctggatatctgcagaattcatggcggcccccggcgcccggcggccgctgctcctgctgctgctggcaggccttgcacatggcgcctcagcactctttgaggatctaatcatgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgttaccttttgttgcaagtgtgactctacgcttcggttgtgcgtacaaagcacacacgtagacattcgtactttggaagacctgttaatgggcacactaggaattgtgtgccccatctgttctcaggatcttaacaacatgttgatccccattgctgtgggcggtgccctggcagggctggtcctcatcgtcctcattgcctacctcattggcaggaagaggagtcacgccggctatcagaccatctagggatccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 19 - DNA, 1878 bp, Mycobacterium tuberculosis
atggctcgtgcggtcgggatcgacctcgggaccaccaactccgtcgtctcggttctggaaggtggcgacccggtcgtcgtcgccaactccgagggctccaggaccaccccgtcaattgtcgcgttcgcccgcaacggtgaggtgctggtcggccagcccgccaagaaccaggcagtgaccaacgtcgatcgcaccgtgcgctcggtcaagcgacacatgggcagcgactggtccatagagattgacggcaagaaatacaccgcgccggagatcagcgcccgcattctgatgaagctgaagcgcgacgccgaggcctacctcggtgaggacattaccgacgcggttatcacgacgcccgcctacttcaatgacgcccagcgtcaggccaccaaggacgccggccagatcgccggcctcaacgtgctgcggatcgtcaacgagccgaccgcggccgcgctggcctacggcctcgacaagggcgagaaggagcagcgaatcctggtcttcgacttgggtggtggcactttcgacgtttccctgctggagatcggcgagggtgtggttgaggtccgtgccacttcgggtgacaaccacctcggcggcgacgactgggaccagcgggtcgtcgattggctggtggacaagttcaagggcaccagcggcatcgatctgaccaaggacaagatggcgatgcagcggctgcgggaagccgccgagaaggcaaagatcgagctgagttcgagtcagtccacctcgatcaacctgccctacatcaccgtcgacgccgacaagaacccgttgttcttagacgagcagctgacccgcgcggagttccaacggatcactcaggacctgctggaccgcactcgcaagccgttccagtcggtgatcgctgacaccggcatttcggtgtcggagatcgatcacgttgtgctcgtgggtggttcgacccggatgcccgcggtgaccgatctggtcaaggaactcaccggcggcaaggaacccaacaagggcgtcaaccccgatgaggttgtcgcggtgggagccgctctgcaggccggcgtcctcaagggcgaggtgaaagacgttctgctgcttgatgttaccccgctgagcctgggtatcgagaccaagggcggggtgatgaccaggctcatcgagcgcaacaccacgatccccaccaagcggtcggagactttcaccaccgccgacgacaaccaaccgtcggtgcagatccaggtctatcagggggagcgtgagatcgccgcgcacaacaagttgctcgggtccttcgagctgaccggcatcccgccggcgccgcgggggattccgcagatcgaggtcactttcgacatcgacgccaacggcattgtgcacgtcaccgccaaggacaagggcaccggcaaggagaacacgatccgaatccaggaaggctcgggcctgtccaaggaagacattgaccgcatgatcaaggacgccgaagcgcacgccgaggaggatcgcaagcgtcgcgaggaggccgatgttcgtaatcaagccgagacattggtctaccagacggagaagttcgtcaaagaacagcgtgaggccgagggtggttcgaaggtacctgaagacacgctgaacaaggttgatgccgcggtggcggaagcgaaggcggcacttggcggatcggatatttcggccatcaagtcggcgatggagaagctgggccaggagtcgcaggctctggggcaagcgatctacgaagcagctcaggctgcgtcacaggccactggcgctgcccaccccggcggcgagccgggcggtgcccaccccggctcggctgatgacgttgtggacgcggaggtggtcgacgacggccgggaggccaagtga

SEQ ID NO: 20 - protein, 621 aa, Mycobacterium tuberculosis
MARAVGIDLGTTNSVVSVLEGGDPVVVANSEGSRTTPSIVAFARNGEVLVGQPAKNQAVTNVDRTVRSVKRHMGSDWSIEIDGKKYTAPEISARILMKLKRDAEAYLGEDITDAVITTPAYFNDAQRQATKDAGQIAQVLRIVNEPTAAAYGLDKGEKEQRILVFDLGGGTFDVSLLEIGEGVVEVRATSGDNHLGGDDWDQRVVDWLVDKFKGTSGIDLTKDKMAMQRLREAAEKAKIELSSSQSTSINLPYITVDADKNPLFLDEQLTRAEFQRITQDLLDRTRKPFQSVIADTGISVSEIDHVVLVGGSTRMPAVTDLVKELTGGKEPNKGVNPDEVVAVGAALQAGVLKGEVKDVLLLDVTPLSLGIETKGGVMTRLIERNTTIPTKRSETFTTADDNQPSVQIQVYQGEREIAAHNKLLGSFELTGIPPAPRGIPQIEVTFDIDANGIVHVTAKDKGTGKENTIRIQEGSGLSKEDIDRMIKDAEAHAEEDRKRREEADVRNQAETLVYQTEKFVKEQREAEGGSKVPEDTLNKVDAAVAEAKAALGGSDISAIKSAMEKLGQESQALGQAIYEAAQAASQATGAAHPGGEPGGAHPGSADDVVDAEVVDDGREAK

SEQ ID NO: 21 - DNA, 2104 bp, Artificial Sequence
  Description of Artificial Sequence Synthetic polynucleotide
atgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcaaggatccatggctcgtgcggtcgggatcgacctcgggaccaccaactccgtcgtctcggttctggaaggtggcgacccggtcgtcgtcgccaactccgagggctccaggaccaccccgtcaattgtcgcgttcgcccgcaacggtgaggtgctggtcggccagcccgccaagaaccaggcagtgaccaacgtcgatcgcaccgtgcgctcggtcaagcgacacatgggcagcgactggtccatagagattgacggcaagaaatacaccgcgccggagatcagcgcccgcattctgatgaagctgaagcgcgacgccgaggcctacctcggtgaggacattaccgacgcggttatcacgacgcccgcctacttcaatgacgcccagcgtcaggccaccaaggacgccggccagatcgccggcctcaacgtgctgcggatcgtcaacgagccgaccgcggccgcgctggcctacggcctcgacaagggcgagaaggagcagcgaatcctggtcttcgacttgggtggtggcactttcgacgtttccctgctggagatcggcgagggtgtggttgaggtccgtgccacttcgggtgacaaccacctcggcggcgacgactgggaccagcgggtcgtcgattggctggtggacaagttcaagggcaccagcggcatcgatctgaccaaggacaagatggcgatgcagcggctgcgggaagccgccgagaaggcaaagatcgagctgagttcgagtcagtccacctcgatcaacctgccctacatcaccgtcgacgccgacaagaacccgttgttcttagacgagcagctgacccgcgcggagttccaacggatcactcaggacctgctggaccgcactcgcaagccgttccagtcggtgatcgctgacaccggcatttcggtgtcggagatcgatcacgttgtgctcgtgggtggttcgacccggatgcccgcggtgaccgatctggtcaaggaactcaccggcggcaaggaacccaacaagggcgtcaaccccgatgaggttgtcgcggtgggagccgctctgcaggccggcgtcctcaagggcgaggtgaaagacgttctgctgcttgatgttaccccgctgagcctgggtatcgagaccaagggcggggtgatgaccaggctcatcgagcgcaacaccacgatccccaccaagcggtcggagactttcaccaccgccgacgacaaccaaccgtcggtgcagatccaggtctatcagggggagcgtgagatcgccgcgcacaacaagttgctcgggtccttcgagctgaccggcatcccgccggcgccgcgggggattccgcagatcgaggtcactttcgacatcgacgccaacggcattgtgcacgtcaccgccaaggacaagggcaccggcaaggagaacacgatccgaatccaggaaggctcgggcctgtccaaggaagacattgaccgcatgatcaaggacgccgaagcgcacgccgaggaggatcgcaagcgtcgcgaggaggccgatgttcgtaatcaagccgagacattggtctaccagacggagaagttcgtcaaagaacagcgtgaggccgagggtggttcgaaggtacctgaagacacgctgaacaaggttgatgccgcggtggcggaagcgaaggcggcacttggcggatcggatatttcggccatcaagtcggcgatggagaagctgggccaggagtcgcaggctctggggcaagcgatctacgaagcagctcaggctgcgtcacaggccactggcgctgcccaccccggctcggctgatgaaagca

SEQ ID NO: 22 - protein, 701 aa, Artificial Sequence
  Description of Artificial Sequence Synthetic polypeptide
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQGSMARAVGIDLGTTNSVVSVLEGGDPVVVANSEGSRTTPSIVAFARNGEVLVGQPAKNQAVTNVDRTVRSVKRHMGSDWSIEIDGKKYTAPEISARILMKLKRDAEAYLGEDITDAVITTPAYFNDAQRQATKDAGQIAGLNVLRIVNEPTAAALAYGLDKGEKEQRILVFDLGGGTFDVSLLEIGEGVVEVRATSGDNHLGGDDWDQRVVDWLVDKFKGTSGIDLTKDKMAMQRLREAAEKAKIELSSSQSTSINLPYITVDADKNPLFLDEQLTRAEFQRITQDLLDRTRKPFQSVIADTGISVSEIDHVVLVGGSTRMPAVTDLVKELTGGKEPNKGVNPDEVVAVGAALQAGVLKGEVKDVLLLDVTPLSLGIETKGGVMTRLIERNTTIPTKRSETFTTADDNQPSVQIQVYQGEREIAAHNKLLGSFELTGIPPAPRGIPQIEVTFDIDANGIVHVTAKDKGTGKENTIRIQEGSGLSKEDIDRMIKDAEAHAEEDRKRREEADVRNQAETLVYQTEKFVKEQREAEGGSKVPEDTLNKVDAAVAEAKAALGGSDISAIKSAMEKLGQESQALGQAIYEAAQAASQATGAAHPGSADES

SEQ ID NO: 23 - DNA, 2760 bp, Pseudomonas aeruginosa
ctgcagctggtcaggccgtttccgcaacgcttgaagtcctggccgatataccggcagggccagccatcgttcgacgaataaagccacctcagccatgatgccctttccatccccagcggaaccccgacatggacgccaaagccctgctcctcggcagcctctgcctggccgccccattcgccgacgcggcgacgctcgacaatgctctctccgcctgcctcgccgcccggctcggtgcaccgcacacggcggagggccagttgcacctgccactcacccttgaggcccggcgctccaccggcgaatgcggctgtacctcggcgctggtgcgatatcggctgctggccaggggcgccagcgccgacagcctcgtgcttcaagagggctgctcgatagtcgccaggacacgccgcgcacgctgaccctggcggcggacgccggcttggcgagcggccgcgaactggtcgtcaccctgggttgtcaggcgcctgactgacaggccgggctgccaccaccaggccgagatggacgccctgcatgtatcctccgatcggcaagcctcccgttcgcacattcaccactctgcaatccagttcataaatcccataaaagccctcttccgctccccgccagcctccccgcatcccgcaccctagacgccccgccgctctccgccggctcgcccgacaagaaaaaccaaccgctcgatcagcctcatccttcacccatcacaggagccatcgcgatgcacctgataccccattggatccccctggtcgccagcctcggcctgctcgccggcggctcgtccgcgtccgccgccgaggaagccttcgacctctggaacgaatgcgccaaagcctgcgtgctcgacctcaaggacggcgtgcgttccagccgcatgagcgtcgacccggccatcgccgacaccaacggccagggcgtgctgcactactccatggtcctggagggcggcaacgacgcgctcaagctggccatcgacaacgccctcagcatcaccagcgacggcctgaccatccgcctcgaaggcggcgtcgagccgaacaagccggtgcgctacagctacacgcgccaggcgcgcggcagttggtcgctgaactggctggtaccgatcggccacgagaagccctcgaacatcaaggtgttcatccacgaactgaacgccggcaaccagctcagccacatgtcgccgatctacaccatcgagatgggcgacgagttgctggcgaagctggcgcgcgatgccaccttcttcgtcagggcgcacgagagcaacgagatgcagccgacgctcgccatcagccatgccggggtcagcgtggtcatggcccagacccagccgcgccgggaaaagcgctggagcgaatgggccagcggcaaggtgttgtgcctgctcgacccgctggacggggtctacaactacctcgcccagcaacgctgcaacctcgacgatacctgggaaggcaagatctaccgggtgctcgccggcaacccggcgaagcatgacctggacatcaaacccacggtcatcagtcatcgcctgcactttcccgagggcggcagcctggccgcgctgaccgcgcaccaggcttgccacctgccgctggagactttcacccgtcatcgccagccgcgcggctgggaacaactggagcagtgcggctatccggtgcagcggctggtcgccctctacctggcggcgcggctgtcgtggaaccaggtcgaccaggtgatccgcaacgccctggccagccccggcagcggcggcgacctgggcgaagcgatccgcgagcagccggagcaggcccgtctggccctgaccctggccgccgccgagagcgagcgcttcgtccggcagggcaccggcaacgacgaggccggcgcggccaacgccgacgtggtgagcctgacctgcccggtcgccgccggtgaatgcgcgggcccggcggacagcggcgacgccctgctggagcgcaactatcccactggcgcggagttcctcggcgacggcggcgacgtcagcttcagcacccgcggcacgcagaactggacggtggagcggctgctccaggcgcaccgccaactggaggagcgcggctatgtgttcgtcggctaccacggcaccttcctcgaagcggcgcaaagcatcgtcttcggcggggtgcgcgcgcgcagccaggacctcgacgcgatctggcgcggtttctatatcgccggcgatccggcgctggcctacggctacgcccaggaccaggaacccgacgcacgcggccggatccgcaacggtgccctgctgcgggtctatgtgccgcgctcgagcctgccgggcttctaccgcaccagcctgaccctggccgcgccggaggcggcgggcgaggtcgaacggctgatcggccatccgctgccgctgcgcctggacgccatcaccggccccgaggaggaaggcgggcgcctggagaccattctcggctggccgctggccgagcgcaccgtggtgattccctcggcgatccccaccgacccgcgcaacgtcggcggcgacctcgacccgtccagcatccccgacaaggaacaggcgatcagcgccctgccggactacgccagccagcccggcaaaccgccgcgcgaggacctgaagtaactgccgcgaccggccggctcccttcgcaggagccggccttctcggggcctggccatacatcaggttttcctgatgccagcccaatcgaatatgaattc

SEQ ID NO: 24 - protein, 628 aa, Pseudomonas aeruginosa
MHLIPHWIPLVASLGLLAGGSSASAAEEAFDLWNECAKACVLDLKDGVRSSRMSVDPAIADTNGQGVLHYSMVLEGGNDALKLAIDNALSITSDGLTIRLEGGVEPNKPVRYSYTRQRSWSLNWLVPIGHEKPSNIKVFIHELNAGNQLSHMSPIYTIEMGDELLAKLARDATFFVRAHESNEMQPTLAISHAGVSVVMAQTQPRREKRWSEWASGKVLCLLDPLDGVYNYLAQQRCNLDDTWEGKIYRVLAGNPAKHDLDIKPTVISHRLHFPEGGSLAALTAHQACHLPLETFTRHRQPRGWEQLEQCGYPVQRLVALYLAARLSWNQVDQVIRNALDGSGGDLGEAIREQPEQARLALTLAAAESFVRQGTGNDEAGAANADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPAYGYAQDQEPDRRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 25 - protein, 167 aa, Pseudomonas aeruginosa
RLHFPEGGSLAALTAHQACHLPLETFTRHRQPRGWEQLEQCGYPVQRLVALYLAARLSWNQVDQVIRNALDGSGGDLGEAIREQPEQARLALTLAAAESFVRQGTGNDEAGAANADVVSLTCPVAAGECAGPADSGDALLERNYPTGAEFLGDGGDVSFSTRGTQNW

SEQ ID NO: 26 - DNA, 870 bp, Artificial Sequence
  Description of Artificial Sequence Synthetic polynucleotide
atgcgcctgcactttcccgagggcggcagcctggccgcgctgaccgcgcaccaggcttgccacctgccgctggagactttcacccgtcatcgccagccgcgcggctgggaacaactggagcagtgcggctatccggtgcagcggctggtcgccctctacctggcggcgcggctgtcgtggaaccaggtcgaccaggtgatccgcaacgccctggccagccccggcagcggcggcgacctgggcgaagcgatccgcgagcagccggagcaggcccgtctggccctgaccctggccgccgccgagagcgagcgcttcgtccggcagggcaccggcaacgacgaggccggcgcggccaacgccgacgtggtgagcctgacctgcccggtcgccgccggtgaatgcgcgggcccggcggacagcggcgacgccctgctggagcgcaactatcccactggcgcggagttcctcggcgacggcggcgacgtcagcttcagcacccgcggcacgcagaacgaattcatgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcaaggatccgagctcggtaccaagcttaagtttaaaccgctgatcagcctcgactgtgccttctag

SEQ ID NO: 27 - protein, 289 aa, Artificial Sequence
  Description of Artificial Sequence Synthetic polypeptide
MRLHFPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANADVVSLTCPVAAGECAGPADSGDALLERNYPTGAEFLGDGGDVSFSTRGTQNEFMHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQGSELGTKLKFKPLISLDCAF

SEQ ID NO: 28 - DNA, 1254 bp, Homo sapiens
atgctgctatccgtgccgctgctgctcggcctcctcggcctggccgtcgccgagcccgccgtctacttcaaggagcagtttctggacggagacgggtggacttcccgctggatcgaatccaaacacaagtcagattttggcaaattcgttctcagttccggcaagttctacggtgacgaggagaaagataaaggtttgcagacaagccaggatgcacgcttttatgctctgtcggccagtttcgagcctttcagcaacaaaggccagacgctggtggtgcagttcacggtgaaacatgagcagaacatcgactgtgggggcggctatgtgaagctgtttcctaatagtttggaccagacagacatgcacggagactcagaatacaacatcatgtttggtcccgacatctgtggccctggcaccaagaaggttcatgtcatcttcaactacaagggcaagaacgtgctgatcaacaaggacatccgttgcaaggatgatgagtttacacacctgtacacactgattgtgcggccagacaacacctatgaggtgaagattgacaacagccaggtggagtccggctccttggaagacgattgggacttcctgccacccaagaagataaaggatcctgatgcttcaaaaccggaagactgggatgagcgggccaagatcgatgatcccacagactccaagcctgaggactgggacaagcccgagcatatccctgaccctgatgctaagaagcccgaggactgggatgaagagatggacggagagtgggaacccccagtgattcagaaccctgagtacaagggtgagtggaagccccggcagatcgacaacccagattacaagggcacttggatccacccagaaattgacaaccccgagtattctcccgatcccagtatctatgcctatgataactttggcgtgctgggcctggacctctggcaggtcaagtctggcaccatctttgacaacttcctcatcaccaacgatgaggcatacgctgaggagtttggcaacgagacgtggggcgtaacaaaggcagcagagaaacaaatgaaggacaaacaggacgaggagcagaggcttaaggaggaggaagaagacaagaaacgcaaagaggaggaggaggcagaggacaaggaggatgatgaggacaaagatgaggatgaggaggatgaggaggacaaggaggaagatgaggaggaagatgtccccggccaggccaaggacgagctgtag

SEQ ID NO: 29 - protein, 417 aa, Homo sapiens
MLLSVPLLLGLLGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAKDEL

SEQ ID NO: 30 - protein, 170 aa, Homo sapiens
MLLSVPLLLGLLGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTH

SEQ ID NO: 31 - protein, 109 aa, Homo sapiens
LYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQ

SEQ ID NO: 32 - protein, 138 aa, Homo sapiens
IDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAKDEL

SEQ ID NO: 33 - DNA, 540 bp, Homo sapiens
atgctgctatccgtgccgctgctgctcggcctcctcggcctggccgtcgccgagcccgccgtctacttcaaggagcagtttctggacggagacgggtggacttcccgctggatcgaatccaaacacaagtcagattttggcaaattcgttctcagttccggcaagttctacggtgacgaggagaaagataaaggtttgcagacaagccaggatgcacgcttttatgctctgtcggccagtttcgagcctttcagcaacaaaggccagacgctggtggtgcagttcacggtgaaacatgagcagaacatcgactgtgggggcggctatgtgaagctgtttcctaatagtttggaccagacagacatgcacggagactcagaatacaacatcatgtttggtcccgacatctgtggccctggcaccaagaaggttcatgtcatcttcaactacaagggcaagaacgtgctgatcaacaaggacatccgttgcaaggatgatgagtttacacacctgtacacactgattgtgcggccagacaac

SEQ ID NO: 34 - DNA, 267 bp, Homo sapiens
acctatgaggtgaagattgacaacagccaggtggagtccggctccttggaagacgattgggacttcctgccacccaagaagataaaggatcctgatgcttcaaaaccggaagactgggatgagcgggccaagatcgatgatcccacagactccaagcctgaggactgggacaagcccgagcatatccctgaccctgatgctaagaagcccgaggactgggatgaagagatggacggagagtgggaacccccagtgattcagaaccct

SEQ ID NO: 35 - DNA, 444 bp, Homo sapiens
gagtacaagggtgagtggaagccccggcagatcgacaacccagattacaagggcacttggatccacccagaaattgacaaccccgagtattctcccgatcccagtatctatgcctatgataactttggcgtgctgggcctggacctctggcaggtcaagtctggcaccatctttgacaacttcctcatcaccaacgatgaggcatacgctgaggagtttggcaacgagacgtggggcgtaacaaaggcagcagagaaacaaatgaaggacaaacaggacgaggagcagaggcttaaggaggaggaagaagacaagaaacgcaaagaggaggaggaggcagaggacaaggaggatgatgaggacaaagatgaggatgaggaggatgaggaggacaaggaggaagatgaggaggaagatgtccccggccaggccaaggacgagctg

SEQ ID NO: 36 - DNA, 5970 bp, Artificial Sequence
  Description of Artificial Sequence Synthetic polynucleotide
gctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccagggcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgacatgctgctatccgtgccgctgctgctcggcctcctcggcctggccgtcgccgagcctgccgtctacttcaaggagcagtttctggacggggacgggtggacttcccgctggatcgaatccaaacacaagtcagattttggcaaattcgttctcagttccggcaagttctacggtgacgaggagaaagataaaggtttgcagacaagccaggatgcacgcttttatgctctgtcggccagtttcgagcctttcagcaacaaaggccagacgctggtggtgcagttcacggtgaaacatgagcagaacatcgactgtgggggcggctatgtgaagctgtttcctaatagtttggaccagacagacatgcacggagactcagaatacaacatcatgtttggtcccgacatctgtggccctggcaccaagaaggttcatgtcatcttcaactacaagggcaagaacgtgctgatcaacaaggacatccgttgcaaggatgatgagtttacacacctgtacacactgattgtgcggccagacaacacctatgaggtgaagattgacaacagccaggtggagtccggctccttggaagacgattgggacttcctgccacccaagaagataaaggatcctgatgcttcaaaaccggaagactgggatgagcgggccaagatcgatgatcccacagactccaagcctgaggactgggacaagcccgagcatatccctgaccctgatgctaagaagcccgaggactgggatgaagagatggacggagagtgggaacccccagtgattcagaaccctgagtacaagggtgagtggaagccccggcagatcgacaacccagattacaagggcacttggatccacccagaaattgacaaccccgagtattctcccgatcccagtatctatgcctatgataactttggcgtgctgggcctggacctctggcaggtcaagtctggcaccatctttgacaacttcctcatcaccaacgatgaggcatacgctgaggagtttggcaacgagacgtggggcgtaacaaaggcagcagagaaacaaatgaaggacaaacaggacgaggagcagaggcttaaggaggaggaagaagacaagaaacgcaaagaggaggaggaggcagaggacaaggaggatgatgaggacaaagatgaggatgaggaggatgaggaggacaaggaggaagatgaggaggaagatgtccccggccaggccaaggacgagctggaattcatgcatggagatacacctacattgcatgaatatatgttagatttgcaaccagagacaactgatctctacggttatgggcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccataaggatccagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccatag

SEQ ID NO: 37 - DNA, 750 bp, Marek′s disease virus
atgggggattctgaaaggcggaaatcggaacggcgtcgttcccttggatatccctctgcatatgatgacgtctcgattcctgctcgcagaccatcaacacgtactcagcgaaatttaaaccaggatgatttgtcaaaacatggaccatttaccgaccatccaacacaaaaacataaatcggcgaaagccgtatcggaagacgtttcgtctaccacccggggtggctttacaaacaaaccccgtaccaagcccggggtcagagctgtacaaagtaataaattcgctttcagtacggctccttcatcagcatctagcacttggagatcaaatacagtggcatttaatcagcgtatgttttgcggagcggttgcaactgtggctcaatatcacgcataccaaggcgcgctcgccctttggcgtcaagatcctccgcgaacaaatgaagaattagatgcatttctttccagagctgtcattaaaattaccattcaagagggtccaaatttgatgggggaagccgaaacctgtgcccgcaaactattggaagagtctggattatcccaggggaacgagaacgtaaagtccaaatctgaacgtacaaccaaatctgaacgtacaagacgcggcggtgaaattgaaatcaaatcgccagatccgggatctcatcgtacacataaccctcgcactcccgcaacttcgcgtcgccatcattcatccgcccgcggatatcgtagcagtgatagcgaataa

SEQ ID NO: 38 - protein, 301 aa, Human herpesvirus
MTSRRSVKSGPREVPRDEYEDLYYTPSSGMASPDSPPDTSRRGALQTRSRQRGEVRFVQYDESDYALYGGSSSEDDEHPEVPRTRRPVSGAVLSGPGPARAPPPPAGSGGAGRTPTTAPRAPRTQRVASKAPAAPAAETTRGRKSAQPESAALPDAPASTAPTRSKTPAQGLARKLHFSTAPPNPDAPWTPRVAGFNKRVFCAAVGRLAAMHARMAAVQLWDMSRPRTDEDLNELLGITTIRVTVCEGKNLLQRANELVNPDVVQDVDAATATRGRSAASRPTERPRAPARSASRPRRPVE

SEQ ID NO: 39 - protein, 418 aa, Artificial Sequence
  Description of Artificial Sequence Synthetic polypeptide
MTSRRSVKSGPREVPRDEYEDLYYTPSSGMASPDSPPDTSRRGALQTRSRQRGEVRFVQYDESDYALYGGSSSEDDEHPEVPRTRRPVSGAVLSGPGPARAPPPPAGSGGAGRTPTTAPRAPRTQRVASKAPAAPAAETTRGRKSAQPESAALPDAPASTAPTRSKTPAQGLARKLHFSTAPPNPDAPWTPRVAGFNKRVFCAAVGRLAAMHARMAAVQLWDMSRPRTDEDLNELLGITTIRVTVCEGKNLLQRANELVNPDVVQDVDAATATRGRSAASRPTERPRAPARSASRPRRPVEGTELGSMHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQDKLKFKPLISLDCAF

SEQ ID NO: 40 - protein, 249 aa, Marek′s disease virus
MGDSERRKSERRRSLGYPSAYDDVSIPARRPSTRTQRNLNQDDLSKHGPFTDHPTQKHKSAKAVSEDVSSTTRGGFTNKPRTKPGVRAVQSNKFAFSTAPSSASSTWRSNTVAFNQRMFCGAVATVAQYHAYQGALALWRQDPPRTNEELDAFLSRAVIKITIQEGPNLMGEAETCARKLLEESGLSQGNENVKSKSERTTKSERTRRGGEIEIKSPDPGSHRTHNPRTPATSRRHHSSARGYRSSDSE

SEQ ID NO: 41 - protein, 96 aa, Human papillomavirus
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQ

SEQ ID NO: 42 - DNA, 21 bp, Artificial Sequence
  Description of Artificial Sequence Synthetic oligonucleotide
ugccuacgaacucuucacctt

SEQ ID NO: 43 - DNA, 21 bp, Artificial Sequence
  Description of Artificial Sequence Synthetic oligonucleotide
ggugaagaguucguaggcatt

SEQ ID NO: 44 - DNA, 627 bp, Mus musculus
atggcatctggacaaggaccaggtcccccgaaggtgggctgcgatgagtccccgtccccttctgaacagcaggttgcccaggacacagaggaggtctttcgaagctacgttttttacctccaccagcaggaacaggagacccaggggcggccgcctgccaaccccgagatggacaacttgcccctggaacccaacagcatcttgggtcaggtgggtcggcagcttgctctcatcggagatgatattaaccggcgctacgacacagagttccagaatttactagaacagcttcagcccacagccgggaatgcctacgaactcttcaccaagatcgcctccagcctatttaagagtggcatcagctggggccgcgtggtggctctcctgggctttggctaccgtctggccctgtacgtctaccagcgtggtttgaccggcttcctgggccaggtgacctgctttttggctgatatcatactgcatcattacatcgccagatggatcgcacagagaggcggttgggtggcagccctgaatttgcgtagagaccccatcctgaccgtaatggtgatttttggtgtggttctgttgggccaattcgtggtacacagattcttcagatcatga

SEQ ID NO: 45 - DNA, 19 bp, Mus musculus
tgcctacgaactcttcacc

SEQ ID NO: 46 - DNA, 21 bp, Artificial Sequence
  Description of Artificial Sequence Synthetic oligonucleotide
uauggagcugcagaggaugtt

SEQ ID NO: 47 - DNA, 21 bp, Artificial Sequence
  Description of Artificial Sequence Synthetic oligonucleotide
cauccucugcagcuccauatt

SEQ ID NO: 48 - DNA, 579 bp, Mus musculus
atggacgggtccggggagcagcttgggagcggcgggcccaccagctctgaacagatcatgaagacaggggcctttttgctacagggtttcatccaggatcgagcagggaggatggctggggagacacctgagctgaccttggagcagccgccccaggatgcgtccaccaagaagctgagcgagtgtctccggcgaattggagatgaactggatagcaatatggagctgcagaggatgattgctgacgtggacacggactccccccgagaggtcttcttccgggtggcagctgacatgtttgctgatggcaacttcaactggggccgcgtggttgccctcttctactttgctagcaaactggtgctcaaggccctgtgcactaaagtgcccgagctgatcagaaccatcatgggctggacactggacttcctccgtgagcggctgcttgtctggatccaagaccagggtggctgggaaggcctcctctcctacttcgggacccccacatggcagacagtgaccatctttgtggctggagtcctcaccgcctcgctcaccatctggaagaagatgggctga

SEQ ID NO: 49 - DNA, 19 bp, Mus musculus
tatggagctgcagaggatg

SEQ ID NO: 50 - DNA, 1491 bp, Homo sapiens
atggacttcagcagaaatctttatgatattggggaacaactggacagtgaagatctggcctccctcaagttcctgagcctggactacattccgcaaaggaagcaagaacccatcaaggatgccttgatgttattccagagactccaggaaaagagaatgttggaggaaagcaatctgtccttcctgaaggagctgctcttccgaattaatagactggatttgctgattacctacctaaacactagaaaggaggagatggaaagggaacttcagacaccaggcagggctcaaatttctgcctacaggttccacttctgccgcatgagctgggctgaagcaaacagccagtgccagacacagtctgtacctttctggcggagggtcgatcatctattaataagggtcatgctctatcagatttcagaagaagtgagcagatcagaattgaggtcttttaagtttcttttgcaagaggaaatctccaaatgcaaactggatgatgacatgaacctgctggatattttcatagagatggagaagagggtcatcctgggagaaggaaagttggacatcctgaaaagagtctgtgcccaaatcaacaagagcctgctgaagataatcaacgactatgaagaattcagcaaaggggaggagttgtgtggggtaatgacaatctcggactctccaagagaacaggatagtgaatcacagactttggacaaagtttaccaaatgaaaagcaaacctcggggatactgtctgatcatcaacaatcacaattttgcaaaagcacgggagaaagtgcccaaacttcacagcattagggacaggaatggaacacacttggatgcaggggctttgaccacgacctttgaagagcttcattttgagatcaagccccacgatgactgcacagtagagcaaatctatgagattttgaaaatctaccaactcatggaccacagtaacatggactgcttcatctgctgtatcctctcccatggagacaagggcatcatctatggcactgatggacaggaggcccccatctatgagctgacatctcagttcactggtttgaagtgcccttcccttgctggaaaacccaaagtgttttttattcaggcttgtcagggggataactaccagaaaggtatacctgttgagactgattcagaggagcaaccctatttagaaatggatttatcatcacctcaaacgagatatatcccggatgaggctgactttctgctggggatggccactgtgaataactgtgtttcctaccgaaaccctgcagagggaacctggtacatccagtcactttgccagagcctgagagagcgatgtcctcgaggcgatgatattctcaccatcctgactgaagtgaactatgaagtaagcaacaaggatgacaagaaaaacatggggaaacagatgcctcagcctactttcacactaagaaaaaaacttgtcttcccttctgattga

SEQ ID NO: 51 - DNA, 23 bp, Artificial Sequence
  Description of Artificial Sequence Synthetic oligonucleotide
aaccucggggauacugucugatt

SEQ ID NO: 52 - DNA, 23 bp, Artificial Sequence
  Description of Artificial Sequence Synthetic oligonucleotide
ucagacaguauccccgagguutt

SEQ ID NO: 53 - DNA, 1251 bp, Homo sapiens
atggacgaagcggatcggcggctcctgcggcggtgccggctgcggctggtggaagagctgcaggtggaccagctctgggacgccctgctgagccgcgagctgttcaggccccatatgatcgaggacatccagcgggcaggctctggatctcggcgggatcaggccaggcagctgatcatagatctggagactcgagggagtcaggctcttcctttgttcatctcctgcttagaggacacaggccaggacatgctggcttcgtttctgcgaactaacaggcaagcagcaaagttgtcgaagccaaccctagaaaaccttaccccagtggtgctcagaccagagattcgcaaaccagaggttctcagaccggaaacacccagaccagtggacattggttctggaggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcagcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcataa

SEQ ID NO: 54 - DNA, 834 bp, Homo sapiens
atggagaacactgaaaactcagtggattcaaaatccattaaaaatttggaaccaaagatcatacatggaagcgaatcaatggactctggaatatccctggacaacagttataaaatggattatcctgagatgggtttatgtataataattaataataagaattttcataaaagcactggaatgacatctcggtctggtacagatgtcgatgcagcaaacctcagggaaacattcagaaacttgaaatatgaagtcaggaataaaaatgatcttacacgtgaagaaattgtggaattgatgcgtgatgtttctaaagaagatcacagcaaaaggagcagttttgtttgtgtgcttctgagccatggtgaagaaggaataatttttggaacaaatggacctgttgacctgaaaaaaataacaaactttttcagaggggatcgttgtagaagtctaactggaaaacccaaacttttcattattcaggcctgccgtggtacagaactggactgtggcattgagacagacagtggtgttgatgatgacatggcgtgtcataaaataccagtggaggccgacttcttgtatgcatactccacagcacctggttattattcttggcgaaattcaaaggatggctcctggttcatccagtcgctttgtgccatgctgaaacagtatgccgacaagcttgaatttatgcacattcttacccgggttaaccgaaaggtggcaacagaatttgagtccttttcctttgacgctacttttcatgcaaagaaacagattccatgtattgtttccatgctcacaaaagaactctatttttatcactaa

SEQ ID NO: 55 - DNA, 750 bp, Homo sapiens
atggcgtacccatacgatgttccagattacgctagcttgagatctaccatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactgcgcacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatga

SEQ ID NO: 56 - protein, 249 aa, Homo sapiens
MAYPYDVPDYASLRSTMSQSNRELVVDFLSYKLSQKGYSWSQFSDVEENRTEAPEGTESEMETPSAINGNPSWHLADSPAVNGATAHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNAAAESRKGQERFNRWFLTGMTVAGVVLLGSLFSRK

SEQ ID NO: 57 - DNA, 6187 bp, Artificial Sequence
  Description of Artificial Sequence Synthetic polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccactgtgctggatatctgcagaattccaccacactggactagtggatctatggcgtacccatacgatgttccagattacgctagcttgagatctaccatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactgcgcacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatgaagatccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaaatgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 58 - DNA, 6452 bp, Artificial Sequence
  Description of Artificial Sequence Synthetic polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccactgtgctggatatctgcagaattcatgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccaggatctatggcgtacccatacgatgttccagattacgctagcttgagatctaccatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactgcgcacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctactgggctcactcttcagtcggaaatgaagatccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 59 - protein, 349 aa, Homo sapiens
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKPGSMAYPYDVPDYASLRSTMSQSNRELVVDFLSYKLSQKGYSWSQFSDVEENRTEAPEGTESEMETPSAINGNPSWHLADSPAVNGATAHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNAAAESRKGQERFNRWFLTGMTVAGVVLLGSLFSRK

SEQ ID NO: 60 - DNA, 750 bp, Homo sapiens
atggcgtacccatacgatgttccagattacgctagcttgagatctaccatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactgcgcacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtagccattcttcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatga

SEQ ID NO: 61 - protein, 249 aa, Homo sapiens
MAYPYDVPDYASLRSTMSQSNRELVVDFLSYKLSQKGYSWSQFSDVEENRTEAPEGTESEMETPSAINGNPSWHLADSPAVNGATAHSSSLDAREVIPMAAVKQALREAGDEFELRYRRAFSDLTSQLHITPGTAYQSFEQVVNELFRDGVAILRIVAFFSFGGALCVESVDKEMQVLVSRIAAWMATYLNDHLEPWIQENGGWDTFVELYGNNAAAESRKGQERFNRWFLTGMTVAGVVLLGSLFSRK

SEQ ID NO: 62 - protein, 349 aa, Artificial Sequence
  Description of Artificial Sequence Synthetic polypeptide
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKPGSMAYPYDVPDYASLRSTMSQSNRELVVDFLSYKLSQKGYSWSQFSDVEENRTEAPEGTESEMETPSAINGNPSWHLADSPAVNGATAHSSSLDAREVIPMAAVKQALREAGDEFELRYRRAFSDLTSQLHITPGTAYQSFEQVVNELFRDGVAILRIVAFFSFGGALCVESVDKEMQVLVSRIAAWMATYLNDHLEPWIQENGGWDTFVELYGNNAAAESRKGQERFNRWFLTGMTVAGVVLLGSLFSRK

SEQ ID NO: 63 - DNA, 6187 bp, Artificial Sequence
  Description of Artificial Sequence Synthetic polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccactgtgctggatatctgcagaattccaccacactggactagtggatctatggcgtacccatacgatgttccagattacgctagcttgagatctaccatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactgcgcacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtagccattcttcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatgaagatccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaaatgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 64 - DNA, 6452 bp, Artificial Sequence
  Description of Artificial Sequence Synthetic polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccactgtgctggatatctgcagaattcatgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccaggatctatggcgtacccatacgatgttccagattacgctagcttgagatctaccatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactgcgcacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtagccattcttcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatgaagatccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 65 - DNA, 12347 bp, Artificial Sequence
  Description of Artificial Sequence Synthetic polynucleotide
atggcggatgtgtgacatacacgacgccaaaagattttgttccagctcctgccacctccgctacgcgagagattaaccacccacgatggccgccaaagtgcatgttgatattgaggctgacagcccattcatcaagtctttgcagaaggcatttccgtcgttcgaggtggagtcattgcaggtcacaccaaatgaccatgcaaatgccagagcattttcgcacctggctaccaaattgatcgagcaggagactgacaaagacacactcatcttggatatcggcagtgcgccttccaggagaatgatgtctacgcacaaataccactgcgtatgccctatgcgcagcgcagaagaccccgaaaggctcgatagctacgcaaagaaactggcagcggcctccgggaaggtgctggatagagagatcgcaggaaaaatcaccgacctgcagaccgtcatggctacgccagacgctgaatctcctaccttttgcctgcatacagacgtcacgtgtcgtacggcagccgaagtggccgtataccaggacgtgtatgctgtacatgcaccaacatcgctgtaccatcaggcgatgaaaggtgtcagaacggcgtattggattgggtttgacaccaccccgtttatgtttgacgcgctagcaggcgcgtatccaacctacgccacaaactgggccgacgagcaggtgttacaggccaggaacataggactgtgtgcagcatccttgactgagggaagactcggcaaactgtccattctccgcaagaagcaattgaaaccttgcgacacagtcatgttctcggtaggatctacattgtacactgagagcagaaagctactgaggagctggcacttaccctccgtattccacctgaaaggtaaacaatcctttacctgtaggtgcgataccatcgtatcatgtgaagggtacgtagttaagaaaatcactatgtgccccggcctgtacggtaaaacggtagggtacgccgtgacgtatcacgcggagggattcctagtgtgcaagaccacagacactgtcaaaggagaaagagtctcattccctgtatgcacctacgtcccctcaaccatctgtgatcaaatgactggcatactagcgaccgacgtcacaccggaggacgcacagaagttgttagtgggattgaatcagaggatagttgtgaacggaagaacacagcgaaacactaacacgatgaagaactatctgcttccgattgtggccgtcgcatttagcaagtgggcgagggaatacaaggcagaccttgatgatgaaaaacctctgggtgtccgagagaggtcacttacttgctgctgcttgtgggcatttaaaacgaggaagatgcacaccatgtacaagaaaccagacacccagacaatagtgaaggtgccttcagagtttaactcgttcgtcatcccgagcctatggtctacaggcctcgcaatcccagtcagatcacgcattaagatgcttttggccaagaagaccaagcgagagttaatacctgttctcgacgcgtcgtcagccagggatgctgaacaagaggagaaggagaggttggaggccgagctgactagagaagccttaccacccctcgtccccatcgcgccggcggagacgggagtcgtcgacgtcgacgttgaagaactagagtatcacgcaggtgcaggggtcgtggaaacacctcgcagcgcgttgaaagtcaccgcacagccgaacgacgtactactaggaaattacgtagttctgtccccgcagaccgtgctcaagagctccaagttggcccccgtgcaccctctagcagagcaggtgaaaataataacacataacgggagggccggcggttaccaggtcgacggatatgacggcagggtcctactaccatgtggatcggccattccggtccctgagtttcaagctttgagcgagagcgccactatggtgtacaacgaaagggagttcgtcaacaggaaactataccatattgccgttcacggaccgtcgctgaacaccgacgaggagaactacgagaaagtcagagctgaaagaactgacgccgagtacgtgttcgacgtagataaaaaatgctgcgtcaagagagaggaagcgtcgggtttggtgttggtgggagagctaaccaaccccccgttccatgaattcgcctacgaagggctgaagatcaggccgtcggcaccatataagactacagtagtaggagtctttggggttccgggatcaggcaagtctgctattattaagagcctcgtgaccaaacacgatctggtcaccagcggcaagaaggagaactgccaggaaatagttaacgacgtgaagaagcaccgcgggaaggggacaagtagggaaaacagtgactccatcctgctaaacgggtgtcgtcgtgccgtggacatcctatatgtggacgaggctttcgctagccattccggtactctgctggccctaattgctcttgttaaacctcggagcaaagtggtgttatgcggagaccccaagcaatgcggattcttcaatatgatgcagcttaaggtgaacttcaaccacaacatctgcactgaagtatgtcataaaagtatatccagacgttgcacgcgtccagtcacggccatcgtgtctacgttgcactacggaggcaagatgcgcacgaccaacccgtgcaacaaacccataatcatagacaccacaggacagaccaagcccaagccaggagacatcgtgttaacatgcttccgaggctgggcaaagcagctgcagttggactaccgtggacacgaagtcatgacagcagcagcatctcagggcctcacccgcaaaggggtatacgccgtaaggcagaaggtgaatgaaaatcccttgtatgcccctgcgtcggagcacgtgaatgtactgctgacgcgcactgaggataggctggtgtggaaaacgctggccggcgatccctggattaaggtcctatcaaacattccacagggtaactttacggccacattggaagaatggcaagaagaacacgacaaaataatgaaggtgattgaaggaccggctgcgcctgtggacgcgttccagaacaaagcgaacgtgtgttgggcgaaaagcctggtgcctgtcctggacactgccggaatcagattgacagcagaggagtggagcaccataattacagcatttaaggaggacagagcttactctccagtggtggccttgaatgaaatttgcaccaagtactatggagttgacctggacagtggcctgttttctgccccgaaggtgtccctgtattacgagaacaaccactgggataacagacctggtggaaggatgtatggattcaatgccgcaacagctgccaggctggaagctagacataccttcctgaaggggcagtggcatacgggcaagcaggcagttatcgcagaaagaaaaatccaaccgctttctgtgctggacaatgtaattcctatcaaccgcaggctgccgcacgccctggtggctgagtacaagacggttaaaggcagtagggttgagtggctggtcaataaagtaagagggtaccacgtcctgctggtgagtgagtacaacctggctttgcctcgacgcagggtcacttggttgtcaccgctgaatgtcacaggcgccgataggtgctacgacctaagtttaggactgccggctgacgccggcaggttcgacttggtctttgtgaacattcacacggaattcagaatccaccactaccagcagtgtgtcgaccacgccatgaagctgcagatgcttgggggagatgcgctacgactgctaaaacccggcggcatcttgatgagagcttacggatacgccgataaaatcagcgaagccgttgtttcctccttaagcagaaagttctcgtctgcaagagtgttgcgcccggattgtgtcaccagcaatacagaagtgttcttgctgttctccaactttgacaacggaaagagaccctctacgctacaccagatgaataccaagctgagtgccgtgtatgccggagaagccatgcacacggccgggtgtgcaccatcctacagagttaagagagcagacatagccacgtgcacagaagcggctgtggttaacgcagctaacgcccgtggaactgtaggggatggcgtatgcagggccgtggcgaagaaatggccgtcagcctttaagggagcagcaacaccagtgggcacaattaaaacagtcatgtgcggctcgtaccccgtcatccacgctgtagcgcctaatttctctgccacgactgaagcggaaggggaccgcgaattggccgctgtctaccgggcagtggccgccgaagtaaacagactgtcactgagcagcgtagccatcccgctgctgtccacaggagtgttcagcggcggaagagataggctgcagcaatccctcaaccatctattcacagcaatggacgccacggacgctgacgtgaccatctactgcagagacaaaagttgggagaagaaaatccaggaagccattgacatgaggacggctgtggagttgctcaatgatgacgtggagctgaccacagacttggtgagagtgcacccggacagcagcctggtgggtcgtaagggctacagtaccactgacgggtcgctgtactcgtactttgaaggtacgaaattcaaccaggctgctattgatatggcagagatactgacgttgtggcccagactgcaagaggcaaacgaacagatatgcctatacgcgctgggcgaaacaatggacaacatcagatccaaatgtccggtgaacgattccgattcatcaacacctcccaggacagtgccctgcctgtgccgctacgcaatgacagcagaacggatcgcccgccttaggtcacaccaagttaaaagcatggtggtttgctcatcttttcccctcccgaaataccatgtagatggggtgcagaaggtaaagtgcgagaaggttctcctgttcgacccgacggtaccttcagtggttagtccgcggaagtatgccgcatctacgacggaccactcagatcggtcgttacgagggtttgacttggactggaccaccgactcgtcttccactgccagcgataccatgtcgctacccagtttgcagtcgtgtgacatcgactcgatctacgagccaatggctcccatagtagtgacggctgacgtacaccctgaacccgcaggcatcgcggacctggcggcagatgtgcaccctgaacccgcagaccatgtggacctcgagaacccgattcctccaccgcgcccgaagagagctgcataccttgcctcccgcgcggcggagcgaccggtgccggcgccgagaaagccgacgcctgccccaaggactgcgtttaggaacaagctgcctttgacgttcggcgactttgacgagcacgaggtcgatgcgttggcctccgggattactttcggagacttcgacgacgtcctgcgactaggccgcgcgggtgcatatattttctcctcggacactggcagcggacatttacaacaaaaatccgttaggcagcacaatctccagtgcgcacaactggatgcggtccaggaggagaaaatgtacccgccaaaattggatactgagagggagaagctgttgctgctgaaaatgcagatgcacccatcggaggctaataagagtcgataccagtctcgcaaagtggagaacatgaaagccacggtggtggacaggctcacatcgggggccagattgtacacgggagcggacgtaggccgcataccaacatacgcggttcggtacccccgccccgtgtactcccctaccgtgatcgaaagattctcaagccccgatgtagcaatcgcagcgtgcaacgaatacctatccagaaattacccaacagtggcgtcgtaccagataacagatgaatacgacgcatacttggacatggttgacgggtcggatagttgcttggacagagcgacattctgcccggcgaagctccggtgctacccgaaacatcatgcgtaccaccagccgactgtacgcagtgccgtcccgtcaccctttcagaacacactacagaacgtgctagcggccgccaccaagagaaactgcaacgtcacgcaaatgcgagaactacccaccatggactcggcagtgttcaacgtggagtgcttcaagcgctatgcctgctccggagaatattgggaagaatatgctaaacaacctatccggataaccactgagaacatcactacctatgtgaccaaattgaaaggcccgaaagctgctgccttgttcgctaagacccacaacttggttccgctgcaggaggttcccatggacagattcacggtcgacatgaaacgagatgtcaaagtcactccagggacgaaacacacagaggaaagacccaaagtccaggtaattcaagcagcggagccattggcgaccgcttacctgtgcggcatccacagggaattagtaaggagactaaatgctgtgttacgccctaacgtgcacacattgtttgatatgtcggccgaagactttgacgcgatcatcgcctctcacttccacccaggagacccggttctagagacggacattgcatcattcgacaaaagccaggacgactccttggctcttacaggtttaatgatcctcgaagatctaggggtggatcagtacctgctggacttgatcgaggcagcctttggggaaatatccagctgtcacctaccaactggcacgcgcttcaagttcggagctatgatgaaatcgggcatgtttctgactttgtttattaacactgttttgaacatcaccatagcaagcagggtactggagcagagactcactgactccgcctgtgcggccttcatcggcgacgacaacatcgttcacggagtgatctccgacaagctgatggcggagaggtgcgcgtcgtgggtcaacatggaggtgaagatcattgacgctgtcatgggcgaaaaacccccatatttttgtgggggattcatagtttttgacagcgtcacacagaccgcctgccgtgtttcagacccacttaagcgcctgttcaagttgggtaagccgctaacagctgaagacaagcaggacgaagacaggcgacgagcactgagtgacgaggttagcaagtggttccggacaggcttgggggccgaactggaggtggcactaacatctaggtatgaggtagagggctgcaaaagtatcctcatagccatggccaccttggcgagggacattaaggcgtttaagaaattgagaggacctgttatacacctctacggcggtcctagattggtgcgttaatacacagaattctgattggatcccaaacgggccctctagactcgagcggccgccactgtgctggatatctgcagaattccaccacactggactagtggatctatggcgtacccatacgatgttccagattacgctagcttgagatctaccatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactgcgcacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcatggttctactgggctcactcttcagtcggaaatgaagatccgagctcggtaccaagcttaagtttgggtaattaattgaattacatccctacgcaaacgttttacggccgccggtggcgcccgcgcccggcggcccgtccttggccgttgcaggccactccggtggctcccgtcgtccccgacttccaggcccagcagatgcagcaactcatcagcgccgtaaatgcgctgacaatgagacagaacgcaattgctcctgctaggcctcccaaaccaaagaagaagaagacaaccaaaccaaagccgaaaacgcagcccaagaagatcaacggaaaaacgcagcagcaaaagaagaaagacaagcaagccgacaagaagaagaagaaacccggaaaaagagaaagaatgtgcatgaagattgaaaatgactgtatcttcgtatgcggctagccacagtaacgtagtgtttccagacatgtcgggcaccgcactatcatgggtgcagaaaatctcgggtggtctgggggccttcgcaatcggcgctatcctggtgctggttgtggtcacttgcattgggctccgcagataagttagggtaggcaatggcattgatatagcaagaaaattgaaaacagaaaaagttagggtaagcaatggcatataaccataactgtataacttgtaacaaagcgcaacaagacctgcgcaattggccccgtggtccgcctcacggaaactcggggcaactcatattgacacattaattggcaataattggaagcttacataagcttaattcgacgaataattggatttttattttattttgcaattggtttttaatatttccaaaaaaaaaaaaaaaaaaaaaaaaaaaaaaaaaaaaaaaaaaaaaaaaaaaaaaaaaaaaaaaaaaaaaactagtgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctagtctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcgcgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggcattctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttctgtctaagcggatgccgggagcagacaagcccgtcagggcgcgtcagcgggtgttggcgggtgtcggggctggcttaactatgcggcatcagagcagattgtactgagagtgcaccatatcgacgctctcccttatgcgactcctgcattaggaagcagcccagtactaggttgaggccgttgagcaccgccgccgcaaggaatggtgcatgcgtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttaactggcttatcgaaattaatacgactcactatagggagaccggaagcttgaattc

SEQ ID NO: 66 - DNA, 12612 bp, Artificial Sequence
  Description of Artificial Sequence Synthetic polynucleotide
atggcggatgtgtgacatacacgacgccaaaagattttgttccagctcctgccacctccgctacgcgagagattaaccacccacgatggccgccaaagtgcatgttgatattgaggctgacagcccattcatcaagtctttgcagaaggcatttccgtcgttcgaggtggagtcattgcaggtcacaccaaatgaccatgcaaatgccagagcattttcgcacctggctaccaaattgatcgagcaggagactgacaaagacacactcatcttggatatcggcagtgcgccttccaggagaatgatgtctacgcacaaataccactgcgtatgccctatgcgcagcgcagaagaccccgaaaggctcgatagctacgcaaagaaactggcagcggcctccgggaaggtgctggatagagagatcgcaggaaaaatcaccgacctgcagaccgtcatggctacgccagacgctgaatctcctaccttttgcctgcatacagacgtcacgtgtcgtacggcagccgaagtggccgtataccaggacgtgtatgctgtacatgcaccaacatcgctgtaccatcaggcgatgaaaggtgtcagaacggcgtattggattgggtttgacaccaccccgtttatgtttgacgcgctagcaggcgcgtatccaacctacgccacaaactgggccgacgagcaggtgttacaggccaggaacataggactgtgtgcagcatccttgactgagggaagactcggcaaactgtccattctccgcaagaagcaattgaaaccttgcgacacagtcatgttctcggtaggatctacattgtacactgagagcagaaagctactgaggagctggcacttaccctccgtattccacctgaaaggtaaacaatcctttacctgtaggtgcgataccatcgtatcatgtgaagggtacgtagttaagaaaatcactatgtgccccggcctgtacggtaaaacggtagggtacgccgtgacgtatcacgcggagggattcctagtgtgcaagaccacagacactgtcaaaggagaaagagtctcattccctgtatgcacctacgtcccctcaaccatctgtgatcaaatgactggcatactagcgaccgacgtcacaccggaggacgcacagaagttgttagtgggattgaatcagaggatagttgtgaacggaagaacacagcgaaacactaacacgatgaagaactatctgcttccgattgtggccgtcgcatttagcaagtgggcgagggaatacaaggcagaccttgatgatgaaaaacctctgggtgtccgagagaggtcacttacttgctgctgcttgtgggcatttaaaacgaggaagatgcacaccatgtacaagaaaccagacacccagacaatagtgaaggtgccttcagagtttaactcgttcgtcatcccgagcctatggtctacaggcctcgcaatcccagtcagatcacgcattaagatgcttttggccaagaagaccaagcgagagttaatacctgttctcgacgcgtcgtcagccagggatgctgaacaagaggagaaggagaggttggaggccgagctgactagagaagccttaccacccctcgtccccatcgcgccggcggagacgggagtcgtcgacgtcgacgttgaagaactagagtatcacgcaggtgcaggggtcgtggaaacacctcgcagcgcgttgaaagtcaccgcacagccgaacgacgtactactaggaaattacgtagttctgtccccgcagaccgtgctcaagagctccaagttggcccccgtgcaccctctagcagagcaggtgaaaataataacacataacgggagggccggcggttaccaggtcgacggatatgacggcagggtcctactaccatgtggatcggccattccggtccctgagtttcaagctttgagcgagagcgccactatggtgtacaacgaaagggagttcgtcaacaggaaactataccatattgccgttcacggaccgtcgctgaacaccgacgaggagaactacgagaaagtcagagctgaaagaactgacgccgagtacgtgttcgacgtagataaaaaatgctgcgtcaagagagaggaagcgtcgggtttggtgttggtgggagagctaaccaaccccccgttccatgaattcgcctacgaagggctgaagatcaggccgtcggcaccatataagactacagtagtaggagtctttggggttccgggatcaggcaagtctgctattattaagagcctcgtgaccaaacacgatctggtcaccagcggcaagaaggagaactgccaggaaatagttaacgacgtgaagaagcaccgcgggaaggggacaagtagggaaaacagtgactccatcctgctaaacgggtgtcgtcgtgccgtggacatcctatatgtggacgaggctttcgctagccattccggtactctgctggccctaattgctcttgttaaacctcggagcaaagtggtgttatgcggagaccccaagcaatgcggattcttcaatatgatgcagcttaaggtgaacttcaaccacaacatctgcactgaagtatgtcataaaagtatatccagacgttgcacgcgtccagtcacggccatcgtgtctacgttgcactacggaggcaagatgcgcacgaccaacccgtgcaacaaacccataatcatagacaccacaggacagaccaagcccaagccaggagacatcgtgttaacatgcttccgaggctgggcaaagcagctgcagttggactaccgtggacacgaagtcatgacagcagcagcatctcagggcctcacccgcaaaggggtatacgccgtaaggcagaaggtgaatgaaaatcccttgtatgcccctgcgtcggagcacgtgaatgtactgctgacgcgcactgaggataggctggtgtggaaaacgctggccggcgatccctggattaaggtcctatcaaacattccacagggtaactttacggccacattggaagaatggcaagaagaacacgacaaaataatgaaggtgattgaaggaccggctgcgcctgtggacgcgttccagaacaaagcgaacgtgtgttgggcgaaaagcctggtgcctgtcctggacactgccggaatcagattgacagcagaggagtggagcaccataattacagcatttaaggaggacagagcttactctccagtggtggccttgaatgaaatttgcaccaagtactatggagttgacctggacagtggcctgttttctgccccgaaggtgtccctgtattacgagaacaaccactgggataacagacctggtggaaggatgtatggattcaatgccgcaacagctgccaggctggaagctagacataccttcctgaaggggcagtggcatacgggcaagcaggcagttatcgcagaaagaaaaatccaaccgctttctgtgctggacaatgtaattcctatcaaccgcaggctgccgcacgccctggtggctgagtacaagacggttaaaggcagtagggttgagtggctggtcaataaagtaagagggtaccacgtcctgctggtgagtgagtacaacctggctttgcctcgacgcagggtcacttggttgtcaccgctgaatgtcacaggcgccgataggtgctacgacctaagtttaggactgccggctgacgccggcaggttcgacttggtctttgtgaacattcacacggaattcagaatccaccactaccagcagtgtgtcgaccacgccatgaagctgcagatgcttgggggagatgcgctacgactgctaaaacccggcggcatcttgatgagagcttacggatacgccgataaaatcagcgaagccgttgtttcctccttaagcagaaagttctcgtctgcaagagtgttgcgcccggattgtgtcaccagcaatacagaagtgttcttgctgttctccaactttgacaacggaaagagaccctctacgctacaccagatgaataccaagctgagtgccgtgtatgccggagaagccatgcacacggccgggtgtgcaccatcctacagagttaagagagcagacatagccacgtgcacagaagcggctgtggttaacgcagctaacgcccgtggaactgtaggggatggcgtatgcagggccgtggcgaagaaatggccgtcagcctttaagggagcagcaacaccagtgggcacaattaaaacagtcatgtgcggctcgtaccccgtcatccacgctgtagcgcctaatttctctgccacgactgaagcggaaggggaccgcgaattggccgctgtctaccgggcagtggccgccgaagtaaacagactgtcactgagcagcgtagccatcccgctgctgtccacaggagtgttcagcggcggaagagataggctgcagcaatccctcaaccatctattcacagcaatggacgccacggacgctgacgtgaccatctactgcagagacaaaagttgggagaagaaaatccaggaagccattgacatgaggacggctgtggagttgctcaatgatgacgtggagctgaccacagacttggtgagagtgcacccggacagcagcctggtgggtcgtaagggctacagtaccactgacgggtcgctgtactcgtactttgaaggtacgaaattcaaccaggctgctattgatatggcagagatactgacgttgtggcccagactgcaagaggcaaacgaacagatatgcctatacgcgctgggcgaaacaatggacaacatcagatccaaatgtccggtgaacgattccgattcatcaacacctcccaggacagtgccctgcctgtgccgctacgcaatgacagcagaacggatcgcccgccttaggtcacaccaagttaaaagcatggtggtttgctcatcttttcccctcccgaaataccatgtagatggggtgcagaaggtaaagtgcgagaaggttctcctgttcgacccgacggtaccttcagtggttagtccgcggaagtatgccgcatctacgacggaccactcagatcggtcgttacgagggtttgacttggactggaccaccgactcgtcttccactgccagcgataccatgtcgctacccagtttgcagtcgtgtgacatcgactcgatctacgagccaatggctcccatagtagtgacggctgacgtacaccctgaacccgcaggcatcgcggacctggcggcagatgtgcaccctgaacccgcagaccatgtggacctcgagaacccgattcctccaccgcgcccgaagagagctgcataccttgcctcccgcgcggcggagcgaccggtgccggcgccgagaaagccgacgcctgccccaaggactgcgtttaggaacaagctgcctttgacgttcggcgactttgacgagcacgaggtcgatgcgttggcctccgggattactttcggagacttcgacgacgtcctgcgactaggccgcgcgggtgcatatattttctcctcggacactggcagcggacatttacaacaaaaatccgttaggcagcacaatctccagtgcgcacaactggatgcggtccaggaggagaaaatgtacccgccaaaattggatactgagagggagaagctgttgctgctgaaaatgcagatgcacccatcggaggctaataagagtcgataccagtctcgcaaagtggagaacatgaaagccacggtggtggacaggctcacatcgggggccagattgtacacgggagcggacgtaggccgcataccaacatacgcggttcggtacccccgccccgtgtactcccctaccgtgatcgaaagattctcaagccccgatgtagcaatcgcagcgtgcaacgaatacctatccagaaattacccaacagtggcgtcgtaccagataacagatgaatacgacgcatacttggacatggttgacgggtcggatagttgcttggacagagcgacattctgcccggcgaagctccggtgctacccgaaacatcatgcgtaccaccagccgactgtacgcagtgccgtcccgtcaccctttcagaacacactacagaacgtgctagcggccgccaccaagagaaactgcaacgtcacgcaaatgcgagaactacccaccatggactcggcagtgttcaacgtggagtgcttcaagcgctatgcctgctccggagaatattgggaagaatatgctaaacaacctatccggataaccactgagaacatcactacctatgtgaccaaattgaaaggcccgaaagctgctgccttgttcgctaagacccacaacttggttccgctgcaggaggttcccatggacagattcacggtcgacatgaaacgagatgtcaaagtcactccagggacgaaacacacagaggaaagacccaaagtccaggtaattcaagcagcggagccattggcgaccgcttacctgtgcggcatccacagggaattagtaaggagactaaatgctgtgttacgccctaacgtgcacacattgtttgatatgtcggccgaagactttgacgcgatcatcgcctctcacttccacccaggagacccggttctagagacggacattgcatcattcgacaaaagccaggacgactccttggctcttacaggtttaatgatcctcgaagatctaggggtggatcagtacctgctggacttgatcgaggcagcctttggggaaatatccagctgtcacctaccaactggcacgcgcttcaagttcggagctatgatgaaatcgggcatgtttctgactttgtttattaacactgttttgaacatcaccatagcaagcagggtactggagcagagactcactgactccgcctgtgcggccttcatcggcgacgacaacatcgttcacggagtgatctccgacaagctgatggcggagaggtgcgcgtcgtgggtcaacatggaggtgaagatcattgacgctgtcatgggcgaaaaacccccatatttttgtgggggattcatagtttttgacagcgtcacacagaccgcctgccgtgtttcagacccacttaagcgcctgttcaagttgggtaagccgctaacagctgaagacaagcaggacgaagacaggcgacgagcactgagtgacgaggttagcaagtggttccggacaggcttgggggccgaactggaggtggcactaacatctaggtatgaggtagagggctgcaaaagtatcctcatagccatggccaccttggcgagggacattaaggcgtttaagaaattgagaggacctgttatacacctctacggcggtcctagattggtgcgttaatacacagaattctgattggatcccaaacgggccctctagactcgagcggccgccactgtgctggatatctgcagaattcatgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccaggatctatggcgtacccatacgatgttccagattacgctagcttgagatctaccatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactgcgcacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatgaagatccaagcttaagtttgggtaattaattgaattacatccctacgcaaacgttttacggccgccggtggcgcccgcgcccggcggcccgtccttggccgttgcaggccactccggtggctcccgtcgtccccgacttccaggcccagcagatgcagcaactcatcagcgccgtaaatgcgctgacaatgagacagaacgcaattgctcctgctaggcctcccaaaccaaagaagaagaagacaaccaaaccaaagccgaaaacgcagcccaagaagatcaacggaaaaacgcagcagcaaaagaagaaagacaagcaagccgacaagaagaagaagaaacccggaaaaagagaaagaatgtgcatgaagattgaaaatgactgtatcttcgtatgcggctagccacagtaacgtagtgtttccagacatgtcgggcaccgcactatcatgggtgcagaaaatctcgggtggtctgggggccttcgcaatcggcgctatcctggtgctggttgtggtcacttgcattgggctccgcagataagttagggtaggcaatggcattgatatagcaagaaaattgaaaacagaaaaagttagggtaagcaatggcatataaccataactgtataacttgtaacaaagcgcaacaagacctgcgcaattggccccgtggtccgcctcacggaaactcggggcaactcatattgacacattaattggcaataattggaagcttacataagcttaattcgacgaataattggatttttattttattttgcaattggtttttaatatttccaaaaaaaaaaaaaaaaaaaaaaaaaaaaaaaaaaaaaaaaaaaaaaaaaaaaaaaaaaaaaaaaaaaaaactagtgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctagtctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcgcgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggcattctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttctgtctaagcggatgccgggagcagacaagcccgtcagggcgcgtcagcgggtgttggcgggtgtcggggctggcttaactatgcggcatcagagcagattgtactgagagtgcaccatatcgacgctctcccttatgcgactcctgcattaggaagcagcccagtactaggttgaggccgttgagcaccgccgccgcaaggaatggtgcatgcgtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttaactggcttatcgaaattaatacgactcactatagggagaccggaagcttgaattc

SEQ ID NO: 67 - DNA, 12347 bp, Artificial Sequence
  Description of Artificial Sequence Synthetic polynucleotide
atggcggatgtgtgacatacacgacgccaaaagattttgttccagctcctgccacctccgctacgcgagagattaaccacccacgatggccgccaaagtgcatgttgatattgaggctgacagcccattcatcaagtctttgcagaaggcatttccgtcgttcgaggtggagtcattgcaggtcacaccaaatgaccatgcaaatgccagagcattttcgcacctggctaccaaattgatcgagcaggagactgacaaagacacactcatcttggatatcggcagtgcgccttccaggagaatgatgtctacgcacaaataccactgcgtatgccctatgcgcagcgcagaagaccccgaaaggctcgatagctacgcaaagaaactggcagcggcctccgggaaggtgctggatagagagatcgcaggaaaaatcaccgacctgcagaccgtcatggctacgccagacgctgaatctcctaccttttgcctgcatacagacgtcacgtgtcgtacggcagccgaagtggccgtataccaggacgtgtatgctgtacatgcaccaacatcgctgtaccatcaggcgatgaaaggtgtcagaacggcgtattggattgggtttgacaccaccccgtttatgtttgacgcgctagcaggcgcgtatccaacctacgccacaaactgggccgacgagcaggtgttacaggccaggaacataggactgtgtgcagcatccttgactgagggaagactcggcaaactgtccattctccgcaagaagcaattgaaaccttgcgacacagtcatgttctcggtaggatctacattgtacactgagagcagaaagctactgaggagctggcacttaccctccgtattccacctgaaaggtaaacaatcctttacctgtaggtgcgataccatcgtatcatgtgaagggtacgtagttaagaaaatcactatgtgccccggcctgtacggtaaaacggtagggtacgccgtgacgtatcacgcggagggattcctagtgtgcaagaccacagacactgtcaaaggagaaagagtctcattccctgtatgcacctacgtcccctcaaccatctgtgatcaaatgactggcatactagcgaccgacgtcacaccggaggacgcacagaagttgttagtgggattgaatcagaggatagttgtgaacggaagaacacagcgaaacactaacacgatgaagaactatctgcttccgattgtggccgtcgcatttagcaagtgggcgagggaatacaaggcagaccttgatgatgaaaaacctctgggtgtccgagagaggtcacttacttgctgctgcttgtgggcatttaaaacgaggaagatgcacaccatgtacaagaaaccagacacccagacaatagtgaaggtgccttcagagtttaactcgttcgtcatcccgagcctatggtctacaggcctcgcaatcccagtcagatcacgcattaagatgcttttggccaagaagaccaagcgagagttaatacctgttctcgacgcgtcgtcagccagggatgctgaacaagaggagaaggagaggttggaggccgagctgactagagaagccttaccacccctcgtccccatcgcgccggcggagacgggagtcgtcgacgtcgacgttgaagaactagagtatcacgcaggtgcaggggtcgtggaaacacctcgcagcgcgttgaaagtcaccgcacagccgaacgacgtactactaggaaattacgtagttctgtccccgcagaccgtgctcaagagctccaagttggcccccgtgcaccctctagcagagcaggtgaaaataataacacataacgggagggccggcggttaccaggtcgacggatatgacggcagggtcctactaccatgtggatcggccattccggtccctgagtttcaagctttgagcgagagcgccactatggtgtacaacgaaagggagttcgtcaacaggaaactataccatattgccgttcacggaccgtcgctgaacaccgacgaggagaactacgagaaagtcagagctgaaagaactgacgccgagtacgtgttcgacgtagataaaaaatgctgcgtcaagagagaggaagcgtcgggtttggtgttggtgggagagctaaccaaccccccgttccatgaattcgcctacgaagggctgaagatcaggccgtcggcaccatataagactacagtagtaggagtctttggggttccgggatcaggcaagtctgctattattaagagcctcgtgaccaaacacgatctggtcaccagcggcaagaaggagaactgccaggaaatagttaacgacgtgaagaagcaccgcgggaaggggacaagtagggaaaacagtgactccatcctgctaaacgggtgtcgtcgtgccgtggacatcctatatgtggacgaggctttcgctagccattccggtactctgctggccctaattgctcttgttaaacctcggagcaaagtggtgttatgcggagaccccaagcaatgcggattcttcaatatgatgcagcttaaggtgaacttcaaccacaacatctgcactgaagtatgtcataaaagtatatccagacgttgcacgcgtccagtcacggccatcgtgtctacgttgcactacggaggcaagatgcgcacgaccaacccgtgcaacaaacccataatcatagacaccacaggacagaccaagcccaagccaggagacatcgtgttaacatgcttccgaggctgggcaaagcagctgcagttggactaccgtggacacgaagtcatgacagcagcagcatctcagggcctcacccgcaaaggggtatacgccgtaaggcagaaggtgaatgaaaatcccttgtatgcccctgcgtcggagcacgtgaatgtactgctgacgcgcactgaggataggctggtgtggaaaacgctggccggcgatccctggattaaggtcctatcaaacattccacagggtaactttacggccacattggaagaatggcaagaagaacacgacaaaataatgaaggtgattgaaggaccggctgcgcctgtggacgcgttccagaacaaagcgaacgtgtgttgggcgaaaagcctggtgcctgtcctggacactgccggaatcagattgacagcagaggagtggagcaccataattacagcatttaaggaggacagagcttactctccagtggtggccttgaatgaaatttgcaccaagtactatggagttgacctggacagtggcctgttttctgccccgaaggtgtccctgtattacgagaacaaccactgggataacagacctggtggaaggatgtatggattcaatgccgcaacagctgccaggctggaagctagacataccttcctgaaggggcagtggcatacgggcaagcaggcagttatcgcagaaagaaaaatccaaccgctttctgtgctggacaatgtaattcctatcaaccgcaggctgccgcacgccctggtggctgagtacaagacggttaaaggcagtagggttgagtggctggtcaataaagtaagagggtaccacgtcctgctggtgagtgagtacaacctggctttgcctcgacgcagggtcacttggttgtcaccgctgaatgtcacaggcgccgataggtgctacgacctaagtttaggactgccggctgacgccggcaggttcgacttggtctttgtgaacattcacacggaattcagaatccaccactaccagcagtgtgtcgaccacgccatgaagctgcagatgcttgggggagatgcgctacgactgctaaaacccggcggcatcttgatgagagcttacggatacgccgataaaatcagcgaagccgttgtttcctccttaagcagaaagttctcgtctgcaagagtgttgcgcccggattgtgtcaccagcaatacagaagtgttcttgctgttctccaactttgacaacggaaagagaccctctacgctacaccagatgaataccaagctgagtgccgtgtatgccggagaagccatgcacacggccgggtgtgcaccatcctacagagttaagagagcagacatagccacgtgcacagaagcggctgtggttaacgcagctaacgcccgtggaactgtaggggatggcgtatgcagggccgtggcgaagaaatggccgtcagcctttaagggagcagcaacaccagtgggcacaattaaaacagtcatgtgcggctcgtaccccgtcatccacgctgtagcgcctaatttctctgccacgactgaagcggaaggggaccgcgaattggccgctgtctaccgggcagtggccgccgaagtaaacagactgtcactgagcagcgtagccatcccgctgctgtccacaggagtgttcagcggcggaagagataggctgcagcaatccctcaaccatctattcacagcaatggacgccacggacgctgacgtgaccatctactgcagagacaaaagttgggagaagaaaatccaggaagccattgacatgaggacggctgtggagttgctcaatgatgacgtggagctgaccacagacttggtgagagtgcacccggacagcagcctggtgggtcgtaagggctacagtaccactgacgggtcgctgtactcgtactttgaaggtacgaaattcaaccaggctgctattgatatggcagagatactgacgttgtggcccagactgcaagaggcaaacgaacagatatgcctatacgcgctgggcgaaacaatggacaacatcagatccaaatgtccggtgaacgattccgattcatcaacacctcccaggacagtgccctgcctgtgccgctacgcaatgacagcagaacggatcgcccgccttaggtcacaccaagttaaaagcatggtggtttgctcatcttttcccctcccgaaataccatgtagatggggtgcagaaggtaaagtgcgagaaggttctcctgttcgacccgacggtaccttcagtggttagtccgcggaagtatgccgcatctacgacggaccactcagatcggtcgttacgagggtttgacttggactggaccaccgactcgtcttccactgccagcgataccatgtcgctacccagtttgcagtcgtgtgacatcgactcgatctacgagccaatggctcccatagtagtgacggctgacgtacaccctgaacccgcaggcatcgcggacctggcggcagatgtgcaccctgaacccgcagaccatgtggacctcgagaacccgattcctccaccgcgcccgaagagagctgcataccttgcctcccgcgcggcggagcgaccggtgccggcgccgagaaagccgacgcctgccccaaggactgcgtttaggaacaagctgcctttgacgttcggcgactttgacgagcacgaggtcgatgcgttggcctccgggattactttcggagacttcgacgacgtcctgcgactaggccgcgcgggtgcatatattttctcctcggacactggcagcggacatttacaacaaaaatccgttaggcagcacaatctccagtgcgcacaactggatgcggtccaggaggagaaaatgtacccgccaaaattggatactgagagggagaagctgttgctgctgaaaatgcagatgcacccatcggaggctaataagagtcgataccagtctcgcaaagtggagaacatgaaagccacggtggtggacaggctcacatcgggggccagattgtacacgggagcggacgtaggccgcataccaacatacgcggttcggtacccccgccccgtgtactcccctaccgtgatcgaaagattctcaagccccgatgtagcaatcgcagcgtgcaacgaatacctatccagaaattacccaacagtggcgtcgtaccagataacagatgaatacgacgcatacttggacatggttgacgggtcggatagttgcttggacagagcgacattctgcccggcgaagctccggtgctacccgaaacatcatgcgtaccaccagccgactgtacgcagtgccgtcccgtcaccctttcagaacacactacagaacgtgctagcggccgccaccaagagaaactgcaacgtcacgcaaatgcgagaactacccaccatggactcggcagtgttcaacgtggagtgcttcaagcgctatgcctgctccggagaatattgggaagaatatgctaaacaacctatccggataaccactgagaacatcactacctatgtgaccaaattgaaaggcccgaaagctgctgccttgttcgctaagacccacaacttggttccgctgcaggaggttcccatggacagattcacggtcgacatgaaacgagatgtcaaagtcactccagggacgaaacacacagaggaaagacccaaagtccaggtaattcaagcagcggagccattggcgaccgcttacctgtgcggcatccacagggaattagtaaggagactaaatgctgtgttacgccctaacgtgcacacattgtttgatatgtcggccgaagactttgacgcgatcatcgcctctcacttccacccaggagacccggttctagagacggacattgcatcattcgacaaaagccaggacgactccttggctcttacaggtttaatgatcctcgaagatctaggggtggatcagtacctgctggacttgatcgaggcagcctttggggaaatatccagctgtcacctaccaactggcacgcgcttcaagttcggagctatgatgaaatcgggcatgtttctgactttgtttattaacactgttttgaacatcaccatagcaagcagggtactggagcagagactcactgactccgcctgtgcggccttcatcggcgacgacaacatcgttcacggagtgatctccgacaagctgatggcggagaggtgcgcgtcgtgggtcaacatggaggtgaagatcattgacgctgtcatgggcgaaaaacccccatatttttgtgggggattcatagtttttgacagcgtcacacagaccgcctgccgtgtttcagacccacttaagcgcctgttcaagttgggtaagccgctaacagctgaagacaagcaggacgaagacaggcgacgagcactgagtgacgaggttagcaagtggttccggacaggcttgggggccgaactggaggtggcactaacatctaggtatgaggtagagggctgcaaaagtatcctcatagccatggccaccttggcgagggacattaaggcgtttaagaaattgagaggacctgttatacacctctacggcggtcctagattggtgcgttaatacacagaattctgattggatcccaaacgggccctctagactcgagcggccgccactgtgctggatatctgcagaattccaccacactggactagtggatctatggcgtacccatacgatgttccagattacgctagcttgagatctaccatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactgcgcacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtagccattcttcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatgaagatccgagctcggtaccaagcttaagtttgggtaattaattgaattacatccctacgcaaacgttttacggccgccggtggcgcccgcgcccggcggcccgtccttggccgttgcaggccactccggtggctcccgtcgtccccgacttccaggcccagcagatgcagcaactcatcagcgccgtaaatgcgctgacaatgagacagaacgcaattgctcctgctaggcctcccaaaccaaagaagaagaagacaaccaaaccaaagccgaaaacgcagcccaagaagatcaacggaaaaacgcagcagcaaaagaagaaagacaagcaagccgacaagaagaagaagaaacccggaaaaagagaaagaatgtgcatgaagattgaaaatgactgtatcttcgtatgcggctagccacagtaacgtagtgtttccagacatgtcgggcaccgcactatcatgggtgcagaaaatctcgggtggtctgggggccttcgcaatcggcgctatcctggtgctggttgtggtcacttgcattgggctccgcagataagttagggtaggcaatggcattgatatagcaagaaaattgaaaacagaaaaagttagggtaagcaatggcatataaccataactgtataacttgtaacaaagcgcaacaagacctgcgcaattggccccgtggtccgcctcacggaaactcggggcaactcatattgacacattaattggcaataattggaagcttacataagcttaattcgacgaataattggatttttattttattttgcaattggtttttaatatttccaaaaaaaaaaaaaaaaaaaaaaaaaaaaaaaaaaaaaaaaaaaaaaaaaaaaaaaaaaaaaaaaaaaaaactagtgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctagtctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcgcgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggcattctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttctgtctaagcggatgccgggagcagacaagcccgtcagggcgcgtcagcgggtgttggcgggtgtcggggctggcttaactatgcggcatcagagcagattgtactgagagtgcaccatatcgacgctctcccttatgcgactcctgcattaggaagcagcccagtactaggttgaggccgttgagcaccgccgccgcaaggaatggtgcatgcgtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttaactggcttatcgaaattaatacgactcactatagggagaccggaagcttgaattc

SEQ ID NO: 68 - DNA, 12612 bp, Artificial Sequence
  Description of Artificial Sequence Synthetic polynucleotide
atggcggatgtgtgacatacacgacgccaaaagattttgttccagctcctgccacctccgctacgcgagagattaaccacccacgatggccgccaaagtgcatgttgatattgaggctgacagcccattcatcaagtctttgcagaaggcatttccgtcgttcgaggtggagtcattgcaggtcacaccaaatgaccatgcaaatgccagagcattttcgcacctggctaccaaattgatcgagcaggagactgacaaagacacactcatcttggatatcggcagtgcgccttccaggagaatgatgtctacgcacaaataccactgcgtatgccctatgcgcagcgcagaagaccccgaaaggctcgatagctacgcaaagaaactggcagcggcctccgggaaggtgctggatagagagatcgcaggaaaaatcaccgacctgcagaccgtcatggctacgccagacgctgaatctcctaccttttgcctgcatacagacgtcacgtgtcgtacggcagccgaagtggccgtataccaggacgtgtatgctgtacatgcaccaacatcgctgtaccatcaggcgatgaaaggtgtcagaacggcgtattggattgggtttgacaccaccccgtttatgtttgacgcgctagcaggcgcgtatccaacctacgccacaaactgggccgacgagcaggtgttacaggccaggaacataggactgtgtgcagcatccttgactgagggaagactcggcaaactgtccattctccgcaagaagcaattgaaaccttgcgacacagtcatgttctcggtaggatctacattgtacactgagagcagaaagctactgaggagctggcacttaccctccgtattccacctgaaaggtaaacaatcctttacctgtaggtgcgataccatcgtatcatgtgaagggtacgtagttaagaaaatcactatgtgccccggcctgtacggtaaaacggtagggtacgccgtgacgtatcacgcggagggattcctagtgtgcaagaccacagacactgtcaaaggagaaagagtctcattccctgtatgcacctacgtcccctcaaccatctgtgatcaaatgactggcatactagcgaccgacgtcacaccggaggacgcacagaagttgttagtgggattgaatcagaggatagttgtgaacggaagaacacagcgaaacactaacacgatgaagaactatctgcttccgattgtggccgtcgcatttagcaagtgggcgagggaatacaaggcagaccttgatgatgaaaaacctctgggtgtccgagagaggtcacttacttgctgctgcttgtgggcatttaaaacgaggaagatgcacaccatgtacaagaaaccagacacccagacaatagtgaaggtgccttcagagtttaactcgttcgtcatcccgagcctatggtctacaggcctcgcaatcccagtcagatcacgcattaagatgcttttggccaagaagaccaagcgagagttaatacctgttctcgacgcgtcgtcagccagggatgctgaacaagaggagaaggagaggttggaggccgagctgactagagaagccttaccacccctcgtccccatcgcgccggcggagacgggagtcgtcgacgtcgacgttgaagaactagagtatcacgcaggtgcaggggtcgtggaaacacctcgcagcgcgttgaaagtcaccgcacagccgaacgacgtactactaggaaattacgtagttctgtccccgcagaccgtgctcaagagctccaagttggcccccgtgcaccctctagcagagcaggtgaaaataataacacataacgggagggccggcggttaccaggtcgacggatatgacggcagggtcctactaccatgtggatcggccattccggtccctgagtttcaagctttgagcgagagcgccactatggtgtacaacgaaagggagttcgtcaacaggaaactataccatattgccgttcacggaccgtcgctgaacaccgacgaggagaactacgagaaagtcagagctgaaagaactgacgccgagtacgtgttcgacgtagataaaaaatgctgcgtcaagagagaggaagcgtcgggtttggtgttggtgggagagctaaccaaccccccgttccatgaattcgcctacgaagggctgaagatcaggccgtcggcaccatataagactacagtagtaggagtctttggggttccgggatcaggcaagtctgctattattaagagcctcgtgaccaaacacgatctggtcaccagcggcaagaaggagaactgccaggaaatagttaacgacgtgaagaagcaccgcgggaaggggacaagtagggaaaacagtgactccatcctgctaaacgggtgtcgtcgtgccgtggacatcctatatgtggacgaggctttcgctagccattccggtactctgctggccctaattgctcttgttaaacctcggagcaaagtggtgttatgcggagaccccaagcaatgcggattcttcaatatgatgcagcttaaggtgaacttcaaccacaacatctgcactgaagtatgtcataaaagtatatccagacgttgcacgcgtccagtcacggccatcgtgtctacgttgcactacggaggcaagatgcgcacgaccaacccgtgcaacaaacccataatcatagacaccacaggacagaccaagcccaagccaggagacatcgtgttaacatgcttccgaggctgggcaaagcagctgcagttggactaccgtggacacgaagtcatgacagcagcagcatctcagggcctcacccgcaaaggggtatacgccgtaaggcagaaggtgaatgaaaatcccttgtatgcccctgcgtcggagcacgtgaatgtactgctgacgcgcactgaggataggctggtgtggaaaacgctggccggcgatccctggattaaggtcctatcaaacattccacagggtaactttacggccacattggaagaatggcaagaagaacacgacaaaataatgaaggtgattgaaggaccggctgcgcctgtggacgcgttccagaacaaagcgaacgtgtgttgggcgaaaagcctggtgcctgtcctggacactgccggaatcagattgacagcagaggagtggagcaccataattacagcatttaaggaggacagagcttactctccagtggtggccttgaatgaaatttgcaccaagtactatggagttgacctggacagtggcctgttttctgccccgaaggtgtccctgtattacgagaacaaccactgggataacagacctggtggaaggatgtatggattcaatgccgcaacagctgccaggctggaagctagacataccttcctgaaggggcagtggcatacgggcaagcaggcagttatcgcagaaagaaaaatccaaccgctttctgtgctggacaatgtaattcctatcaaccgcaggctgccgcacgccctggtggctgagtacaagacggttaaaggcagtagggttgagtggctggtcaataaagtaagagggtaccacgtcctgctggtgagtgagtacaacctggctttgcctcgacgcagggtcacttggttgtcaccgctgaatgtcacaggcgccgataggtgctacgacctaagtttaggactgccggctgacgccggcaggttcgacttggtctttgtgaacattcacacggaattcagaatccaccactaccagcagtgtgtcgaccacgccatgaagctgcagatgcttgggggagatgcgctacgactgctaaaacccggcggcatcttgatgagagcttacggatacgccgataaaatcagcgaagccgttgtttcctccttaagcagaaagttctcgtctgcaagagtgttgcgcccggattgtgtcaccagcaatacagaagtgttcttgctgttctccaactttgacaacggaaagagaccctctacgctacaccagatgaataccaagctgagtgccgtgtatgccggagaagccatgcacacggccgggtgtgcaccatcctacagagttaagagagcagacatagccacgtgcacagaagcggctgtggttaacgcagctaacgcccgtggaactgtaggggatggcgtatgcagggccgtggcgaagaaatggccgtcagcctttaagggagcagcaacaccagtgggcacaattaaaacagtcatgtgcggctcgtaccccgtcatccacgctgtagcgcctaatttctctgccacgactgaagcggaaggggaccgcgaattggccgctgtctaccgggcagtggccgccgaagtaaacagactgtcactgagcagcgtagccatcccgctgctgtccacaggagtgttcagcggcggaagagataggctgcagcaatccctcaaccatctattcacagcaatggacgccacggacgctgacgtgaccatctactgcagagacaaaagttgggagaagaaaatccaggaagccattgacatgaggacggctgtggagttgctcaatgatgacgtggagctgaccacagacttggtgagagtgcacccggacagcagcctggtgggtcgtaagggctacagtaccactgacgggtcgctgtactcgtactttgaaggtacgaaattcaaccaggctgctattgatatggcagagatactgacgttgtggcccagactgcaagaggcaaacgaacagatatgcctatacgcgctgggcgaaacaatggacaacatcagatccaaatgtccggtgaacgattccgattcatcaacacctcccaggacagtgccctgcctgtgccgctacgcaatgacagcagaacggatcgcccgccttaggtcacaccaagttaaaagcatggtggtttgctcatcttttcccctcccgaaataccatgtagatggggtgcagaaggtaaagtgcgagaaggttctcctgttcgacccgacggtaccttcagtggttagtccgcggaagtatgccgcatctacgacggaccactcagatcggtcgttacgagggtttgacttggactggaccaccgactcgtcttccactgccagcgataccatgtcgctacccagtttgcagtcgtgtgacatcgactcgatctacgagccaatggctcccatagtagtgacggctgacgtacaccctgaacccgcaggcatcgcggacctggcggcagatgtgcaccctgaacccgcagaccatgtggacctcgagaacccgattcctccaccgcgcccgaagagagctgcataccttgcctcccgcgcggcggagcgaccggtgccggcgccgagaaagccgacgcctgccccaaggactgcgtttaggaacaagctgcctttgacgttcggcgactttgacgagcacgaggtcgatgcgttggcctccgggattactttcggagacttcgacgacgtcctgcgactaggccgcgcgggtgcatatattttctcctcggacactggcagcggacatttacaacaaaaatccgttaggcagcacaatctccagtgcgcacaactggatgcggtccaggaggagaaaatgtacccgccaaaattggatactgagagggagaagctgttgctgctgaaaatgcagatgcacccatcggaggctaataagagtcgataccagtctcgcaaagtggagaacatgaaagccacggtggtggacaggctcacatcgggggccagattgtacacgggagcggacgtaggccgcataccaacatacgcggttcggtacccccgccccgtgtactcccctaccgtgatcgaaagattctcaagccccgatgtagcaatcgcagcgtgcaacgaatacctatccagaaattacccaacagtggcgtcgtaccagataacagatgaatacgacgcatacttggacatggttgacgggtcggatagttgcttggacagagcgacattctgcccggcgaagctccggtgctacccgaaacatcatgcgtaccaccagccgactgtacgcagtgccgtcccgtcaccctttcagaacacactacagaacgtgctagcggccgccaccaagagaaactgcaacgtcacgcaaatgcgagaactacccaccatggactcggcagtgttcaacgtggagtgcttcaagcgctatgcctgctccggagaatattgggaagaatatgctaaacaacctatccggataaccactgagaacatcactacctatgtgaccaaattgaaaggcccgaaagctgctgccttgttcgctaagacccacaacttggttccgctgcaggaggttcccatggacagattcacggtcgacatgaaacgagatgtcaaagtcactccagggacgaaacacacagaggaaagacccaaagtccaggtaattcaagcagcggagccattggcgaccgcttacctgtgcggcatccacagggaattagtaaggagactaaatgctgtgttacgccctaacgtgcacacattgtttgatatgtcggccgaagactttgacgcgatcatcgcctctcacttccacccaggagacccggttctagagacggacattgcatcattcgacaaaagccaggacgactccttggctcttacaggtttaatgatcctcgaagatctaggggtggatcagtacctgctggacttgatcgaggcagcctttggggaaatatccagctgtcacctaccaactggcacgcgcttcaagttcggagctatgatgaaatcgggcatgtttctgactttgtttattaacactgttttgaacatcaccatagcaagcagggtactggagcagagactcactgactccgcctgtgcggccttcatcggcgacgacaacatcgttcacggagtgatctccgacaagctgatggcggagaggtgcgcgtcgtgggtcaacatggaggtgaagatcattgacgctgtcatgggcgaaaaacccccatatttttgtgggggattcatagtttttgacagcgtcacacagaccgcctgccgtgtttcagacccacttaagcgcctgttcaagttgggtaagccgctaacagctgaagacaagcaggacgaagacaggcgacgagcactgagtgacgaggttagcaagtggttccggacaggcttgggggccgaactggaggtggcactaacatctaggtatgaggtagagggctgcaaaagtatcctcatagccatggccaccttggcgagggacattaaggcgtttaagaaattgagaggacctgttatacacctctacggcggtcctagattggtgcgttaatacacagaattctgattggatcccaaacgggccctctagactcgagcggccgccactgtgctggatatctgcagaattcatgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccaggatctatggcgtacccatacgatgttccagattacgctagcttgagatctaccatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactgcgcacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtagccattcttcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatgaagatccaagcttaagtttgggtaattaattgaattacatccctacgcaaacgttttacggccgccggtggcgcccgcgcccggcggcccgtccttggccgttgcaggccactccggtggctcccgtcgtccccgacttccaggcccagcagatgcagcaactcatcagcgccgtaaatgcgctgacaatgagacagaacgcaattgctcctgctaggcctcccaaaccaaagaagaagaagacaaccaaaccaaagccgaaaacgcagcccaagaagatcaacggaaaaacgcagcagcaaaagaagaaagacaagcaagccgacaagaagaagaagaaacccggaaaaagagaaagaatgtgcatgaagattgaaaatgactgtatcttcgtatgcggctagccacagtaacgtagtgtttccagacatgtcgggcaccgcactatcatgggtgcagaaaatctcgggtggtctgggggccttcgcaatcggcgctatcctggtgctggttgtggtcacttgcattgggctccgcagataagttagggtaggcaatggcattgatatagcaagaaaattgaaaacagaaaaagttagggtaagcaatggcatataaccataactgtataacttgtaacaaagcgcaacaagacctgcgcaattggccccgtggtccgcctcacggaaactcggggcaactcatattgacacattaattggcaataattggaagcttacataagcttaattcgacgaataattggatttttattttattttgcaattggtttttaatatttccaaaaaaaaaaaaaaaaaaaaaaaaaaaaaaaaaaaaaaaaaaaaaaaaaaaaaaaaaaaaaaaaaaaaaactagtgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctagtctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcgcgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggcattctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttctgtctaagcggatgccgggagcagacaagcccgtcagggcgcgtcagcgggtgttggcgggtgtcggggctggcttaactatgcggcatcagagcagattgtactgagagtgcaccatatcgacgctctcccttatgcgactcctgcattaggaagcagcccagtactaggttgaggccgttgagcaccgccgccgcaaggaatggtgcatgcgtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttaactggcttatcgaaattaatacgactcactatagggagaccggaagcttgaattc

SEQ ID NO: 69 - DNA, 4832 bp, Artificial Sequence
  Description of Artificial Sequence Synthetic polynucleotide
gtcgacttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctggatcgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcattttggcaaagaattgtaatacgactcactatagggcgaattcggatccagatctatggcgtacccatacgatgttccagattacgctagcttgagatctaccatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactgcgcacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatgaagatcttattaaagcagaacttgtttattgcagcttataatggttacaaataaagcaatagcatcacaaatttcacaaataaagcatttttttcactgcattctagttgtggtttgtccaaactcatcaatgtatcttatcatgtctggtcgactctagactcttccgcttcctcgctcactgactcgctgcgctcggtcgttcggctgcggcgagcggtatcagctcactcaaaggcggtaatacggttatccacagaatcaggggataacgcaggaaagaacatgtgagcaaaggccagcaaaaggccaggaaccgtaaaaaggccgcgttgctggcgttttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcttttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaatt

SEQ ID NO: 70 - DNA, 4832 bp, Artificial Sequence
  Description of Artificial Sequence Synthetic polynucleotide
gtcgacttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctggatcgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcattttggcaaagaattgtaatacgactcactatagggcgaattcggatccagatctatggcgtacccatacgatgttccagattacgctagcttgagatctaccatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactgcgcacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtagccattcttcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatgaagatcttattaaagcagaacttgtttattgcagcttataatggttacaaataaagcaatagcatcacaaatttcacaaataaagcatttttttcactgcattctagttgtggtttgtccaaactcatcaatgtatcttatcatgtctggtcgactctagactcttccgcttcctcgctcactgactcgctgcgctcggtcgttcggctgcggcgagcggtatcagctcactcaaaggcggtaatacggttatccacagaatcaggggataacgcaggaaagaacatgtgagcaaaggccagcaaaaggccaggaaccgtaaaaaggccgcgttgctggcgttttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcttttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaatt

SEQ ID NO: 71 - DNA, 1499 bp, Homo sapiens
atgacttttaacagttttgaaggatctaaaacttgtgtacctgcagacatcaataaggaagaagaatttgtagaagagtttaatagattaaaaacttttgctaattttccaagtggtagtcctgtttcagcatcaacactggcacgagcagggtttctttatactggtgaaggagataccgtgcggtgctttagttgtcatgcagctgtagatagatggcaatatggagactcagcagttggaagacacaggaaagtatccccaaattgcagatttatcaacggcttttatcttgaaaatagtgccacgcagtctacaaattctggtatccagaatggtcagtacaaagttgaaaactatctgggaagcagagatcattttgccttagacaggccatctgagacacatgcagactatcttttgagaactgggcaggttgtagatatatcagacaccatatacccgaggaaccctgccatgtattgtgaagaagctagattaaagtcctttcagaactggccagactatgctcacctaaccccaagagagttagcaagtgctggactctactacacaggtattggtgaccaagtgcagtgcttttgttgtggtggaaaactgaaaaattgggaaccttgtgatcgtgcctggtcagaacacaggcgacactttcctaattgcttctttgttttgggccggaatcttaatattcgaagtgaatctgatgctgtgagttctgataggaatttcccaaattcaacaaatcttccaagaaatccatccatggcagattatgaagcacggatctttacttttgggacatggatatactcagttaacaaggagcagcttgcaagagctggattttatgctttaggtgaaggtgataaagtaaagtgctttcactgtggaggagggctaactgattggaagcccagtgaagacccttgggaacaacatgctaaatggtatccagggtgcaaatatctgttagaacagaagggacaagaatatataaacaatattcatttaactcattcacttgaggagtgtctggtaagaactactgagaaaacaccatcactaactagaagaattgatgataccatcttccaaaatcctatggtacaagaagctatacgaatggggttcagtttcaaggacattaagaaaataatggaggaaaaaattcagatatctgggagcaactataaatcacttgaggttctggttgcagatctagtgaatgctcagaaagacagtatgcaagatgagtcaagtcagacttcattacagaaagagattagtactgaagagcagctaaggcgcctgcaagaggagaagctttgcaaaatctgtatggatagaaatattgctatcgtttttgttccttgtggacatctagtcacttgtaaacaatgtgctgaagcagttgacaagtgtcccatgtgctacacagtcattactttcaagcaaaaaatttttatgtcttaatctaa

SEQ ID NO: 72 - protein, 497 aa, Homo sapiens [sequence omitted]

SEQ ID NO: 73 - DNA, 5575 bp, Artificial Sequence
  Description of Artificial Sequence Synthetic polynucleotide
gtcgacttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctggatcgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaataatcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcattttggcaaagaattgtaatacgactcactatagggcgaattcggatccatgacttttaacagttttgaaggatctaaaacttgtgtacctgcagacatcaataaggaagaagaatttgtagaagagtttaatagattaaaaacttttgctaattttccaagtggtagtcctgtttcagcatcaacactggcacgagcagggtttctttatactggtgaaggagataccgtgcggtgctttagttgtcatgcagctgtagatagatggcaatatggagactcagcagttggaagacacaggaaagtatccccaaattgcagatttatcaacggcttttatcttgaaaatagtgccacgcagtctacaaattctggtatccagaatggtcagtacaaagttgaaaactatctgggaagcagagatcattttgccttagacaggccatctgagacacatgcagactatcttttgagaactgggcaggttgtagatatatcagacaccatatacccgaggaaccctgccatgtattgtgaagaagctagattaaagtcctttcagaactggccagactatgctcacctaaccccaagagagttagcaagtgctggactctactacacaggtattggtgaccaagtgcagtgcttttgttgtggtggaaaactgaaaaattgggaaccttgtgatcgtgcctggtcagaacacaggcgacactttcctaattgcttctttgttttgggccggaatcttaatattcgaagtgaatctgatgctgtgagttctgataggaatttcccaaattcaacaaatcttccaagaaatccatccatggcagattatgaagcacggatctttacttttgggacatggatatactcagttaacaaggagcagcttgcaagagctggattttatgctttaggtgaaggtgataaagtaaagtgctttcactgtggaggagggctaactgattggaagcccagtgaagacccttgggaacaacatgctaaatggtatccagggtgcaaatatctgttagaacagaagggacaagaatatataaacaatattcatttaactcattcacttgaggagtgtctggtaagaactactgagaaaacaccatcactaactagaagaattgatgataccatcttccaaaatcctatggtacaagaagctatacgaatggggttcagtttcaaggacattaagaaaataatggaggaaaaaattcagatatctgggagcaactataaatcacttgaggttctggttgcagatctagtgaatgctcagaaagacagtatgcaagatgagtcaagtcagacttcattacagaaagagattagtactgaagagcagctaaggcgcctgcaagaggagaagctttgcaaaatctgtatggatagaaatattgctatcgtttttgttccttgtggacatctagtcacttgtaaacaatgtgctgaagcagttgacaagtgtcccatgtgctacacagtcattactttcaagcaaaaaatttttatgtcttaatctaaagatcttattaaagcagaacttgtttattgcagcttataatggttacaaataaagcaatagcatcacaaatttcacaaataaagcatttttttcactgcattctagttgtggtttgtccaaactcatcaatgtatcttatcatgtctggtcgactctagactcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcttttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaatt

SEQ ID NO: 74 - DNA, 1395 bp, Homo sapiens
atggacttcagcagaaatctttatgatattggggaacaactggacagtgaagatctggcctccctcaagttcctgagcctggactacattccgcaaaggaagcaagaacccatcaaggatgccttgatgttattccagagactccaggaaaagagaatgttggaggaaagcaatctgtccttcctgaaggagctgctcttccgaattaatagactggatttgctgattacctacctaaacactagaaaggaggagatggaaagggaacttcagacaccaggcagggctcaaatttctgcctacagggtcatgctctatcagatttcagaagaagtgagcagatcagaattgaggtcttttaagtttcttttgcaagaggaaatctccaaatgcaaactggatgatgacatgaacctgctggatattttcatagagatggagaagagggtcatcctgggagaaggaaagttggacatcctgaaaagagtctgtgcccaaatcaacaagagcctgctgaagataatcaacgactatgaagaattcagcaaaggggaggagttgtgtggggtaatgacaatctcggactctccaagagaacaggatagtgaatcacagactttggacaaagtttaccaaatgaaaagcaaacctcggggatactgtctgatcatcaacaatcacaattttgcaaaagcacgggagaaagtgcccaaacttcacagcattagggacaggaatggaacacacttggatgcaggggctttgaccacgacctttgaagagcttcattttgagatcaagccccacgatgactgcacagtagagcaaatctatgagattttgaaaatctaccaactcatggaccacagtaacatggactgcttcatctgctgtatcctctcccatggagacaagggcatcatctatggcactgatggacaggaggcccccatctatgagctgacatctcagttcactggtttgaagtgcccttcccttgctggaaaacccaaagtgttttttattcaggcttgtcagggggataactaccagaaaggtatacctgttgagactgattcagaggagcaaccctatttagaaatggatttatcatcacctcaaacgagatatatcccggatgaggctgactttctgctggggatggccactgtgaataactgtgtttcctaccgaaaccctgcagagggaacctggtacatccagtcactttgccagagcctgagagagcgatgtcctcgaggcgatgatattctcaccatcctgactgaagtgaactatgaagtaagcaacaaggatgacaagaaaaacatggggaaacagatgcctcagcctactttcacactaagaaaaaaacttgtcttcccttctgattga

SEQ ID NO: 75 - protein, 464 aa, Homo sapiens
MDFSRNLYDIGEQLDSEDLASLKFLSLDYIPQRKQEPIKDALMLFQRLQEKRMLEESNLSFLKELLFRINRLDLLITYLNTRKEEMERELQTPGRAQISAYRVMLYQISEEVSRSELRSFKFLLQEEISKCKLDDDMNLLDIFIEMEKRVILGEGKLDILKRVCAQINKSLLKIINDYEEFSKGEELCGVMTISDSPREQDSESQTLDKVYQMKSKPRGYCLIINNHNFAKAREKVPKLHSIRDRNGTHLDAGALTTTFEELHFEIKPHDDCTVEQIYEILKIYQLMDHSNMDCFICCILSHGDKGIIYGTDGQEAPIYELTSQFTGLKCPSLAGKPKVFFIQACQGDNYQKGIPVETDSEEQPYLEMDLSSPQTRYIPDEADFLLGMATVNNCVSYRNPAEGTWYIQSLCQSLRERCPRGDDILTILTEVNYEVSNKDDKKNMGKQMPQPTFTLRKKLVFPSD

SEQ ID NO: 76 - DNA, 5471 bp, Artificial Sequence
  Description of Artificial Sequence Synthetic polynucleotide
gtcgacttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctggatcgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcattttggcaaagaattgtaatacgactcactatagggcgaattcatggacttcagcagaaatctttatgatattggggaacaactggacagtgaagatctggcctccctcaagttcctgagcctggactacattccgcaaaggaagcaagaacccatcaaggatgccttgatgttattccagagactccaggaaaagagaatgttggaggaaagcaatctgtccttcctgaaggagctgctcttccgaattaatagactggatttgctgattacctacctaaacactagaaaggaggagatggaaagggaacttcagacaccaggcagggctcaaatttctgcctacagggtcatgctctatcagatttcagaagaagtgagcagatcagaattgaggtcttttaagtttcttttgcaagaggaaatctccaaatgcaaactggatgatgacatgaacctgctggatattttcatagagatggagaagagggtcatcctgggagaaggaaagttggacatcctgaaaagagtctgtgcccaaatcaacaagagcctgctgaagataatcaacgactatgaagaattcagcaaaggggaggagttgtgtggggtaatgacaatctcggactctccaagagaacaggatagtgaatcacagactttggacaaagtttaccaaatgaaaagcaaacctcgggatactgtctgatcatcaacaatcacaattttgcaaaagcacgggagaaagtgccccaaacttcacagcattagggacaggaatggaacacacttggatgcaggggctttgaccacgacctttgaagagcttcattttgagatcaagccccacgatgactgcacagtagagcaaatctatgagattttgaaaatctaccaactcatggaccacagtaacatggactgcttcatctgctgtatcctctcccatggagacaagggcatcatctatggcactgatggacaggaggcccccatctatgagctgacatctcagttcactggtttgaagtgcccttcccttgctggaaaacccaaagtgttttttattcaggcttgtcagggggataactaccagaaaggtatacctgttgagactgattcagaggagcaaccctatttagaaatggatttatcatcacctcaaacgagatatatcccggatgaggctgactttctgctggggatggccactgtgaataactgtgtttcctaccgaaaccctgcagagggaacctggtacatccagtcactttgccagagcctgagagagcgatgtcctcgaggcgatgatattctcaccatcctgactgaagtgaactatgaagtaagcaacaaggatgacaagaaaaacatggggaaacagatgcctcagcctactttcacactaagaaaaaaacttgtcttcccttctgattgaggatccagatcttattaaagcagaacttgtttattgcagcttataatggttacaaataaagcaatagcatcacaaatttcacaaataaagcatttttttcactgcattctagttgtggtttgtccaaactcatcaatgtatcttatcatgtctggtcgactctagactcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcttttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaatt

SEQ ID NO: 77 - DNA, 618 bp, Homo sapiens
atggcgcacgctgggagaacagggtacgataaccgggagatagtgatgaagtacatccattataagctgtcgcagaggggctacgagtgggatgcgggagatgtgggcgccgcgcccccgggggccgcccccgcaccgggcatcttctcctcccagcccgggcacacgccccatccagccgcatcccgggacccggtcgccaggacctcgccgctgcagaccccggctgcccccggcgccgccgcggggcctgcgctcagcccggtgccacctgtggtccacctgaccctccgccaggccggcgacgacttctcccgccgctaccgccgcgacttcgccgagatgtccagccagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggtaggtgcacttggtgatgtgagtctgggctga

SEQ ID NO: 78 - protein, 205 aa, Homo sapiens
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWVGALGDVSLG

SEQ ID NO: 79 - DNA, 4699 bp, Artificial Sequence
  Description of Artificial Sequence Synthetic polynucleotide
gtcgacttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctggatcgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcattttggcaaagaattgtaatacgactcactatagggcgaattcggatccagatctatggcgcacgctgggagaacagggtacgataaccgggagatagtgatgaagtacatccattataagctgtcgcagaggggctacgagtgggatgcgggagatgtgggcgccgcgcccccgggggccgcccccgcaccgggcatcttctcctcccagcccgggcacacgccccatccagccgcatcccgggacccggtcgccaggacctcgccgctgcagaccccggctgcccccggcgccgccgcggggcctgcgctcagcccggtgccacctgtggtccacctgaccctccgccaggccggcgacgacttctcccgccgctaccgccgcgacttcgccgagatgtccagccagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggtaggtgcacttggtgatgtgagtctgggctgaagatcttattaaagcagaacttgtttattgcagcttataatggttacaaataaagcaatagcatcacaaatttcacaaataaagcatttttttcactgcattctagttgtggtttgtccaaactcatcaatgtatcttatcatgtctggtcgactctagactcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcttttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaatt

SEQ ID NO: 80 - DNA, 5471 bp, Artificial Sequence
  Description of Artificial Sequence Synthetic polynucleotide
gtcgacttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctggatcgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcattttggcaaagaattgtaatacgactcactatagggcgaattcggatccatggacttcagcagaaatctttatgatattggggaacaactggacagtgaagatctggcctccctcaagttcctgagcctggactacattccgcaaaggaagcaagaacccatcaaggatgccttgatgttattccagagactccaggaaaagagaatgttggaggaaagcaatctgtccttcctgaaggagctgctcttccgaattaatagactggatttgctgattacctacctaaacactagaaaggaggagatggaaagggaacttcagacaccaggcagggctcaaatttctgcctacagggtcatgctctatcagatttcagaagaagtgagcagatcagaattgaggtcttttaagtttcttttgcaagaggaaatctccaaatgcaaactggatgatgacatgaacctgctggatattttcatagagatggagaagagggtcatcctgggagaaggaaagttggacatcctgaaaagagtctgtgcccaaatcaacaagagcctgctgaagataatcaacgactatgaagaattcagcaaaggggaggagttgtgtggggtaatgacaatctcggactctccaagagaacaggatagtgaatcacagactttggacaaagtttaccaaatgaaaagcaaacctcggggatactgtctgatcatcaacaatcacaattttgcaaaagcacgggagaaagtgcccaaacttcacagcattagggacaggaatggaacacacttggatgcaggggctttgaccacgacctttgaagagcttcattttgagatcaagccccacgatgactgcacagtagagcaaatctatgagattttgaaaatctaccaactcatggaccacagtaacatggactgcttcatctgctgtatcctctcccatggagacaagggcatcatctatggcactgatggacaggaggcccccatctatgagctgacatctcagttcactggtttgaagtgcccttcccttgctggaaaacccaaagtgttttttattcaggcttctcagggggataactaccagaaaggtatacctgttgagactgattcagaggagcaaccctatttagaaatggatttatcatcacctcaaacgagatatatcccggatgaggctgactttctgctggggatggccactgtgaataactgtgtttcctaccgaaaccctgcagagggaacctggtacatccagtcactttgccagagcctgagagagcgatgtcctcgaggcgatgatattctcaccatcctgactgaagtgaactatgaagtaagcaacaaggatgacaagaaaaacatggggaaacagatgcctcagcctactttcacactaagaaaaaaacttgtcttcccttctgattgaagatcttattaaagcagaacttgtttattgcagcttataatggttacaaataaagcaatagcatcacaaatttcacaaataaagcatttttttcactgcattctagttgtggtttgtccaaactcatcaatgtatcttatcatgtctggtcgactctagactcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcttttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaatt

SEQ ID NO: 81 - protein, 464 aa, Homo sapiens
MDFSRNLYDIGEQLDSEDLASLKFLSLDYIPQRKQEPIKDALMLFQRLQEKRMLEESNLSFLKELLFRINRLDLLITYLNTRKEEMERELQTPGRAQISAYRVMLYQISEEVSRSELRSFKFLLQEEISKCKLDDDMNLLDIFIEMEKRVILGEGKLDILKRVCAQINKSLLKIINDYEEFSKGEELCGVMTISDSPREQDSESQTLDKVYQMKSKPRGYCLIINNHNFAKAREKVPKLHSIRDRNGTHLDAGALTTTFEELHFEIKPHDDCTVEQIYEILKIYQLMDHSNMDCFICCILSHGDKGIIYGTDGQEAPIYELTSQFTGLKCPSLAGKPKVFFIQASQGDNYQKGIPVETDSEEQPYLEMDLSSPQTRYIPDEADFLLGMATVNNCVSYRNPAEGTWYIQSLCQSLRERCPRGDDILTILTEVNYEVSNKDDKKNMGKQMPQPTFTLRKKLVFPSD

SEQ ID NO: 82 - DNA, 5327 bp, Artificial Sequence
  Description of Artificial Sequence Synthetic polynucleotide
gtcgacttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctggatcgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcattttggcaaagaattgtaatacgactcactatagggcgaattcggatccatggacgaagcggatcggcggctcctgcggcggtgccggctgcggctggtggaagagctgcaggtggaccagctctgggacgccctgctgagccgcgagctgttcaggccccatatgatcgaggacatccagcgggcaggctctggatctcggcgggatcaggccaggcagctgatcatagatctggagactcgagggagtcaggctcttcctttgttcatctcctgcttagaggacacaggccaggacatgctggcttcgtttctgcgaactaacaggcaagcagcaaagttgtcgaagccaaccctagaaaaccttaccccagtggtgctcagaccagagattcgcaaaccagaggttctcagaccggaaacacccagaccagtggacattggttctggaggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcagcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctctggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcataaagatcttattaaagcagaacttgtttattgcagcttataatggttacaaataaagcaatagcatcacaaatttcacaaataaagcatttttttcactgcattctagttgtggtttgtccaaactcatcaatgtatcttatcatgtctggtcgactctagactcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcttttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaatt

SEQ ID NO: 83 - protein, 416 aa, Homo sapiens
MDEADRRLLRRCRLRLVEELQVDQLWDALLSRELFRPHMIEDIQRAGSGSRRDQARQLIIDLETRGSQALPLFISCLEDTGQDMLASFLRTNRQAAKLSKPTLENLTPVVLRPEIRKPEVLRPETPRPVDIGSGGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQASGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 84 - DNA, 1819 bp, Mus sp.
gaattccgggctggattgagaagccgcaactgtgactctgcatcatgaatactctgtctgaaggaaatggcacctttgccatccatcttttgaagatgctatgtcaaagcaacccttccaaaaatgtatgttattctcctgcgagcatctcctctgctctagctatggttctcttgggtgcaaagggacagacggcagtccagatatctcaggcacttggtttgaataaagaggaaggcatccatcagggtttccagttgcttctcaggaagctgaacaagccagacagaaagtactctcttagagtggccaacaggctctttgcagacaaaacttgtgaagtcctccaaacctttaaggagtcctctcttcacttctatgactcagagatggagcagctctcctttgctgaagaagcagaggtgtccaggcaacacataaacacatgggtctccaaacaaactgaaggtaaaattccagagttgttgtcaggtggctccgtcgattcagaaaccaggctggttctcatcaatgccttatattttaaaggaaagtggcatcaaccatttaacaaagagtacacaatggacatgccctttaaaataaacaaggatgagaaaaggccagtgcagatgatgtgtcgtgaagacacatataacctcgcctatgtgaaggaggtgcaggcgcaagtgctggtgatgccatatgaaggaatggagctgagcttggtggttctgctcccagatgagggtgtggacctcagcaaggtggaaaacaatctcacttttgagaagttaacagcctggatggaagcagattttatgaagagcactgatgttgaggttttccttccaaaatttaaactccaagaggattatgacatggagtctctgtttcagcgcttgggagtggtggatgtcttccaagaggacaaggctgacttatcaggaatgtctccagagagaaacctgtgtgtgtccaagtttgttcaccagagtgtagtggagatcaatgaggaaggcacagaggctgcagcagcctctgccatcatagaattttgctgtgcctcttctgtcccaacattctgtgctgaccaccccttccttttcttcatcaggcacaacaaagcaaacagcatcctgttctgtggcaggttctcatctccataaagacacatatactacacagggagagttctctcttcagtatccctaccactcctacagctctgtcaagatgggcaagtagggggaagtcatgttctaagatgaagacactttccttctctgtcagcctgatcttataatgcctgcattcaactctccctgtcttgaatgcatctatgccctttaccaggttatgtctaatgatgccaaataccttctgctatgctattgattgatagcctagccagtaatttatagccagttagaactgacttgactgtgcaagaatgctataatggagctagagagaaggcacaaacactaggaaaggttgctgtttttgcagaggacacagggacatttcccaccactcacatggctgcttacaacctctggaaattccagtttctgtccatgacttgattcctttctttggcttctactggctccagcatcctgcacatacatgtatcgtcattcagttacacacaaacaagtaaaattttaaaaataaataaaaatttaaagagagagtctaaaattttagtaatggttagataatagctgctattgtgcctttttcaggttttaatgtcattattcttgtgtataaagtcaataatttataggaaaacatcagtgccccggaattc

SEQ ID NO: 85 - protein, 374 aa, Mus sp.
MNTLSEGNGTFAIHLLKMLCQSNPSKNVCYSPASISSALAMVLLGAKGQTAVQISQALGLNKEEGIHQGFQLLLRKLNKPDRKYSLRVANRLFADKTCEVLQTFKESSLHFYDSEMEQLSFAEEAEVSRQHINTWVSKQTEGKIPELLSGGSVDSETRLVLINALYFKGKWHQPFMKEYTMDMPFKINKDEKRPVQMMCREDTYNLAYVKEVQAQVLVMPYEGMELSLVVLLPDEGVDLSKVENNLTFEKLTAWMEADFMKSTDVEVFLPKFKLQEDYDMESLFQRLGVVDVFQEDKADLSGMSPERNLCVSKFVHQSVVEINEEGTEAAAASAIIEFCCASSVPTFCADHPFLFFIRHNKANSILFCGRFSSP

SEQ ID NO: 86 - DNA, 1125 bp, Mus sp.
atgaatactctgtctgaaggaaatggcacctttgccatccatcttttgaagatgctatgtcaaagcaacccttccaaaaatgtatgttattctcctgcgagcatctcctctgctctagctatggttctcttgggtgcaaagggacagacggcagtccagatatctcaggcacttggtttgaataaagaggaaggcatccatcagggtttccagttgcttctcaggaagctgaacaagccagacagaaagtactctcttagagtggccaacaggctctttgcagacaaaacttgtgaagtcctccaaacctttaaggagtcctctcttcacttctatgactcagagatggagcagctctcctttgctgaagaagcagaggtgtccaggcaacacataaacacatgggtctccaaacaaactgaaggtaaaattccagagttgttgtcaggtggctccgtcgattcagaaaccaggctggttctcatcaatgccttatattttaaaggaaagtggcatcaaccatttaacaaagagtacacaatggacatgccctttaaaataaacaaggatgagaaaaggccagtgcagatgatgtgtcgtgaagacacatataacctcgcctatgtgaaggaggtgcaggcgcaagtgctggtgatgccatatgaaggaatggagctgagcttggtggttctgctcccagatgagggtgtggacctcagcaaggtggaaaacaatctcacttttgagaagttaacagcctggatggaagcagattttatgaagagcactgatgttgaggttttccttccaaaatttaaactccaagaggattatgacatggagtctctgtttcagcgcttgggagtggtggatgtcttccaagaggacaaggctgacttatcaggaatgtctccagagagaaacctgtgtgtgtccaagtttgttcaccagagtgtagtggagatcaatgaggaaggcagagaggctgcagcagcctctgccatcatagaattttgctgtgcctcttctgtcccaacattctgtgctgaccaccccttccttttcttcatcaggcacaacaaagcaaacagcatcctgttctgtggcaggttctcatctccataa

SEQ ID NO: 87 - protein, 374 aa, Mus sp.
MNTLSEGNGTFAIHLLKMLCQSNPSKNVCYSPASISSALAMVLLGAKGQTAVQISQALGLNKEEGIHQGFQLLLRKLNKPDRKYSLRVANRLFADKTCEVLQTFKESSLHFYDSEMEQLSFAEEAEVSRQHINTWVSKQTEGKIPELLSGGSVDSETRLVLINALYFKGKWHQPFNKEYTMDMPFKINKDEKRPVQMMCREDTYNLAYVKEVQAQVLVMPYEGMELSLVVLLPDEGVDLSKVENNLTFEKLTAWMEADFMKSTDVEVFLPKFKLQEDYDMESLFQRLGVVDVFQEDKADLSGMSPERNLCVSKFVHQSVVEINEEGREAAAASAIIEFCCASSVPTFCADHPFLFFIRHNKANSILFCGRFSSP

SEQ ID NO: 88 - DNA, 6539 bp, Artificial Sequence
  Description of Artificial Sequence Synthetic polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccactgtgctggatatctgcagaattcatgaatactctgtctgaaggaaatggcacctttgccatccatcttttgaagatgctatgtcaaagcaacccttccaaaaatgtatgttattctcctgcgagcatctcctctgctctagctatggttctcttgggtgcaaagggacagacggcagtccagatatctcaggcacttggtttgaataaagaggaaggcatccatcagggtttccagttgcttctcaggaagctgaacaagccagacagaaagtactctcttagagtggccaacaggctctttgcagacaaaacttgtgaagtcctccaaacctttaaggagtcctctcttcacttctatgactcagagatggagcagctctcctttgctgaagaagcagaggtgtccaggcaacacataaacacatgggtctccaaacaaactgaaggtaaaattccagagttgttgtcaggtggctccgtcgattcagaaaccaggctggttctcatcaatgccttatattttaaaggaaagtggcatcaaccatttaacaaagagtacacaatggacatgccctttaaaataaacaaggatgagaaaaggccagtgcagatgatgtgtcgtgaagacacatataacctcgcctatgtgaaggaggtgcaggcgcaagtgctggtgatgccatatgaaggaatggagctgagcttggtggttctgctcccagatgagggtgtggacctcagcaaggtggaaaacaatctcacttttgagaagttaacagcctggatggaagcagattttatgaagagcactgatgttgaggttttccttccaaaatttaaactccaagaggattatgacatggagtctctgtttcagcgcttgggagtggtggatgtcttccaagaggacaaggctgacttatcaggaatgtctccagagagaaacctgtgtgtgtccaagtttgttcaccagagtgtagtggagatcaatgaggaaggcacagaggctgcagcagcctctgccatcatagaattttgctgtgcctcttctgtcccaacattctgtgctgaccaccccttccttttcttcatcaggcacaacaaagcaaacagcatcctgttctgtggcaggttctcatctccataaggatccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 89 - DNA, 6539 bp, Artificial Sequence
  Description of Artificial Sequence Synthetic polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccactgtgctggatatctgcagaattcatgaatactctgtctgaaggaaatggcacctttgccatccatcttttgaagatgctatgtcaaagcaacccttccaaaaatgtatgttattctcctgcgagcatctcctctgctctagctatggttctcttgggtgcaaagggacagacggcagtccagatatctcaggcacttggtttgaataaagaggaaggcatccatcagggtttccagttgcttctcaggaagctgaacaagccagacagaaagtactctcttagagtggccaacaggctctttgcagacaaaacttgtgaagtcctccaaacctttaaggagtcctctcttcacttctatgactcagagatggagcagctctcctttgctgaagaagcagaggtgtccaggcaacacataaacacatgggtctccaaacaaactgaaggtaaaattccagagttgttgtcaggtggctccgtcgattcagaaaccaggctggttctcatcaatgccttatattttaaaggaaagtggcatcaaccatttaacaaagagtacacaatggacatgccctttaaaataaacaaggatgagaaaaggccagtgcagatgatgtgtcgtgaagacacatataacctcgcctatgtgaaggaggtgcaggcgcaagtgctggtgatgccatatgaaggaatggagctgagcttggtggttctgctcccagatgagggtgtggacctcagcaaggtggaaaacaatctcacttttgagaagttaacagcctggatggaagcagattttatgaagagcactgatgttgaggttttccttccaaaatttaaactccaagaggattatgacatggagtctctgtttcagcgcttgggagtggtggatgtcttccaagaggacaaggctgacttatcaggaatgtctccagagagaaacctgtgtgtgtccaagtttgttcaccagagtgtagtggagatcaatgaggaaggcagagaggctgcagcagcctctgccatcatagaattttgctgtgcctcttctgtcccaacattctgtgctgaccaccccttccttttcttcatcaggcacaacaaagcaaacagcatcctgttctgtggcaggttctcatctccataaggatccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 90 - DNA, 810 bp, Homo sapiens
atggatgaccagcgcgaccttatctccaacaatgagcaactgcccatgctgggccggcgccctggggccccggagagcaagtgcagccgcggagccctgtacacaggcttttccatcctggtgactctgctcctcgctggccaggccaccaccgcctacttcctgtaccagcagcagggccggctggacaaactgacagtcacctcccagaacctgcagctggagaacctgcgcatgaagcttgccaagttcgtggctgcctggaccctgaaggctgccgctgccctgccccaggggcccatgcagaatgccaccaagtatggcaacatgacagaggaccatgtgatgcacctgctccagaatgctgaccccctgaaggtgtacccgccactgaaggggagcttcccggagaacctgagacaccttaagaacaccatggagaccatagactggaaggtctttgagagctggatgcaccattggctcctgtttgaaatgagcaggcactccttggagcaaaagcccactgacgctccaccgaaagtactgaccaagtgccaggaagaggtcagccacatccctgctgtccacccgggttcattcaggcccaagtgcgacgagaacggcaactatctgccactccagtgctatgggagcatcggctactgctggtgtgtcttccccaacggcacggaggtccccaacaccagaagccgcgggcaccataactgcagtgagtcactggaactggaggacccgtcttctgggctgggtgtgaccaagcaggatctgggcccagtccccatgtga

SEQ ID NO: 91 - protein, 269 aa, Homo sapiens
MDDQRDLISNNEQLPMLGRRPGAPESKCSRGALYTGFSILVTLLLAGQATTAYFLYQQQGRLDKLTVTSQNLQLENLRMKLAKFVAAWTLKAAAALPQGPMQNATKYGNMTEDHVMHLLQNADPLKVYPPLKGSFPENLRHLKNTMETIDWKVFESWMHHWLLFEMSRHSLEQKPTDAPPKVLTKCQEEVSHIPAVHPGSFRPKCDENGNYLPLQCYGSIGYCWCVFPNGTEVPNTRSRGHHNCSESLELEDPSSGLGVTKQDLGPVPM

SEQ ID NO: 92 - protein, 17 aa, Mus sp.
KPVSQMRMATPLLMRPM

SEQ ID NO: 93 - protein, 13 aa, Artificial Sequence
  Description of Artificial Sequence Synthetic peptide
AKFVAAWTLKAAA

SEQ ID NO: 94 - DNA, 3392 bp, Homo sapiens
atgcgttgcctggctccacgccctgctgggtcctacctgtcagagccccaaggcagctcacagtgtgccaccatggagttggggcccctagaaggtggctacctggagcttcttaacagcgatgctgacccctgtgcctctaccacttctatgaccagatggacctggctggagaagaagagattgagctctactcagaacccgacacagacaccatcaactgcgaccagttcagcaggctgttgtgtgacatggaaggtgatgaagagaccagggaggcttatgccaatatcgcggaactggaccagtatgtcttccaggactcccagctggagggcctgagcaaggacattttcaagcacataggaccagatgaagtgatcggtgagagtatggagatgccagcagaagttgggcagaaaagtcagaaaagacccttcccagaggagcttccggcagacctgaagcactggaagccagctgagccccccactgtggtgactggcagtctcctagtgggaccagtgagcgactgctccaccctgccctgcctgccactgcctgcgctgttcaaccaggagccagcctccggccagatgcgcctggagaaaaccgaccagattcccatgcctttctccagttcctcgttgagctgcctgaatctccctgagggacccatccagtttgtccccaccatctccactctgccccatgggctctggcaaatctctgaggctggaacaggggtctccagtatattcatctaccatggtgaggtgccccaggccagccaagtaccccctcccagtggattcactgtccacggcctcccaacatctccagaccggccaggctccaccagccccttcgctccatcagccactgacctgcccagcatgcctgaacctgccctgacctcccgagcaaacatgacagagcacaagacgtcccccacccaatgcccggcagctggagaggtctccaacaagcttccaaaatggcctgagccggtggagcagttctaccgctcactgcaggacacgtatggtgccgagcccgcaggcccggatggcatcctagtggaggtggatctggtgcaggccaggctggagaggagcagcagcaagagcctggagcgggaactggccaccccggactgggcagaacggcagctggcccaaggaggcctggctgaggtgctgttggctgccaaggagcaccggcggccgcgtgagacacgagtgattgctgtgctgggcaaagctggtcagggcaagagctattgggctggggcagtgagccgggcctgggcttgtggccggcttccccagtacgactttgtcttctctgtcccctgccattgcttgaaccgtccgggggatgcctatggcctgcaggatctgctcttctccctgggcccacagccactcgtggcggccgatgaggttttcagccacatcttgaagagacctgaccgcgttctgctcatcctagacggcttcgaggagctggaagcgcaagatggcttcctgcacagcacgtgcggaccggcaccggcggagccctgctccctccgggggctgctggccggccttttccagaagaagctgctccgaggttgcaccctcctcctcacagcccggccccggggccgcctggtccagagcctgagcaaggccgacgccctatttgagctgtccggcttctccatggagcaggcccaggcatacgtgatgcgctactttgagagctcagggatgacagagcaccaagacagagccctgacgctcctccgggaccggccacttcttctcagtcacagccacagccctactttgtgccgggcagtgtgccagctctcagaggccctgctggagcttggggaggacgccaagctgccctccacgctcacgggactctatgtcggcctgctgggccgtgcagccctcgacagcccccccggggccctggcagagctggccaagctggcctgggagctgggccgcagacatcaaagtaccctacaggaggaccagttcccatccgcagacgtgaggacctgggcgatggccaaaggcttagtccaacacccaccgcgggccgcagagtccgagctggccttccccagcttcctcctgcaatgcttcctgggggccctgtggctggctctgagtggcgaaatcaaggacaaggagctcccgcagtacctagcattgaccccaaggaagaagaggccctatgacaactggctggagggcgtgccacgctttctggctgggctgatcttccagcctcccgcccgctgcctgggagccatactcgggccatcggcggctgcctcggtggacaggaagcagaaggtgcttgcgaggtacctgaagcggctgcagccggggacactgcgggcgcggcagctgctggagctgctgcactgcgcccacgaggccgaggaggctggaatttggcagcacgtggtacaggagctccccggccgcctctcttttctgggcacccgcctcacgcctcctgatgcacatgtactgggcaaggccttggaggcggcgggccaagacttctccctggacctccgcagcactggcatttgcccctctggattggggagcctcgtgggactcagctgtgtcacccgtttcagggctgccttgagcgacacggtggcgctgtgggagtccctgcagcagcatggggagaccaagctacttcaggcagcagaggagaagttcaccatcgagcctttcaaagccaagtccctgaaggatgtggaagacctgggaaagcttgtgcagactcagaggacgagaagttcctcggaagacacagctggggagctccctgctgttcgggacctaaagaaactggagtttgcgctgggccctgtctcaggcccccaggctttccccaaactggtgcggatcctcacggccttttcctccctgcagcatctggacctggatgcgctgagtgagaacaagatcggggacgagggtgtctcgcagctctcagccaccttcccccagctgaagtccttggaaaccctcaatctgtcccagaacaacatcactgacctgggtgcctacaaactcgccgaggccctgccttcgctcgctgcatccctgctcaggctaagcttgtacaataactgcatctgcgacgtgggagccgagagcttggctcgtgtgcttccggacatggtgtccctccgggtgatggacgtccagtacaacaagttcacggctgccggggcccagcagctcgctgccagccttcggaggtgtcctcatgtggagacgctggcgatgtggacgcccaccatcccattcagtgtccaggaacacctgcaacaacaggattcacggatcagcctgagatga

SEQ ID NO: 95 - protein, 1130 aa, Homo sapiens
MRCLAPRPAGSYLSEPQGSSQCATMELGPLEGGYLELLNSDADPLCLYHFYDQMDLAGEEEIELYSEPDTDTINCDQFSRLLCDMEGDEETREAYANIAELDQYVFQDSQLEGLSKDIFKHIGPDEVIGESMEMPAEVGQKSQKRPFPEELPADLKHWKPAEPPTVVTGSLLVGPVSDCSTLPCLPLPALFNQEPASGQMRLEKTDQIPMPFSSSSLSCLNLPEGPIQFVPTISTLPHGLWQISEAGTGVSSIFIYHGEVPQASQVPPPSGFTVHGLPTSPDRPGSTSPFAPSATDLPSMPEPALTSRANMTEHKTSPTQCPAAGEVSNKLPKWPEPVEQFYRSLQDTYGAEPAGPDGILVEVDLVQARLERSSSKSLERELATPDWAERQLAQGGLAEVLLAAKEHRRPRETRVIAVLGKAGQGKSYWAGAVSRAWACGRLPQYDFVFSVPCHCLNRPGDAYGLQDLLFSLGPQPLVAADEVFSHILKRPDRVLLILDGFEELEAQDGFLHSTCGPAPAEPCSLRGLLAGLFQKKLLRGCTLLLTARPRGRLVQSLSKADALFELSGFSMEQAQAYVMRYFESSGMTEHQDRALTLLRDRPLLLSHSHSPTLCRAVCQLSEALLELGEDAKLPSTLTGLYVGLLGRAALDSPPGALAELAKLAWELGRRHQSTLQEDQFPSADVRTWAMAKGLVQHPPRAAESELAFPSFLLQCFLGALWLALSGEIKDKELPQYLALTPRKKRPYDNWLEGVPRFLAGLIFQPPARCLGALLGPSAAASVDRKQKVLARYLKRLQPGTLRARQLLELLHCAHEAEEAGIWQHVVQELPGRLSFLGTRLTPPDAHVLGKALEAAGQDFSLDLRSTGICPSGLGSLVGLSCVTRFRAALSDTVALWESLQQHGETKLLQAAEEKFTIEPFKAKSLKDVEDLGKLVQTQRTRSSSEDTAGELPAVRDLKKLEFALGPVSGPQAFPKLVRILTAFSSLQHLDLDALSENKIGDEGVSQLSATFPQLKSLETLNLSQNNITDLGAYKLAEALPSLAASLLRLSLYNNCICDVGAESLARVLPDMVSLRVMDVQYNKFTAAGAQQLAASLRRCPHVETLAMWTPTIPFSVQEHLQQQDSRISLR

SEQ ID NO: 96 - protein, 17 aa, Human herpesvirus
YLQQNWWTLLVDLLWLL

SEQ ID NO: 97 - protein, 16 aa, Homo sapiens
IGHVYIFATCLGLSYD

SEQ ID NO: 98 - protein, 8 aa, Mycoplasma penetrans
IYIFAACL

SEQ ID NO: 99 - protein, 15 aa, Unknown
  Description of Unknown Six-transmembrane epithelial antigen of prostate peptide
HQQYFYKIPILVINK

SEQ ID NO: 100 - protein, 15 aa, Unknown
  Description of Unknown Six-transmembrane epithelial antigen of prostate peptide
LLNWAYQQVQQNKED

SEQ ID NO: 101 - protein, 14 aa, Homo sapiens
EFHACWPAFTVLGE

SEQ ID NO: 102 - protein, 15 aa, Homo sapiens
WQPFLKDHRISTFKN

SEQ ID NO: 103 - protein, 15 aa, Human papillomavirus
LFVVYRDSIPHAACH

SEQ ID NO: 104 - protein, 15 aa, Human papillomavirus
GLYNLLIRCLRCQKP

SEQ ID NO: 105 - protein, 13 aa, Homo sapiens
LWWVNNQSLPVSP

SEQ ID NO: 106 - protein, 25 aa, Mycobacterium tuberculosis
FSKLPASTIDELKTNSSLLTSILTY

SEQ ID NO: 107 - protein, 28 aa, Mycobacterium tuberculosis
GNADVVCGGVSTANATVYMIDSVLMPPA

SEQ ID NO: 108 - protein, 13 aa, Homo sapiens
GSPYVSRLLGICL

SEQ ID NO: 109 - protein, 17 aa, Homo sapiens
KVPIKWMALESILRRRF

SEQ ID NO: 110 - protein, 25 aa, Homo sapiens
PGVLLKEFTVSGNILTIRLTAADHR

SEQ ID NO: 111 - protein, 15 aa, Clostridium tetani
VSIDKFRIFCKANPK

SEQ ID NO: 112 - protein, 16 aa, Clostridium tetani
LKFIIKRYTPNNEIDS

SEQ ID NO: 113 - protein, 15 aa, Clostridium tetani
IREDNNITLKLDRCN

SEQ ID NO: 114 - protein, 21 aa, Clostridium tetani
FNNFTVSFWLRVPKVSASHLE

SEQ ID NO: 115 - protein, 14 aa, Clostridium tetani
QYIKANSKFIGITE

SEQ ID NO: 116 - protein, 20 aa, Hepatitis B virus
PHHTALRQAILCWGELMTLA

SEQ ID NO: 117 - protein, 13 aa, Influenza A virus
PKYVKQNTLKLAT

SEQ ID NO: 118 - protein, 15 aa, Hepatitis B virus
FFLLTRILTIPQSLD

SEQ ID NO: 119 - protein, 16 aa, Influenza A virus
YSGPLKAEIAQRLEDV

SEQ ID NO: 120 - protein, 18 aa, Plasmodium falciparum
EKKIAKMEKASSVFNVVN

SEQ ID NO: 121 - protein, 4 aa, Human papillomavirus
QDKL

SEQ ID NO: 122 - protein, 8 aa, Artificial Sequence
  Description of Artificial Sequence Synthetic peptide
VYDFFVWL

SEQ ID NO: 123 - DNA, 48 bp, Artificial Sequence
  Description of Artificial Sequence Synthetic oligonucleotide
ccggtttgtatgctgtgtatgacttttttgtgtggctcggaggaggtg

SEQ ID NO: 124 - DNA, 48 bp, Artificial Sequence
  Description of Artificial Sequence Synthetic oligonucleotide
ctagcacctcctccgagccacacaaaaaagtcatacacagcatacaaa

SEQ ID NO: 125 - DNA, 30 bp, Artificial Sequence
  Description of Artificial Sequence Synthetic primer
aaagaattcatggatgaccaacgcgacctc

SEQ ID NO: 126 - DNA, 30 bp, Artificial Sequence
  Description of Artificial Sequence Synthetic primer
aaaggatcctcacagggtgacttgacccag

SEQ ID NO: 127 - DNA, 42 bp, Artificial Sequence
  Description of Artificial Sequence Synthetic primer
tccaggcagccacgaacttggcaagcttcatgcgaaggctct

SEQ ID NO: 128 - DNA, 42 bp, Artificial Sequence
  Description of Artificial Sequence Synthetic primer
ctggaccctgaaggctgccgctatggataacatgctccttgg

SEQ ID NO: 129 - DNA, 39 bp, Artificial Sequence
  Description of Artificial Sequence Synthetic primer
gccaagttcgtggctgcctggaccctgaaggctgccgct

SEQ ID NO: 130 - DNA, 29 bp, Artificial Sequence
  Description of Artificial Sequence Synthetic primer
aaatctagaatggcggcccccggcgcccg

SEQ ID NO: 131 - DNA, 29 bp, Artificial Sequence
  Description of Artificial Sequence Synthetic primer
ggggaattctagatcctcaaagagtgctg

SEQ ID NO: 132 - DNA, 48 bp, Artificial Sequence
  Description of Artificial Sequence Synthetic oligonucleotide
aattcgccaagttcgtggctgcctggaccctgaaggctgccgcttgaa

SEQ ID NO: 133 - DNA, 48 bp, Artificial Sequence
  Description of Artificial Sequence Synthetic oligonucleotide
agctttcaagcggcagccttcagggtccaggcagccacgaacttggcg

SEQ ID NO: 134 - DNA, 69 bp, Artificial Sequence
  Description of Artificial Sequence Synthetic primer
aaagaattcgccaagttcgtggctgcctggaccctgaaggctgccgctcttaacaacatgttgatcccc

SEQ ID NO: 135 - DNA, 29 bp, Artificial Sequence
  Description of Artificial Sequence Synthetic primer
tttggatccctagatggtctgatagccgg

SEQ ID NO: 136 - protein, 9 aa, Human papillomavirus
RAHYNIVTF

SEQ ID NO: 137 - protein, 17 aa, Gallus gallus
ISQAVHAAHAEINEAGR

SEQ ID NO: 138 - protein, 8 aa, Human papillomavirus
YDFAFRDL

SEQ ID NO: 139 - protein, 386 aa, Gallus gallus
MGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKTFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 140 - protein, 26 aa, Human papillomavirus
LSRHFMHQKRTAMFQDPQERPRKLPQ

SEQ ID NO: 141 - protein, 31 aa, Human papillomavirus
AMFQDPQERPRKLPQLCTELQTTIHDIILEC

SEQ ID NO: 142 - protein, 22 aa, Human papillomavirus
PTLHEYMLDLQPETTDLYCYEQ

SEQ ID NO: 143 - protein, 11 aa, Human papillomavirus
HEYMLDLQPET

SEQ ID NO: 144 - protein, 15 aa, Human papillomavirus
TLHEYMLDLQPETTD

SEQ ID NO: 145 - protein, 14 aa, Human papillomavirus
EYMLDLQPETTDLY

SEQ ID NO: 146 - protein, 17 aa, Human papillomavirus
DEIDGPAGQAEPDRAHY

SEQ ID NO: 147 - protein, 15 aa, Human papillomavirus
GPAGQAEPDRAHYNI

SEQ ID NO: 148 - protein, 4 aa, Artificial Sequence
  Description of Artificial Sequence Synthetic peptide
QDKL

SEQ ID NO: 149 - protein, 3 aa, Artificial Sequence
  Description of Artificial Sequence Synthetic peptide
QKP

SEQ ID NO: 150 - DNA, 9 bp, Artificial Sequence
  Description of Artificial Sequence Synthetic primer
cagaaacca

SEQ ID NO: 151 - protein, 4 aa, Human papillomavirus
  MOD_RES (2)..(3): Any amino acid
CXXC

SEQ ID NO: 152 - protein, 8 aa, Artificial Sequence
  Description of Artificial Sequence Synthetic peptide
SIINFEKL

SEQ ID NO: 153 - protein, 17 aa, Artificial Sequence
  Description of Artificial Sequence Synthetic peptide
LSQAVHAAHAEINEAGR

(19) United States
(12) Patent Application Publication
(10) Pub. No.: US 2015/0182621 A1
(43) Pub. Date: Jul. 2, 2015

(54) COMPOSITIONS AND METHODS FOR ENHANCING ANTIGEN-SPECIFIC IMMUNE RESPONSES

(71) Applicant: The Johns Hopkins University, Baltimore, MD (US)

(72) Inventors: Tzyy-Choou Wu, Stevenson, MD (US); Chien-Fu Hung, Timonium, MD (US); Richard Roden, Severna Park, MD (US)

(21) Appl. No.: 14/453,313

(22) Filed: Aug. 6, 2014

Related U.S. Application Data
(63) Continuation-in-part of application No. 13/318,028, filed on Jan. 20, 2012.
(60) Provisional application No. 61/173,413, filed on Apr. 28, 2009.

(51) Int. Cl. A61K 39/39; A61K 39/12; C12N 7/00; A61K 39/285

(52) U.S. Cl. CPC A61K 39/39; A61K 39/285; A61K 39/12; C12N 7/00; A61K 2039/55561

(57) ABSTRACT

Methods for treating or preventing recurrence of hyper proliferating diseases, e.g., cancer and persistent viral infections, are described. A method may comprise priming a mammal by administering to the mammal an effective amount of a nucleic acid composition encoding an antigen or a biologically active homolog thereof and boosting the mammal by administering to the mammal an effective amount of an oncolytic virus comprising a nucleic acid encoding the antigen or the biologically active homolog thereof.

### CROSS-REFERENCE TO RELATED APPLICATIONS

This application is a continuation-in-part of U.S. application Ser. No. 13/318,028, filed on Apr. 28, 2010, which claims the benefit of U.S. Provisional Application No. 61/173,413, filed on Apr. 28, 2009, the content of which is specifically incorporated by reference herein in its entirety.

### GOVERNMENTAL SUPPORT

This invention was made with government support under grant numbers P50 CA 098252 and RO1 CA 114425, awarded by the U.S. National Cancer Institute. The government has certain rights in this invention.

### BACKGROUND

Cancer immunotherapeutics have shown promise for the treatment of a number of tumors and hyper proliferative diseases, but their utility is limited in situations where the tumor is relatively large or rapidly growing. For example, advanced stage cancers are extremely difficult to treat and rarely result in a cure. Efforts to improve early detection and treatment of advanced stage cancers have been relatively unsuccessful. Existing therapies for advanced disease, such as chemotherapy and radiation therapy, have not improved the overall survival of patients with locally advanced or metastatic disease (Early Breast Cancer Trialists' Collaborative Group, Lancet, 339:1-15 (1992); Baum et al., Salmon S E, ed., Adjuvant therapy of cancer V1. Philadelphia: WB. Saunders, 269-74 (1990); Swain, S. M., Surg. Clin. North Am., 70:1061-80 (1990)). Therefore, there is a strong need to develop innovative therapeutic approaches for the control of hyper proliferative diseases, particularly if they have progressed to an advanced stage.

### SUMMARY OF THE INVENTION

In one embodiment, the invention is directed, at least in part, to a method of inducing or enhancing an antigen-specific immune response in a mammal, comprising the steps of: (a) priming the mammal by systemic administration to the mammal, e.g. via intra muscular injection, of an effective amount of a nucleic acid composition encoding the antigen or a biologically active homolog thereof; and (b) boosting the mammal by administering to the mammal an effective amount of an oncolytic virus comprising a nucleic acid encoding the antigen or the biologically active homolog thereof, thereby inducing or enhancing the antigen-specific immune response. In some embodiments, the antigen is a tumor-associated antigen (TAA), foreign to the mammal, and/or includes ovalbumin, HPV E6, and HPV E7, or “detoxified” versions of E6 and/or E7 containing inactivating mutations. In yet another embodiment, the antigen comprises an ovalbumin protein comprising an amino acid sequence at least 90% identical to an amino acid sequence of SEQ ID NO:139. In still other embodiments, the antigen comprises an HPV E7 protein comprising an amino acid sequence at least 90% identical to an amino acid sequence selected from the group consisting of LSRHFMHQKRTAMFQDPQERPRKLPQ and AMFQDPQERPRKLPQLCTELQTTIHDIILEC. In one embodiment, the antigen comprises an HPV E7 protein comprising an amino acid sequence at least 90% identical to an amino acid sequence selected from the group consisting of PTLHEYMLDLQPETTDLYCYEQ, HEYMLDLQPET, TLHEYMLDLQPETTD, EYMLDLQPETTDLY, DEIDGPAGQAEPDRAHY and GPAGQAEPDRAHYNI.

In certain embodiments, the nucleic acid composition is a DNA vaccine. In some embodiments, the nucleic acid composition is administered from the group consisting of intradermally, intraperitoneally, intravaginally, intramuscularly, subcutaneously, intracervically and intravenously. In certain embodiments, the mammal is a human having a tumor and wherein the nucleic acid composition is administered intratumorally or peritumorally. In some embodiments, the oncolytic virus is selected from the group consisting of vaccinia virus (including Wyeth strain and New York strain, and modified vaccinia virus Ankara), adenovirus, herpes simplex virus, poxvirus, vesicular stomatitits virus, measles virus, Newcastle disease virus, influenza virus, and reovirus. In yet another embodiment, the oncolytic virus is thymidine kinase negative. In certain embodiments, the oncolytic virus is administered from the group consisting of intradermally, intraperitoneally, intracervicovaginally, intramuscularly, sucbcutaneously and intravenously or into the genital tract or anal cavity or into the oral cavity or oropharynx or a lymph node. In some embodiments, the mammal is a human having a tumor and wherein the oncolytic virus is administered intratumorally or peritumorally. In still other embodiments, the nucleic acid composition is present within an oncolytic virus. In other embodiments, the oncolytic virus of step (a) is the same as or is different from the oncolytic virus of step (b). In yet other embodiments, step (a) is performed before step (b), step (a) and step (b) are performed at the same time, or step (a) is performed after step (b). In still another embodiment, step (a) and/or step (b) is repeated at least once. In one embodiment, the dosage of oncolytic virus used in step (a) and/or step (b) is a range that includes 1×10̂7 pfu.

In certain embodiments, the nucleic acid composition or oncolytic virus is administered intracervicovaginally. In some embodiments, the oncolytic virus is administered intracervicovaginally. In some embodiments, the nucleic acid composition is administered intramuscularly and the oncolytic virus is administered intracervicovaginally. In other embodiments, both the nucleic acid composition and the oncolytic virus are administered intracervicovaginally.

In certain embodiments, the antigen-specific immune response is greater in magnitude than an antigen-specific immune response induced by administration of the nucleic acid composition alone. In other embodiments, the antigen-specific immune response is mediated at least in part by CD8⁺ cytotoxic T lymphocytes (CTL). In other embodiments, the antigen-specific immune response is mediated at least in part by CD8⁺ cytotoxic T lymphocytes (CTL) and/or peritumoral stromal cells. In yet another embodiment, the method also includes administering an effective amount of a chemotherapeutic agent.

In still other embodiments, the method includes screening the mammal for the presence of antibodies against the antigen. In some embodiments, the mammal is a human. In other embodiments, the mammal is afflicted with cancer.

The instant invention is also directed at least in part to a method for treating or preventing advanced stage cancer in a mammal comprising (a) priming the mammal by administering to the mammal an effective amount of a nucleic acid composition encoding the antigen or a biologically active homolog thereof; and (b) boosting the mammal by administering to the mammal an effective amount of an oncolytic virus comprising a nucleic acid encoding the antigen or the biologically active homolog thereof, thereby inducing or enhancing the antigen-specific immune response. In some embodiments, the advanced stage cancer is a cancer described herein, including melanoma or thymoma. In some embodiments, the cancer is cervical cancer or an HPV E6 or E7 positive cancer.

The instant invention is also directed at least in part to a kit comprising a priming composition and a boosting composition, the kit comprising; (a) a priming composition comprising DNA encoding an immunogenic foreign antigen and a pharmaceutically acceptable carrier; and (b) a boosting composition comprising a virus encoding said foreign antigen and a pharmaceutically acceptable carrier.

### BRIEF DESCRIPTION OF THE DRAWINGS

FIGS. 1A-1B. Luminescence imaging demonstrating vaccinia infection in mice. Groups of C57BL/6 mice (5 per group) were subcutaneously challenged with 5×10⁴/mouse of TC-1 tumor cells. When tumor size reached about 8-10 mm, mice were treated with either i.t. or i.p. injection of Vac-luc at 1×10⁷ pfu/mouse. (A) Representative bioluminescence signal for each group over time. (B) Bar graph depicting the ratios of signal intensity of intratumoral (i.t.) over intraperitoneal (i.p.) administrations in mice treated with Vac-luc over time.

FIGS. 2A-2C. In vivo tumor treatment experiments with B16 tumors. (A) Diagrammatic representation of the prime-boost treatment regimen. Groups of C57BL/6 mice (5 per group) were subcutaneously challenged with 5×10⁴/mouse of B16/F10 tumor cells. 5 days after tumor challenge, mice were immunized with either 2 μg/mouse of pcDNA3 DNA or pcDNA3 expressing ovalbumin (p-OVA) by gene gun. On day 12, mice were boosted by intratumoral injection of 1×10⁷ pfu/mouse of either wild-type vaccinia (Vac-WT) or vaccinia encoding ovalbumin (Vac-OVA). B16 tumor-bearing mice treated with 1×PBS were used as a control. (B) Line graph depicting the tumor volume in B16 tumor bearing mice treated with the different prime-boost regimens. Numbers in parentheses indicate complete tumor rejection rates. (C) Kaplan & Meier survival analysis of B16 tumor bearing mice treated with the different treatment regimens. Data shown are representative of two experiments performed (mean±SD).

FIGS. 3A-3C. In vivo tumor treatment experiments with TC-1 tumors. (A) Diagrammatic representation of the prime-boost treatment regimen. Groups of C57BL/6 mice (5 per group) were subcutaneously challenged with 5×10⁴/mouse of TC-1 tumor cells. 5 days after tumor challenge, mice were immunized with either 2 pig/mouse of pcDNA3 DNA or pcDNA3 expressing ovalbumin (p-OVA) by gene gun. On day 12, mice were boosted by intratumoral injection of 1×10⁷ pfu/mouse of either wild-type vaccinia (Vac-WT) or vaccinia encoding ovalbumin (Vac-OVA). TC-1 tumor-bearing mice treated with 1×PBS were used as a control. (B) Line graph depicting the tumor volume in TC-1 tumor bearing mice treated with the different prime-boost regimens. Numbers in parentheses indicate complete tumor rejection rates. (C) Kaplan & Meier survival analysis of TC-1 tumor bearing mice treated with the different treatment regimens. Data shown are representative of two experiments performed (mean±SD).

FIGS. 4A-4C. In vivo tumor treatment experiments. Groups of C57BL/6 mice (5 per group) were subcutaneously challenged with 5×10⁴/mouse of TC-1 tumor cells. 5 days after tumor challenge, mice were immunized with 2 μg/mouse of pcDNA3 expressing CRT/E7 (p-CRT/E7) by gene gun. On day 12, mice were boosted by intraperitoneal or intratumoral injection of 1×10⁷ pfu/mouse of either wild-type vaccinia (Vac-WT) or vaccinia encoding CRT/E7 (Vac-CRT/E7). TC-1 tumor-bearing mice treated with 1×PBS were used as a control. (A) Diagrammatic representation of the prime-boost treatment regimen. (B) Line graph depicting the tumor volume in TC-1 tumor bearing mice treated with the different prime-boost regimens. Numbers in parentheses indicate complete tumor rejection rates. (C) Kaplan & Meier survival analysis of TC-1 tumor challenged mice treated with the different treatment regimens. * indicates p<0.05. Data shown are representative of two experiments performed (mean±SD).

FIGS. 5A-5D. Intracellular cytokine staining followed by flow cytometry analysis to determine the number of OVA-specific CD8⁺ T cells in tumor-bearing mice treated with the different prime-boost regimens. Groups of C57BL/6 mice (5 per group) were challenged subcutaneously with 5×10⁴/mouse of B16/F10 tumor cells. 5 days after tumor challenge, mice were immunized with either pcDNA3 or p-OVA DNA by gene gun and boosted by intratumoral injection of either Vac-WT or Vac-OVA as shown in FIG. 2. TC-1 tumor-bearing mice treated with PBS were used as a control. 7 days after vaccinia infection, cells from the spleens (A & B) and tumors (C & D) of mice were harvested, incubated overnight with the OVA peptide and stained for CD8 and intracellular IFN-γ and then characterized for OVA-specific CD8⁺ T cells using intracellular IFN-γ staining followed by flow cytometry analysis. A & C. Representative flow cytometry data showing the percentage of OVA-specific IFNγ⁺ CD8⁺ T cells in the (A) spleens and (C) tumors of mice treated with the different prime boost regimens. B & D. Bar graph depicting the numbers of OVA-specific IFN-γ-secreting CD8⁺ T cells per 2×10⁵ pooled cells in the (B) spleens and (D) tumors of treated mice. Data shown are representative of two experiments performed (mean±SD).

FIGS. 6A-6B. Intracellular cytokine staining followed by flow cytometry analysis to determine the number of E7-specific CD8⁺ T cells in tumor-bearing mice treated with the different prime-boost regimens. Groups of C57BL/6 mice (5 per group) were subcutaneously challenged with 5×10⁴/mouse of TC-1 tumor cells. 5 days after tumor challenge, mice were immunized with 2 μg/mouse of pcDNA3 expressing CRT/E7 (p-CRT/E7) by gene gun. On day 12, mice were boosted by intraperitoneal or intratumoral injection of 1×10⁷ pfu/mouse of either wild-type vaccinia (Vac-WT) or vaccinia encoding CRT/E7 (Vac-CRT/E7). TC-1 tumor-bearing mice treated with PBS were used as a control. 7 days after vaccinia infection, cells from the spleens (A) and tumors (B) of mice were harvested and stained for CD8 and intracellular IFN-γ and then characterized for E7-specific CD8⁺ T cells using intracellular IFN-γ staining followed by flow cytometry analysis. Bar graph depicting the numbers of OVA-specific IFN-γ-secreting CD8⁺ T cells per 2×10⁵ pooled cells in the (A) spleens and (B) tumors of treated mice. Data shown are representative of two experiments performed (mean±SD).

FIGS. 7A-7B. Intracellular cytokine staining followed by flow cytometry analysis to determine the number of OVA-specific CD4⁺ T cells in tumor-bearing mice treated with the different prime-boost regimens. Groups of C57BL/6 mice (5 per group) were challenged subcutaneously with 5×10⁴/mouse of B16/F10 tumor cells. 5 days after tumor challenge, mice were immunized with either pcDNA3 or p-OVA DNA by gene gun and boosted by intratumoral injection of either Vac-WT or Vac-OVA as shown in FIG. 2. TC-1 tumor-bearing mice treated with 1×PBS were used as a control. 7 days after vaccinia infection, cells from the spleens (A) and tumors (B) of mice were harvested and stained for CD8 and intracellular IFN-γ and then characterized for OVA-specific CD4⁺ T cells using intracellular IFN-γ staining followed by flow cytometry analysis. Bar graph depicting the numbers of OVA-specific IFN-γ-secreting CD4⁺ T cells per 2×10⁵ pooled cells in the (A) spleens and (B) tumors of treated mice. Data shown are representative of two experiments performed (mean±SD).

FIGS. 8A-8B. In vivo antibody depletion experiments. C57BL/6 mice (5 per group) were subcutaneously challenged with 5×10⁴/mouse of B16/F10 or TC-1 tumor cells. 5 days after tumor challenge, mice were immunized with 2 pg/mouse of pcDNA3 expressing ovalbumin (p-OVA) by gene gun. On day 12, mice were boosted by intratumoral injection of 1×10⁷ pfu/mouse of vaccinia encoding ovalbumin (Vac-OVA). Mice were depleted of CD4⁺ or CD8⁺ T cells using antibodies every alternate day starting from D5 for 3 doses followed by once a week until the end of the experiment. Tumor-bearing mice treated with 1×PBS were used as a control. Kaplan & Meier survival analysis of (A) B 16 or (B) TC-1 tumor bearing mice treated with the different treatment regimens.

FIGS. 9A-9B. In vitro cytotoxicity assay. (A) Schematic diagram of the experimental design for the cytotoxicity assay. Luciferase-expressing TC-1 tumor cells (2×10⁴/well) were added to 96-well plates. 24 hours later, Vac-WT or Vac-OVA (MOI=0.5) was added to each well. 48 hours later, the complete medium was changed and activated OT-1 T cells were added to each well at an E:T ratio of 1:1. Bioluminescence imaging was performed 4 hours later. The degree of CTL-mediated killing of the tumor cells was indicated by the decrease of luminescence activity using the IVIS luminescence imaging system series 200. Bioluminescence signals were acquired for 10 seconds. (B) Representative luminescence images of 96-well plates and bar graphs depicting the luminescence intensity in each well containing tumor cells with different treatments (mean±SD).

FIGS. 10A-10B. Characterization of vaccinia infectivity of CD31⁺ cells in tumor. (A) Flow cytometry data demonstrating the percentage of CD31⁺ cells in the tumor infected with vaccinia. Groups of C57BL/6 mice (5 per group) were subcutaneously challenged with 5×10⁴/mouse of TC-1 tumor cells. When tumor size reached about 8-10 mm, mice were treated with either intratumorally (i.t.) or intraperitoneally (i.p.) with Vac-GFP at 1×10⁷ pfu/mouse. Tumors were harvested 24 hours after virus injection, stained for CD31 and characterized by flow cytometry analysis. (B) Representative bar graphs depicting the number of CD31⁺AAD⁻ cells per 3×10⁵ cells derived from the tumors in the different treatment groups (mean±SD). Cells derived from explanted tumors (2×10⁴/well) were added to 96-well plates. 24 hours later, Vac-WT or Vac-OVA (MOI=0.5) were added to each well and 48 hours later, activated OT-1 T cells (E:T ratio 1:1) were added to each well. 4 hours later, the cells were stained with PE labeled anti-mouse CD31 mAb and FITC labeled 7-AAD and analyzed by flow cytometry analysis.

FIGS. 11A-11B. C57BL/6 mice (5 per group) were intramuscularly (IM) vaccinated with pNGVL4a-sig/E7(detox)/HSP70 DNA (50 μg per mouse) prime followed six days later by TA-HPV (1×10⁷ per mouse) boost intraperitoneally, DNA prime followed by DNA boost, TA-HPV only or received no vaccination. One week after the last immunization, splenocytes were analyzed by flow cytometry. A. Representative flow cytometry data showing the relative number of IFNγ⁺ CD8⁺ E7-specific cells out of in 10⁵ total splenocytes from mice treated with the different prime boost regimens. B. Bar graph depicting flow cytometry results. Values are shown as mean±SD, *p<0.05, **p<0.01, ns, not significant.

FIGS. 12A-12D. C57BL/6 mice (5 per group) were vaccinated with pNGVL4a-sig/E7(detox)/HSP70 (50 μg per mouse) prime followed six days later by TA-HPV (1×10⁷ per mouse) boost with the regimen administered either intracervicovaginally (ICV) or IM. One week after the second immunization, splenocytes and blood were analyzed by flow cytometry. A & C. Representative flow cytometry data showing the percentage of E7⁺ CD8⁺ T cells in the (A) spleen and peripheral blood (C) for mice receiving either no treatment (naïve) or the intracervicovaginal tract administration (ICV) or intramuscular administration (IM) of pNGVL4a-sig/E7(detox)/HSP70 DNA followed six days later by TA-HPV. B & D. Bar graph depicting flow cytometry results obtained from splenocytes (B), or peripheral blood cells (D). Values are shown as mean±SD, *p<0.05, **p<0.01, ns, not significant.

FIGS. 13A-13C. C57BL/6 mice (5 per group) were vaccinated with pNGVL4a-sig/E7(detox)/HSP70 (50 μg per mouse) prime followed six days later by TA-HPV (1×10⁷ per mouse) boost either ICV or IM. One week after the second immunization, cervicovaginal tissues and ILN were analyzed by flow cytometry. A. Bar graph depicting flow cytometry results obtained for CD8⁺ cervicovaginal tract cells. B. Bar graph depicting flow cytometry results obtained for E7-specific cervicovaginal tract cells. C. Bar graph depicting flow cytometry results obtained for E7-specific iliac lymph node cells. Values are shown as mean±SD, *p<0.05, **p<0.01, ns, not significant.

FIGS. 14A-14B. C57BL/6 mice (5 per group) were vaccinated with pNGVL4a-sig/E7(detox)/HSP70 (50 μg per mouse) prime followed six days later by TA-HPV (1×10⁷ per mouse) boost either ICV or IM. One week after the second immunization, cervicovaginal tissues were analyzed by flow cytometry using E7 peptide-loaded tetramer staining. A. Bar graph depicting flow cytometry results obtained for α4β⁺ cervicovaginal tract cells. α4β7 is a surface marker of T cells that binds with MAdCAM-1 in cervicovaginal tissue. B. Bar graph depicting flow cytometry results obtained for CCR9⁺ cervicovaginal tract cells. CCR9 is a chemokine receptor that binds CCL25. Values are shown as mean±SD, *p<0.05, **p<0.01, ns, not significant.

FIGS. 15A-15B. C57BL/6 mice (5 per group) were vaccinated with pNGVL4a-sig/E7(detox)/HSP70 (50 μg per mouse) prime followed six days later by TA-HPV (1×10⁷ per mouse) boost either ICV or IM. One week after the second immunization, cervicovaginal tissues were analyzed by flow cytometry using E7 peptide-loaded tetramer staining. A. Bar graph depicting flow cytometry results obtained for α4β7⁺ iliac lymph node cells. α4β7 is a surface marker of T cells that binds with MAdCAM-1 in cervicovaginal tissue. B. Bar graph depicting flow cytometry results obtained for CCR9⁺ iliac lymph node cells. Values are shown as mean±SD, *p<0.05, **p<0.01, ns, not significant.

FIGS. 16A-16B. C57BL/6 mice (5 per group) were vaccinated with pNGVL4a-sig/E7(detox)/HSP70 (50 μg per mouse) prime followed six days later by TA-HPV (1×10⁷ per mouse) boost by ICV administration. One week after the second immunization, tissue-infiltrating lymphocytes from cervicovaginal tissues and spleens were isolated and analyzed by flow cytometry using CD8, E7 peptide-loaded tetramer and α4β7, CCR9 and CD103 staining. The E7 peptide-loaded tetramer positive CD8+ T cells were gated for further analysis of α4β7, CCR9 and CD103 expression. A. Representative flow cytometry data showing the number of α4β7⁺, CCR9⁺, CD103⁺ cells in the spleen and cervicovaginal tract. B. Bar graph depicting flow cytometry results showing the percentage of α4β7⁺, CCR9⁺, CD103⁺ cells among E7-specific CD8+ T cells in the spleen and cervicovaginal tract. Values are shown as mean±SD, *p<0.05, **p<0.01.

FIGS. 17A-17D. C57BL/6 mice (5 per group) were challenged with luciferase-expressing TC-1 cells (2×10⁴ per mouse) in the submucosa of the cervicovaginal tract. One day later, mice were immunized with pNGVL4a-sig/E7(detox)/HSP70 and 6 days later, mice were immunized with TA-HPV either IM or ICV. The signal in the cervicovaginal tract was monitored by luminescence on day 7 and day 14 after injection of TC-1 luciferase-expressing cells. A. Luminescence images of representative mice challenged with luciferase-expressing TC-1 tumor and treated according to the various treatment regimens. B. Bar graph showing luminescence intensity, values are shown as mean±SD, *p<0.05, **p<0.01, ns, not significant. C. Kaplan-Meier survival analysis of mice in various treatment groups. D The graph in D shows luminescence activity.

FIGS. 18A-18D. C57BL/6 mice (5 per group) were vaccinated with pNGVL4a-sig/E7(detox)/HSP70 (50 μg per mouse) twice with 7 day interval intramuscularly or intracervicovaginally, followed by TA-HPV boost intramuscularly or intracervicovaginally 7 days after the second DNA vaccination. 7 days after the last immunization, mice were sacrificed and splenocytes and cervicovaginal cells were isolated and analyzed by flow cytometry. A. Representative flow cytometry analysis and B, Bar graph showing the number of E7-specific CD8+ T cells in splenocytes. C, Representative flow cytometry and D, Bar graph showing the number of E7-specific CD8+ T cells in the cervicovaginal cells. Values are shown as mean±SD, *p<0.05, **p<0.01, ns, not significant.

### DETAILED DESCRIPTION

### Partial List of Abbreviations

7-AAD, 7-Aminoactinomycin D; Abs, antibodies; ANOVA, analysis of variance; APC, antigen presenting cell; CRT, calreticulin; CTL, cytotoxic T lymphocyte; DC, dendritic cell; E6, HPV oncoprotein E6; E7, HPV oncoprotein E7; ELISA, enzyme-linked immunosorbent assay; FACS, fluorescence-activated cell sorting; FBS, fetal bovine serum; FITC, fluorescein isothiocyanate; HPV, human papillomavirus; IFN y, interferon-γ; i.m. or IM, intramuscular(ly); i.t., intratumoral(ly); i.v., intravenous(ly); ICV, intracervicoviginal(ly); luc, luciferase; mAB, monoclonal antibody; MOI, multiplicity of infection; OVA, ovalbumin; p-, plasmid-; PBS, phosphate-buffered saline; PCR, polymerase chain reaction; pfu, plaque-forming unit; RPMI, Roswell Park Memorial Institute 1640 tissue culture medium; SD, standard deviation; TAA, tumor-associate antigen; Vac, vaccinia virus; WT, wild-type.

Provided herein are methods and compositions for increasing or stimulating an immune response, e.g., for treating and/or preventing recurrence of a hyper proliferating disease, e.g., cancer, or treating and/or preventing a persistent viral infection. In one embodiment, a method comprises priming a mammal by administering to the mammal an effective amount of a composition, including a nucleic acid composition, encoding an antigen or a biologically active homolog thereof and boosting the mammal by administering to the mammal an effective amount of an oncolytic virus comprising a nucleic acid encoding the antigen or the biologically active homolog thereof. Such methods may be used for therapeutic and/or preventative purposes. Other compositions that may additionally be administered include a protein and/or nucleic acid(s) encoding a protein that enhances the immune system, but do not comprise an antigen, e.g., those that prolong the life of antigen presenting cells, as further described herein.

Other methods may comprise administering a chemotherapeutic agent or drug, e.g., a drug that is not a nucleic acid vaccine, such as a drug that induces apoptosis of cancer cells. Any other combinations of one or more of a nucleic acid encoding an antigen; one or more oncolytic viruses encoding the antigen; one or more immune system enhancing protein(s) and or nucleic acid(s) encoding such a protein; and one or more drugs, e.g., chemotherapeutic drugs, may also be used for stimulating an immune response in a mammal. At least some of the methods may also be used to enhance the efficacy of another treatment, e.g., a treatment that comprises administering an immune system enhancing response in a mammal Administration of the priming step(s) may be performed at the same time, before or after administration of one or more other agents, e.g., boosting step(s).

Nucleic Acid Vaccines

Vaccines that may be administered to a mammal include any vaccine, e.g., a nucleic acid vaccine (e.g., a DNA vaccine). In an embodiment of the invention, a nucleic acid vaccine will encode an antigen, e.g., an antigen against which an immune response is desired. Other nucleic acids that may be used are those that increase or enhance an immune reaction, but which do not encode an antigen against which an immune reaction is desired. These vaccines are further described below.

Exemplary antigens include proteins or fragments thereof from a pathogenic organism, e.g., a bacterium or virus or other microorganism, as well as proteins or fragments thereof from a cell, e.g., a cancer cell. In one embodiment, the antigen is from a virus, such as class human papilloma virus (HPV), e.g., E7 or E6 or any genotype, serotype, or variant of HPV. These proteins are also oncogenic proteins, which are important in the induction and maintenance of cellular transformation and co-expressed in most HPV-containing cervical cancers and their precursor lesions. Therefore, cancer vaccines, such as the compositions of the invention, that target E7 or E6 can be used to control of HPV-associated neoplasms (Wu, T-C, Curr Opin Immunol. 6:746-54, 1994). Similarly, gene shuffled variants of the E7 and E6 proteins may be used to treat HPV-associated diseases (Oosterhuis, K. et al, Int J Cancer 129:397-06, 2011).

However, as noted, the present invention is not limited to the exemplified antigen(s). Rather, one of skill in the art will appreciate that the same results are expected for any antigen (and epitopes thereof) for which a T cell-mediated response is desired. The response so generated will be effective in providing protective or therapeutic immunity, or both, directed to an organism or disease in which the epitope or antigenic determinant is involved—for example as a cell surface antigen of a pathogenic cell or an envelope or other antigen of a pathogenic virus, or a bacterial antigen, or an antigen expressed as or as part of a pathogenic molecule.

Exemplary antigens and their sequences are set forth below.

E7 Protein from HPV-16

The E7 nucleic acid sequence (SEQ ID NO:8) and amino acid sequence (SEQ ID NO:9) from HPV-16 are shown below (see GenBank Accession No. NC_{—}001526).

| | |
|---|---|
| atg cat gga gat aca cct aca ttg cat gaa | 60 |
| tat atg tta gat ttg caa cca gag aca act | |
| Met His Gly Asp Thr Pro Thr Leu His Glu | 20 |
| Tyr Met Leu Asp Leu Gln Pro Glu Thr Thr | |
| gat ctc tac tgt tat gag caa tta aat gac | 120 |
| agc tca gag gag gag gat gaa ata gat ggt | |
| Asp Leu Tyr Cys Tyr Glu Gln Leu Asn Asp | 40 |
| Ser Ser Glu Glu Glu Asp Glu Ile Asp Gly | |
| cca get gga caa gca gaa ccg gac aga gcc | 180 |
| cat tac aat att gta acc ttt tgt tgc aag | |
| Pro Ala Gly Gln Ala Glu Pro Asp Arg Ala | 60 |
| His Tyr Asn Ile Val Thr Phe Cys Cys Lys | |
| tgt gac tct acg ctt cgg ttg tgc gta caa | 240 |
| agc aca cac gta gac att cgt act ttg gaa | |
| Cys Asp Ser Thr Leu Arg Leu Cys Val Gln | 80 |
| Ser Thr His Val Asp Ile Arg Thr Leu Glu | |
| gac ctg tta atg ggc aca cta gga att gtg | 297 |
| tgc ccc atc tgt tct cag gat aag ctt | |
| Asp Leu Leu Met Gly Thr Leu Gly Ile Val | 99 |
| Cys Pro Ile Cys Ser Gln Asp Lys Leu | |

In single letter code, the wild type E7 amino acid sequence (SEQ ID NO:9) is:

| | |
|---|---|
| MHGDTPTLHE YMLDLQPETT DLYCYEQLND SSEEEDEIDG | 99 |
| PAGQAEPDRA HYNIVTFCCK CDSTLRLCVQ STHVDIRTLE | |
| DLLMGTLGIV CPICSQDKL | |

In another embodiment (See GenBank Accession No. AF125673, nucleotides 562-858 and the E7 amino acid sequence), the C-terminal four amino acids QDKL (and their codons) above are replaced with the three amino acids QKP (and the codons cag aaa cca), yielding a protein of 98 residues.

When an oncoprotein or an epitope thereof is the immunizing moiety, it is preferable to reduce the tumorigenic risk of the vaccine itself. Because of the potential oncogenicity of the HPV E7 protein, the E7 protein may be used in a “detoxified” form.

To reduce oncogenic potential of E7 in a construct of this invention, one or more of the following positions of E7 is mutated:

| | | Preferred | nt Position | Amino acid |
|---|---|---|---|---|
| Original | Mutant | codon | (in SEQ ID | (in SEQ ID |
| residue | residue | mutation | NO: 8) | NO: 9) |
| Cys | Gly | TGT→GGT | 70 | 24 |
| | (or Ala) | | | |
| Glu | Gly | GAG→GGG | 77 | 26 |
| | (or Ala) | (or GCG) | | |
| Cys | Gly | TGC→GGC | 271 | 91 |
| | (or Ala) | | | |

In one embodiment, the E7 (detox) mutant sequence has the following two mutations:

a TGT→GGT mutation resulting in a Cys→Gly substitution at position 24 of SEQ ID NO: 9 a and GAG→GGG mutation resulting in a Glu→Gly substitution at position 26 of the wild type E7. This mutated amino acid sequence is shown below with the replacement residues underscored:

(SEQ ID NO: 10)

| | |
|---|---|
| MHGDTPTLHE YMLDLQPETT DLYGYEGLND SSEEEDEIDG | 97 |
| PAGQAEPDRA HYNIVTFCCK CDSTLRLCVQ STHVDIRTLE | |
| DLLMGTLGIV CPICSQKP | |

These substitutions completely eliminate the capacity of the E7 to bind to Rb, and thereby nullify its transforming activity. Any nucleotide sequence that encodes the above E7 or E7(detox) polypeptide, or an antigenic fragment or epitope thereof, can be used in the present compositions and methods, including the E7 and E7(detox) sequences are shown above.

E6 Protein from HPV-16

The wild type E6 nucleotide (SEQ ID NO:11) and amino acid sequences (SEQ ID NO:12) are shown below (see GenBank accession Nos. K02718 and NC_{—}001526):

| | |
|---|---|
| atg cac caa aag aga act gca atg ttt cag gac cca | 60 |
| cag gag cga ccc aga aag tta cca | |
| Met His Gln Lys Arg Thr Ala Met Phe Gln Asp Pro | 20 |
| Gln Glu Arg Pro Arg Lys Leu Pro | |
| cag tta tgc aca gag ctg caa aca act ata cat gat | 120 |
| ata ata tta gaa tgt gtg tac tgc | |
| Gln Leu Cys Thr Glu Leu Gln Thr Thr Ile His Asp | 40 |
| Ile Ile Leu Glu Cys Val Tyr Cys | |
| aag caa cag tta ctg cga cgt gag gta tat gac ttt | 180 |
| gct ttt cgg gat tta tgc ata gta | |
| Lys Gln Gln Leu Leu Arg Arg Glu Val Tyr Asp Phe | 60 |
| Ala Phe Arg Asp Leu Cys Ile Val | |
| tat aga gat ggg aat cca tat gct gta tgt gat aaa | 240 |
| tgt tta aag ttt tat tct aaa att | |
| Tyr Arg Asp Gly Asn Pro Tyr Ala Val Cys Asp Lys | 80 |
| Cys Leu Lys Phe Tyr Ser Lys Ile | |
| agt gag tat aga cat tat tgt tat agt ttg tat gga | 300 |
| aca aca tta gaa cag caa tac aac | |
| Ser Glu Tyr Arg His Tyr Cys Tyr Ser Leu Tyr Gly | 100 |
| Thr Thr Leu Glu Gln Gln Tyr Asn | |
| aaa ccg ttg tgt gat ttg tta att agg tgt att aac | 360 |
| tgt caa aag cca ctg tgt cct gaa | |
| Lys Pro Leu Cys Asp Leu Leu Ile Arg Cys Ile Asn | 120 |
| Cys Gln Lys Pro Leu Cys Pro Glu | |
| gaa aag caa aga cat ctg gac aaa aag caa aga ttc | 420 |
| cat aat ata agg ggt cgg tgg acc | |
| Glu Lys Gln Arg His Leu Asp Lys Lys Gln Arg Phe | 140 |
| His Asn Ile Arg Gly Arg Trp Thr | |
| ggt cga tgt atg tct tgt tgc aga tca tca aga aca | 474 |
| cgt aga gaa acc cag ctg taa | |
| Gly Arg Cys Met Ser Cys Cys Arg Ser Ser Arg Thr | 158 |
| Arg Arg Glu Thr Gln Leu stop | |

This polypeptide has 158 amino acids and is shown below in single letter code (SEQ ID NO:12):

| | |
|---|---|
| MHQKRTAMFQ DPQERPRKLP QLCTELQTTI HDIILECVYC | 158 |
| KQQLLRREVY DFAFRDLCIV YRDGNPYAVC DKCLKFYSKI | |
| SEYRHYCYSL YGTTLEQQYN KPLCDLLIRC INCQKPLCPE | |
| EKQRHLDKKQ RFHNIRGRWT GRCMSCCRSS RTRRETQL | |

E6 proteins from cervical cancer-associated HPV types such as HPV-16 induce proteolysis of the p53 tumor suppressor protein through interaction with E6-AP. Human mammary epithelial cells (MECs) immortalized by E6 display low levels of p53. HPV-16 E6, as well as other cancer-related papillomavirus E6 proteins, also binds the cellular protein E6BP (ERC-55). As with E7, described below a non-oncogenic mutated form of E6 may be used, referred to as “E6(detox).” Several different E6 mutations and publications describing them are discussed below.

The amino acid residues to be mutated are underscored in the E6 amino acid sequence above. Some studies of E6 mutants are based upon a shorter E6 protein of 151 nucleic acids, wherein the N-terminal residue was considered to be the Met at position 8 in the wild type E6. That shorter version of E6 is shown below as (SEQ ID NO:13):

MFQDPQERPR KLPQLCTELQ TTIHDIILEC VYCKQQLLRR
EVYDFAFRDL CIVYRDGNPY AVCDKCLKFY SKISEYRHYC
YSLYGTTLEQ QYNKPLCDLL IRCINCQKPL CPEEKQRHLD
KKQRFHNIRG RWTGRCMSCC RSSRTRRETQ L

To reduce oncogenic potential of E6 in a construct, one or more of the following positions of E6 is mutated:

| Original | Mutant | aa position in | aa position in |
|---|---|---|---|
| residue | residue | SEQ ID NO: 12 | SEQ ID NO: 13 |
| Cys | Gly (or Ala) | 70 | 63 |
| Cys | Gly (or Ala) | 113 | 106 |
| Ile | Thr | 135 | 128 |

Nguyen et al., J Virol. 6:13039-48, 2002, described a mutant of HPV-16 E6 deficient in binding α-helix partners which displays reduced oncogenic potential in vivo. This mutant, which includes a replacement of Ile with Thr as position 128 (of SEQ ID NO: 13), may be used in accordance with the present invention to make an E6 DNA vaccine that has a lower risk of being oncogenic. This E6(I¹²⁸T) mutant is defective in its ability to bind at least a subset of α-helix partners, including E6AP, the ubiquitin ligase that mediates E6-dependent degradation of the p53 protein.

Cassetti M C et al., Vaccine 22:520-52, 2004, examined the effects of mutations four or five amino acid positions in E6 and E7 to inactivate their oncogenic potential. The following mutations were examined: E6-C⁶³G and E6 C¹⁰⁶G (positions based on the wild type E6); E7-C²⁴G, E7-E²⁶G, and E7 C⁹¹G (positions based on the wild type E7). Venezuelan equine encephalitis virus replicon particle (VRP) vaccines encoding mutant or wild type E6 and E7 proteins elicited comparable CTL responses and generated comparable antitumor responses in several HPV16 E6(+)E7(+) tumor challenge models: protection from either C3 or TC-1 tumor challenge was observed in 100% of vaccinated mice. Eradication of C3 tumors was observed in approximately 90% of the mice. The predicted inactivation of E6 and E7 oncogenic potential was confirmed by demonstrating normal levels of both p53 and Rb proteins in human mammary epithelial cells infected with VRPs expressing mutant E6 and E7 genes.

The HPV16 E6 protein contains two zinc fingers important for structure and function; one cysteine (C) amino acid position in each pair of C—X—X—C (where X is any amino acid) zinc finger motifs may be mutated at E6 positions 63 and 106 (based on the wild type E6). Mutants are created, for example, using the Quick Change Site-Directed Mutagenesis Kit (Stratagene, La Jolla, Calif.). HPV16 E6 containing a single point mutation in the codon for Cys¹⁰⁶ in the wild type E6 (=Cys 113 in the wild type E6). Cys¹⁰⁶ neither binds nor facilitates degradation of p53 and is incapable of immortalizing human mammary epithelial cells (MEC), a phenotype dependent upon p53 degradation. A single amino acid substitution at position Cys⁶³ of the wild type E6 (=Cys⁷⁰ in the wild type E6) destroys several HPV16 E6 functions: p53 degradation, E6TP-1 degradation, activation of telomerase, and, consequently, immortalization of primary epithelial cells.

Any nucleotide sequence that encodes these E6 polypeptides, one of the mutants thereof, or an antigenic fragment or epitope thereof, can be used in the present invention. Other mutations can be tested and used in accordance with the methods described herein including those described in Cassetti et al., supra. These mutations can be produced from any appropriate starting sequences by mutation of the coding DNA.

The present invention also includes the use of a tandem E6-E7 vaccine, using one or more of the mutations described herein to render the oncoproteins inactive with respect to their oncogenic potential in vivo. VRP vaccines (described in Cassetti et al., supra) comprised fused E6 and E7 genes in one open reading frame which were mutated at four or five amino acid positions. Thus, the present constructs may include one or more epitopes of E6 and E7, which may be arranged in their native order or shuffled in any way that permits the expressed protein to bear the E6 and E7 antigenic epitopes in an immunogenic form. DNA encoding amino acid spacers between E6 and E7 or between individual epitopes of these proteins may be introduced into the vector, provided again, that the spacers permit the expression or presentation of the epitopes in an immunogenic manner after they have been expressed by transduced host cells.

### Ovalbumin (OVA)

An amino acid sequences encoding a representative OVA (SEQ ID NO:139) is shown below.

MGSIGAASMEFCFDVFKELKVHHANENIFYCPIAIMSALAMVYLGAK
DSTRTQINKVVRFDKLPGFGDSIEAQCGTSVNVHSSLRDILNQITKP
NDVYSFSLASRLYAEERYPILPEYLQCVKELYRGGLEPINFQTAADQ
ARELINSWVESQTNGIIRNVLQPSSVDSQTAMVLVNAIVFKGLWEKT
FKDEDTQAMPFRVTEQESKPVQMMYQIGLFRVASMASEKMKILELPF
ASGTMSMLVLLPDEVSGLEQLESIINFEKLTEWTSSNVMEERKIKVY
LPRMKMEEKYNLTSVLMAMGITDVFSSSANLSGISSAESLKISQAVH
AAHAEINEAGREVVGSAEAGVDAASVSEEFRADHPFLFCIKHIATNA
VLFFGRCVSP

### Other Exemplary Antigens

Exemplary antigens are epitopes of pathogenic microorganisms against which the host is defended by effector T cells responses, including CTL and delayed type hypersensitivity. These typically include viruses, intracellular parasites such as malaria, and bacteria that grow intracellularly such as Mycobacterium and Listeria species. Thus, the types of antigens included in the vaccine compositions of this invention may be any of those associated with such pathogens as well as tumor-specific antigens. It is noteworthy that some viral antigens are also tumor antigens in the case where the virus is a causative factor in the tumor.

In fact, the two most common cancers worldwide, hepatoma and cervical cancer, are associated with viral infection. Hepatitis B virus (HBV) (Beasley, R. P. et al., Lancet 2:1129-1133 (1981) has been implicated as etiologic agent of hepatomas. About 80-90% of cervical cancers express the E6 and E7 antigens (discussed above and exemplified herein) from one of four “high risk” human papillomavirus types: HPV-16, HPV-18, HPV-31 and HPV-45 (Gissmann, L. et al., Ciba Found Symp. 120:190-207, 1986; Beaudenon, S., et al. Nature 321:246-9, 1986, incorporated by reference herein). The HPV E6 and E7 antigens are the most promising targets for virus associated cancers in immunocompetent individuals because of their ubiquitous expression in cervical cancer. In addition to their importance as targets for therapeutic cancer vaccines, virus-associated tumor antigens are also ideal candidates for prophylactic vaccines. Indeed, introduction of prophylactic HBV vaccines in Asia have decreased the incidence of hepatoma (Chang, M H et al. New Engl. J. Med. 336, 1855-1859 (1997), representing a great impact on cancer prevention.

Among the most important viruses in chronic human viral infections are HPV, HBV, hepatitis C Virus (HCV), retroviruses such as human immunodeficiency virus (HIV-1 and HIV-2), herpes viruses such as Epstein Barr Virus (EBV), cytomegalovirus (CMV), HSV-1 and HSV-2, and influenza virus. Useful antigens include HBV surface antigen or HBV core antigen; ppUL83 or pp89 of CMV; antigens of gp120, gp41 or p24 proteins of HIV-1; ICP27, gD2, gB of HSV; or influenza hemagglutinin or nucleoprotein (Anthony, L S et al., Vaccine 1999; 17:373-83). Other antigens associated with pathogens that can be utilized as described herein are antigens of various parasites, including malaria, e.g., malaria peptide based on repeats of NANP.

In certain embodiments, the invention includes methods using foreign antigens in which individuals may have existing T cell immunity (such as influenza, tetanus toxin, herpes etc). In other embodiments, the skilled artisan would readily be able to determine whether a subject has existing T cell immunity to a specific antigen according to well known methods available in the art and use a foreign antigen to which the subject does not already have an existing T cell immunity against.

In alternative embodiments, the antigen is from a pathogen that is a bacterium, such as Bordetella pertussis; Ehrlichia chaffeensis; Staphylococcus aureus; Toxoplasma gondii; Legionella pneumophila; Brucella suis; Salmonella enterica; Mycobacterium avium; Mycobacterium tuberculosis; Listeria monocytogenes; Chlamydia trachomatis; Chlamydia pneumoniae; Rickettsia rickettsii; or, a fungus, such as, e.g., Paracoccidioides brasiliensis; or other pathogen, e.g., Plasmodium falciparum.

As used herein, the term “cancer” and includes, but is not limited to, solid tumors and blood borne tumors, as well as precancerous conditions, such as persistent viral infections, that increase a patient's risk of developing cancer. The term cancer includes diseases of the skin, tissues, organs, bone, cartilage, blood and vessels. A term used to describe cancer that is far along in its growth, also referred to as “late stage cancer” or “advanced stage cancer,” is cancer that is metastatic, e.g., cancer that has spread from its primary origin to another part of the body. In certain embodiments, advanced stage cancer includes stages 3 and 4 cancers. Cancers are ranked into stages depending on the extent of their growth and spread through the body; stages correspond with severity. Determining the stage of a given cancer helps doctors to make treatment recommendations, to form a likely outcome scenario for what will happen to the patient (prognosis), and to communicate effectively with other doctors. In other embodiments, the cancer is a low grade or high intraepithelial neoplasia such as CIN grades 1-3, ASCUS, AGUS or LSIL/HSIL, VIN1-3, AIN1-3, PIN1-3.

There are multiple staging scales in use. One of the most common ranks cancers into five progressively more severe stages: 0, I, II, III, and IV. Stage 0 cancer is cancer that is just beginning, involving just a few cells. Stages I, II, III, and IV represent progressively more advanced cancers, characterized by larger tumor sizes, more tumors, the aggressiveness with which the cancer grows and spreads, and the extent to which the cancer has spread to infect adjacent tissues and body organs.

Another popular staging system is known as the TNM system, a three dimensional rating of cancer extensiveness. Using the TNM system, doctors rate the cancers they find on each of three scales, where T stands for tumor size, N stands for lymph node involvement, and M stands for metastasis (the degree to which cancer has spread beyond its original locations). Larger scores on each of the three scales indicate more advanced cancer. For example, a large tumor that has not spread to other body parts might be rated T3, N0, M0, while a smaller but more aggressive cancer might be rated T2, N2, M1 suggesting a medium sized tumor that has spread to local lymph nodes and has just gotten started in a new organ location.

Cancers that may treated by methods and compositions of the invention include, but are not limited to, cancer cells from the anus, bladder, blood, bone, bone marrow, brain, breast, colon, esophagus, gastrointestine, gum, head, kidney, liver, lung, nasopharynx, neck, oral cavity, oropharynx, ovary, penis, prostate, skin, stomach, testis, tongue, cervix, uterus, vagina or vulva. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In addition to its applicability to human cancer and infectious diseases, the present invention is also intended for use in treating animal diseases in the veterinary medicine context. Thus, the approaches described herein may be readily applied by one skilled in the art to treatment of veterinary herpes virus infections including equine herpes viruses, bovine viruses such as bovine viral diarrhea virus (for example, the E2 antigen), bovine herpes viruses, Marek's disease virus in chickens and other fowl; animal retroviral and lentiviral diseases (e.g., feline leukemia, feline immunodeficiency, simian immunodeficiency viruses, etc.); pseudorabies and rabies; and the like.

As for tumor antigens, any tumor-associated or tumor-specific antigen (or tumor cell derived epitope) (collectively, TAA) that can be recognized by T cells, including CTL, can be used. These include, without limitation, mutant p53, HER2/neu or a peptide thereof, or any of a number of melanoma-associated antigens such as MAGE-1, MAGE-3, MART-1/Melan-A, tyrosinase, gp75, gp100, BAGE, GAGE-1, GAGE-2, GnT-V, and p15 (see, for example, U.S. Pat. No. 6,187,306, incorporated herein by reference).

It is not necessary to include a full length antigen in a nucleic acid vaccine; it suffices to include a fragment that will be presented by MHC class I and/or II. A nucleic acid may include 1, 2, 3, 4, 5 or more antigens, which may be the same or different ones.

### Approaches for Mutagenesis of E6, E7, and Other Antigens

Mutants of the antigens described here may be created, for example, using the Quick Change Site-Directed Mutagenesis Kit (Stratagene, La Jolla, Calif.) or direct synthesis and Gibson assembly. Generally, antigens that may be used herein may be proteins or peptides that differ from the naturally-occurring proteins or peptides but yet retain the necessary epitopes for functional activity. Additionally, a consensus sequence among two or more different HPV types may be selected as an antigen. In certain embodiments, an antigen may comprise, consist essentially of, or consist of an amino acid sequence that is at least about 50%, 55%, 60%, 65%, 70%, 75%, 80%, 85%, 90%, 95%, 96%, 97%, 98%, or 99% identical to that of the naturally-occurring antigen or a fragment thereof. In certain embodiments, an antigen may also comprise, consist essentially of, or consist of an amino acid sequence that is encoded by a nucleotide sequence that is at least about 50%, 55%, 60%, 65%, 70%, 75%, 80%, 85%, 90%, 95%, 96%, 97%, 98%, or 99° A identical to a nucleotide sequence encoding the naturally-occurring antigen or a fragment thereof. In certain embodiments, an antigen may also comprise, consist essentially of, or consist of an amino acid sequence that is encoded by a nucleic acid that hybridizes under high stringency conditions to a nucleic acid encoding the naturally-occurring antigen or a fragment thereof. Hybridization conditions are further described herein.

In one embodiment, an exemplary protein may comprise, consist essentially of, or consist of, an amino acid sequence that is at least about 50%, 55%, 60%, 65%, 70%, 75%, 80%, 85%, 90%, 95%, 96%, 97%, 98%, or 99° A identical to that of a viral protein, including for example E6 or E7, such as an E6 or E7 sequence provided herein. Where the E6 or E7 protein is a detox E6 or E7 protein, the amino acid sequence of the protein may comprise, consist essentially of, or consist of an amino acid sequence that is at least about 50%, 55%, 60%, 65%, 70%, 75%, 80%, 85%, 90%, 95%, 96%, 97%, 98%, or 99% identical to that of an E6 or E7 protein, wherein the amino acids that render the protein a “detox” protein are present.

### Exemplary DNA Vaccines Encoding an Immunogenicity-Potentiating Polypeptide (IPP) and an Antigen

In one embodiment, a nucleic vaccine encodes a fusion protein comprising an antigen and a second protein, e.g., an IPP. An IPP may act in potentiating an immune response by promoting: processing of the linked antigenic polypeptide via the MHC class I pathway or targeting of a cellular compartment that increases the processing. This basic strategy may be combined with an additional strategy pioneered by the present inventors and colleagues, that involve linking DNA encoding another protein, generically termed a “targeting polypeptide,” to the antigen-encoding DNA. Again, for the sake of simplicity, the DNA encoding such a targeting polypeptide will be referred to herein as a “targeting DNA.” That strategy has been shown to be effective in enhancing the potency of the vectors carrying only antigen-encoding DNA. See for example, the following PCT publications by Wu et al: WO 01/29233; WO 02/009645; WO 02/061113; WO 02/074920; and WO 02/12281, all of which are incorporated by reference in their entirety. The other strategies include the use of DNA encoding polypeptides that promote or enhance:
(a) development, accumulation or activity of antigen presenting cells or targeting of antigen to compartments of the antigen presenting cells leading to enhanced antigen presentation;
(b) intercellular transport and spreading of the antigen; or
(c) any combination of (a) and (b).
(d) sorting of the lysosome-associated membrane protein type 1 (Sig/LAMP-1).
   The strategy includes use of:
(a) a viral intercellular spreading protein selected from the group of herpes simplex virus-1 VP22 protein, Marek's disease virus UL49 (see WO 02/09645 and U.S. Pat. No. 7,318,928), protein or a functional homologue or derivative thereof;
(b) calreticulin (CRT) and other endoplasmic reticulum chaperone polypeptides selected from the group of CRT-like molecules ER60, GRP94, gp96, or a functional homologue or derivative thereof (see WO 02/12281 and U.S. Pat. No. 7,3442,002);
(c) a cytoplasmic translocation polypeptide domains of a pathogen toxin selected from the group of domain II of Pseudomonas exotoxin ETA or a functional homologue or derivative thereof (see published US application 20040086845);
(d) a polypeptide that targets the centrosome compartment of a cell selected from γ-tubulin or a functional homologue or derivative thereof; or
(e) a polypeptide that stimulates dendritic cell precursors or activates dendritic cell activity selected from the group of GM-CSF, Flt3-ligand extracellular domain, or a functional homologue or derivative thereof; or
(f) a costimulatory signal, such as a B7 family protein, including B7-DC (see U.S. Ser. No. 09/794,210), B7.1, B7.2, soluble CD40, etc.).
(g) an anti-apoptotic polypeptide selected from the group consisting of (1) BCL-xL, (2) BCL2, (3) XIAP, (4) FLICEc-s, (5) dominant-negative caspase-8, (6) dominant negative caspase-9, (7) SPI-6, and (8) a functional homologue or derivative of any of (1)-(7). (See WO 2005/047501).

The following publications, all of which are incorporated by reference in their entirety, describe IPPs: Kim T W et al., J Clin Invest 112: 109-117, 2003; Cheng W F et al., J Clin Invest 108: 669-678, 2001; Hung C F et al., Cancer Res 61:3698-3703, 2001; Chen C H et al., 2000, supra; U.S. Pat. No. 6,734,173; published patent applications WO05/081716, WO05/047501, WO03/085085, WO02/12281, WO02/074920, WO02/061113, WO02/09645, and WO01/29233. Comparative studies of these IPPs using HPV E6 as the antigen are described in Peng, S. et al., J Biomed Sci. 12:689-700 2005.

An antigen may be linked N-terminally or C-terminally to an IPP. Exemplary IPPs and fusion constructs encoding such are described below.

Lysosomal Associated Membrane Protein 1 (LAMP-1)

The DNA sequence encoding the E7 protein fused to the translocation signal sequence and LAMP-1 domain (Sig-E7-LAMP-1) [SEQ ID NO: 16] is:

ATGGCGGCCCCCGGCGCCCGGCGGCCGCTGCTCCTGCTGCTGCTGGC
AGGCCTTGCACATGGCGCCTCAGCACTCTTTGAGGATCTAATCATGC
ATGGAGATACACCTACATTGCATGAATATATGTTAGATTTGCAACCA
GAGACAACTGATCTCTACTGTTATGAGCAATTAAATGACAGCTCAGA
GGAGGAGGATGAAATAGATGGTCCAGCTGGACAAGCAGAACCGGACA
GAGCCCATTACAATATTGTTACCTTTTGTTGCAAGTGTGACTCTACG
CTTCGGTTGTGCGTACAAAGCACACACGTAGACATTCGTACTTTGGA
AGACCTGTTAATGGGCACACTAGGAATTGTGTGCCCCATCTGTTCTC
AGGATCTTAACAACATGTTGATCCCCATTGCTGTGGGCGGTGCCCTG
GCAGGGCTGGTCCTCATCGTCCTCATTGCCTACCTCATTGGCAGGAA
GAGGAGTCACGCCGGCTATCAGACCATCTAG.

The amino acid sequence of Sig/E7/LAMP-1 [SEQ ID NO: 17] is:

MAAPGARRPL LLLLLAGLAH GASALFEDLI MHGDTPTLHE
YMLDLQPETT DLYCYEQLND SSEEEDEIDG PAGQAEPDRA
HYNIVTFCCK CDSTLRLCVQ STHVDIRTLE DLLMGTLGIV
CPICSQDLNN MLIPIAVGGA LAGLVLIVLI AYLIGRKRSH
AGYQTI.

The nucleotide sequence of the immunogenic vector pcDNA3-Sig/E7/LAMP-1 [SEQ ID NO: 18] is shown below with the SigE7-LAMP-1 coding sequence in lower case and underscored:

GACGGATCGGGAGATCTCCCGATCCCCTATGGTCGACTCTCAGTACAATCTGCT
CTGATGCCGCATAGTTAAGCCAGTATCTGCTCCCTGCTTGTGTGTTGGAGGTCG
CTGAGTAGTGCGCGAGCAAAATTTAAGCTACAACAAGGCAAGGCTTGACCGA
CAATTGCATGAAGAATCTGCTTAGGGTTAGGCGTTTTGCGCTGCTTCGCGATGT
ACGGGCCAGATATACGCGTTGACATTGATTATTGACTAGTTATTAATAGTAATC
AATTACGGGGTCATTAGTTCATAGCCCATATATGGAGTTCCGCGTTACATAA
CTTACGGTAAATGGCCCGCCTGGCTGACCGCCCAACGACCCCCGCCCATTGACG
TCAATAATGACGTATGTTCCCATAGTAACGCCAATAGGGACTTTCCATTGACGT
CAATGGGTGGACTATTTACGGTAAACTGCCCACTTGGCAGTACATCAAGTGT
ATCATATGCCAAGTACGCCCCCTATTGACGTCAATGACGGTAAATGGCCCGCCT
GGCATTATGCCCAGTACATGACCTTATGGGACTTTCCTACTTGGCAGTACATCT
ACGTATTAGTCATCGCTATTACCATGGTGATGCGGTTTTGGCAGTACATCAA
TGGGCGTGGATAGCGGTTTGACTCACGGGGATTTCCAAGTCTCCACCCCATTGA
CGTCAATGGGAGTTTGTTTTGGCACCAAAATCAACGGGACTTTCCAAAATGTCG
TAACAACTCCGCCCCATTGACGCAAATGGGCGGTAGGCGTGTACGGTGGGAG
GTCTATATAAGCAGAGCTCTCTGGCTAACTAGAGAACCCACTGCTTACTGGCTT
ATCGAAATTAATACGACTCACTATAGGGAGACCCAAGCTGGCTAGCGTTTAAAC
GGGCCCTCTAGACTCGAGCGGCCGCCACTGTGCTGGATATCTGCAGAATTCa
tggcggcccccggcgcccggcggccgctgctcctgctgctgctggcaggccttgcacatggcgcctcagcactctttgag
gatctaatcatgcatggagatacacctacattgcatgaatatatgttagatttgcaaccagagacaactgatctctactg
ttatgagcaattaaatgacagctcagaggaggaggatgaaatagatggtccagctggacaagcagaaccggacagagccc
attacaatattgttaccttttgttgcaagtgtgactctacgcttcggttgtgcgtacaaagcacacacgtagacattcgt
actttggaagacctgttaatgggcacactaggaattgtgtgccccatctgttctcaggatcttaacaacatgttgatccc
cattgctgtgggcggtgccctggcagggctggtcctcatcgtcctcattgcctacctcattggcaggaagaggagtcacg
ccggctatcagaccatctagGGATCCGAGCTCGGTACCAAGCTTAAGTTTAAACCGCTGAT
CAGCCTCGACTGTGCCTTCTAGTTGCCAGCCATCTGTTGTTTGCCCCTCCCCCGT
GCCTTCCTTGACCCTGGAAGGTGCCACTCCCACTGTCCTTTCCTAATAAAATGA
GGAAATTGCATCGCATTGTCTGAGTAGGTGTCATTCTATTCTGGGGGGTGGGGT
GGGGCAGGACAGCAAGGGGGAGGATTGGGAAGACAATAGCAGGCATGCTGGG
GATGCGGTGGGCTCTATGGCTTCTGAGGCGGAAAGAACCAGCTGGGGCTCTAG
GGGGTATCCCCACGCGCCCTGTAGCGGCGCATTAAGCGCGGCGGGTGTGGTGG
TTACGCGCAGCGTGACCGCTACACTTGCCAGCGCCCTAGCGCCCGCTCCTTTCG
CTTTCTTCCCTTCCTTTCTCGCCACGTTCGCCGGCTTTCCCCGTCAAGCTCTAAAT
CGGGGCATCCCTTTAGGGTTCCGATTTAGTGCTTTACGGCACCTCGACCCCAAA
AAACTTGATTAGGGTGATGGTTCACGTAGTGGGCCATCGCCCTGATAGACGGTT
TTTCGCCCTTTGACGTTGGAGTCCACGTTCTTTAATAGTGGACTCTTGTTCCAAA
CTGGAACAACACTCAACCCTATCTCGGTCTATTCTTTTGATTTATAAGGGATTTT
GGGGATTTCGGCCTATTGGTTAAAAAATGAGCTGATTTAACAAAAATTTAACGC
GAATTAATTCTGTGGAATGTGTGTCAGTTAGGGTGTGGAAAGTCCCCAGGCTCC
CCAGGCAGGCAGAAGTATGCAAAGCATGCATCTCAATTAGTCAGCAACCAGGT
GTGGAAAGTCCCCAGGCTCCCCAGCAGGCAGAAGTATGCAAAGCATGCATCTC
AATTAGTCAGCAACCATAGTCCCGCCCCTAACTCCGCCCATCCCGCCCCTAACT
CCGCCCAGTTCCGCCCATTCTCCGCCCCATGGCTGACTAATTTTTTTTATTTATG
CAGAGGCCGAGGCCGCCTCTGCCTCTGAGCTATTCCAGAAGTAGTGAGGAGGC
TTTTTTGGAGGCCTAGGCTTTTGCAAAAAGCTCCCGGGAGCTTGTATATCCATTT
TCGGATCTGATCAAGAGACAGGATGAGGATCGTTTCGCATGATTGAACAAGAT
GGATTGCACGCAGGTTCTCCGGCCGCTTGGGTGGAGAGGCTATTCGGCTATGAC
TGGGCACAACAGACAATCGGCTGCTCTGATGCCGCCGTGTTCCGGCTGTCAGCG
CAGGGGCGCCCGGTTCTTTTTGTCAAGACCGACCTGTCCGGTGCCCTGAATGAA
CTGCAGGACGAGGCAGCGCGGCTATCGTGGCTGGCCACGACGGGCGTTCCTTG
CGCAGCTGTGCTCGACGTTGTCACTGAAGCGGGAAGGGACTGGCTGCTATTGGG
CGAAGTGCCGGGGCAGGATCTCCTGTCATCTCACCTTGCTCCTGCCGAGAAAGT
ATCCATCATGGCTGATGCAATGCGGCGGCTGCATACGCTTGATCCGGCTACCTG
CCCATTCGACCACCAAGCGAAACATCGCATCGAGCGAGCACGTACTCGGATGG
AAGCCGGTCTTGTCGATCAGGATGATCTGGACGAAGAGCATCAGGGGCTCGCG
CCAGCCGAACTGTTCGCCAGGCTCAAGGCGCGCATGCCCGACGGCGAGGATCT
CGTCGTGACCCATGGCGATGCCTGCTTGCCGAATATCATGGTGGAAAATGGCCG
CTTTTCTGGATTCATCGACTGTGGCCGGCTGGGTGTGGCGGACCGCTATCAGGA
CATAGCGTTGGCTACCCGTGATATTGCTGAAGAGCTTGGCGGCGAATGGGCTGA
CCGCTTCCTCGTGCTTTACGGTATCGCCGCTCCCGATTCGCAGCGCATCGCCTTC
TATCGCCTTCTTGACGAGTTCTTCTGAGCGGGACTCTGGGGTTCGAAATGACC
GACCAAGCGACGCCCAACCTGCCATCACGAGATTTCGATTCCACCGCCGCCTTC
TATGAAAGGTTGGGCTTCGGAATCGTTTTCCGGGACGCCGGCTGGATGATCCTC
CAGCGCGGGGATCTCATGCTGGAGTTCTTCGCCCACCCCAACTTGTTTATTG
CAGCTTATAATGGTTACAAATAAAGCAATAGCATCACAAATTTCACAAATAAA
GCATTTTTTTCACTGCATTCTAGTTGTGGTTTGTCCAAACTCATCAATGTATCTT
ATCATGTCTGTATACCGTCGACCTCTAGCTAGAGCTTGGCGTAATCATGGTC
ATAGCTGTTTCCTGTGTGAAATTGTTATCCGCTCACAATTCCACACAACATACG
AGCCGGAAGCATAAAGTGTAAAGCCTGGGGTGCCTAATGAGTGAGCTAACTCA
CATTAATTGCGTTGCGCTCACTGCCCGCTTTCCAGTCGGGAAACCTGTCGTGC
CAGCTGCATTAATGAATCGGCCAACGCGCGGGGAGAGGCGGTTTGCGTATTGG
GCGCTCTTCCGCTTCCTCGCTCACTGACTCGCTGCGCTCGGTCGTTCGGCTGCGG
CGAGCGGTATCAGCTCACTCAAAGGCGGTAATACGGTTATCCACAGAATCAG
GGGATAACGCAGGAAAGAACATGTGAGCAAAAGGCCAGCAAAAGGCCAGGAA
CCGTAAAAAGGCCGCGTTGCTGGCGTTTTTCCATAGGCTCCGCCCCCCTGACGA
GCATCACAAAAATCGACGCTCAAGTCAGAGGTGGCGAAACCCGACAGGACTAT
AAAGATACCAGGCGTTTCCCCCTGGAAGCTCCCTCGTGCGCTCTCCTGTTCCGA
CCCTGCCGCTTACCGGATACCTGTCCGCCTTTCTCCCTTCGGGAAGCGTGGCGCT
TTCTCAATGCTCACGCTGTAGGTATCTCAGTTCGGTGTAGGTCGTTCGCTCCAAG
CTGGGCTGTGTGCACGAACCCCCCGTTCAGCCCGACCGCTGCGCCTTATCCGGT
AACTATCGTCTTGAGTCCAACCCGGTAAGACACGACTTATCGCCACTGGCAGCA
GCCACTGGTAACAGGATTAGCAGAGCGAGGTATGTAGGCGGTGCTACAGAGTT
CTTGAAGTGGTGGCCTAACTACGGCTACACTAGAAGGACAGTATTTGGTATCTG
CGCTCTGCTGAAGCCAGTTACCTTCGGAAAAAGAGTTGGTAGCTCTTGATCCGG
CAAACAAACCACCGCTGGTAGCGGTGGTTTTTTTGTTTGCAAGCAGCAGATTAC
GCGCAGAAAAAAAGGATCTCAAGAAGATCCTTTGATCTTTTCTACGGGGTCTGA
CGCTCAGTGGAACGAAAACTCACGTTAAGGGATTTTGGTCATGAGATTATCAAA
AAGGATCTTCACCTAGATCCTTTTAAATTAAAAATGAAGTTTTAAATCAATCTA
AAGTATATATGAGTAAACTTGGTCTGACAGTTACCAATGCTTAATCAGTGAGGC
ACCTATCTCAGCGATCTGTCTATTTCGTTCATCCATAGTTGCCTGACTCCCCGTC
GTGTAGATAACTACGATACGGGAGGGCTTACCATCTGGCCCCAGTGCTGCAATG
ATACCGCGAGACCCACGCTCACCGGCTCCAGATTTATCAGCAATAAACCAGCCA
GCCGGAAGGGCCGAGCGCAGAAGTGGTCCTGCAACTTTATCCGCCTCCATCCAG
TCTATTAATTGTTGCCGGGAAGCTAGAGTAAGTAGTTCGCCAGTTAATAGTTTG
CGCAACGTTGTTGCCATTGCTACAGGCATCGTGGTGTCACGCTCGTCGTTTGGT
ATGGCTTCATTCAGCTCCGGTTCCCAACGATCAAGGCGAGTTACATGATCCCCC
ATGTTGTGCAAAAAAGCGGTTAGCTCCTTCGGTCCTCCGATCGTTGTCAGAAGT
AAGTTGGCCGCAGTGTTATCACTCATGGTTATGGCAGCACTGCATAATTCTCTT
ACTGTCATGCCATCCGTAAGATGCTTTTCTGTGACTGGTGAGTACTCAACCAAG
TCATTCTGAGAATAGTGTATGCGGCGACCGAGTTGCTCTTGCCCGGCGTCAATA
CGGGATAATACCGCGCCACATAGCAGAACTTTAAAAGTGCTCATCATTGGAAA
ACGTTCTTCGGGGCGAAAACTCTCAAGGATCTTACCGCTGTTGAGATCCAGTTC
GATGTAACCCACTCGTGCACCCAACTGATCTTCAGCATCTTTTACTTTCACCAGC
GTTTCTGGGTGAGCAAAAACAGGAAGGCAAAATGCCGCAAAAAAGGGAATAA
GGGCGACACGGAAATGTTGAATACTCATACTCTTCCTTTTTCAATATTATTGAA
GCATTTATCAGGGTTATTGTCTCATGAGCGGATACATATTTGAATGTATTTAGA
AAAATAAACAAATAGGGGTTCCGCGCACATTTCCCCGAAAAGTGCCACCTGAC
GTC,

HSP70 from M. tuberculosis

The nucleotide sequence encoding HSP70 (SEQ ID NO: 19) is (nucleotides 10633-12510 of the M. tuberculosis genome in GenBank NC_{—}000962):

atggctcg tgcggtcggg atcgacctcg ggaccaccaa ctccgtcgtc tcggttctgg aaggtggcga cccggtcgtc
gtcgccaact ccgagggctc caggaccacc ccgtcaattg tcgcgttcgc ccgcaacggt gaggtgctgg tcggccagcc
cgccaagaac caggcagtga ccaacgtcga tcgcaccgtg cgctcggtca agcgacacat gggcagcgac
tggtccatag agattgacgg caagaaatac accgcgccgg agatcagcgc ccgcattctg atgaagctga agcgcgacgc
cgaggcctac ctcggtgagg acattaccga cgcggttatc acgacgcccg cctacttcaa tgacgcccag cgtcaggcca
ccaaggacgc cggccagatc gccggcctca acgtgctgcg gatcgtcaac gagccgaccg cggccgcgct
ggcctacggc ctcgacaagg gcgagaagga gcagcgaatc ctggtcttcg acttgggtgg tggcactttc gacgtttccc
tgctggagat cggcgagggt gtggttgagg tccgtgccac ttcgggtgac aaccacctcg gcggcgacga ctgggaccag
cgggtcgtcg attggctggt ggacaagttc aagggcacca gcggcatcga tctgaccaag gacaagatgg cgatgcagcg
gctgcgggaa gccgccgaga aggcaaagat cgagctgagt tcgagtcagt ccacctcgat caacctgccc tacatcaccg
tcgacgccga caagaacccg ttgttcttag acgagcagct gacccgcgcg gagttccaac ggatcactca ggacctgctg
gaccgcactc gcaagccgtt ccagtcggtg atcgctgaca ccggcatttc ggtgtcggag atcgatcacg ttgtgctcgt
gggtggttcg acccggatgc ccgcggtgac cgatctggtc aaggaactca ccggcggcaa ggaacccaac
aagggcgtca accccgatga ggttgtcgcg gtgggagccg ctctgcaggc cggcgtcctc aagggcgagg
tgaaagacgt tctgctgctt gatgttaccc cgctgagcct gggtatcgag accaagggcg gggtgatgac caggctcatc
gagcgcaaca ccacgatccc caccaagcgg tcggagactt tcaccaccgc cgacgacaac caaccgtcgg
tgcagatcca ggtctatcag ggggagcgtg agatcgccgc gcacaacaag ttgctcgggt ccttcgagct gaccggcatc
ccgccggcgc cgcgggggat tccgcagatc gaggtcactt tcgacatcga cgccaacggc attgtgcacg tcaccgccaa
ggacaagggc accggcaagg agaacacgat ccgaatccag gaaggctcgg gcctgtccaa ggaagacatt
gaccgcatga tcaaggacgc cgaagcgcac gccgaggagg atcgcaagcg tcgcgaggag gccgatgttc
gtaatcaagc cgagacattg gtctaccaga cggagaagtt cgtcaaagaa cagcgtgagg ccgagggtgg ttcgaaggta
cctgaagaca cgctgaacaa ggttgatgcc gcggtggcgg aagcgaaggc ggcacttggc ggatcggata tttcggccat
caagtcggcg atggagaagc tgggccagga gtcgcaggct ctggggcaag cgatctacga agcagctcag
gctgcgtcac aggccactgg cgctgcccac cccggcggcg agccgggcgg tgcccacccc ggctcggctg
atgacgttgt ggacgcggag gtggtcgacg acggccggga ggccaagtga

The amino acid sequence of HSP70 [SEQ ID NO: 20] is:

MARAVGIDLG TTNSVVSVLE GGDPVVVANS EGSRTTPSIV
AFARNGEVLV
GQPAKNQAVT NVDRTVRSVK RHMGSDWSIE IDGKKYTAPE
ISARILMKLK
RDAEAYLGED ITDAVITTPA YFNDAQRQAT KDAGQIAGLN
VLRIVNEPTA
AALAYGLDKG EKEQRILVFD LGGGTFDVSL LEIGEGVVEV
RATSGDNHLG
GDDWDQRVVD WLVDKFKGTS GIDLTKDKMA MQRLREAAEK
AKIELSSSQS
TSINLPYITV DADKNPLFLD EQLTRAEFQR ITQDLLDRTR
KPFQSVIADT
GISVSEIDHV VLVGGSTRMP AVTDLVKELT GGKEPNKGVN
PDEVVAVGAA
LQAGVLKGEV KDVLLLDVTP LSLGIETKGG VMTRLIERNT
TIPTKRSETF
TTADDNQPSV QIQVYQGERE IAAHNKLLGS FELTGIPPAP
RGIPQIEVTF
DIDANGIVHV TAKDKGTGKE NTIRIQEGSG LSKEDIDRMI
KDAEAHAEED
RKRREEADVR NQAETLVYQT EKFVKEQREA EGGSKVPEDT
LNKVDAAVAE
AKAALGGSDI SAIKSAMEKL GQESQALGQA IYEAAQAASQ
ATGAAHPGGE
PGGAHPGSAD DVVDAEVVDD GREAK

The E7-Hsp70 chimera/fusion polypeptide sequences (Nucleotide sequence SEQ ID NO: 21 and amino acid sequence SEQ ID NO: 22) are provided below. The E7 coding sequence is shown in upper case and underscored.

1/1 31/11
ATG CAT GGA GAT ACA CCT ACA TTG CAT GAA TAT ATG TTA GAT TTG CAA CCA GAG ACA ACT
Met His Gly Asp Thr Pro Thr Leu His Glu Tyr Met Leu Asp Leu Gln Pro Glu Thr Thr
61/21 91/31
GAT CTC TAC TGT TAT GAG CAA TTA AAT GAC AGC TCA GAG GAG GAG GAT GAA ATA GAT GGT
Asp Leu Tyr Cys Tyr Glu Gln Leu Asn Asp Ser Ser Glu Glu Glu Asp Glu Ile Asp Gly
121/41 151/51
CCA GCT GGA CAA GCA GAA CCG GAC AGA GCC CAT TAC AAT ATT GTA ACC TTT TGT TGC AAG
Pro Ala Gly Gln Ala Glu Pro Asp Arg Ala His Tyr Asn Ile Val Thr Phe Cys Cys Lys
181/61 211/71
TGT GAC TCT ACG CTT CGG TTG TGC GTA CAA AGC ACA CAC GTA GAC ATT CGT ACT TTG GAA
Cys Asp Ser Thr Leu Arg Leu Cys Val Gln Ser Thr His Val Asp Ile Arg Thr Leu Glu
241/81 271/91
GAC CTG TTA ATG GGC ACA CTA GGA ATT GTG TGC CCC ATC TGT TCT CAA GGA TCC atg gct
Asp Leu Leu Met Gly Thr Leu Gly Ile Val Cys Pro Ile Cys Ser Gln Gly Ser Met ala
301/101 331/111
cgt gcg gtc ggg atc gac ctc ggg acc acc aac tcc gtc gtc tcg gtt ctg gaa ggt ggc
Arg Ala Val Gly Ile Asp Leu Gly Thr Thr Asn Ser Val Val Ser Val Leu Glu Gly Gly
361/121 391/131
gac ccg gtc gtc gtc gcc aac tcc gag ggc tcc agg acc acc ccg tca att gtc gcg ttc
Asp Pro Val Val Val Ala Asn Ser Glu Gly Ser Arg Thr Thr Pro Ser Ile Val Ala Phe
421/141 451/151
gcc cgc aac ggt gag gtg ctg gtc ggc cag ccc gcc aag aac cag gca gtg acc aac gtc
Ala Arg Asn Gly Glu Val Leu Val Gly Gln Pro Ala Lys Asn Gln Ala Val Thr Asn Val
481/161 511/171
gat cgc acc gtg cgc tcg gtc aag cga cac atg ggc agc gac tgg tcc ata gag att gac
Asp Arg Thr Val Arg Ser Val Lys Arg His Met Gly Ser Asp Trp Ser Ile Glu Ile Asp
541/181 571/191
ggc aag aaa tac acc gcg ccg gag atc agc gcc cgc att ctg atg aag ctg aag cgc gac
Gly Lys Lys Tyr Thr Ala Pro Glu Ile Ser Ala Arg Ile Leu Met Lys Leu Lys Arg Asp
601/201 631/211
gcc gag gcc tac ctc ggt gag gac att acc gac gcg gtt atc acg acg ccc gcc tac ttc
Ala Glu Ala Tyr Leu Gly Glu Asp Ile Thr Asp Ala Val Ile Thr Thr Pro Ala Tyr Phe
661/221 691/231
aat gac gcc cag cgt cag gcc acc aag gac gcc ggc cag atc gcc ggc ctc aac gtg ctg
Asn Asp Ala Gln Arg Gln Ala Thr Lys Asp Ala Gly Gln Ile Ala Gly Leu Asn Val Leu
721/241 751/251
cgg atc gtc aac gag ccg acc gcg gcc gcg ctg gcc tac ggc ctc gac aag ggc gag aag
Arg Ile Val Asn Glu Pro Thr Ala Ala Ala Leu Ala Tyr Gly Leu Asp Lys Gly Glu Lys
781/261 811/271
gag cag cga atc ctg gtc ttc gac ttg ggt ggt ggc act ttc gac gtt tcc ctg ctg gag
Glu Gln Arg Ile Leu Val Phe Asp Leu Gly Gly Gly Thr Phe Asp Val Ser Leu Leu Glu
841/281 871/291
atc ggc gag ggt gtg gtt gag gtc cgt gcc act tcg ggt gac aac cac ctc ggc ggc gac
Ile Gly Glu Gly Val Val Glu Val Arg Ala Thr Ser Gly Asp Asn His Leu Gly Gly Asp
901/301 931/311
gac tgg gac cag cgg gtc gtc gat tgg ctg gtg gac aag ttc aag ggc acc agc ggc atc
Asp Trp Asp Gln Arg Val Val Asp Trp Leu Val Asp Lys Phe Lys Gly Thr Ser Gly Ile
961/321 991/331
gat ctg acc aag gac aag atg gcg atg cag cgg ctg cgg gaa gcc gcc gag aag gca aag
Asp Leu Thr Lys Asp Lys Met ala Met Gln Arg Leu Arg Glu Ala Ala Glu Lys Ala Lys
1021/341 1051/351
atc gag ctg agt tcg agt cag tcc acc tcg atc aac ctg ccc tac atc acc gtc gac gcc
Ile Glu Leu Ser Ser Ser Gln Ser Thr Ser Ile Asn Leu Pro Tyr Ile Thr Val Asp Ala
1081/361 1111/371
gac aag aac ccg ttg ttc tta gac gag cag ctg acc cgc gcg gag ttc caa cgg atc act
Asp Lys Asn Pro Leu Phe Leu Asp Glu Gln Leu Thr Arg Ala Glu Phe Gln Arg Ile Thr
1141/381 1171/391
cag gac ctg ctg gac cgc act cgc aag ccg ttc cag tcg gtg atc gct gac acc ggc att
Gln Asp Leu Leu Asp Arg Thr Arg Lys Pro Phe Gln Ser Val Ile Ala Asp Thr Gly Ile
1201/401 1231/411
tcg gtg tcg gag atc gat cac gtt gtg ctc gtg ggt ggt tcg acc cgg atg ccc gcg gtg
Ser Val Ser Glu Ile Asp His Val Val Leu Val Gly Gly Ser Thr Arg Met Pro Ala Val
1261/421 1291/431
acc gat ctg gtc aag gaa ctc acc ggc ggc aag gaa ccc aac aag ggc gtc aac ccc gat
Thr Asp Leu Val Lys Glu Leu Thr Gly Gly Lys Glu Pro Asn Lys Gly Val Asn Pro Asp
1321/441 1351/451
gag gtt gtc gcg gtg gga gcc gct ctg cag gcc ggc gtc ctc aag ggc gag gtg aaa gac
Glu Val Val Ala Val Gly Ala Ala Leu Gln Ala Gly Val Leu Lys Gly Glu Val Lys Asp
1381/461 1411/471
gtt ctg ctg ctt gat gtt acc ccg ctg agc ctg ggt atc gag acc aag ggc ggg gtg atg
Val Leu Leu Leu Asp Val Thr Pro Leu Ser Leu Gly Ile Glu Thr Lys Gly Gly Val Met
1441/481 1471/491
acc agg ctc atc gag cgc aac acc acg atc ccc acc aag cgg tcg gag act ttc acc acc
Thr Arg Leu Ile Glu Arg Asn Thr Thr Ile Pro Thr Lys Arg Ser Glu Thr Phe Thr Thr
1501/501 1531/511
gcc gac gac aac caa ccg tcg gtg cag atc cag gtc tat cag ggg gag cgt gag atc gcc
Ala Asp Asp Asn Gln Pro Ser Val Gln Ile Gln Val Tyr Gln Gly Glu Arg Glu Ile Ala
1561/521 1591/531
gcg cac aac aag ttg ctc ggg tcc ttc gag ctg acc ggc atc ccg ccg gcg ccg cgg ggg
Ala His Asn Lys Leu Leu Gly Ser Phe Glu Leu Thr Gly Ile Pro Pro Ala Pro Arg Gly
1621/541 1651/551
att ccg cag atc gag gtc act ttc gac atc gac gcc aac ggc att gtg cac gtc acc gcc
Ile Pro Gln Ile Glu Val Thr Phe Asp Ile Asp Ala Asn Gly Ile Val His Val Thr Ala
1681/561 1711/571
aag gac aag ggc acc ggc aag gag aac acg atc cga atc cag gaa ggc tcg ggc ctg tcc
Lys Asp Lys Gly Thr Gly Lys Glu Asn Thr Ile Arg Ile Gln Glu Gly Ser Gly Leu Ser
1741/581 1771/591
aag gaa gac att gac cgc atg atc aag gac gcc gaa gcg cac gcc gag gag gat cgc aag
Lys Glu Asp Ile Asp Arg Met Ile Lys Asp Ala Glu Ala His Ala Glu Glu Asp Arg Lys
1801/601 1831/611
cgt cgc gag gag gcc gat gtt cgt aat caa gcc gag aca ttg gtc tac cag acg gag aag
Arg Arg Glu Glu Ala Asp Val Arg Asn Gln Ala Glu Thr Leu Val Tyr Gln Thr Glu Lys
1861/621 1891/631
ttc gtc aaa gaa cag cgt gag gcc gag ggt ggt tcg aag gta cct gaa gac acg ctg aac
Phe Val Lys Glu Gln Arg Glu Ala Glu Gly Gly Ser Lys Val Pro Glu Asp Thr Leu Asn
1921/641 1951/651
aag gtt gat gcc gcg gtg gcg gaa gcg aag gcg gca ctt ggc gga tcg gat att tcg gcc
Lys Val Asp Ala Ala Val Ala Glu Ala Lys Ala Ala Leu Gly Gly Ser Asp Ile Ser Ala
1981/661 2011/671
atc aag tcg gcg atg gag aag ctg ggc cag gag tcg cag gct ctg ggg caa gcg atc tac
Ile Lys Ser Ala Met Glu Lys Leu Gly Gln Glu Ser Gln Ala Leu Gly Gln Ala Ile Tyr
2041/681 2071/691
gaa gca gct cag gct gcg tca cag gcc act ggc gct gcc cac ccc ggc tcg gct gat gaA
GLU ALA ALA GLN ALA ALA SER GLN ALA THR GLY ALA ALA HIS PRO GLY SER ALA ASP GLU
2101/701
AGC a
Ser.

ETA(dII) from Pseudomonas aeruginosa

The complete coding sequence for Pseudomonas aeruginosa exotoxin type A (ETA)-SEQ ID NO: 23-GenBank Accession No. K01397, is shown below:

ctgcagctgg tcaggccgtt tccgcaacgc ttgaagtcct ggccgatata ccggcagggc cagccatcgt tcgacgaata
aagccacctc agccatgatg ccctttccat ccccagcgga accccgacat ggacgccaaa gccctgctcc tcggcagcct
ctgcctggcc gccccattcg ccgacgcggc gacgctcgac aatgctctct ccgcctgcct cgccgcccgg ctcggtgcac
cgcacacggc ggagggccag ttgcacctgc cactcaccct tgaggcccgg cgctccaccg gcgaatgcgg
ctgtacctcg gcgctggtgc gatatcggct gctggccagg ggcgccagcg ccgacagcct cgtgcttcaa gagggctgct
cgatagtcgc caggacacgc cgcgcacgct gaccctggcg gcggacgccg gcttggcgag cggccgcgaa
ctggtcgtca cccigggttg tcaggcgcct gactgacagg ccgggctgcc accaccaggc cgagatggac gccctgcatg
tatcctccga tcggcaagcc tcccgttcgc acattcacca ctctgcaatc cagttcataa atcccataaa agccctcttc
cgctccccgc cagcctcccc gcatcccgca ccctagacgc cccgccgctc tccgccggct cgcccgacaa
gaaaaaccaa ccgctcgatc agcctcatcc ttcacccatc acaggagcca tcgcgatgca cctgataccc cattggatcc
ccctggtcgc cagcctcggc ctgctcgccg gcggctcgtc cgcgtccgcc gccgaggaag ccttcgacct ctggaacgaa
tgcgccaaag cctgcgtgct cgacctcaag gacggcgtgc gttccagccg catgagcgtc gacccggcca tcgccgacac
caacggccag ggcgtgctgc actactccat ggtcctggag ggcggcaacg acgcgctcaa gctggccatc
gacaacgccc tcagcatcac cagcgacggc ctgaccatcc gcctcgaagg cggcgtcgag ccgaacaagc
cggtgcgcta cagctacacg cgccaggcgc gcggcagttg gtcgctgaac tggctggtac cgatcggcca
cgagaagccc tcgaacatca aggtgttcat ccacgaactg aacgccggca accagctcag ccacatgtcg ccgatctaca
ccatcgagat gggcgacgag ttgctggcga agctggcgcg cgatgccacc ttcttcgtca gggcgcacga
gagcaacgag atgcagccga cgctcgccat cagccatgcc ggggtcagcg tggtcatggc ccagacccag
ccgcgccggg aaaagcgctg gagcgaatgg gccagcggca aggtgttgtg cctgctcgac ccgctggacg
gggtctacaa ctacctcgcc cagcaacgct gcaacctcga cgatacctgg gaaggcaaga tctaccgggt gctcgccggc
aacccggcga agcatgacct ggacatcaaa cccacggtca tcagtcatcg cctgcacttt cccgagggcg gcagcctggc
cgcgctgacc gcgcaccagg cttgccacct gccgctggag actttcaccc gtcatcgcca gccgcgcggc tgggaacaac
tggagcagtg cggctatccg gtgcagcggc tggtcgccct ctacctggcg gcgcggctgt cgtggaacca ggtcgaccag
gtgatccgca acgccctggc cagccccggc agcggcggcg acctgggcga agcgatccgc gagcagccgg
agcaggcccg tctggccctg accctggccg ccgccgagag cgagcgcttc gtccggcagg gcaccggcaa
cgacgaggcc ggcgcggcca acgccgacgt ggtgagcctg acctgcccgg tcgccgccgg tgaatgcgcg
ggcccggcgg acagcggcga cgccctgctg gagcgcaact atcccactgg cgcggagttc ctcggcgacg
gcggcgacgt cagcttcagc acccgcggca cgcagaactg gacggtggag cggctgctcc aggcgcaccg
ccaactggag gagcgcggct atgtgttcgt cggctaccac ggcaccttcc tcgaagcggc gcaaagcatc gtcttcggcg
gggtgcgcgc gcgcagccag gacctcgacg cgatctggcg cggtttctat atcgccggcg atccggcgct ggcctacggc
tacgcccagg accaggaacc cgacgcacgc ggccggatcc gcaacggtgc cctgctgcgg gtctatgtgc
cgcgctcgag cctgccgggc ttctaccgca ccagcctgac cctggccgcg ccggaggcgg cgggcgaggt
cgaacggctg atcggccatc cgctgccgct gcgcctggac gccatcaccg gccccgagga ggaaggcggg
cgcctggaga ccattctcgg ctggccgctg gccgagcgca ccgtggtgat tccctcggcg atccccaccg acccgcgcaa
cgtcggcggc gacctcgacc cgtccagcat ccccgacaag gaacaggcga tcagcgccct gccggactac
gccagccagc ccggcaaacc gccgcgcgag gacctgaagt aactgccgcg accggccggc tcccttcgca
ggagccggcc ttctcggggc ctggccatac atcaggtttt cctgatgcca gcccaatcga atatgaattc 2760

The amino acid sequence of ETA (SEQ ID NO: 24), GenBank Accession No. K01397, is:

MHLIPHWIPL VASLGLLAGG SSASAAEEAF DLWNECAKAC
VLDLKDGVRS
SRMSVDPAIA DTNGQGVLHY SMVLEGGNDA LKLAIDNALS
ITSDGLTIRL
EGGVEPNKPV RYSYTRQARG SWSLNWLVPI GHEKPSNIKV
FIHELNAGNQ
LSHMSPIYTI EMGDELLAKL ARDATFFVRA HESNEMQPTL
AISHAGVSVV
MAQTQPRREK RWSEWASGKV LCLLDPLDGV YNYLAQQRCN
LDDTWEGKIY
RVLAGNPAKH DLDIKPTVIS HRLHFPEGGS LAALTAHQAC
HLPLETFTRH
RQPRGWEQLE QCGYPVQRLV ALYLAARLSW NQVDQVIRNA
LASPGSGGDL
GEAIREQPEQ ARLALTLAAA ESERFVRQGT GNDEAGAANA
DVVSLTCPVA
AGECAGPADS GDALLERNYP TGAEFLGDGG DVSFSTRGTQ
NWTVERLLQA
HRQLEERGYV FVGYHGTFLE AAQSIVFGGV RARSQDLDAI
WRGFYIAGDP
ALAYGYAQDQ EPDARGRIRN GALLRVYVPR SSLPGFYRTS
LTLAAPEAAG
EVERLIGHPL PLRLDAITGP EEEGGRLETI LGWPLAERTV
VIPSAIPTDP
RNVGGDLDPS SIPDKEQAIS ALPDYASQPG KPPREDLK 638

Residues 1-25 (italicized) above represent the signal peptide. The first residue of the mature polypeptide, Ala, is bolded/underscored. The mature polypeptide is residues 26-638 of SEQ ID NO: 24.

Domain II (ETA(II)), translocation domain (underscored above) spans residues 247-417 of the mature polypeptide (corresponding to residues 272-442 of SEQ ID NO: 24) and is presented below separately as SEQ ID NO: 25.

RLHFPEGGSL AALTAHQACH LPLETFTRHR QPRGWEQLEQ
CGYPVQRLVA LYLAARLSWN QVDQVIRNAL ASPGSGGDLG
EAIREQPEQA RLALTLAAAE SERFVRQGTG NDEAGAANAD
VVSLTCPVAA GECAGPADSG DALLERNYPT GAEFLGDGGD
VSFSTRGTQN W 171

The construct in which ETA(dII) is fused to HPV-16 E7 is shown below (nucleotides; SEQ ID NO: 26 and amino acids; SEQ ID NO: 27). The ETA(dII) sequence appears in plain font, extra codons from plasmid pcDNA3 are italicized. Nucleotides between ETA(dII) and E7 are also bolded (and result in the interposition of two amino acids between ETA(dII) and E7). The E7 amino acid sequence is underscored (ends with Gln at position 269).

1/1 31/11
atg cgc ctg cac ttt ccc gag ggc ggc agc ctg gcc gcg ctg acc gcg cac cag gct tgc
Met arg leu his phe pro glu gly gly ser leu ala ala leu thr ala his gln ala cys
61/21 91/31
cac ctg ccg ctg gag act ttc acc cgt cat cgc cag ccg cgc ggc tgg gaa caa ctg gag
His Leu Pro Leu Glu Thr Phe Thr Arg His Arg Gln Pro Arg Gly Trp Glu Gln Leu Glu
121/41 151/51
cag tgc ggc tat ccg gtg cag cgg ctg gtc gcc ctc tac ctg gcg gcg cgg ctg tcg tgg
Gln Cys Gly Tyr Pro Val Gln Arg Leu Val Ala Leu Tyr Leu Ala Ala Arg Leu Ser Trp
181/61 211/71
aac cag gtc gac cag gtg atc cgc aac gcc ctg gcc agc ccc ggc agc ggc ggc gac ctg
Asn Gln Val Asp Gln Val Ile Arg Asn Ala Leu Ala Ser Pro Gly Ser Gly Gly Asp Leu
241/81 271/91
ggc gaa gcg atc cgc gag cag ccg gag cag gcc cgt ctg gcc ctg acc ctg gcc gcc gcc
Gly Glu Ala Ile Arg Glu Gln Pro Glu Gln Ala Arg Leu Ala Leu Thr Leu Ala Ala Ala
301/101 331/111
gag agc gag cgc ttc gtc cgg cag ggc acc ggc aac gac gag gcc ggc gcg gcc aac gcc
Glu Ser Glu Arg Phe Val Arg Gln Gly Thr Gly Asn Asp Glu Ala Gly Ala Ala Asn Ala
361/121 391/131
gac gtg gtg agc ctg acc tgc ccg gtc gcc gcc ggt gaa tgc gcg ggc ccg gcg gac agc
Asp Val Val Ser Leu Thr Cys Pro Val Ala Ala Gly Glu Cys Ala Gly Pro Ala Asp Ser
421/141 451/151
ggc gac gcc ctg ctg gag cgc aac tat ccc act ggc gcg gag ttc ctc ggc gac ggc ggc
Gly Asp Ala Leu Leu Glu Arg Asn Tyr Pro Thr Gly Ala Glu Phe Leu Gly Asp Gly Gly
481/161 511/171
gac gtc agc ttc agc acc cgc ggc acg cag [symbol] atg cat gga gat aca cct aca
Asp Val Ser Phe Ser Thr Arg Gly Thr Gln [symbol] Met His Gly Asp Thr Pro Thr
541/181 571/191
ttg cat gaa tat atg tta gat ttg caa cca gag aca act gat ctc tac tgt tat gag caa
Leu His Glu Tyr Met Leu Asp Leu Gln Pro Glu Thr Thr Asp Leu Tyr Cys Tyr Glu Gln
601/201 631/211
tta aat gac agc tca gag gag gag gat gaa ata gat ggt cca gct gga caa gca gaa ccg
Leu Asn Asp Ser Ser Glu Glu Glu Asp Glu Ile Asp Gly Pro Ala Gly Gln Ala Glu Pro
661/221 691/231
gac aga gcc cat tac aat att gta acc ttt tgt tgc aag tgt gac tct acg ctt cgg ttg
Asp Arg Ala His Tyr Asn Ile Val Thr Phe Cys Cys Lys Cys Asp Ser Thr Leu Arg Leu
721/241 751/251
tgc gta caa agc aca cac gta gac att cgt act ttg gaa gac ctg tta atg ggc aca cta
Cys Val Gln Ser Thr His Val Asp Ile Arg Thr Leu Glu Asp Leu Leu Met Gly Thr Leu
781/261 811/271
gga att gtg tgc ccc atc tgt tct caa gga tcc gag ctc ggt acc aag ctt aag ttt aaa
Gly Ile Val Cys Pro Ile Cys Ser Gln Gly Ser Glu Leu Gly Thr Lys Leu Lys Phe Lys
841/281
ccg ctg atc agc ctc gac tgt gcc ttc tag

### Pro Leu Ile Ser Leu Asp Cys Ala Phe AMB

The nucleotide sequence of the pcDNA3 vector encoding E7 and HSP70 (pcDNA3-E7-Hsp70) (SEQ ID NO: 3).

```
atg acc tct cgc cgc tcc gtg aag tcg ggt ccg cgg gag gtt ccg cgc
48
Met Thr Ser Arg Arg Ser Val Lys Ser Gly Pro Arg Glu Val Pro Arg
1 5 10 15
gat gag tac gag gat ctg tac tac acc ccg tct tca ggt atg gcg agt
96
Asp Glu Tyr Glu Asp Leu Tyr Tyr Thr Pro Ser Ser Gly Met Ala Ser
                        20 25 30
ccc gat agt ccg cct gac acc tcc cgc cgt ggc gcc cta cag aca cgc
144
Pro Asp Ser Pro Pro Asp Thr Ser Arg Arg Gly Ala Leu Gln Thr Arg
                35 40 45
tcg cgc cag agg ggc gag gtc cgt ttc gtc cag tac gac gag tcg gat
192
Ser Arg Gln Arg Gly Glu Val Arg Phe Val Gln Tyr Asp Glu Ser Asp
        50 55 60
tat gcc ctc tac ggg ggc tcg tct tcc gaa gac gac gaa cac ccg gag
240
Tyr Ala Leu Tyr Gly Gly Ser Ser Ser Glu Asp Asp Glu His Pro Glu
65 70 75 80
gtc ccc cgg acg cgg cgt ccc gtt tcc ggg gcg gtt ttg tcc ggc ccg
288
Val Pro Arg Thr Arg Arg Pro Val Ser Gly Ala Val Leu Ser Gly Pro
                                85 90 95
ggg cct gcg cgg gcg cct ccg cca ccc gct ggg tcc gga ggg gcc gga
336
Gly Pro Ala Arg Ala Pro Pro Pro Pro Ala Gly Ser Gly Gly Ala Gly
                        100 105 110
cgc aca ccc acc acc gcc ccc cgg gcc ccc cga acc cag cgg gtg gcg
384
Arg Thr Pro Thr Thr Ala Pro Arg Ala Pro Arg Thr Gln Arg Val Ala
                115 120 125
tct aag gcc ccc gcg gcc ccg gcg gcg gag acc acc cgc ggc agg aaa
432
Ser Lys Ala Pro Ala Ala Pro Ala Ala Glu Thr Thr Arg Gly Arg Lys
        130 135 140
tcg gcc cag cca gaa tcc gcc gca ctc cca gac gcc ccc gcg tcg acg
480
Ser Ala Gln Pro Glu Ser Ala Ala Leu Pro Asp Ala Pro Ala Ser Thr
145 150 155 160
gcg cca acc cga tcc aag aca ccc gcg cag ggg ctg gcc aga aag ctg
528
Ala Pro Thr Arg Ser Lys Thr Pro Ala Gln Gly Leu Ala Arg Lys Leu
                                165 170 175
cac ttt agc acc gcc ccc cca aac ccc gac gcg cca tgg acc ccc cgg
576
His Phe Ser Thr Ala Pro Pro Asn Pro Asp Ala Pro Trp Thr Pro Arg
                        180 185 190
gtg gcc ggc ttt aac aag cgc gtc ttc tgc gcc gcg gtc ggg cgc ctg
624
Val Ala Gly Phe Asn Lys Arg Val Phe Cys Ala Ala Val Gly Arg Leu
                195 200 205
gcg gcc atg cat gcc cgg atg gcg gct gtc cag ctc tgg gac atg tcg
672
Ala Ala Met His Ala Arg Met Ala Ala Val Gln Leu Trp Asp Met Ser
        210 215 220
cgt ccg cgc aca gac gaa gac ctc aac gaa ctc ctt ggc atc acc acc
720
Arg Pro Arg Thr Asp Glu Asp Leu Asn Glu Leu Leu Gly Ile Thr Thr
225 230 235 240
atc cgc gtg acg gtc tgc gag ggc aaa aac ctg ctt cag cgc gcc aac
768
Ile Arg Val Thr Val Cys Glu Gly Lys Asn Leu Leu Gln Arg Ala Asn
                                245 250 255
gag ttg gtg aat cca gac gtg gtg cag gac gtc gac gcg gcc acg gcg
816
Glu Leu Val Asn Pro Asp Val Val Gln Asp Val Asp Ala Ala Thr Ala
                        260 265 270
act cga ggg cgt tct gcg gcg tcg cgc ccc acc gag cga cct cga gcc
864
Thr Arg Gly Arg Ser Ala Ala Ser Arg Pro Thr Glu Arg Pro Arg Ala
                275 280 285
cca gcc cgc tcc gct tct cgc ccc aga cgg ccc gtc gag ggt acc gag
912
Pro Ala Arg Ser Ala Ser Arg Pro Arg Arg Pro Val Glu Gly Thr Glu
        290 295 300
ctc gga tcc atg cat gga gat aca cct aca ttg cat gaa tat atg tta
960
Leu Gly Ser Met His Gly Asp Thr Pro Thr Leu His Glu Tyr Met Leu
305 310 315 320
gat ttg caa cca gag aca act gat ctc tac tgt tat gag caa tta aat
1008
Asp Leu Gln Pro Glu Thr Thr Asp Leu Tyr Cys Tyr Glu Gln Leu Asn
                                325 330 335
gac agc tca gag gag gag gat gaa ata gat ggt cca gct gga caa gca
1056
Asp Ser Ser Glu Glu Glu Asp Glu Ile Asp Gly Pro Ala Gly Gln Ala
                        340 345 350
gaa ccg gac aga gcc cat tac aat att gta acc ttt tgt tgc aag tgt
1104
Glu Pro Asp Arg Ala His Tyr Asn Ile Val Thr Phe Cys Cys Lys Cys
                355 360 365
gac tct acg ctt cgg ttg tgc gta caa agc aca cac gta gac att cgt
1152
Asp Ser Thr Leu Arg Leu Cys Val Gln Ser Thr His Val Asp Ile Arg
        370 375 380
act ttg gaa gac ctg tta atg ggc aca cta gga att gtg tgc ccc atc
1200
Thr Leu Glu Asp Leu Leu Met Gly Thr Leu Gly Ile Val Cys Pro Ile
385 390 395 400
tgt tct cag gat aag ctt aag ttt aaa ccg ctg atc agc ctc gac tgt
1248
Cys Ser Gln Asp Lys Leu Lys Phe Lys Pro Leu Ile Ser Leu Asp Cys
                                405 410 415
gcc ttc tag
1257
Ala Phe
```

The nucleic acid sequence of plasmid construct pcDNA3-ETA(dII)/E7 (SEQ ID NO: 4). ETA(dII)/E7 is ligated into the EcoRI/BamHI sites of pcDNA3 vector. The nucleotides encoding ETA(dII)/E7 are shown in upper case and underscored. Plasmid sequence is lower case.

### Calreticulin (CRT)

Calreticulin (CRT), a well-characterized ˜46 kDa protein was described briefly above, as were a number of its biological and biochemical activities. As used herein, “calreticulin” or “CRT” refers to polypeptides and nucleic acids molecules having substantial identity to the exemplary human CRT sequences as described herein or homologues thereof, such as rabbit and rat CRT—well-known in the art. A CRT polypeptide is a polypeptide comprising a sequence identical to or substantially identical to the amino acid sequence of CRT. An exemplary nucleotide and amino acid sequence for a CRT used in the present compositions and methods are presented below. The terms “calreticulin” or “CRT” encompass native proteins as well as recombinantly produced modified proteins that, when fused with an antigen (at the DNA or protein level) promote the induction of immune responses and promote angiogenesis, including a CTL response. Thus, the terms “calreticulin” or “CRT” encompass homologues and allelic variants of human CRT, including variants of native proteins constructed by in vitro techniques, and proteins isolated from natural sources. The CRT polypeptides of the invention, and sequences encoding them, also include fusion proteins comprising non-CRT sequences, particularly MHC class I-binding peptides; and also further comprising other domains, e.g., epitope tags, enzyme cleavage recognition sequences, signal sequences, secretion signals and the like.

A human CRT coding sequence is shown below (SEQ ID NO: 28):

```
      1 atgctgctat ccgtgccgct gctgctcggc ctcctcggcc tggccgtcgc cgagcccgcc
    61 gtctacttca aggagcagtt tctggacgga gacgggtgga cttcccgctg gatcgaatcc
  121 aaacacaagt cagattttgg caaattcgtt ctcagttccg gcaagttcta cggtgacgag
  181 gagaaagata aaggtttgca gacaagccag gatgcacgct tttatgctct gtcggccagt
  241 ttcgagcctt tcagcaacaa aggccagacg ctggtggtgc agttcacggt gaaacatgag
  301 cagaacatcg actgtggggg cggctatgtg aagctgtttc ctaatagttt ggaccagaca
  361 gacatgcacg gagactcaga atacaacatc atgtttggtc ccgacatctg tggccctggc
  421 accaagaagg ttcatgtcat cttcaactac aagggcaaga acgtgctgat caacaaggac
  481 atccgttgca aggatgatga gtttacacac ctgtacacac tgattgtgcg gccagacaac
  541 acctatgagg tgaagattga caacagccag gtggagtccg gctccttgga agacgattgg
  601 gacttcctgc cacccaagaa gataaaggat cctgatgctt caaaaccgga agactgggat
  661 gagcgggcca agatcgatga tcccacagac tccaagcctg aggactggga caagcccgag
  721 catatccctg accctgatgc taagaagccc gaggactggg atgaagagat ggacggagag
  781 tgggaacccc cagtgattca gaaccctgag tacaagggtg agtggaagcc ccggcagatc
  841 gacaacccag attacaaggg cacttggatc cacccagaaa ttgacaaccc cgagtattct
  901 cccgatccca gtatctatgc ctatgataac tttggcgtgc tgggcctgga cctctggcag
  961 gtcaagtctg gcaccatctt tgacaacttc ctcatcacca acgatgaggc atacgctgag
1021 gagtttggca acgagacgtg gggcgtaaca aaggcagcag agaaacaaat gaaggacaaa
1081 caggacgagg agcagaggct taaggaggag gaagaagaca agaaacgcaa agaggaggag
1141 gaggcagagg acaaggagga tgatgaggac aaagatgagg atgaggagga tgaggaggac
1201 aaggaggaag atgaggagga agatgtcccc ggccaggcca aggacgagct gtag 1251
```

The amino acid sequence of the human CRT protein encoded by SEQ ID NO: 28 is set forth below (SEQ ID NO: 29). This amino acid sequence is highly homologous to GenBank Accession No. NM 004343.

```
    1 MLLSVPLLLG LLGLAVAEPA VYFKEQFLDG DGWTSRWIES
        KHKSDFGKFV LSSGKFYGDE
  61 EKDKGLQTSQ DARFYALSAS FEPFSNKGQT LVVQFTVKHE
        QNIDCGGGYV KLFPNSLDQT
121 DMHGDSEYNI MFGPDICGPG TKKVHVIFNY KGKNVLINKD
        IRCKDDEFTH LYTLIVRPDN
181 TYEVKIDNSQ VESGSLEDDW DFLPPKKIKD PDASKPEDWD
        ERAKIDDPTD SKPEDWDKPE
241 HIPDPDAKKP EDWDEEMDGE WEPPVIQNPE YKGEWKPRQI
        DNPDYKGTWI HPEIDNPEYS
301 PDPSIYAYDN FGVLGLDLWQ VKSGTIFDNF LITNDEAYAE
        EFGNETWGVT KAAEKQMKDK
361 QDEEQRLKEE EEDKKRKEEE EAEDKEDDED KDEDEEDEED
        KEEDEEEDVP GQAKDEL 417
```

The amino acid sequence of the rabbit and rat CRT proteins are set forth in GenBank Accession Nos. P1553 and NM 022399, respectively. An alignment of human, rabbit and rat CRT shows that these proteins are highly conserved, and most of the amino acid differences between species are conservative in nature. Most of the variation is found in the alignment of the approximately 36 C-terminal residues. Thus, for the present invention, human CRT may be used as well as, DNA encoding any homologue of CRT from any species that has the requisite biological activity (as an IPP) or any active domain or fragment thereof, may be used in place of human CRT or a domain thereof. The present inventors and colleagues (Cheng et al., supra; incorporated by reference in its entirety) that DNA vaccines encoding each of the N, P, and C domains of CRT chimerically linked to HPV-16 E7 elicited potent antigen-specific CD8+ T cell responses and antitumor immunity in mice vaccinated i.d., by gene gun administration. N-CRT/E7, P-CRT/E7 or C-CRT/E7 DNA each exhibited significantly increased numbers of E7-specific CD8⁺ T cell precursors and impressive antitumor effects against E7-expressing tumors when compared with mice vaccinated with E7 DNA (antigen only). N-CRT DNA administration also resulted in anti-angiogenic antitumor effects. Thus, cancer therapy using DNA encoding N-CRT linked to a tumor antigen may be used for treating tumors through a combination of antigen-specific immunotherapy and inhibition of angiogenesis.

The constructs comprising CRT or one of its domains linked to E7 is illustrated schematically below.

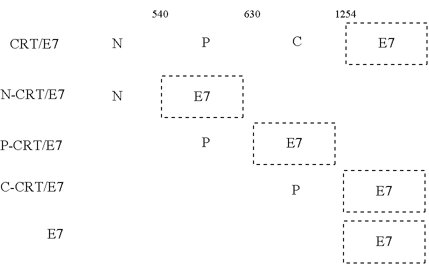

The amino acid sequences of the 3 human CRT domains are shown as annotations of the full length protein (SEQ ID NO: 29). The N domain comprises residues 1-170 (normal text); the P domain comprises residues 171-269 (underscored); and the C domain comprises residues 270-417 (bold/italic)

```
    1 MLLSVPLLLG LLGLAVAEPA VYFKEQFLDG DGWTSRWIES
        KHKSDFGKFV LSSGKFYGDE
  61 EKDKGLQTSQ DARFYALSAS FEPFSNKGQT LVVQFTVKHE
        QNIDCGGGYV KLFPNSLDQT
121 DMHGDSEYNI MFGPDICGPG TKKVHVIFNY KGKNVLINKD
        IRCKDDEFTH LYTLIVRPDN
181 TYEVKIDNSQ VESGSLEDDW DFLPPKKIKD PDASKPEDWD
        ERAKIDDPTD SKPEDWDKPE
241 HIPDPDAKKP EDWDEEMDGE WEPPVIQNPE YKGEWKPRQI
        DNPDYKGTWI HPEIDNPEYS
301 PDPSIYAYDN FGVLGLDLWQ VKSGTIFDNF LITNDEAYAE
        EFGNETWGVT KAAEKQMKDK
361 QDEEQRLKEE EEDKKRKEEE EAEDKEDDED KDEDEEDEED
        KEEDEEEDVP GQAKDEL 417
```

The sequences of the three domains are shown as separate polypeptides below:

### Human N-CRT (SEQ ID NO: 30)

```
    1 MLLSVPLLLG LLGLAVAEPA VYFKEQFLDG DGWTSRWIES
        KHKSDFGKFV LSSGKFYGDE
  61 EKDKGLQTSQ DARFYALSAS FEPFSNKGQT LVVQFTVKHE
        QNIDCGGGYV KLFPNSLDQT
121 DMHGDSEYNI MFGPDICGPG TKKVHVIFNY KGKNVLINKD
        IRCKDDEFTH 170
```

### Human P-CRT (SEQ ID NO: 31)

```
  1 LYTLIVRPDN TYEVKIDNSQ VESGSLEDDW DFLPPKKIKD
      PDASKPEDWD ERAKIDDPTD
61 SKPEDWDKPE HIPDPDAKKP EDWDEEMDGE WEPPVIQNPE
      YKGEWKPRQ 109
```

### Human C-CRT (SEQ ID NO: 32)

```
    1 IDNPDYKGTW IHPEIDNPEY SPDPSIYAYD NFGVLGLDLW
        QVKSGTIFDN FLITNDEAYA
  61 EEFGNETWGV TKAAEKQMKD KQDEEQRLKE EEEDKKRKEE
        EEAEDKEDDE DKDEDEEDEE
121 DKEEDEEEDV PGQAKDEL 138
```

The present vectors may comprises DNA encoding one or more of these domain sequences, which are shown by annotation of SEQ ID NO: 28, below, wherein the N-domain sequence is upper case, the P-domain sequence is lower case/italic/underscored, and the C domain sequence is lower case. The stop codon is also shown but not counted.

| 1 ATGCTGCTAT CCGTGCCGCT GCTGCTCGGC CTCCTCGGCC TGGCCGTCGC CGAGCCCGCC | |
|---|---|
| 61 GTCTACTTCA AGGAGCAGTT TCTGGACGGA GACGGGTGGA CTTCCCGCTG GATCGAATCC | |
| 121 AAACACAAGT CAGATTTTGG CAAATTCGTT CTCAGTTCCG GCAAGTTCTA CGGTGACGAG | |
| 181 GAGAAAGATA AAGGTTTGCA GACAAGCCAG GATGCACGCT TTTATGCTCT GTCGGCCAGT | |
| 241 TTCGAGCCTT TCAGCAACAA AGGCCAGACG CTGGTGGTGC AGTTCACGGT GAAACATGAG | |
| 301 CAGAACATCG ACTGTGGGGG CGGCTATGTG AAGCTGTTTC CTAATAGTTT GGACCAGACA | |
| 361 GACATGCACG GAGACTCAGA ATACAACATC ATGTTTGGTC CCGACATCTG TGGCCCTGGC | |
| 421 ACCAAGAAGG TTCATGTCAT CTTCAACTAC AAGGGCAAGA ACGTGCTGAT CAACAAGGAC | |
| 481 ATCCGTTGCA AGGATGATGA GTTTACACAC CTGTACACAC TGATTGTGCG GCCAGACAAC | |
| 541 acctatgagg tgaagattga caacagccag gtggagtccg gctccttgga agacgattgg | |
| 601 gacttcctgc cacccaagaa gataaaggat cctgatgctt caaaaccgga agactgggat | |
| 661 gagcgggcca agatcgatga tcccacagac tccaagcctg aggactggga caagcccgag | |
| 721 catatccctg accctgatgc taagaagccc gaggactggg atgaagagat ggacggagag | |
| 781 tgggaacccc cagtgattca gaaccctgag tacaagggtg agtggaagcc ccggcagatc | |
| 841 gacaacccag attacaaggg cacttggatc cacccagaaa ttgacaaccc cgagtattct | |
| 901 cccgatccca gtatctatgc ctatgataac tttggcgtgc tgggcctgga cctctggcag | |
| 961 gtcaagtctg gcaccatctt tgacaacttc ctcatcacca acgatgaggc atacgctgag | |
| 1021 gagtttggca acgagacgtg gggcgtaaca aaggcagcag agaaacaaat gaaggacaaa | |
| 1081 caggacgagg agcagaggct taaggaggag gaagaagaca agaaacgcaa agaggaggag | |
| 1141 gaggcagagg acaaggagga tgatgaggac aaagatgagg atgaggagga tgaggaggac | |
| 1201 aaggaggaag atgaggagga agatgtcccc ggccaggcca aggacgagct gtag | 1251 |

The coding sequence for each separate domain is provided below:

### Human N-CRT DNA (SEQ ID NO: 33)

```
    1 ATGCTGCTAT CCGTGCCGCT GCTGCTCGGC CTCCTCGGCC TGGCCGTCGC CGAGCCCGCC
  61 GTCTACTTCA AGGAGCAGTT TCTGGACGGA GACGGGTGGA CTTCCCGCTG GATCGAATCC
121 AAACACAAGT CAGATTTTGG CAAATTCGTT CTCAGTTCCG GCAAGTTCTA CGGTGACGAG
181 GAGAAAGATA AAGGTTTGCA GACAAGCCAG GATGCACGCT TTTATGCTCT GTCGGCCAGT
241 TTCGAGCCTT TCAGCAACAA AGGCCAGACG CTGGTGGTGC AGTTCACGGT GAAACATGAG
301 CAGAACATCG ACTGTGGGGG CGGCTATGTG AAGCTGTTTC CTAATAGTTT GGACCAGACA
361 GACATGCACG GAGACTCAGA ATACAACATC ATGTTTGGTC CCGACATCTG TGGCCCTGGC
421 ACCAAGAAGG TTCATGTCAT CTTCAACTAC AAGGGCAAGA ACGTGCTGAT CAACAAGGAC
481 ATCCGTTGCA AGGATGATGA GTTTACACAC CTGTACACAC TGATTGTGCG GCCAGACAAC
```

### Human P-CRT DNA (SEQ ID NO: 34)

| 1 acctatgagg tgaagattga caacagccag gtggagtccg gctccttgga agacgattgg | |
|---|---|
| 61 gacttcctgc cacccaagaa gataaaggat cctgatgctt caaaaccgga agactgggat | |
| 121 gagcgggcca agatcgatga tcccacagac tccaagcctg aggactggga caagcccgag | |
| 181 catatccctg accctgatgc taagaagccc gaggactggg atgaagagat ggacggagag | |
| 241 tgggaacccc cagtgattca gaaccct | 267 |

### Human C-CRT DNA (SEQ ID NO: 35)

| 1 gagtacaagg gtgagtggaa gccccggcag atcgacaacc cagattacaa gggcacttgg | |
|---|---|
| 61 atccacccag aaattgacaa ccccgagtat tctcccgatc ccagtatcta tgcctatgat | |
| 121 aactttggcg tgctgggcct ggacctctgg caggtcaagt ctggcaccat ctttgacaac | |
| 181 ttcctcatca ccaacgatga ggcatacgct gaggagtttg gcaacgagac gtggggcgta | |
| 241 acaaaggcag cagagaaaca aatgaaggac aaacaggacg aggagcagag gcttaaggag | |
| 301 gaggaagaag acaagaaacg caaagaggag gaggaggcag aggacaagga ggatgatgag | |
| 361 gacaaagatg aggatgagga ggatgaggag gacaaggagg aagatgagga ggaagatgtc | |
| 421 cccggccagg ccaaggacga gctg | 444 |

Alternatively, any nucleotide sequences that encodes these domains may be used in the present constructs. Thus, for use in humans, the sequences may be further codon-optimized.

The present construct may employ combinations of one or more CRT domains, in any of a number of orientations. Using the designations N^{CRT}, P^{CRT} and C^{CRT} to designate the domains, the following are but a few examples of the combinations that may be used in the DNA vaccine vectors of the present invention (where it is understood that Ag can be any antigen, including E7(detox) or E6 (detox).

N^{CRT}-P^{CRT}-Ag; N^{CRT}-P^{CRT}-Ag; N^{CRT}-C^{CRT}-Ag; N^{CRT}-N^{CRT}-Ag; N^{CRT}-N^{CRT}-N^{CRT}-Ag; P^{CRT}-P^{CRT}-Ag; P^{CRT}-C^{CRT}-Ag; P^{CRT}-N^{CRT}-Ag; C^{CRT}-P^{CRT}-Ag; N^{CRT}-P^{CRT}-Ag; etc.

The present invention may employ shorter polypeptide fragments of CRT or CRT domains provided such fragments can enhance the immune response to an antigen with which they are paired. Shorter peptides from the CRT or domain sequences shown above that have the ability to promote protein processing via the MHC-1 class I pathway are also included, and may be defined by routine experimentation.

The present invention may also employ shorter nucleic acid fragments that encode CRT or CRT domains provided such fragments are functional, e.g., encode polypeptides that can enhance the immune response to an antigen with which they are paired (e.g., linked). Nucleic acids that encode shorter peptides from the CRT or domain sequences shown above and are functional, e.g., have the ability to promote protein processing via the MHC-1 class I pathway, are also included, and may be defined by routine experimentation.

A polypeptide fragment of CRT may include at least or about 50, 100, 200, 300, or 400 amino acids. A polypeptide fragment of CRT may also include at least or about 25, 50, 75, 100, 25-50, 50-100, or 75-125 amino acids from a CRT domain selected from the group N-CRT, P-CRT, and C-CRT. A polypeptide fragment of CRT may include residues 1-50, 50-75, 75-100, 100-125, 125-150, 150-170 of the N-domain (e.g., of SEQ ID NO: 30). A polypeptide fragment of CRT may include residues 1-50, 50-75, 75-100, 100-109 of the P-domain (e.g., of SEQ ID NO: 31). A polypeptide fragment of CRT may include residues 1-50, 50-75, 75-100, 100-125, 125-138 of the C-domain (e.g., of SEQ ID NO: 32). A nucleic acid fragment of CRT may encode at least or about 50, 100, 200, 300, or 400 amino acids. A nucleic acid fragment of CRT may also encode at least or about 25, 50, 75, 100, 25-50, 50-100, or 75-125 amino acids from a CRT domain selected from the group N-CRT, P-CRT, and C-CRT. A nucleic acid fragment of CRT may encode residues 1-50, 50-75, 75-100, 100-125, 125-150, 150-170 of the N-domain (e.g., of SEQ ID NO: 30). A nucleic acid fragment of CRT may encode residues 1-50, 50-75, 75-100, 100-109 of the P-domain (e.g., of SEQ ID NO: 31). A nucleic acid fragment of CRT may encode residues 1-50, 50-75, 75-100, 100-125, 125-138 of the C-domain (e.g., of SEQ ID NO: 32).

Polypeptide “fragments” of CRT, as provided herein, do not include full-length CRT. Likewise, nucleic acid “fragments” of CRT, as provided herein, do not include a full-length CRT nucleic acid sequence and do not encode a full-length CRT polypeptide.

In one embodiment, a vector construct of a complete chimeric nucleic acid of the invention, is shown below (SEQ ID NO: 36). The sequence is annotated to show plasmid-derived nucleotides (lower case letters), CRT-derived nucleotides (upper case bold letters), and HPV-E7-derived nucleotides (upper case, italicized/underlined letters). Note that 5 plasmid nucleotides are found between the CRT and E7 coding sequences and that the stop codon for the E7 sequence is double underscored. This plasmid is also referred to as pNGVL4a-CRT/E7(detox).

| 1 gctccgcccc cctgacgagc atcacaaaaa tcgacgctca agtcagaggt ggcgaaaccc | |
|---|---|
| 61 gacaggacta taaagatacc aggcgtttcc ccctggaagc tccctcgtgc gctctcctgt | |
| 121 tccgaccctg ccgcttaccg gatacctgtc cgcctttctc ccttcgggaa gcgtggcgct | |
| 181 ttctcatagc tcacgctgta ggtatctcag ttcggtgtag gtcgttcgct ccaagctggg | |
| 241 ctgtgtgcac gaaccccccg ttcagcccga ccgctgcgcc ttatccggta actatcgtct | |
| 301 tgagtccaac ccggtaagac acgacttatc gccactggca gcagccactg gtaacaggat | |
| 361 tagcagagcg aggtatgtag gcggtgctac agagttcttg aagtggtggc ctaactacgg | |
| 421 ctacactaga agaacagtat ttggtatctg cgctctgctg aagccagtta ccttcggaaa | |
| 481 aagagttggt agctcttgat ccggcaaaca aaccaccgct ggtagcggtg gtttttttgt | |
| 541 ttgcaagcag cagattacgc gcagaaaaaa aggatctcaa gaagatcctt tgatcttttc | |
| 601 tacggggtct gacgctcagt ggaacgaaaa ctcacgttaa gggattttgg tcatgagatt | |
| 661 atcaaaaagg atcttcacct agatcctttt aaattaaaaa tgaagtttta aatcaatcta | |
| 721 aagtatatat gagtaaactt ggtctgacag ttaccaatgc ttaatcagtg aggcacctat | |
| 781 ctcagcgatc tgtctatttc gttcatccat agttgcctga ctcggggggg gggggcgctg | |
| 841 aggtctgcct cgtgaagaag gtgttgctga ctcataccag ggcaacgttg ttgccattgc | |
| 901 tacaggcatc gtggtgtcac gctcgtcgtt tggtatggct tcattcagct ccggttccca | |
| 961 acgatcaagg cgagttacat gatcccccat gttgtgcaaa aaagcggtta gctccttcgg | |
| 1021 tcctccgatc gttgtcagaa gtaagttggc cgcagtgtta tcactcatgg ttatggcagc | |
| 1081 actgcataat tctcttactg tcatgccatc cgtaagatgc ttttctgtga ctggtgagta | |
| 1141 ctcaaccaag tcattctgag aatagtgtat gcggcgaccg agttgctctt gcccggcgtc | |
| 1201 aatacgggat aataccgcgc cacatagcag aactttaaaa gtgctcatca ttggaaaacg | |
| 1261 ttcttcgggg cgaaaactct caaggatctt accgctgttg agatccagtt cgatgtaacc | |
| 1321 cactcgtgca cctgaatcgc cccatcatcc agccagaaag tgagggagcc acggttgatg | |
| 1381 agagctttgt tgtaggtgga ccagttggtg attttgaact tttgctttgc cacggaacgg | |
| 1441 tctgcgttgt cgggaagatg cgtgatctga tccttcaact cagcaaaagt tcgatttatt | |
| 1501 caacaaagcc gccgtcccgt caagtcagcg taatgctctg ccagtgttac aaccaattaa | |
| 1561 ccaattctga ttagaaaaac tcatcgagca tcaaatgaaa ctgcaattta ttcatatcag | |
| 1621 gattatcaat accatatttt tgaaaaagcc gtttctgtaa tgaaggagaa aactcaccga | |
| 1681 ggcagttcca taggatggca agatcctggt atcggtctgc gattccgact cgtccaacat | |
| 1741 caatacaacc tattaatttc ccctcgtcaa aaataaggtt atcaagtgag aaatcaccat | |
| 1801 gagtgacgac tgaatccggt gagaatggca aaagcttatg catttctttc cagacttgtt | |
| 1861 caacaggcca gccattacgc tcgtcatcaa aatcactcgc atcaaccaaa ccgttattca | |
| 1921 ttcgtgattg cgcctgagcg agacgaaata cgcgatcgct gttaaaagga caattacaaa | |
| 1981 caggaatcga atgcaaccgg cgcaggaaca ctgccagcgc atcaacaata ttttcacctg | |
| 2041 aatcaggata ttcttctaat acctggaatg ctgttttccc ggggatcgca gtggtgagta | |
| 2101 accatgcatc atcaggagta cggataaaat gcttgatggt cggaagaggc ataaattccg | |
| 2161 tcagccagtt tagtctgacc atctcatctg taacatcatt ggcaacgcta cctttgccat | |
| 2221 gtttcagaaa caactctggc gcatcgggct tcccatacaa tcgatagatt gtcgcacctg | |
| 2281 attgcccgac attatcgcga gcccatttat acccatataa atcagcatcc atgttggaat | |
| 2341 ttaatcgcgg cctcgagcaa gacgtttccc gttgaatatg gctcataaca ccccttgtat | |
| 2401 tactgtttat gtaagcagac agttttattg ttcatgatga tatattttta tcttgtgcaa | |
| 2461 tgtaacatca gagattttga gacacaacgt ggctttcccc ccccccccat tattgaagca | |
| 2521 tttatcaggg ttattgtctc atgagcggat acatatttga atgtatttag aaaaataaac | |
| 2581 aaataggggt tccgcgcaca tttccccgaa aagtgccacc tgacgtctaa gaaaccatta | |
| 2641 ttatcatgac attaacctat aaaaataggc gtatcacgag gccctttcgt ctcgcgcgtt | |
| 2701 tcggtgatga cggtgaaaac ctctgacaca tgcagctccc ggagacggtc acagcttgtc | |
| 2761 tgtaagcgga tgccgggagc agacaagccc gtcagggcgc gtcagcgggt gttggcgggt | |
| 2821 gtcggggctg gcttaactat gcggcatcag agcagattgt actgagagtg caccatatgc | |
| 2881 ggtgtgaaat accgcacaga tgcgtaagga gaaaataccg catcagattg gctattggcc | |
| 2941 attgcatacg ttgtatccat atcataatat gtacatttat attggctcat gtccaacatt | |
| 3001 accgccatgt tgacattgat tattgactag ttattaatag taatcaatta cggggtcatt | |
| 3061 agttcatagc ccatatatgg agttccgcgt tacataactt acggtaaatg gcccgcctgg | |
| 3121 ctgaccgccc aacgaccccc gcccattgac gtcaataatg acgtatgttc ccatagtaac | |
| 3181 gccaataggg actttccatt gacgtcaatg ggtggagtat ttacggtaaa ctgcccactt | |
| 3241 ggcagtacat caagtgtatc atatgccaag tacgccccct attgacgtca atgacggtaa | |
| 3301 atggcccgcc tggcattatg cccagtacat gaccttatgg gactttccta cttggcagta | |
| 3361 catctacgta ttagtcatcg ctattaccat ggtgatgcgg ttttggcagt acatcaatgg | |
| 3421 gcgtggatag cggtttgact cacggggatt tccaagtctc caccccattg acgtcaatgg | |
| 3481 gagtttgttt tggcaccaaa atcaacggga ctttccaaaa tgtcgtaaca actccgcccc | |
| 3541 attgacgcaa atgggcggta ggcgtgtacg gtgggaggtc tatataagca gagctcgttt | |
| 3601 agtgaaccgt cagatcgcct ggagacgcca tccacgctgt tttgacctcc atagaagaca | |
| 3661 ccgggaccga tccagcctcc gcggccggga acggtgcatt ggaacgcgga ttccccgtgc | |
| 3721 caagagtgac gtaagtaccg cctatagact ctataggcac acccctttgg ctcttatgca | |
| 3781 tgctatactg tttttggctt ggggcctata cacccccgct tccttatgct ataggtgatg | |
| 3841 gtatagctta gcctataggt gtgggttatt gaccattatt gaccactcca acggtggagg | |
| 3901 gcagtgtagt ctgagcagta ctcgttgctg ccgcgcgcgc caccagacat aatagctgac | |
| 3961 agactaacag actgttcctt tccatgggtc ttttctgcag tcaccgtcgt cgacATGCTG | |
| 4021 CTATCCGTGC CGCTGCTGCT CGGCCTCCTC GGCCTGGCCG TCGCCGAGCC TGCCGTCTAC | |
| 4081 TTCAAGGAGC AGTTTCTGGA CGGGGACGGG TGGACTTCCC GCTGGATCGA ATCCAAACAC | |
| 4141 AAGTCAGATT TTGGCAAATT CGTTCTCAGT TCCGGCAAGT TCTACGGTGA CGAGGAGAAA | |
| 4201 GATAAAGGTT TGCAGACAAG CCAGGATGCA CGCTTTTATG CTCTGTCGGC CAGTTTCGAG | |
| 4261 CCTTTCAGCA ACAAAGGCCA GACGCTGGTG GTGCAGTTCA CGGTGAAACA TGAGCAGAAC | |
| 4321 ATCGACTGTG GGGGCGGCTA TGTGAAGCTG TTTCCTAATA GTTTGGACCA GACAGACATG | |
| 4381 CACGGAGACT CAGAATACAA CATCATGTTT GGTCCCGACA TCTGTGGCCC TGGCACCAAG | |
| 4441 AAGGTTCATG TCATCTTCAA CTACAAGGGC AAGAACGTGC TGATCAACAA GGACATCCGT | |
| 4501 TGCAAGGATG ATGAGTTTAC ACACCTGTAC ACACTGATTG TGCGGCCAGA CAACACCTAT | |
| 4561 GAGGTGAAGA TTGACAACAG CCAGGTGGAG TCCGGCTCCT TGGAAGACGA TTGGGACTTC | |
| 4621 CTGCCACCCA AGAAGATAAA GGATCCTGAT GCTTCAAAAC CGGAAGACTG GGATGAGCGG | |
| 4681 GCCAAGATCG ATGATCCCAC AGACTCCAAG CCTGAGGACT GGGACAAGCC CGAGCATATC | |
| 4741 CCTGACCCTG ATGCTAAGAA GCCCGAGGAC TGGGATGAAG AGATGGACGG AGAGTGGGAA | |
| 4801 CCCCCAGTGA TTCAGAACCC TGAGTACAAG GGTGAGTGGA AGCCCCGGCA GATCGACAAC | |
| 4861 CCAGATTACA AGGGCACTTG GATCCACCCA GAAATTGACA ACCCCGAGTA TTCTCCCGAT | |
| 4921 CCCAGTATCT ATGCCTATGA TAACTTTGGC GTGCTGGGCC TGGACCTCTG GCAGGTCAAG | |
| 4981 TCTGGCACCA TCTTTGACAA CTTCCTCATC ACCAACGATG AGGCATACGC TGAGGAGTTT | |
| 5041 GGCAACGAGA CGTGGGGCGT AACAAAGGCA GCAGAGAAAC AAATGAAGGA CAAACAGGAC | |
| 5101 GAGGAGCAGA GGCTTAAGGA GGAGGAAGAA GACAAGAAAC GCAAAGAGGA GGAGGAGGCA | |
| 5161 GAGGACAAGG AGGATGATGA GGACAAAGAT GAGGATGAGG AGGATGAGGA GGACAAGGAG | |
| 5221 GAAGATGAGG AGGAAGATGT CCCCGGCCAG GCCAAGGACG AGCTG [symbol] [symbol] | |
| 5281 [symbol] [symbol] | |
| 5341 [symbol] [symbol] | |
| 5401 [symbol] [symbol] | |
| 5461 [symbol] [symbol] | |
| 5521 [symbol] TAAgg atccagatct | |
| 5581 ttttccctct gccaaaaatt atggggacat catgaagccc cttgagcatc tgacttctgg | |
| 5641 ctaataaagg aaatttattt tcattgcaat agtgtgttgg aattttttgt gtctctcact | |
| 5701 cggaaggaca tatgggaggg caaatcattt aaaacatcag aatgagtatt tggtttagag | |
| 5761 tttggcaaca tatgcccatt cttccgcttc ctcgctcact gactcgctgc gctcggtcgt | |
| 5821 tcggctgcgg cgagcggtat cagctcactc aaaggcggta atacggttat ccacagaatc | |
| 5881 aggggataac gcaggaaaga acatgtgagc aaaaggccag caaaaggcca ggaaccgtaa | |
| 5941 aaaggccgcg ttgctggcgt ttttccatag | 5970 |

The Table below describes the structure of the above plasmid.

| Plasmid | | |
|---|---|---|
| Position | Genetic Construct | Source of Construct |
| 5970-0823 | E. coli ORI (ColEl) | pBR/E. coli -derived |
| 0837-0881 | portion of transposase | Common plasmid sequence |
| | (tpnA) | Tn5/Tn903 |
| 0882-1332 | β-Lactamase (Amp^{R}) | pBRpUC derived plasmid |
| 1331-2496 | AphA (Kan^{R}) | Tn903 |
| 2509-2691 | P3 Promoter DNA binding | Tn3/pBR322 |
| | site | |
| 2692-2926 | pUC backbone | Common plasmid sequence |
| | | pBR322-derived |
| 2931-4009 | NF1 binding and promoter | HHV-5(HCMV UL-10 lE1 |
| | | gene) |
| 4010-4014 | Poly-cloning site | Common plasmid sequence |
| 4015-5265 | Calreticulin (CRT) | Human Calreticulin |
| 5266-5271 | GAATTC plasmid | Remain after cloning |
| | sequence | |
| 5272-5568 | dE7 gene (detoxified | HPV-16 (E7 gene) incl. |
| | partial) | stop codon |
| 5569-5580 | Poly-cloning site | Common plasmid sequence |
| 551-5970 | Poly-Adenylation site | Mammalian signal, pHCMV- |
| | | derived |

In some embodiments, an alternative to CRT is another ER chaperone polypeptide exemplified by ER60, GRP94 or gp96, well-characterized ER chaperone polypeptide that representatives of the HSP90 family of stress-induced proteins (see WO 02/012281, incorporated herein by reference). The term “endoplasmic reticulum chaperone polypeptide” as used herein means any polypeptide having substantially the same ER chaperone function as the exemplary chaperone proteins CRT, tapasin, ER60 or calnexin. Thus, the term includes all functional fragments or variants or mimics thereof. A polypeptide or peptide can be routinely screened for its activity as an ER chaperone using assays known in the art. While the invention is not limited by any particular mechanism of action, in vivo chaperones promote the correct folding and oligomerization of many glycoproteins in the ER, including the assembly of the MHC class I heterotrimeric molecule (heavy (H) chain, (32m, and peptide). They also retain incompletely assembled MHC class I heterotrimeric complexes in the ER (Hauri FEBS Lett. 476:32-37, 2000).

Intercellular Spreading Proteins

The potency of naked DNA vaccines may be enhanced by their ability to amplify and spread in vivo. VP22, a herpes simplex virus type 1 (HSV-1) protein and its “homologues” in other herpes viruses, such as the avian Marek's Disease Virus (MDV) have the property of intercellular transport that provide an approach for enhancing vaccine potency. The present inventors have previously created novel fusions of VP22 with a model antigen, human papillomavirus type 16 (HPV-16) E7, in a DNA vaccine which generated enhanced spreading and MHC class I presentation of antigen. These properties led to a dramatic increase in the number of E7-specific CD8+ T cell precursors in vaccinated mice (at least 50-fold) and converted a less effective DNA vaccine into one with significant potency against E7-expressing tumors. In comparison, a non-spreading mutant, VP22(1-267), failed to enhance vaccine potency. Results presented in U.S. Patent Application publication No. 20040028693 (U.S. Pat. No. 7,318,928), hereby incorporated by reference in its entirety, show that the potency of DNA vaccines is dramatically improved through enhanced intercellular spreading and MHC class I presentation of the antigen.

A similar study linking MDV-1 UL49 to E7 also led to a dramatic increase in the number of E7-specific CD8+ T cell precursors and potency response against E7-expressing tumors in vaccinated mice. Mice vaccinated with a MDV-1 UL49 DNA vaccine stimulated E7-specific CD8+ T cell precursor at a level comparable to that induced by HSV-1 VP22/E7. Thus, fusion of MDV-1UL49 DNA to DNA encoding a target antigen gene significantly enhances the DNA vaccine potency.

In one embodiment, the spreading protein may be a viral spreading protein, including a herpes virus VP22 protein. Exemplified herein are fusion constructs that comprise herpes simplex virus-1 (HSV-1) VP22 (abbreviated HVP22) and its homologue from Marek's disease virus (MDV) termed MDV-VP22 or MVP-22. Also included in the invention are homologues of VP22 from other members of the herpesviridae or polypeptides from nonviral sources that are considered to be homologous and share the functional characteristic of promoting intercellular spreading of a polypeptide or peptide that is fused or chemically conjugated thereto.

DNA encoding HVP22 has the sequence SEQ ID NO: 7 as nucleotides 1-921 of the longer sequence SEQ ID NO: 6 (which is the full length nucleotide sequence of a vector that comprises HVP22). DNA encoding MDV-VP22 is SEQ ID NO: 37 shown below:

| 1 atg ggg gat tct gaa agg cgg aaa tcg gaa cgg cgt cgt tcc ctt gga | |
|---|---|
| 48 tat ccc tct gca tat gat gac gtc tcg att cct gct cgc aga cca tca | |
| 96 aca cgt act cag cga aat tta aac cag gat gat ttg tca aaa cat gga | |
| 144 cca ttt acc gac cat cca aca caa aaa cat aaa tcg gcg aaa gcc gta | |
| 192 tcg gaa gac gtt tcg tct acc acc cgg ggt ggc ttt aca aac aaa ccc | |
| 240 cgt acc aag ccc ggg gtc aga gct gta caa agt aat aaa ttc gct ttc | |
| 288 agt acg gct cct tca tca gca tct agc act tgg aga tca aat aca gtg | |
| 336 gca ttt aat cag cgt atg ttt tgc gga gcg gtt gca act gtg gct caa | |
| 384 tat cac gca tac caa ggc gcg ctc gcc ctt tgg cgt caa gat cct ccg | |
| 432 cga aca aat gaa gaa tta gat gca ttt ctt tcc aga gct gtc att aaa | |
| 480 att acc att caa gag ggt cca aat ttg atg ggg gaa gcc gaa acc tgt | |
| 528 gcc cgc aaa cta ttg gaa gag tct gga tta tcc cag ggg aac gag aac | |
| 576 gta aag tcc aaa tot gaa cgt aca acc aaa tct gaa cgt aca aga cgc | |
| 624 ggc ggt gaa att gaa atc aaa tcg cca gat ccg gga tct cat cgt aca | |
| 672 cat aac cct cgc act ccc gca act tcg cgt cgc cat cat tca tcc gcc | |
| 720 cgc gga tat cgt agc agt gat agc gaa taa | 747 |

The amino acid sequence of HVP22 polypeptide is SEQ ID NO: 38 as amino acid residues 1-301 of SEQ ID NO: 39 (the full length amino acid encoded by the vector).

The amino acid sequence of the MDV-VP22, SEQ ID NO: 40, is below:

| 2 Met Gly Asp Ser Glu Arg Arg Lys Ser Glu Arg Arg Arg Ser Leu Gly | |
|---|---|
| 16 Tyr Pro Ser Ala Tyr Asp Asp Val Ser Ile Pro Ala Arg Arg Pro Ser | |
| 32 Thr Arg Thr Gln Arg Asn Leu Asn Gln Asp Asp Leu Ser Lys His Gly | |
| 48 Pro Phe Thr Asp His Pro Thr Gln Lys His Lys Ser Ala Lys Ala Val | |
| 64 Ser Glu Asp Val Ser Ser Thr Thr Arg Gly Gly Phe Thr Asn Lys Pro | |
| 80 Arg Thr Lys Pro Gly Val Arg Ala Val Gln Ser Asn Lys Phe Ala Phe | |
| 96 Ser Thr Ala Pro Ser Ser Ala Ser Ser Thr Trp Arg Ser Asn Thr Val | |
| 112 Ala Phe Asn Gln Arg Met Phe Cys Gly Ala Val Ala Thr Val Ala Gln | |
| 128 Tyr His Ala Tyr Gln Gly Ala Leu Ala Leu Trp Arg Gln Asp Pro Pro | |
| 144 Arg Thr Asn Glu Glu Leu Asp Ala Phe Leu Ser Arg Ala Val Ile Lys | |
| 160 Ile Thr Ile Gln Glu Gly Pro Asn Leu Met Gly Glu Ala Glu Thr Cys | |
| 176 Ala Arg Lys Leu Leu Glu Glu Ser Gly Leu Ser Gln Gly Asn Glu Asn | |
| 192 Val Lys Ser Lys Ser Glu Arg Thr Thr Lys Ser Glu Arg Thr Arg Arg | |
| 208 Gly Gly Glu Ile Glu Ile Lys Ser Pro Asp Pro Gly Ser His Arg Thr | |
| 224 His Asn Pro Arg Thr Pro Ala Thr Ser Arg Arg His His Ser Ser Ala | |
| 240 Arg Gly Tyr Arg Ser Ser Asp Ser Glu | 249 |

A DNA clone pcDNA3 VP22/E7, that includes the coding sequence for HVP22 and the HPV-16 protein, E7 (plus some additional vector sequence) is SEQ ID NO: 6.

The amino acid sequence of E7 (SEQ ID NO: 41) is residues 308-403 of SEQ ID NO: 39. This particular clone has only 96 of the 98 residues present in E7. The C-terminal residues of wild-type E7, Lys and Pro, are absent from this construct. This is an example of a deletion variant as the term is described below. Such deletion variants (e.g., terminal truncation of two or a small number of amino acids) of other antigenic polypeptides are examples of the embodiments intended within the scope of the fusion polypeptides of this invention.

### Homologues of IPPs

Homologues or variants of IPPs described herein, may also be used, provided that they have the requisite biological activity. These include various substitutions, deletions, or additions of the amino acid or nucleic acid sequences. Due to code degeneracy, for example, there may be considerable variation in nucleotide sequences encoding the same amino acid sequence.

A functional derivative of an IPP retains measurable IPP-like activity, including that of promoting immunogenicity of one or more antigenic epitopes fused thereto by promoting presentation by class I pathways. “Functional derivatives” encompass “variants” and “fragments” regardless of whether the terms are used in the conjunctive or the alternative herein.

The term “chimeric” or “fusion” polypeptide or protein refers to a composition comprising at least one polypeptide or peptide sequence or domain that is chemically bound in a linear fashion with a second polypeptide or peptide domain. One embodiment of this invention is an isolated or recombinant nucleic acid molecule encoding a fusion protein comprising at least two domains, wherein the first domain comprises an IPP and the second domain comprises an antigenic epitope, e.g., an MHC class I-binding peptide epitope. The “fusion” can be an association generated by a peptide bond, a chemical linking, a charge interaction (e.g., electrostatic attractions, such as salt bridges, H-bonding, etc.) or the like. If the polypeptides are recombinant, the “fusion protein” can be translated from a common mRNA. Alternatively, the compositions of the domains can be linked by any chemical or electrostatic means. The chimeric molecules of the invention (e.g., targeting polypeptide fusion proteins) can also include additional sequences, e.g., linkers, epitope tags, enzyme cleavage recognition sequences, signal sequences, secretion signals, and the like. Alternatively, a peptide can be linked to a carrier simply to facilitate manipulation or identification/location of the peptide.

Also included is a “functional derivative” of an IPP, which refers to an amino acid substitution variant, a “fragment” of the protein. A functional derivative of an IPP retains measurable activity that may be manifested as promoting immunogenicity of one or more antigenic epitopes fused thereto or co-administered therewith. “Functional derivatives” encompass “variants” and “fragments” regardless of whether the terms are used in the conjunctive or the alternative herein.

A functional homologue must possess the above biochemical and biological activity. In view of this functional characterization, use of homologous proteins including proteins not yet discovered, fall within the scope of the invention if these proteins have sequence similarity and the recited biochemical and biological activity.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In one embodiment, the method of alignment includes alignment of Cys residues.

In one embodiment, the length of a sequence being compared is at least 30%, at least 40%, at least 50%, at least 60%, and at least 70%, 80%, 90%, 95%, 96%, 97%, 98%, or 99% of the length of the IPP reference sequence. The amino acid residues (or nucleotides) at corresponding amino acid (or nucleotide) positions are then compared. When a position in the first sequence is occupied by the same amino acid residue (or nucleotide) as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In one embodiment, the percent identity between two amino acid sequences is determined using the Needleman and Wunsch (J. Mol. Biol. 48:444-453 (1970) algorithm which has been incorporated into the GAP program in the GCG software package (available at http://www.gcg.com), using either a Blossom 62 matrix or a PAM250 matrix, and a gap weight of 16, 14, 12, 10, 8, 6, or 4 and a length weight of 1, 2, 3, 4, 5, or 6. In yet another embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4.

The nucleic acid and protein sequences of the present invention can further be used as a “query sequence” to perform a search against public databases, for example, to identify other family members or related sequences. Such searches can be performed using the NBLAST and XBLAST programs (version 2.0) of Altschul et al. (1990) J. Mol. Biol. 215:403-10. BLAST nucleotide searches can be performed with the NBLAST program, score=100, wordlength=12 to obtain nucleotide sequences homologous to IPP nucleic acid molecules. BLAST protein searches can be performed with the XBLAST program, score=50, wordlength=3 to obtain amino acid sequences homologous to IPP protein molecules. To obtain gapped alignments for comparison purposes, Gapped BLAST can be utilized as described in Altschul et al. (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov.

Thus, a homologue of an IPP or of an IPP domain described above is characterized as having (a) functional activity of native IPP or domain thereof and (b) amino acid sequence similarity to a native IPP protein or domain thereof when determined as above, of at least about 50%, 55%, 60%, 65%, 70%, 75%, 80%, 85%, 90%, 95%, 96%, 97%, 98%, or 99%.

It is within the skill in the art to obtain and express such a protein using DNA probes based on the disclosed sequences of an IPP. Then, the fusion protein's biochemical and biological activity can be tested readily using art-recognized methods such as those described herein, for example, a T cell proliferation, cytokine secretion or a cytolytic assay, or an in vivo assay of tumor protection or tumor therapy. A biological assay of the stimulation of antigen-specific T cell reactivity will indicate whether the homologue has the requisite activity to qualify as a “functional” homologue.

A “variant” refers to a molecule substantially identical to either the full protein or to a fragment thereof in which one or more amino acid residues have been replaced (substitution variant) or which has one or several residues deleted (deletion variant) or added (addition variant). A “fragment” of an IPP refers to any subset of the molecule, that is, a shorter polypeptide of the full-length protein.

A number of processes can be used to generate fragments, mutants and variants of the isolated DNA sequence. Small subregions or fragments of the nucleic acid encoding the spreading protein, for example 1-30 bases in length, can be prepared by standard, chemical synthesis. Antisense oligonucleotides and primers for use in the generation of larger synthetic fragment.

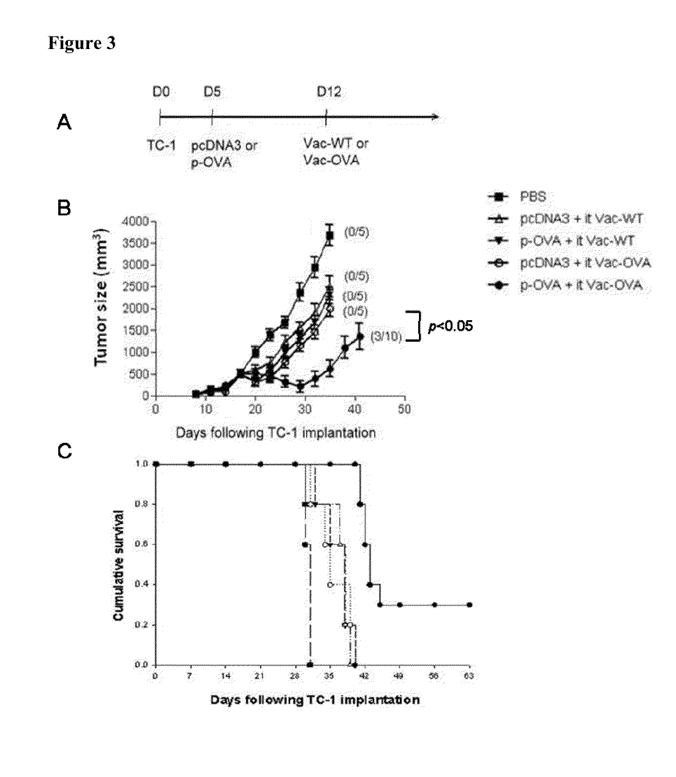

A one group of variants are those in which at least one amino acid residue and in certain embodiments only one, has been substituted by different residue. For a detailed description of protein chemistry and structure, see Schulz, G E et al., Principles of Protein Structure, Springer-Verlag, New York, 1978, and Creighton, T. E., Proteins: Structure and Molecular Properties, W.H. Freeman & Co., San Francisco, 1983, which are hereby incorporated by reference. The types of substitutions that may be made in the protein molecule may be based on analysis of the frequencies of amino acid changes between a homologous protein of different species, such as those presented in Table 1-2 of Schulz et al. (supra) and FIG. 3-9 of Creighton (supra). Based on such an analysis, conservative substitutions are defined herein as exchanges within one of the following five groups:

1. Small aliphatic, nonpolar or slightly polar residues Ala, Ser, Thr (Pro, Gly);
2. Polar, negatively charged residues and their amides Asp, Asn, Glu, Gln;
3. Polar, positively charged residues His, Arg, Lys;
4. Large aliphatic, nonpolar residues Met, Leu, Ile, Val (Cys)
5. Large aromatic residues Phe, Tyr, Trp.

The three amino acid residues in parentheses above have special roles in protein architecture. Gly is the only residue lacking a side chain and thus imparts flexibility to the chain. Pro, because of its unusual geometry, tightly constrains the chain. Cys can participate in disulfide bond formation, which is important in protein folding.

More substantial changes in biochemical, functional (or immunological) properties are made by selecting substitutions that are less conservative, such as between, rather than within, the above five groups. Such changes will differ more significantly in their effect on maintaining (a) the structure of the peptide backbone in the area of the substitution, for example, as a sheet or helical conformation, (b) the charge or hydrophobicity of the molecule at the target site, or (c) the bulk of the side chain. Examples of such substitutions are (i) substitution of Gly and/or Pro by another amino acid or deletion or insertion of Gly or Pro; (ii) substitution of a hydrophilic residue, e.g., Ser or Thr, for (or by) a hydrophobic residue, e.g., Leu, Ile, Phe, Val or Ala; (iii) substitution of a Cys residue for (or by) any other residue; (iv) substitution of a residue having an electropositive side chain, e.g., Lys, Arg or His, for (or by) a residue having an electronegative charge, e.g., Glu or Asp; or (v) substitution of a residue having a bulky side chain, e.g., Phe, for (or by) a residue not having such a side chain, e.g., Gly.

Most acceptable deletions, insertions and substitutions according to the present invention are those that do not produce radical changes in the characteristics of the wild-type or native protein in terms of its relevant biological activity, e.g., its ability to stimulate antigen specific T cell reactivity to an antigenic epitope or epitopes that are fused to the protein. However, when it is difficult to predict the exact effect of the substitution, deletion or insertion in advance of doing so, one skilled in the art will appreciate that the effect can be evaluated by routine screening assays such as those described here, without requiring undue experimentation.

Exemplary fusion proteins provided herein comprise an IPP protein or homolog thereof and an antigen. For example, a fusion protein may comprise, consist essentially of, or consist of an IPP or an IPP fragment, e.g., N-CRT, P-CRT and/or C-CRT, or an amino acid sequence that is at least about 50%, 55%, 60%, 65%, 70%, 75%, 80%, 85%, 90%, 95%, 96%, 97%, 98%, or 99% identical to the amino acid sequence of the IPP or IPP fragment, wherein the IPP fragment is functionally active as further described herein, linked to an antigen. A fusion protein may also comprise an IPP or an IPP fragment and at least 1, 2, 3, 4, 5, 6, 7, 8, 9, 10 or more amino acids, or about 1-5, 1-10, 1-15, 1-20, 1-25, 1-30, 1-50 amino acids, at the N- and/or C-terminus of the IPP fragment. These additional amino acids may have an amino acid sequence that is unrelated to the amino acid sequence at the corresponding position in the IPP protein.

Homologs of an IPP or an IPP fragments may also comprise, consist essentially of, or consist of an amino acid sequence that differs from that of an IPP or IPP fragment by the addition, deletion, or substitution, e.g., conservative substitution, of at least about 1, 2, 3, 4, 5, 6, 7, 8, 9 or 10 amino acids, or from about 1-5, 1-10, 1-15 or 1-20 amino acids. Homologs of an IPP or IPP fragments may be encoded by nucleotide sequences that are at least about 50%, 55%, 60%, 65%, 70%, 75%, 80%, 85%, 90%, 95%, 96%, 97%, 98%, or 99% identical to the nucleotide sequence encoding an IPP or IPP fragment, such as those described herein.

Yet other homologs of an IPP or IPP fragments are encoded by nucleic acids that hybridize under stringent hybridization conditions to a nucleic acid that encodes an IPP or IPP fragment. For example, homologs may be encoded by nucleic acids that hybridize under high stringency conditions of 0.2 to 1×SSC at 65° C. followed by a wash at 0.2×SSC at 65° C. to a nucleic acid consisting of a sequence described herein. Nucleic acids that hybridize under low stringency conditions of 6×SSC at room temperature followed by a wash at 2×SSC at room temperature to nucleic acid consisting of a sequence described herein or a portion thereof can be used. Other hybridization conditions include 3×SSC at 40 or 50° C., followed by a wash in 1 or 2×SSC at 20, 30, 40, 50, 60, or 65° C. Hybridizations can be conducted in the presence of formaldehyde, e.g., 10%, 20%, 30% 40% or 50%, which further increases the stringency of hybridization. Theory and practice of nucleic acid hybridization is described, e.g., in S. Agrawal (ed.) Methods in Molecular Biology, volume 20; and Tijssen (1993) Laboratory Techniques in biochemistry and molecular biology-hybridization with nucleic acid probes, e.g., part I chapter 2 “Overview of principles of hybridization and the strategy of nucleic acid probe assays,” Elsevier, New York provide a basic guide to nucleic acid hybridization.

A fragment of a nucleic acid sequence is defined as a nucleotide sequence having fewer nucleotides than the nucleotide sequence encoding the full length CRT polypeptide, antigenic polypeptide, or the fusion thereof. This invention includes such nucleic acid fragments that encode polypeptides which retain the ability of the fusion polypeptide to induce increases in frequency or reactivity of T cells, including CD8+ T cells, that are specific for the antigen part of the fusion polypeptide.

Nucleic acid sequences of this invention may also include linker sequences, natural or modified restriction endonuclease sites and other sequences that are useful for manipulations related to cloning, expression or purification of encoded protein or fragments. For example, a fusion protein may comprise a linker between the antigen and the IPP protein.

Other nucleic acid vaccines that may be used include single chain trimers (SCT), as further described in the Examples and in references cited therein, all of which are specifically incorporated by reference herein.

### Backbone of Nucleic Acid Vaccine

A nucleic acid, e.g., DNA vaccine may comprise an “expression vector” or “expression cassette,” i.e., a nucleotide sequence which is capable of affecting expression of a protein coding sequence in a host compatible with such sequences. Expression cassettes include at least a promoter operably linked with the polypeptide coding sequence; and, optionally, with other sequences, e.g., transcription termination signals. Additional factors necessary or helpful in effecting expression may also be included, e.g., enhancers.

“Operably linked” means that the coding sequence is linked to a regulatory sequence in a manner that allows expression of the coding sequence. Known regulatory sequences are selected to direct expression of the desired protein in an appropriate host cell. Accordingly, the term “regulatory sequence” includes promoters, enhancers and other expression control elements. Such regulatory sequences are described in, for example, Goeddel, Gene Expression Technology. Methods in Enzymology, vol. 185, Academic Press, San Diego, Calif. (1990)).

A promoter region of a DNA or RNA molecule binds RNA polymerase and promotes the transcription of an “operably linked” nucleic acid sequence. As used herein, a “promoter sequence” is the nucleotide sequence of the promoter which is found on that strand of the DNA or RNA which is transcribed by the RNA polymerase. Two sequences of a nucleic acid molecule, such as a promoter and a coding sequence, are “operably linked” when they are linked to each other in a manner which permits both sequences to be transcribed onto the same RNA transcript or permits an RNA transcript begun in one sequence to be extended into the second sequence. Thus, two sequences, such as a promoter sequence and a coding sequence of DNA or RNA are operably linked if transcription commencing in the promoter sequence will produce an RNA transcript of the operably linked coding sequence. In order to be “operably linked” it is not necessary that two sequences be immediately adjacent to one another in the linear sequence.

In one embodiment, certain promoter sequences of the present invention must be operable in mammalian cells and may be either eukaryotic or viral promoters. Certain promoters are also described in the Examples, and other useful promoters and regulatory elements are discussed below. Suitable promoters may be inducible, repressible or constitutive. A “constitutive” promoter is one which is active under most conditions encountered in the cell's environmental and throughout development. An “inducible” promoter is one which is under environmental or developmental regulation. A “tissue specific” promoter is active in certain tissue types of an organism. An example of a constitutive promoter is the viral promoter MSV-LTR, which is efficient and active in a variety of cell types, and, in contrast to most other promoters, has the same enhancing activity in arrested and growing cells. Other viral promoters include that present in the CMV-LTR (from cytomegalovirus) (Bashart, M. et al., Cell 41:521, 1985) or in the RSV-LTR (from Rous sarcoma virus) (Gorman, C. M., Proc. Natl. Acad. Sci. USA 79:6777, 1982). Also useful are the promoter of the mouse metallothionein I gene (Hamer, D, et al., J. Mol. Appl. Gen. 1:273-88, 1982; the TK promoter of Herpes virus (McKnight, S, Cell 31:355-65, 1982); the SV40 early promoter (Benoist, C., et al., Nature 290:304-10, 1981); and the yeast gal4 gene promoter (Johnston, S A et al., Proc. Natl. Acad. Sci. USA 79:6971-5, 1982); Silver, P A, et al., Proc. Natl. Acad. Sci. (USA) 81:5951-5, 1984)). Other illustrative descriptions of transcriptional factor association with promoter regions and the separate activation and DNA binding of transcription factors include: Keegan et al., Nature 231:699, 1986; Fields et al., Nature 340:245, 1989; Jones, Cell 61:9, 1990; Lewin, Cell 61:1161, 1990; Ptashne et al., Nature 346:329, 1990; Adams et al., Cell 72:306, 1993.

The promoter region may further include an octamer region which may also function as a tissue specific enhancer, by interacting with certain proteins found in the specific tissue. The enhancer domain of the DNA construct of the present invention is one which is specific for the target cells to be transfected, or is highly activated by cellular factors of such target cells. Examples of vectors (plasmid or retrovirus) are disclosed, e.g., in Roy-Burman et al., U.S. Pat. No. 5,112,767, incorporated by reference. For a general discussion of enhancers and their actions in transcription, see, Lewin, B M, Genes IV, Oxford University Press pp. 552-576, 1990 (or later edition). Particularly useful are retroviral enhancers (e.g., viral LTR) that is placed upstream from the promoter with which it interacts to stimulate gene expression. For use with retroviral vectors, the endogenous viral LTR may be rendered enhancer-less and substituted with other desired enhancer sequences which confer tissue specificity or other desirable properties such as transcriptional efficiency.

Thus, expression cassettes include plasmids, recombinant viruses, any form of a recombinant “naked DNA” vector, and the like, such as pNGVL4a. A “vector” comprises a nucleic acid which can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors include replicons (e.g., RNA replicons), bacteriophages) to which fragments of DNA may be attached and become replicated. Vectors thus include, but are not limited to RNA, autonomous self-replicating circular or linear DNA or RNA, e.g., plasmids, viruses, and the like (U.S. Pat. No. 5,217,879, incorporated by reference), and includes both the expression and nonexpression plasmids. Where a recombinant cell or culture is described as hosting an “expression vector” this includes both extrachromosomal circular and linear DNA and DNA that has been incorporated into the host chromosome(s). Where a vector is being maintained by a host cell, the vector may either be stably replicated by the cells during mitosis as an autonomous structure, or is incorporated within the host's genome.

Exemplary virus vectors that may be used include recombinant adenoviruses (Horowitz, M S, In: Virology, Fields, B N et al., eds, Raven Press, NY, 1990, p. 1679; Berkner, K L, Biotechniques 6:616-29, 1988; Strauss, S E, In: The Adenoviruses, Ginsberg, H S, ed., Plenum Press, NY, 1984, chapter 11) and herpes simplex virus (HSV). Advantages of adenovirus vectors for human gene delivery include the fact that recombination is rare, no human malignancies are known to be associated with such viruses, the adenovirus genome is double stranded DNA which can be manipulated to accept foreign genes of up to 7.5 kb in size, and live adenovirus is a safe human vaccine organisms. Adeno-associated virus is also useful for human therapy (Samulski, R J et al., EMBO J. 10:3941, 1991) according to the present invention.

Another vector which can express the DNA molecule of the present invention, and is useful in the present therapeutic setting is vaccinia virus, which can be rendered non-replicating (U.S. Pat. Nos. 5,225,336; 5,204,243; 5,155,020; 4,769,330; Fuerst, T R et al., Proc. Natl. Acad. Sci. USA 86:2549-53, 1992; Chakrabarti, S et al., Mol Cell Biol 5:3403-9, 1985, each of which are incorporated by reference). Descriptions of recombinant vaccinia viruses and other viruses containing heterologous DNA and their uses in immunization and DNA therapy are reviewed in: Moss, B, Curr Opin Genet Dev 3:86-90, 1993; Moss, B, Biotechnol. 20:345-62, 1992).

Other viral vectors that may be used include viral or non-viral vectors, including adeno-associated virus vectors, retrovirus vectors, lentivirus vectors, and plasmid vectors. Exemplary types of viruses include HSV (herpes simplex virus), AAV (adeno associated virus), HIV (human immunodeficiency virus), BIV (bovine immunodeficiency virus), and MLV (murine leukemia virus).

A DNA vaccine may also use a replicon, e.g., an RNA replicon, a self-replicating RNA vector. In one embodiment, a replicon is one based on a Sindbis virus RNA replicon, e.g., SINrep5. The present inventors tested E7 in the context of such a vaccine and showed (see Wu et al, U.S. patent application Ser. No. 10/343,719) that a Sindbis virus RNA vaccine encoding HSV-1 VP22 linked to E7 significantly increased activation of E7-specific CD8 T cells, resulting in potent antitumor immunity against E7-expressing tumors. The Sindbis virus RNA replicon vector used in these studies, SINrep5, has been described (Bredenbeek, P J et al., 1993, J. Virol. 67:6439-6446).

Generally, RNA replicon vaccines may be derived from alphavirus vectors, such as Sindbis virus (Hariharan, M J et al., 1998. J Virol 72:950-8.), Semliki Forest virus (Berglund, P M et al., 1997. AIDS Res Hum Retroviruses 13:1487-95; Ying, H T et al., 1999. Nat Med 5:823-7) or Venezuelan equine encephalitis virus (Pushko, P M et al., 1997. Virology 239:389-401). These self-replicating and self-limiting vaccines may be administered as either (1) RNA or (2) DNA which is then transcribed into RNA replicons in cells transfected in vitro or in vivo (Berglund, P C et al., 1998. Nat Biotechnol 16:562-5; Leitner, W W et al., 2000. Cancer Res 60:51-5). An exemplary Semliki Forest virus is pSCA1 (DiCiommo, D P et al., J Biol Chem 1998; 273:18060-6).

The plasmid vector pcDNA3 or a functional homolog thereof (SEQ ID NO: 1) may be used in a DNA vaccine. In other embodiments, pNGVL4a (SEQ ID NO: 2) is used. pNGVL4a, one plasmid backbone for the present invention was originally derived from the pNGVL3 vector, which has been approved for human vaccine trials. The pNGVL4a vector includes two immunostimulatory sequences (tandem repeats of CpG dinucleotides) in the noncoding region. Whereas any other plasmid DNA that can transform either APCs, including DC's or other cells which, via cross-priming, transfer the antigenic moiety to DCs, is useful in the present invention, pNGFVLA4a may be used because of the fact that it has already been approved for human therapeutic use.

The following references set forth principles and current information in the field of basic, medical and veterinary virology and are incorporated by reference: Fields Virology, Fields, B N et al., eds., Lippincott Williams & Wilkins, NY, 1996; Principles of Virology: Molecular Biology, Pathogenesis, and Control, Flint, S. J. et al., eds., Amer Soc Microbiol, Washington DC, 1999; Principles and Practice of Clinical Virology, 4th Edition, Zuckerman. A. J. et al., eds, John Wiley & Sons, NY, 1999; The Hepatitis C Viruses, by Hagedorn, C H et al., eds., Springer Verlag, 1999; Hepatitis B Virus: Molecular Mechanisms in Disease and Novel Strategies for Therapy, Koshy, R. et al., eds, World Scientific Pub Co, 1998; Veterinary Virology, Murphy, F. A. et al., eds., Academic Press, NY, 1999; Avian Viruses: Function and Control, Ritchie, B. W., Iowa State University Press, Ames, 2000; Virus Taxonomy: Classification and Nomenclature of Viruses: Seventh Report of the International Committee on Taxonomy of Viruses, by M. H. V. Van Regenmortel, M H V et al., eds., Academic Press; NY, 2000.

In addition to naked DNA or viral vectors, engineered bacteria may be used as vectors. A number of bacterial strains including Salmonella, BCG and Listeria monocytogenes (LM) (Hoiseth et al., Nature 291:238-9, 1981; Poirier, T P et al., J Exp Med 168:25-32, 1988); Sadoff, J C et al., Science 240:336-8, 1988; Stover, C K et al., Nature 351:456-60, 1991; Aldovini, A et al., Nature 351:479-82, 1991). These organisms display two promising characteristics for use as vaccine vectors: (1) enteric routes of infection, providing the possibility of oral vaccine delivery; and (2) infection of monocytes/macrophages thereby targeting antigens to professional APCs.

In addition to virus-mediated gene transfer in vivo, physical means well-known in the art can be used for direct transfer of DNA, including administration of plasmid DNA (Wolff et al., 1990, supra) and particle-bombardment mediated gene transfer (Yang, N-S, et al., Proc Natl Acad Sci USA 87:9568, 1990; Williams, R S et al., Proc Natl Acad Sci USA 88:2726, 1991; Zelenin, A V et al., FEBS Lett 280:94, 1991; Zelenin, A V et al., FEBS Lett 244:65, 1989); Johnston, S A et al., In Vitro Cell Dev Biol 27:11, 1991). Furthermore, electroporation, a well-known means to transfer genes into cells in vitro, can be used to transfer DNA molecules according to the present invention to tissues in vivo (Titomirov, A V et al., Biochim Biophys Acta 1088:131, 1991).

“Carrier mediated gene transfer” has also been described (Wu, C H et al., J Biol Chem 264:16985, 1989; Wu, G Y et al., J Biol Chem 263:14621, 1988; Soriano, P et al., Proc Nat. Acad Sci USA 80:7128, 1983; Wang, C-Y et al., Pro. Natl Acad Sci USA 84:7851, 1982; Wilson, J M et al., J Biol Chem 267:963, 1992). In one embodiment, carriers are targeted liposomes (Nicolau, C et al., Proc Natl Acad Sci USA 80:1068, 1983; Soriano et al., supra) such as immunoliposomes, which can incorporate acylated mAbs into the lipid bilayer (Wang et al., supra). Polycations such as asialoglycoprotein/polylysine (Wu et al., 1989, supra) may be used, where the conjugate includes a target tissue-recognizing molecule (e.g., asialo-orosomucoid for liver) and a DNA binding compound to bind to the DNA to be transfected without causing damage, such as polylysine. This conjugate is then complexed with plasmid DNA of the present invention.

Plasmid DNA used for transfection or microinjection may be prepared using methods well-known in the art, for example using the Quiagen procedure (Quiagen), followed by DNA purification using known methods, such as the methods exemplified herein.

Such expression vectors may be used to transfect host cells (in vitro, ex vivo or in vivo) for expression of the DNA and production of the encoded proteins which include fusion proteins or peptides. In one embodiment, a DNA vaccine is administered to or contacted with a cell, e.g., a cell obtained from a subject (e.g., an antigen presenting cell), and administered to a subject, wherein the subject is treated before, after or at the same time as the cells are administered to the subject.

The term “isolated” as used herein, when referring to a molecule or composition, such as a translocation polypeptide or a nucleic acid coding therefor, means that the molecule or composition is separated from at least one other compound (protein, other nucleic acid, etc.) or from other contaminants with which it is natively associated or becomes associated during processing. An isolated composition can also be substantially pure. An isolated composition can be in a homogeneous state and can be dry or in aqueous solution. Purity and homogeneity can be determined, for example, using analytical chemical techniques such as polyacrylamide gel electrophoresis (PAGE) or high performance liquid chromatography (HPLC). Even where a protein has been isolated so as to appear as a homogenous or dominant band in a gel pattern, there are trace contaminants which co-purify with it.

Host cells transformed or transfected to express the fusion polypeptide or a homologue or functional derivative thereof are within the scope of the invention. For example, the fusion polypeptide may be expressed in yeast, or mammalian cells such as Chinese hamster ovary cells (CHO) or human cells. In one embodiment, cells for expression according to the present invention are APCs or DCs. Other suitable host cells are known to those skilled in the art.

### Other Nucleic Acids for Potentiating Immune Responses

Methods of administrating a chemotherapeutic drug and a vaccine may further comprise administration of one or more other constructs, e.g., to prolong the life of antigen presenting cells. Exemplary constructs are described in the following two sections. Such constructs may be administered simultaneously or at the same time as a DNA vaccine. Alternatively, they may be administered before or after administration of the DNA vaccine or chemotherapeutic drug.

Potentiation of Immune Responses Using siRNA Directed at Apoptotic Pathways

Administration to a subject of a DNA vaccine and a chemotherapeutic drug may be accompanied by administration of one or more other agents, e.g., constructs. In one embodiment, a method comprises further administering to a subject an siRNA directed at an apoptotic pathway, such as described in WO 2006/073970, which is incorporated herein in its entirety.

The present inventors have previously designed siRNA sequences that hybridize to, and block expression of the activation of Bak and Bax proteins that are central players in the apoptosis signalling pathway. The present invention is also directed to the methods of treating tumors or hyper proliferative disease involving the administration of siRNA molecules (sequences), vectors containing or encoding the siRNA, expression vectors with a promoter operably linked to the siRNA coding sequence that drives transcription of siRNA sequences that are “specific” for sequences Bak and Bax nucleic acid. siRNAs may include single stranded “hairpin” sequences because of their stability and binding to the target mRNA.

Since Bak and Bax are involved, among other death proteins, in apoptosis of APCs, particularly DCs, the present siRNA sequences may be used in conjunction with a broad range of DNA vaccine constructs encoding antigens to enhance and promote the immune response induced by such DNA vaccine constructs, particularly CD8+ T cell mediated immune responses typified by CTL activation and action. This is believed to occur as a result of the effect of the siRNA in prolonging the life of antigen-presenting DCs which may otherwise be killed in the course of a developing immune response by the very same CTLs that the DCs are responsible for inducing.

In addition to Bak and Bax, additional targets for siRNAs designed in an analogous manner include caspase 8, caspase 9 and caspase 3. The present invention includes compositions and methods in which siRNAs targeting any two or more of Bak, Bax, caspase 8, caspase 9 and caspase 3 are used in combination, optionally simultaneously (along with a DNA immunogen that encodes an antigen), to administer to a subject. Such combinations of siRNAs may also be used to transfect DCs (along with antigen loading) to improve the immunogenicity of the DCs as cellular vaccines by rendering them resistant to apoptosis.

siRNAs suppress gene expression through a highly regulated enzyme-mediated process called RNA interference (RNAi) (Sharp, P. A., Genes Dev. 15:485-90, 2001; Bernstein, E et al., Nature 409:363-66, 2001; Nykanen, A et al., Cell 107:309-21, 2001; Elbashir et al., Genes Dev. 15:188-200, 2001). RNA interference is the sequence-specific degradation of homologues in an mRNA of a targeting sequence in an siNA. As used herein, the term siNA (small, or short, interfering nucleic acid) is meant to be equivalent to other terms used to describe nucleic acid molecules that are capable of mediating sequence specific RNAi (RNA interference), for example short (or small) interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically-modified siRNA, post-transcriptional gene silencing RNA (ptgsRNA), translational silencing, and others. RNAi involves multiple RNA-protein interactions characterized by four major steps: assembly of siRNA with the RNA-induced silencing complex (RISC), activation of the RISC, target recognition and target cleavage. These interactions may bias strand selection during siRNA-RISC assembly and activation, and contribute to the overall efficiency of RNAi (Khvorova, A et al., Cell 115:209-216 (2003); Schwarz, D S et al. 115:199-208 (2003)))

Considerations to be taken into account when designing an RNAi molecule include, among others, the sequence to be targeted, secondary structure of the RNA target and binding of RNA binding proteins. Methods of optimizing siRNA sequences will be evident to the skilled worker. Typical algorithms and methods are described in Vickers et al. (2003) J Biol Chem 278:7108-7118; Yang et al. (2003) Proc Natl Acad Sci USA 99:9942-9947; Far et al. (2003) Nuc. Acids Res. 31:4417-4424; and Reynolds et al. (2004) Nature Biotechnology 22:326-330, all of which are incorporated by reference in their entirety.

The methods described in Far et al., supra, and Reynolds et al., supra, may be used by those of ordinary skill in the art to select targeted sequences and design siRNA sequences that are effective at silencing the transcription of the relevant mRNA. Far et al. suggests options for assessing target accessibility for siRNA and supports the design of active siRNA constructs. This approach can be automated, adapted to high throughput and is open to include additional parameters relevant to the biological activity of siRNA. To identify siRNA-specific features likely to contribute to efficient processing at each of the steps of RNAi noted above. Reynolds et al., supra, present a systematic analysis of 180 siRNAs targeting the mRNA of two genes. Eight characteristics associated with siRNA functionality were identified: low G/C content, a bias towards low internal stability at the sense strand 3′-terminus, lack of inverted repeats, and sense strand base preferences (positions 3, 10, 13 and 19). Application of an algorithm incorporating all eight criteria significantly improves potent siRNA selection. This highlights the utility of rational design for selecting potent siRNAs that facilitate functional gene knockdown.

Candidate siRNA sequences against mouse and human Bax and Bak are selected using a process that involves running a BLAST search against the sequence of Bax or Bak (or any other target) and selecting sequences that “survive” to ensure that these sequences will not be cross matched with any other genes.

siRNA sequences selected according to such a process and algorithm may be cloned into an expression plasmid and tested for their activity in abrogating Bak/Bax function cells of the appropriate animal species. Those sequences that show RNAi activity may be used by direct administration bound to particles, or recloned into a viral vector such as a replication-defective human adenovirus serotype 5 (Ad5).

One advantage of this viral vector is the high titer obtainable (in the range of 10¹⁰) and therefore the high multiplicities-of infection that can be attained. For example, infection with 100 infectious units/cell ensures all cells are infected. Another advantage of this virus is the high susceptibility and infectivity and the host range (with respect to cell types). Even if expression is transient, cells would survive, possibly replicate, and continue to function before Bak/Bax activity would recover and lead to cell death. In one embodiment, constructs include the following:

For Bak:
(SEQ ID NO: 42)
5′P-UGCCUACGAACUCUUCACCdTdT-3′ (sense)
(SEQ ID NO: 43)
5′P-GGUGAAGAGUUCGUAGGCAdTdT-3′ (antisense),

The nucleotide sequence encoding the Bak protein (including the stop codon) (GenBank accession No. NM_{—}007523 is shown below (SEQ ID NO: 44) with the targeted sequence in upper case, underscored.

| atggcatctggacaaggaccaggtcccccgaaggtgggctgcgatgagtccccgtccccttctgaacagcaggttgcccaggac | |
|---|---|
| acagaggaggtattcgaagctacgttttttacctccaccagcaggaacaggagacccaggggcggccgcctgccaaccccgag | |
| atggacaacttgcccctggaacccaacagcatcttgggtcaggtgggtcggcagatgctctcatcggagatgatattaaccggcg | |
| ctacgacacagagttccagaatttactagaacagcttcagcccacagccgggaaTGCCTACGAACTCTTCACC | |
| aagatcgcctccagcctatttaagagtggcatcagctggggccgcgtggtggctctcctgggctttggctaccgtctggccctgtac | |
| gtctaccagcgtggtttgaccggcttcctgggccaggtgacctgctttttggctgatatcatactgcatcattacatcgccagatggatc | |
| gcacagagaggcggttgggtggcagccctgaatttgcgtagagaccccatcctgaccgtaatggtgatttttggtgtggttctgttgg | |
| gccaattcgtggtacacagattcttcagatcatga | 637 |

The targeted sequence of Bak, TGCCTACGAACTCTTCACC is SEQ ID NO: 45

For Bax:
(SEQ ID NO: 46)
5′P-UAUGGAGCUGCAGAGGAUGdTdT-3′ (sense)
(SEQ ID NO: 47)
5′P-CAUCCUCUGCAGCUCCAUAdTdT-3′ (antisense)

The nucleotide sequence encoding Bax (including the stop codon) (GenBank accession No. L22472 is shown below (SEQ ID NO: 48) with the targeted sequence shown in upper case and underscored

| atggacgggtccggggagcagcttgggagcggcgggcccaccagctctgaacagatcatgaagacaggggcctttttgctacag | |
|---|---|
| ggtttcatccaggatcgagcagggaggatggctggggagacacctgagctgaccttggagcagccgccccaggatgcgtccacc | |
| aagaagctgagcgagtgtaccggcgaattggagatgaactggatagcaaTATGGAGCTGCAGAGGATGatt | |
| gctgacgtggacacggactccccccgagaggtcttcttccgggtggcagagacatgtttgctgatggcaacttcaactggggccg | |
| cgtggttgccctcttctactttgctagcaaactggtgctcaaggccagtgcactaaagtgcccgagctgatcagaaccatcatgggc | |
| tggacactggacttcctccgtgagcggctgcttgtaggatccaagaccagggtggctgggaaggcctcctacctacttcgggacc | |
| cccacatggcagacagtgaccatattgtggctggagtcctcaccgcctcgctcaccatctggaagaagatgggctga | 589 |

The targeted sequence of Bax, TATGGAGCTGCAGAGGATG is SEQ ID NO: 49

In a one embodiment, the inhibitory molecule is a double stranded nucleic acid (i.e., an RNA), used in a method of RNA interference. The following show the “paired” 19 nucleotide structures of the siRNA sequences shown above, where the symbol [symbol] :

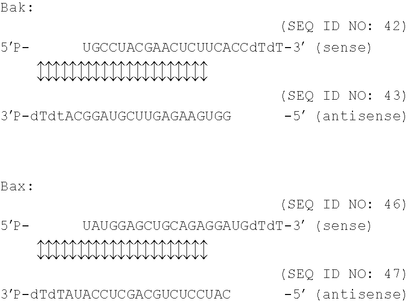

### Other Pro-Apoptotic Proteins to be Targeted

1. Caspase 8: The nucleotide sequence of human caspase-8 is shown below (SEQ ID NO: 50). GenBank Access. #NM_{—}001228. One target sequence for RNAi is underscored. Others may be identified using methods such as those described herein (and in reference cited herein, primarily Far et al., supra and Reynolds et al., supra).

| atg gac ttc agc aga aat ctt tat gat att ggg gaa caa ctg gac agt gaa gat ctg gcc tcc ctc aag ttc | |
|---|---|
| ctg agc ctg gac tac att ccg caa agg aag caa gaa ccc atc aag gat gcc ttg atg tta ttc cag aga ctc | |
| cag gaa aag aga atg ttg gag gaa agc aat ctg tcc ttc ctg aag gag ctg ctc ttc cga att aat aga ctg | |
| gat ttg ctg att acc tac cta aac act aga aag gag gag atg gaa agg gaa ctt cag aca cca ggc agg gct | |
| caa att tct gcc tac agg ttc cac ttc tgc cgc atg agc tgg gct gaa gca aac agc cag tgc cag aca cag | |
| tct gta cct ttc tgg cgg agg gtc gat cat cta tta ata agg gtc atg ctc tat cag att tca gaa gaa gtg agc | |
| aga tca gaa ttg agg tct ttt aag ttt ctt ttg caa gag gaa atc tcc aaa tgc aaa ctg gat gat gac atg aac | |
| ctg ctg gat att ttc ata gag atg gag aag agg gtc atc ctg gga gaa gga aag ttg gac atc ctg aaa aga | |
| gtc tgt gcc caa atc aac aag agc ctg ctg aag ata atc aac gac tat gaa gaa ttc agc aaa ggg gag gag | |
| ttg tgt ggg gta atg aca atc tcg gac tct cca aga gaa cag gat agt gaa tca cag act ttg gac aaa gtt | |
| tac caa atg aaa agc aaa cct cgg gga tac tgt ctg atc atc aac aat cac aat ttt gca aaa gca cgg gag | |
| aaa gtg ccc aaa ctt cac agc att agg gac agg aat gga aca cac ttg gat gca ggg gct ttg acc acg acc | |
| ttt gaa gag ctt cat ttt gag atc aag ccc cac gat gac tgc aca gta gag caa atc tat gag att ttg aaa atc | |
| tac caa ctc atg gac cac agt aac atg gac tgc ttc atc tgc tgt atc ctc tcc cat gga gac aag ggc atc | |
| atc tat ggc act gat gga cag gag gcc ccc atc tat gag ctg aca tct cag ttc act ggt ttg aag tgc cct | |
| tcc ctt gct gga aaa ccc aaa gtg ttt ttt att cag gct tgt cag ggg gat aac tac cag aaa ggt ata cct gtt | |
| gag act gat tca gag gag caa ccc tat tta gaa atg gat tta tca tca cct caa acg aga tat atc ccg gat | |
| gag gct gac ttt ctg ctg ggg atg gcc act gtg aat aac tgt gtt tcc tac cga aac cct gca gag gga acc | |
| tgg tac atc cag tca ctt tgc cag agc ctg aga gag cga tgt cct cga ggc gat gat att ctc acc atc ctg | |
| act gaa gtg aac tat gaa gta agc aac aag gat gac aag aaa aac atg ggg aaa cag atg cct cag cct | |
| act ttc aca cta aga aaa aaa ctt gtc ttc cct tct gat tga | 1491 |

The sequences of sense and antisense siRNA strands for targeting this sequence (including dTdT 3′ overhangs, are:

(SEQ ID NO: 51)
5′-AACCUCGGGGAUACUGUCUGAdTdT-3′ (sense)
(SEQ ID NO: 52)
5′-UCAGACAGUAUCCCCGAGGUUdTdT-3′ (antisense)

2. Caspase 9: The nucleotide sequence of human caspase-9 is shown below (SEQ ID NO: 53). See GenBank Access. #NM_{—}001229. The sequence below is of “variant α” which is longer than a second alternatively spliced variant β, which lacks the underscored part of the sequence shown below (and which is anti-apoptotic). Target sequences for RNAi, expected to fall in the underscored segment, are identified using known methods such as those described herein and in Far et al., supra and Reynolds et al., supra) and siNAs, such as siRNAs, are designed accordingly.

| atg gac gaa gcg gat cgg cgg ctc ctg cgg cgg tgc cgg ctg cgg ctg gtg gaa gag ctg cag gtg gac | |
|---|---|
| cag ctc tgg gac gcc ctg ctg agc cgc gag ctg ttc agg ccc cat atg atc gag gac atc cag cgg gca | |
| ggc tct gga tct cgg cgg gat cag gcc agg cag ctg atc ata gat ctg gag act cga ggg agt cag gct ctt | |
| cct ttg ttc atc tcc tgc tta gag gac aca ggc cag gac atg ctg gct tcg ttt ctg cga act aac agg caa | |
| gca gca aag ttg tcg aag cca acc cta gaa aac ctt acc cca gtg gtg ctc aga cca gag att cgc aaa cca | |
| gag gtt ctc aga ccg gaa aca ccc aga cca gtg gac att ggt tct gga gga ttt ggt gat gtc ggt gct ctt | |
| gag agt ttg agg gga aat gca gat ttg gct tac atc ctg agc atg gag ccc tgt ggc cac tgc ctc att atc | |
| aac aat gtg aac ttc tgc cgt gag tcc ggg ctc cgc acc cgc act ggc tcc aac atc gac tgt gag aag ttg | |
| cgg cgt cgc ttc tcc tcg ctg cat ttc atg gtg gag gtg aag ggc gac ctg act gcc aag aaa atg gtg ctg | |
| gct ttg ctg gag ctg gcg cag cag gac cac ggt gct ctg gac tgc tgc gtg gtg gtc att ctc tct cac ggc | |
| tgt cag gcc agc cac ctg cag ttc cca ggg gct gtc tac ggc aca gat gga tgc cct gtg tcg gtc gag aag | |
| att gtg aac atc ttc aat ggg acc agc tgc ccc agc ctg gga ggg aag ccc aag ctc ttt ttc atc cag gcc | |
| tgt ggt ggg gag cag aaa gac cat ggg ttt gag gtg gcc tcc act tcc cct gaa gac gag tcc cct ggc agt | |
| aac ccc gag cca gat gcc acc ccg ttc cag gaa ggt ttg agg acc ttc gac cag ctg gac gcc ata tct agt | |
| ttg ccc aca ccc agt gac atc ttt gtg tcc tac tct act ttc cca ggt ttt gtt tcc tgg agg gac ccc aag agt | |
| ggc tcc tgg tac gtt gag acc ctg gac gac atc ttt gag cag tgg gct cac tct gaa gac ctg cag tcc ctc | |
| ctg ctt agg gtc gct aat gct gtt tcg gtg aaa ggg att tat aaa cag atg cct ggt tgc ttt aat ttc ctc cgg | |
| aaa aaa ctt ttc ttt aaa aca tca taa | 1191 |

3. Caspase 3: The nucleotide sequence of human caspase-3 is shown below (SEQ ID NO: 54). See GenBank Access. #NM_{—}004346. The sequence below is of “variant α” which is the longer of two alternatively spliced variants, all of which encode the full protein. Target sequences for RNAi are identified using known methods such as those described herein and in Far et al., supra and Reynolds et al., supra) and siNAs, such as siRNAs, are designed accordingly.

| atg gag aac act gaa aac tca gtg gat tca aaa tcc att aaa aat ttg gaa cca aag atc ata cat gga agc | |
|---|---|
| gaa tca atg gac tct gga ata tcc ctg gac aac agt tat aaa atg gat tat cct gag atg ggt tta tgt ata ata | |
| att aat aat aag aat ttt cat aaa agc act gga atg aca tct cgg tct ggt aca gat gtc gat gca gca aac ctc | |
| agg gaa aca ttc aga aac ttg aaa tat gaa gtc agg aat aaa aat gat ctt aca cgt gaa gaa att gtg gaa | |
| ttg atg cgt gat gtt tct aaa gaa gat cac agc aaa agg agc agt ttt gtt tgt gtg ctt ctg agc cat ggt gaa | |
| gaa gga ata att ttt gga aca aat gga cct gtt gac ctg aaa aaa ata aca aac ttt ttc aga ggg gat cgt tgt | |
| aga agt cta act gga aaa ccc aaa ctt ttc att att cag gcc tgc cgt ggt aca gaa ctg gac tgt ggc att | |
| gag aca gac agt ggt gtt gat gat gac atg gcg tgt cat aaa ata cca gtg gag gcc gac ttc ttg tat gca | |
| tac tcc aca gca cct ggt tat tat tct tgg cga aat tca aag gat ggc tcc tgg ttc atc cag tcg ctt tgt gcc | |
| atg ctg aaa cag tat gcc gac aag ctt gaa ttt atg cac att ctt acc cgg gtt aac cga aag gtg gca aca | |
| gaa ttt gag tcc ttt tcc ttt gac gct act ttt cat gca aag aaa cag att cca tgt att gtt tcc atg ctc aca | |
| aaa gaa ctc tat ttt tat cac taa | 834 |

Long double stranded interfering RNAs, such a miRNAs, appear to tolerate mismatches more readily than do short double stranded RNAs. In addition, as used herein, the term RNAi is meant to be equivalent to other terms used to describe sequence specific RNA interference, such as post transcriptional gene silencing, or an epigenetic phenomenon. For example, siNA molecules of the invention can be used to epigenetically silence genes at both the post-transcriptional level or the pre-transcriptional level. In a non-limiting example, epigenetic regulation of gene expression by siNA molecules of the invention can result from siNA mediated modification of chromatin structure and thereby alter gene expression (see, for example, Allshire Science 297:1818-19, 2002; Volpe et al., Science 297:1833-37, 2002; Jenuwein, Science 297:2215-18, 2002; and Hall et al., Science 297, 2232-2237, 2002.)

An siNA can be designed to target any region of the coding or non-coding sequence of an mRNA. An siNA is a double-stranded polynucleotide molecule comprising self-complementary sense and antisense regions, wherein the antisense region comprises nucleotide sequence that is complementary to nucleotide sequence in a target nucleic acid molecule or a portion thereof and the sense region has a nucleotide sequence corresponding to the target nucleic acid sequence or a portion thereof. The siNA can be assembled from two separate oligonucleotides, where one strand is the sense strand and the other is the antisense strand, wherein the antisense and sense strands are self-complementary. The siNA can be assembled from a single oligonucleotide, where the self-complementary sense and antisense regions of the siNA are linked by means of a nucleic acid based or non-nucleic acid-based linker(s). The siNA can be a polynucleotide with a hairpin secondary structure, having self-complementary sense and antisense regions. The siNA can be a circular single-stranded polynucleotide having two or more loop structures and a stem comprising self-complementary sense and antisense regions, wherein the circular polynucleotide can be processed either in vivo or in vitro to generate an active siNA molecule capable of mediating RNAi. The siNA can also comprise a single stranded polynucleotide having nucleotide sequence complementary to nucleotide sequence in a target nucleic acid molecule or a portion thereof (or can be an siNA molecule that does not require the presence within the siNA molecule of nucleotide sequence corresponding to the target nucleic acid sequence or a portion thereof), wherein the single stranded polynucleotide can further comprise a terminal phosphate group, such as a 5′-phosphate (see for example Martinez et al. (2002) Cell 110, 563-574 and Schwarz et al. (2002) Molecular Cell 10, 537-568), or 5′,3′-diphosphate.

In certain embodiments, the siNA molecule of the invention comprises separate sense and antisense sequences or regions, wherein the sense and antisense regions are covalently linked by nucleotide or non-nucleotide linkers molecules as is known in the art, or are alternately non-covalently linked by ionic interactions, hydrogen bonding, Van der Waal's interactions, hydrophobic interactions, and/or stacking interactions.

As used herein, siNA molecules need not be limited to those molecules containing only ribonucleotides but may also further encompass deoxyribonucleotides (as in the siRNAs which each include a dTdT dinucleotide) chemically-modified nucleotides, and non-nucleotides. In certain embodiments, the siNA molecules of the invention lack 2′-hydroxy (2′-OH) containing nucleotides. In certain embodiments, siNAs do not require the presence of nucleotides having a 2′-hydroxy group for mediating RNAi and as such, siNAs of the invention optionally do not include any ribonucleotides (e.g., nucleotides having a 2′-OH group). Such siNA molecules that do not require the presence of ribonucleotides within the siNA molecule to support RNAi can however have an attached linker or linkers or other attached or associated groups, moieties, or chains containing one or more nucleotides with 2′-OH groups. Optionally, siNA molecules can comprise ribonucleotides at about 5, 10, 20, 30, 40, or 50% of the nucleotide positions. If modified, the siNAs of the invention can also be referred to as “short interfering modified oligonucleotides” or “siMON.” Other chemical modifications, e.g., as described in Int'l Patent Publications WO 03/070918 and WO 03/074654, both of which are incorporated by reference, can be applied to any siNA sequence of the invention.

In one embodiment a molecule mediating RNAi has a 2 nucleotide 3′ overhang (dTdT in the sequences disclosed herein). If the RNAi molecule is expressed in a cell from a construct, for example from a hairpin molecule or from an inverted repeat of the desired sequence, then the endogenous cellular machinery will create the overhangs.

Methods of making siRNAs are conventional. In vitro methods include processing the polyribonucleotide sequence in a cell-free system (e.g., digesting long dsRNAs with RNAse III or Dicer), transcribing recombinant double stranded DNA in vitro, and chemical synthesis of nucleotide sequences homologous to Bak or Bax sequences. See, e.g., Tuschl et al., Genes & Dev. 13:3191-3197, 1999. In vivo methods include
(1) transfecting DNA vectors into a cell such that a substrate is converted into siRNA in vivo. See, for example, Kawasaki et al., Nucleic Acids Res 31:700-07, 2003; Miyagishi et al., Nature Biotechnol 20:497-500, 2003; Lee et al., Nature Biotechnol 20:500-05, 2002; Brummelkamp et al., Science 296:550-53, 2002; McManus et al., RNA 8:842-50, 2002; Paddison et al., Genes Dev 16:948-58, 2002; Paddison et al., Proc Natl Acad Sci USA 99:1443-48, 2002; Paul et al., Nature Biotechnol 20:505-08, 2002; Sui et al., Proc Natl Acad Sci USA 99:5515-20, 2002; Yu et al., Proc Natl Acad Sci USA 99:6047-52, 2002)
(2) expressing short hairpin RNAs from plasmid systems using RNA polymerase III (pol III) promoters. See, for example, Kawasaki et al., supra; Miyagishi et al., supra; Lee et al., supra; Brummelkamp et al., supra; McManus et al., supra), Paddison et al., supra (both); Paul et al., supra, Sui et al., supra; and Yu et al., supra; and/or
(3) expressing short RNA from tandem promoters. See, for example, Miyagishi et al., supra; Lee et al., supra).

When synthesized in vitro, a typical micromolar scale RNA synthesis provides about 1 mg of siRNA, which is sufficient for about 1000 transfection experiments using a 24-well tissue culture plate format. In general, to inhibit Bak or Bax expression in cells in culture, one or more siRNAs can be added to cells in culture media, typically at about 1 ng/ml to about 10 μg siRNA/ml.

For reviews and more general description of inhibitory RNAs, see Lau et al., Sci Amer August 2003: 34-41; McManus et al., Nature Rev Genetics 3, 737-47, 2002; and Dykxhoorn et al., Nature Rev Mol Cell Bio 4:457-467, 2003. For further guidance regarding methods of designing and preparing siRNAs, testing them for efficacy, and using them in methods of RNA interference (both in vitro and in vivo), see, e.g., Allshire, Science 297:1818-19, 2002; Volpe et al., Science 297:1833-37, 2002; Jenuwein, Science 297:2215-18, 2002; Hall et al., Science 297 2232-37, 2002; Hutvagner et al., Science 297:2056-60, 2002; McManus et al. RNA 8:842-850, 2002; Reinhart et al., Genes Dev. 16:1616-26, 2002; Reinhart et al., Science 297:1831, 2002; Fire et al. (1998) Nature 391:806-11, 2002; Moss, Curr Biol 11:R772-5, 2002:Brummelkamp et al., supra; Bass, Nature 411 428-9, 2001; Elbashir et al., Nature 411:494-8; U.S. Pat. No. 6,506,559; Published US Pat App. 20030206887; and PCT applications WO99/07409, WO99/32619, WO 00/01846, WO 00/44914, WO00/44895, WO01/29058, WO01/36646, WO01/75164, WO01/92513, WO 01/29058, WO01/89304, WO01/90401, WO02/16620, and WO02/29858, all of which are incorporated by reference.

Ribozymes and siNAs can take any of the forms, including modified versions, described for antisense nucleic acid molecules; and they can be introduced into cells as oligonucleotides (single or double stranded), or in the form of an expression vector.

In one embodiment, an antisense nucleic acid, siNA (e.g., siRNA) or ribozyme comprises a single stranded polynucleotide comprising a sequence that is at least about 90% (e.g., at least about 93%, 95%, 97%, 98% or 99%) identical to a target segment (such as those indicted for Bak and Bax above) or a complement thereof. As used herein, a DNA and an RNA encoded by it are said to contain the same “sequence,” taking into account that the thymine bases in DNA are replaced by uracil bases in RNA.

Active variants (e.g., length variants, including fragments; and sequence variants) of the nucleic acid-based inhibitors discussed herein are also within the scope of the invention. An “active” variant is one that retains an activity of the inhibitor from which it is derived (i.e., the ability to inhibit expression). It is to test a variant to determine for its activity using conventional procedures.

As for length variants, an antisense nucleic acid or siRNA may be of any length that is effective for inhibition of a gene of interest. Typically, an antisense nucleic acid is between about 6 and about 50 nucleotides (e.g., at least about 12, 15, 20, 25, 30, 35, 40, 45 or 50 nt), and may be as long as about 100 to about 200 nucleotides or more. Antisense nucleic acids having about the same length as the gene or coding sequence to be inhibited may be used. When referring to length, the terms bases and base pairs (bp) are used interchangeably, and will be understood to correspond to single stranded (ss) and double stranded (ds) nucleic acids. The length of an effective siNA is generally between about 15 by and about 29 bp in length, between about 19 and about 29 bp (e.g., about 15, 17, 19, 21, 23, 25, 27 or 29 bp), with shorter and longer sequences being acceptable. Generally, siNAs are shorter than about 30 bases to prevent eliciting interferon effects. For example, an active variant of an siRNA having, for one of its strands, the 19 nucleotide sequence of any of SEQ ID NOs: 42, 43, 46, and 47 herein can lack base pairs from either, or both, of ends of the dsRNA; or can comprise additional base pairs at either, or both, ends of the ds RNA, provided that the total of length of the siRNA is between about 19 and about 29 bp, inclusive. One embodiment of the invention is an siRNA that “consists essentially of” sequences represented by SEQ ID NOs: 42, 43, 46, and 47 or complements of these sequence. The term “consists essentially of” is an intermediate transitional phrase, and in this case excludes, for example, sequences that are long enough to induce a significant interferon response. An siRNA of the invention may consist essentially of between about 19 and about 29 bp in length.

As for sequence variants, in one embodiment, an inhibitory nucleic acid, whether an antisense molecule, a ribozyme (the recognition sequences), or an siNA, comprises a strand that is complementary (100% identical in sequence) to a sequence of a gene that it is designed to inhibit. However, 100% sequence identity is not required to practice the present invention. Thus, the invention has the advantage of being able to tolerate naturally occurring sequence variations, for example, in human c-met, that might be expected due to genetic mutation, polymorphism, or evolutionary divergence. Alternatively, the variant sequences may be artificially generated. Nucleic acid sequences with small insertions, deletions, or single point mutations relative to the target sequence can be effective inhibitors.

The degree of sequence identity may be optimized by sequence comparison and alignment algorithms well-known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). In one embodiment, at least about 90% sequence identity may be used (e.g., at least about 92%, 95%, 98% or 99%), or even 100% sequence identity, between the inhibitory nucleic acid and the targeted sequence of targeted gene.

Alternatively, an active variant of an inhibitory nucleic acid of the invention is one that hybridizes to the sequence it is intended to inhibit under conditions of high stringency. For example, the duplex region of an siRNA may be defined functionally as a nucleotide sequence that is capable of hybridizing with a portion of the target gene transcript under high stringency conditions (e.g., 400 mM NaCl, 40 mM PIPES pH 6.4, 1 mM EDTA, 50° C. or 70° C., hybridization for 12-16 hours), followed generally by washing.

DC-1 cells or BM-DCs presenting a given antigen X, when not treated with the siRNAs of the invention, respond to sufficient numbers X-specific CD8+ CTL by apoptotic cell death. In contrast, the same cells transfected with the siRNA or infected with a viral vector encoding the present siRNA sequences survive better despite the delivery of killing signals.

Delivery and expression of the siRNA compositions of the present invention inhibit the death of DCs in vivo in the process of a developing T cell response, and thereby promote and stimulate the generation of an immune response induced by immunization with an antigen-encoding DNA vaccine vector. These capabilities have been exemplified by showing that:
(1) co-administration of DNA vaccines encoding HPV-16 E7 with siRNA targeted to Bak and Bax prolongs the lives of antigen-presenting DCs in the draining lymph nodes, thereby enhancing antigen-specific CD8⁺ T cell responses, and eliciting potent antitumor effects against an E7-expressing tumor in vaccinated subjects.
(2) DCs transfected with siRNA targeting Bak and Bax resist killing by T cells in vivo. E7-loaded DCs transfected with Bak/Bax siRNA so that Bak and Bax protein expression is downregulated resist apoptotic death induced by T cells in vivo. When administered to subjects, these DCs generate stronger antigen-specific immune responses and manifest therapeutic effects (compared to DCs transfected with control siRNA).
   Thus, siRNA constructs are useful as a part of the nucleic acid vaccination and chemotherapy regimen described in this application.

### Potentiation of Immune Responses Using Anti-Apoptotic Proteins

Administration to a subject of a DNA vaccine and a chemotherapeutic drug may also be accompanied by administration of a nucleic acid encoding an anti-apoptotic protein, as described in WO2005/047501 and in U.S. Patent Application Publication No. 20070026076, both of which are incorporated by reference.

The present inventors have previously designed and disclosed an immunotherapeutic strategy that combines antigen-encoding DNA vaccine compositions with additional DNA vectors comprising anti-apoptotic genes including bcl-2, bc-lxL, XIAP, dominant negative mutants of caspase-8 and caspase-9, the products of which are known to inhibit apoptosis (Wu, et al. U.S. Patent Application Publication No. 20070026076, incorporated herein by reference). Serine protease inhibitor 6 (SPI-6) which inhibits granzyme B, may also be employed in compositions and methods to delay apoptotic cell death of DCs. The present inventors have shown that the harnessing of an additional biological mechanism, that of inhibiting apoptosis, significantly enhances T cell responses to DNA vaccines comprising antigen-coding sequences, as well as linked sequences encoding such IPPs.

Intradermal vaccination by gene gun efficiently delivers a DNA vaccine into DCs of the skin, resulting in the activation and priming of antigen-specific T cells in vivo. DCs, however, have a limited life span, hindering their long-term ability to prime antigen-specific T cells. According to the present invention, a strategy that combines combination therapy with methods to prolong the survival of DNA-transduced DCs enhances priming of antigen-specific T cells and thereby, increase DNA vaccine potency. Co-delivery of DNA encoding inhibitors of apoptosis (BCL-xL, BCL-2, XIAP, dominant negative caspase-9, or dominant negative caspase-8) with DNA encoding an antigen (exemplified as HPV-16 E7 protein) prolongs the survival of transduced DCs. More importantly, vaccinated subjects exhibited significant enhancement in antigen-specific CD8+ T cell immune responses, resulting in a potent antitumor effect against antigen-expressing tumors. Among these anti-apoptotic factors, BCL-XL demonstrated the greatest enhancement of both antigen-specific immune responses and antitumor effects. Thus, co-administration of a combination therapy including a DNA vaccine with one or more DNA constructs encoding anti-apoptotic proteins provides a way to enhance DNA vaccine potency.

Serine protease inhibitor 6 (SPI-6), also called Serpinb9, inhibits granzyme B, and may thereby delay apoptotic cell death in DCs. Intradermal co-administration of DNA encoding SPI-6 with DNA constructs encoding E7 linked to various IPPs significantly increased E7-specific CD8+ T cell and CD4+ Th1 cell responses and enhanced anti-tumor effects when compared to vaccination without SPI-6. Thus, in certain embodiments, combined methods are used that enhance MHC class I and II antigen processing with delivery of SPI-6 to potentiate immunity.

A similar approach employs DNA-based alphaviral RNA replicon vectors, also called suicidal DNA vectors. To enhance the immune response to an antigen, e.g., HPV E7, a DNA-based Semliki Forest virus vector, pSCA1, the antigen DNA is fused with DNA encoding an anti-apoptotic polypeptide such BCL-xL, a member of the BCL-2 family. pSCA1 encoding a fusion protein of an antigen polypeptide and/BCL-xL delays cell death in transfected DCs and generates significantly higher antigen-specific CD8+ T-cell-mediated immunity. The antiapoptotic function of BCL-xL is important for the enhancement of antigen-specific CD8+ T-cell responses. Thus, in one embodiment, delaying cell death induced by an otherwise desirable suicidal DNA vaccine enhances its potency.

Thus, the present invention is also directed to combination therapies including administering a chemotherapeutic drug with a nucleic acid composition useful as an immunogen, comprising a combination of: (a) first nucleic acid vector comprising a first sequence encoding an antigenic polypeptide or peptide, which first vector optionally comprises a second sequence linked to the first sequence, which second sequence encodes an immunogenicity-potentiating polypeptide (IPP); b) a second nucleic acid vector encoding an anti-apoptotic polypeptide, wherein, when the second vector is administered with the first vector to a subject, a T cell-mediated immune response to the antigenic polypeptide or peptide is induced that is greater in magnitude and/or duration than an immune response induced by administration of the first vector alone. The first vector above may comprise a promoter operatively linked to the first and/or the second sequence.

In the above compositions the anti-apoptotic polypeptide may be selected from the group consisting of (a) BCL-xL, (b) BCL2, (c) XIAP, (d) FLICEc-s, (e) dominant-negative caspase-8, (f) dominant negative caspase-9, (g) SPI-6, and (h) a functional homologue or a derivative of any of (a)-(g). The anti-apoptotic DNA may be physically linked to the antigen-encoding DNA. Examples of this are provided in U.S. Patent Application publication No. 20070026076, incorporated by reference, primarily in the form of suicidal DNA vaccine vectors. Alternatively, the anti-apoptotic DNA may be administered separately from, but in combination with the antigen-encoding DNA molecule. Even more examples of the co-administration of these two types of vectors are provided in U.S. patent application Ser. No. 10/546,810 (publication number US 2007-0026076).

Exemplary nucleotide and amino acid sequences of anti-apoptotic and other proteins are provided in the sequence listing. Biologically active homologs of these proteins and constructs may also be used. Biologically active homologs is to be understood as described herein in the context of other proteins, e.g., IPPs.

The coding sequence for BCL-xL as present in the pcDNA3 vector of the present invention is SEQ ID NO:55; the amino acid sequence of BCL-xL is SEQ ID NO:56; the sequence pcDNA3-BCL-xL is SEQ ID NO:57 (the BCL-xL coding sequence corresponds to nucleotides 983 to 1732); a pcDNA3 vector combining E7 and BCL-xL, designated pcDNA3-E7/BCL-xL is SEQ ID NO:58 (the Eland BCL-xL sequences correspond to nucleotides 960 to 2009); the amino acid sequence of the E7-BCL-xL chimeric or fusion polypeptide is SEQ ID NO: 59; a mutant BCL-xL (“mtBCL-xL”) DNA sequence is SEQ ID NO: 60; the amino acid sequence of mtBCL-xL is SEQ ID NO: 61; the amino acid sequence of the E7-mtBCL-xL chimeric or fusion polypeptide is SEQ ID NO: 62; in the pcDNA-mtBCL-xL [SEQ ID NO: 63] vector, this mutant sequence is inserted in the same position that BCL-xL is inserted in SEQ ID NO: 57 and in the pcDNA-E7/mtBCL-XL [SEQ ID NO: 64], this sequence is inserted in the same position as the BCL-xL sequence is in SEQ ID NO: 58; the sequence of the suicidal DNA vector pSCA1-BCL-xL is SEQ ID NO: 65 (the BCL-xL sequence corresponds to nucleotides 7483 to 8232); the sequence of the “combined” vector, pSCA1-E7/BCL-xL is SEQ ID NO: 66 (the sequence of E7 and BCL-xL corresponds to nucleotides 7461 to 8510); the sequence of pSCA1-mtBCL-xL [SEQ ID NO: 67] is the same as that for the wild type BCL-xL except that the mtBCL-xL sequence is inserted in the same position as the wild type sequence in the pSCA1-mtBCL-xL vector; the sequence pSCA1-E7/mtBCL-xL [SEQ ID NO: 68] is the same as that for the wild type pSCA1-E7/BCL-xL above, except that the mtBCL-xL sequence is inserted in the same position as the wild type sequence; the sequence of the vector pSG5-BCL-xL is SEQ ID NO: 69 (the BCL-xL coding sequence corresponds to nucleotides 1061 to 1810); the sequenced of the vector pSG5-mtBCL-xL is SEQ ID NO: 70 with the mutant BCL-xL sequence has the mtBCL-xL, shown above, inserted in the same location as for the wild type vector immediately above; the nucleotide sequence of the DNA encoding the XIAP anti-apoptotic protein is SEQ ID NO: 71; the amino acid of the vector comprising the XIAP anti-apoptotic protein coding sequence is SEQ ID NO: 72; the nucleotide sequence of the vector comprising the XIAP anti-apoptotic protein coding sequence, designated PSG5-XIAP is shown in SEQ ID NO: 73 (with the XIAP corresponding to nucleotides 1055 to 2553); the sequence of DNA encoding the anti-apoptotic protein FLICEc-s is SEQ ID NO: 74; the amino acid sequence of the anti-apoptotic protein FLICEc-s is SEQ ID NO: 75; the PSG5 vector encoding the anti-apoptotic protein FLICEc-s, designated PSG5-FLICEc-s, has the sequence SEQ ID NO: 76 (with the FLICEc-s sequence corresponding to nucleotides 1049 to 2443); the sequence of DNA encoding the anti-apoptotic protein Bcl2 is SEQ ID NO: 77; the amino acid sequence of Bcl2 is SEQ ID NO: 78; the PSG5 vector encoding Bcl2, designated PSG5-BCL2, has the sequence SEQ ID NO: 79 (with the Bcl2 sequence corresponding to nucleotides 1061 to 1678); the pSG5-dn-caspase-8 vector is SEQ ID NO: 80 (encoding the dominant-negative caspase-8 corresponding to nucleotides 1055 to 2449); the amino acid sequence of dn-caspase-8 is SEQ ID NO: 81; the pSG5-dn-caspase-9 vector is SEQ ID NO: 82 (encoding the dominant-negative caspase-9 as nucleotides 1055 to 2305); the amino acid sequence of dn-caspase-9 is SEQ ID NO: 83); the nucleotide sequence of murine serine protease inhibitor 6 (SPI-6, deposited in GENEBANK as NM 009256) is SEQ ID NO: 84; the amino acid sequence of the SPI-6 protein is SEQ ID NO: 85; the nucleic acid sequence of the mutant SPI-6 (mtSPI6) is SEQ ID NO: 86; the amino acid sequence of the mutant SPI-6 protein (mtSPI-6) is SEQ ID NO: 87; the sequence of the pcDNA3-Spi6 vector is SEQ ID NO: 88 (the SPI-6 sequence corresponds to nucleotides 960 to 2081); and the sequence of the mutant vector pcDNA3-mtSpi6 vector [SEQ ID NO: 89] is the same as that above, except that the mtSPI-6 sequence is inserted in the same location in place of the wild type SPI-6.

Biologically active homologs of these nucleic acids and proteins may be used. Biologically active homologs are to be understood as described in the context of other proteins, e.g., IPPs, herein. For example, a vector may encode an anti-apoptotic protein that is at least about 90%, 95%, 98% or 99% identical to that of a sequence set forth herein.

### Oncolytic Viruses

Oncolytic viruses not only comprise a class of vectors able to encode and express a particular antigen to which an antigen-specific immune response is desired, but it also mediates killing of cancer cells. The term “oncolytic” and “oncolytic viruses” refer to cancer killing, i.e. “onco” meaning cancer and “lytic” meaning “killing”. As used herein, where oncolytic refers to an “oncolytic virus” and an “OV,” this virus represents a virus that may kill a cancer cell. In principle any virus capable of selective replication in neoplastic cells including cells of tumors, neoplasms, carcinomas, sarcomas, and the like may be utilized in the invention. Selective replication in neoplastic cells means that the virus replicates at least 1×10⁴, 1×10⁵, 1×10⁶, or more efficient in at least three cell lines established from different tumors compared to cells from at least three different non-tumorigenic tissues.

Oncolytic viruses may additionally or alternatively be targeted to specific tissues or tumor tissues. This can be achieved for example through transcriptional targeting of viral genes (e.g. WO 96/39841, incorporated by reference) or through modification of viral proteins that are involved in the cellular binding and uptake mechanisms during the infection process (e.g. WO 2004033639 or WO 2003068809, all of which are incorporated by reference).

A wide range of viruses are contemplated as oncolytic viruses in the present invention, such as but not limited to herpes viruses, Adenovirus, Adeno-associated virus, influenza virus, reovirus, vesicular stomatitis virus (VSV), Newcastle virus, vaccinia virus, poliovirus, measles virus, mumps virus, sindbis virus (SrN) and sendai virus (SV). Tables 1-6 below provide an overview of examples previously published oncolytic viruses (taken from www.oncolyticVirus.org).

TABLE 1
Oncolytic viruses targeting oncogenic
ras or defective Interferon pathways.

| Virus (Company, | Viral gene | Cellular | | Refer- |
|---|---|---|---|---|
| if known) | defect | Target | Tumor models | ences |
| Influenza A | NS1 | PKR | Melanoma | (1) |
| HSV1mutants: | ICP34.5 | Protein | Brain, | (2, 3) |
| R3616, 1716, | | phosphatase | Colorectal, | |
| G207 (Medigene, | | 1a, Defective | ovarian, lung, | |
| Inc.), MGH1 | | interferon | prostate, breast | |
| | | signaling. | | |
| Reovirus | None | Overactive | Brain, ovarian, | (4-7). |
| (Oncolytics | | Ras pathway | breast, | |
| Biotech., Inc.) | | | colorectal | |
| VSV | None | Defective | Melanoma | (8) |
| | | Interferon | | |
| | | signaling | | |
| Newcastle | None | Overactive | Fibrosarcoma, | (9) |
| disease virus | | Ras pathway | Neuroblastoma | |
| (Provirus) | | | | |

TABLE 2
Oncolytic viruses targeting defective p16 tumor suppressor pathways.

| Virus (Company, | | | | | |
|---|---|---|---|---|---|
| if known) | Mutated viral gene | | Cellular target | Effect | References |
| Adenovirus D24 | E1A-CR2 | | PRB | Viral replication | (10, 11) |
| and dl922-947 | domain | | | restricted to pRB- | |
| (Onyx | | | | defective mutants | |
| Pharmaceuticals) | | | | | |
| Adenovirus | E1A-CR1 and | | PRB, p300, p107, | In keratinocytes, | (12) |
| CB106 | CR2 domains | | p130 | viral replication | |
| | | | | restricted to | |
| | | | | papillomavirus | |
| | | | | E6/E7 expressors | |
| Adenovirus | a) | E1A-CR1 | PRB and | Increased | (13) |
| ONYX- | b) | E2F promoter | upregulated E2F | dependence of | |
| 411(Onyx | | driving E1A | transcription | virus replication | |
| Pharmaceuticals) | | and E4 genes | factor | on overactive | |
| | c) | E3 deletion | | E2F | |
| HSV: hrR3, | Ul39 (ICP6) | | RR activity | Viral replication | (14, 15) |
| rRp450, | | | elevating dNTP | depends on dNTP | |
| HSV1yCD, | | | pools | pools | |
| MGH1, G207 | | | | | |
| Medigene, Inc.), | | | | | |
| G47Δ | | | | | |
| HSV Myb34.5 | a) | UL39 (ICP6) | RR activity | Increased viral | (16, 17) |
| (Prestwick | b) | B-Myb (E2F- | elevating dNTP | replicative | |
| Scientific, Inc.) | | responsive) | pools and | dependence on | |
| | | promoter | upregulated E2F | E2F activity | |
| | | driving γ34.5 | transcription | | |
| | | gene) | factor | | |

| | | | | |
|---|---|---|---|---|
| Vaccinia vvDD- | TK gene | Elevated dTTP | Viral replication | (18, 19) |
| GFP | | (due to cellular | restricted to cells | |
| | | TK?) | with dTTP pools | |

TABLE 3
Oncolytic viruses targeting defective p53 tumor suppressor pathway.

| Virus (Company, | | | | | |
|---|---|---|---|---|---|
| if known) | Mutated viral gene | | Cellular target | Effect | References |
| Adenovirus | E1B-55 Kd | | p53 | Viral replication | (20) |
| ONYX-015 | | | | restricted to p53- | |
| (Onyx | | | | defective mutants | |
| Pharmaceuticals) | | | | | |
| Adenovirus | 1) | p53 promoter | p53, p300. | Expression of E2 | (22) |
| 01/PEME (Canji) | | driving | | and subsequent | |
| | | expression of | | viral genes | |
| | | E2F | | dependent on loss | |
| | | antagonist | | of p53 function; | |
| | 2) | E1A-CR1 | | wild-type p53 | |
| | | p300 binding- | | function | |
| | | domain | | enhanced by | |
| | 3) | E3 deletion | | p300 | |
| | 4) | Extra Major | | coactivation; | |
| | | Late | | increased | |
| | | Promoter | | adenoviral | |
| | | driving | | release and cell | |
| | | expression of | | death by | |
| | | E3-11.6 Kd | | adenoviral death | |
| | | | | protein (21) | |

| | | | | |
|---|---|---|---|---|
| AAV | AAV unusual | p53/p21 | Lack of G2/M | (23) |
| | DNA structure is | | arrest in p53- | |
| | precipitating | | defective cells, | |
| | factor | | infected with | |
| | | | AAV, causes cell | |
| | | | death | |

TABLE 4
Targeting of oncolytic viruses with tumor-specific promoters.

| Virus (Company, | Tumor-specific | | | | |
|---|---|---|---|---|---|
| if known) | Promoter | | Viral gene | Effect | References |
| Adenovirus | PSA (prostate) | | E1A | Replication | (24) |
| CV706 (Calydon, | | | | restricted to | |
| Inc.) | | | | prostate tissue | |
| Adenovirus | a) | Rat probasin | E1A and E1B | Same as above | (25, 26) |
| CN787 (Calydon, | | promoter for | | | |
| Inc.) | | E1A | | | |
| | b) | PSA for E1B | | | |

| | | | | |
|---|---|---|---|---|
| Adenovirus | AFP | E1A and E1B | Replication | (27) |
| CV980 (Calydon, | (hepatocellular | | restricted to | |
| Inc.) | carcinoma) | | hepatic tumors. | |
| Adenovirus | E2F1 promoter | E1A and E4 | Increased | (13) |
| ONYX-411 | (most tumors) | | dependence of | |
| (Onyx | | | virus replication | |
| Pharmaceuticals) | | | on overactive | |
| | | | E2F | |
| Adenovirus | p53 promoter | E2F antagonist. | Expression of E2 | (22) |
| 01/PEME (Canji | (most tumors) | | and subsequent | |
| Inc.) | | | viral genes | |
| | | | dependent on loss | |
| | | | of p53 function | |
| CG8840 (Cell | Uroplakin II | E1A and E1B | Replication | (28) |
| Genesys, Inc.) | (bladder) | | restricted to | |
| | | | bladder cancer | |
| KD1-SPB | Surfactant protein | E4 | Replication | (29) |
| | B | | improved in lung | |
| | | | tumors | |
| HSV Myb34.5 | B-Myb promoter | g34.5 (ICP34.5) | Improved | (16, 17) |
| (Prestwick | (most tumors) | | replication in | |
| Scientific, Inc.) | | | tumors | |
| HSV DF3g34.5 | DF3 promoter | g34.5 (ICP34.5) | Improved | (30) |
| | | | replication in | |
| | | | MUC1-positive | |
| | | | pancreatic and | |
| | | | breast tumor | |
| | | | cells. | |
| HSV G92A | Albumin | ICP4 | Replication | (31) |
| | promoter | | restricted in | |
| | | | hepatoma | |

TABLE 5
Targeting with “tumor-selective” infection.

| | Redirected viral | | | |
|---|---|---|---|---|
| Virus | ligand | Cellular target | Effect | References |
| Dual Adenovirus | Bispecific- | EGFR | Redirects viral | (32) |
| system: | antibody binding | | infection to | |
| AdsCAR-EGF + | adenovirus fiber | | EGFR-expressing | |
| Δ24 | to EGFR | | cells | |
| Adenovirus: | H1-loop in Fiber | Integrin | Redirects viral | (33) |
| Ad5-D24RGD | of Ad modified | | infection to | |
| | by incorporation | | integrin- | |
| | of RGD | | expressing cells. | |
| D24 or ONYX- | Infusion of | EGFR | Redirects viral | (34) |
| 015 | bispecific | | infection to | |
| | antibodies to | | EGFR-expressing | |
| | fiber and EGFR | | cells | |
| Ad 5/35 | Fiber of | Unknown | Redirects viral | (35) |
| | adenovirus | | infection away | |
| | serotype 35 | | from CAR and | |
| | substituted into | | towards an | |
| | adenovirus | | unidentified | |
| | serotype 5 | | cellular receptor | |
| | | | present in human | |
| | | | breast cancer | |

TABLE 6
Other mechanisms of oncolytic virus targeting.

| | | | | Oncolytic | |
|---|---|---|---|---|---|
| Virus | Defect in viral gene | | Effect | mechanism | References |
| Vaccinia vvDD- | Vaccinia Growth | | Cannot prime | Only dividing | (18) |
| GFP | Factor | | neighboring cells | tumor cells will | |
| | | | to divide | replicate, because | |
| | | | | normal cells are | |
| | | | | not “primed” by | |
| | | | | VGF | |
| Poliovirus | Substitutes | | Loss of | Tumor cells can | (36) |
| PV1(RIPO) | poliovirus IRES | | neurovirulence, | still propagate | |
| | element with | | because neurons | virus | |
| | rhinovirus 2 | | cannot translate | | |
| HSV1: rRp450 | ICP6 | CYP2B1 | Cyclophosphamide > | Predominant | (15) |
| | | | Phosphoramide | anticancer action + | |
| | | | Mustard | immunosuppressive | |
| | | | | effects. | |
| Adenovirus: | E1B55kD | Fused TK- | Ganciclovir > | Combination of | (50) |
| FGR | | CD gene | GCV-Phosphate + | FGR, GCV, 5FC | |
| | | | 5-fluorocytosine > | and radiation | |
| | | | 5fluorouracil | shows | |
| | | | | predominant | |
| | | | | anticancer action | |
| HSV1: Fu-10 | Unknown | Fusogenic | Not applicable | Enhanced fusion | (51) |
| | | glycol- | | of cell membranes | |
| | | protein | | caused by | |
| | | | | replicating virus | |
| | | | | increases | |
| | | | | anticancer effect | |
| Adenovirus: | E3 | Interferon | Not applicable | Increased | (52) |
| ad5/IFN | | | | anticancer effect | |
| | | | | compared to | |
| | | | | control E3-deleted | |
| | | | | adenovirus | |
| Adenovirus; | E1B55KD | TK | Ganciclovir > | Contradictory | (53, 54) |
| Ad.TK^{RC}, Ad.OW34 | | | GCV-Phosphate | anticancer effects | |
| Adenovirus: | E3-19K | TK | Ganciclovir > | Increased | (55) |
| Ig.Ad5E1⁺.E3TK | | | GCV-Phosphate | anticancer effect | |
| | | | | in glioma | |
| HSV1: Mix of | ICP6 and | IL2 | Not applicable | At low dose, the | (56) |
| G207 + | ICP34.5 | | | mix was more | |
| Defective HSv- | | | | effective than | |
| IL2 | | | | either virus alone | |
| HSV1: NV1042 | Complex | IL12 | Not applicable | Increased | (57) |
| | | | | anticancer effect | |
| HSV1: Mix of | ICP6 and | Soluble | Not applicable | Increased | (58) |
| G207 + | ICP34.5 | B7-1 | | anticancer effect | |
| Defective HSV- | | | | | |
| soluble B7-1 | | | | | |
| HSV1: | ICP6 | Yeast | 5-fluorocytosine > | Increased | (59) |
| HSV1yCD | | cytosine | 5-fluorouracil | anticancer effect | |
| | | deaminase | | minimal antiviral | |
| | | | | effect | |
| Vaccinia: VCD | TK | Bacterial | 5-fluorocytosine > | Increased effect at | (60) |
| | | cytosine | 5-fluorouracil | low viral dose | |
| | | deaminase | | | |
| HSV1 | ICP34.5 | IL4, IL12, | Not applicable | Increased | (61, 62) |
| | | IL10 | | anticancer effect | |
| | | | | for IL12 and IL4, | |
| | | | | but antagonistic | |
| | | | | effect for IL10 | |

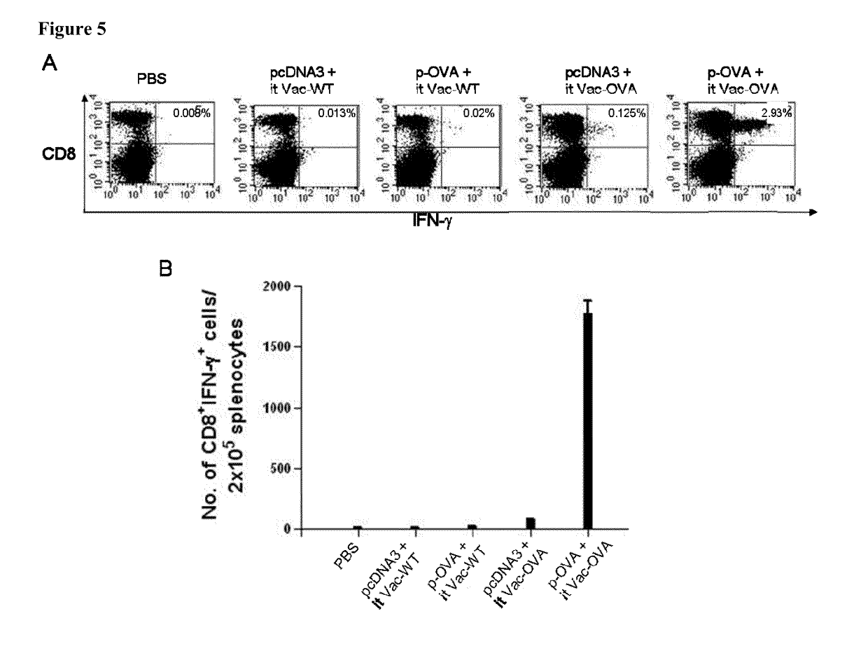

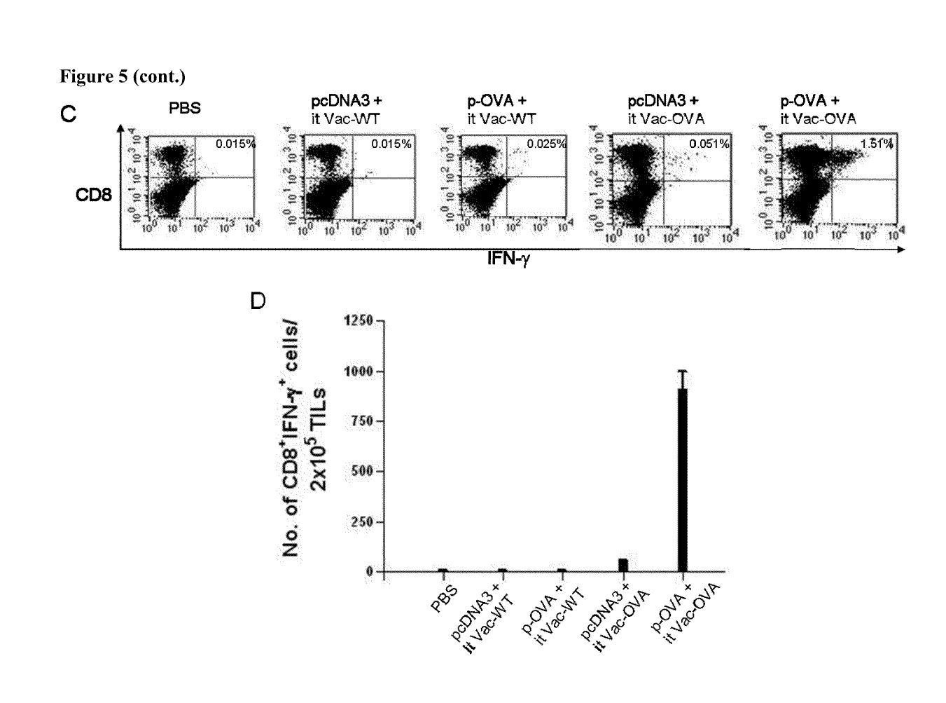

TABLE 7
Oncolytic viruses that express anti-cancer cDNAs.

| | Viral gene | Anticancer | Prodrug > | | |
|---|---|---|---|---|---|
| Virus | defect | cDNA | Metabolite | Effect | Reference |
| HSV1: hrR3, | ICP6 | TK | Ganciclovir > GCV- | Predominant | (42-49) |
| MGH1, G207 | and/or | | Phosphate | anticancer action | |
| (Medigene, Inc.) | ICP34.5 | | | in some situations, | |
| | | | | but increased | |
| | | | | antiviral action in | |
| | | | | others (FIG. 5) | |

Methods for producing and purifying the oncolytic virus used according to the invention are described in the publications cited below.
1. Bergmann M et al., 2001, Cancer Res, 61: 8188-8193;
2. Leib, D A et al., 2000, Proc Natl Acad Sci 97: 6097-6101;
3. Farassati, F et al., 2001, Nat Cell Biol, 3: 745-750;
4. Strong, J E et al. 1998, Embo J, 17: 3351-3362;
5. Coffey, M C et al., 1998, Science, 282: 1332-1334;
6. Norman, K L et al, 2002, Hum Gene Ther, 13: 641-652;
7. Wilcox, M E et al., 2001, J Natl Cancer Inst, 93: 903-912;
8. Stojdl, D F et al., 2000 Nat Med, 6: 821-825;
9. Lorence, R M et al., 1994, J Natl Cancer Inst, 86: 1228-1233;
10. Fueyo, J et al., 2000, Oncogene, 19: 2-12;
11. Heise, C. et al., 2000, Nat Med, 6: 1134-1139;
12. Balague, C. et al., 2001, J Virol, 75: 7602-7611;
13. Johnson, L. et al., 2002, Cancer Cell, 1: 325-337;
14. Carroll, N M. et al., 1996, Ann Surg, 224: 323-329; discussion 329-330;
15. Chase, M. et al., 1998, Nat Biotechnol, 16: 444-448;
16. Chung, R Y et al., 1999, J Virol, 73: 7556-7564;
17. Nakamura, H et al., 2002, J Clin Invest, 109: 871-882;
18. McCart, J A et al., 2001, Cancer Res, 61: 8751-8757;
19. Puhlmann, M et al., 1999, Hum Gene Ther, 10: 649-657;
20. Bischoff, J R et al., 1996, Science, 274: 373-376;
21. Tollefson, A E et al., 1996, J Virol, 70: 2296-2306;
22. Ramachandra, M et al., 2001, Nat Biotechnol, 19: 1035-1041;
23. Raj, K et al., 2001, Nature, 412: 914-917;
24. Rodriguez, R et al., 1997, Cancer Res, 57: 2559-2563;
25. Yu, D C et al., 1999, Cancer Res, 59: 4200-4203;
26. Chen, Y et al., 2001, Cancer Res, 61: 5453-5460;
27. Li, Y et al., 2001, Cancer Res, 61: 6428-6436;
28. Zhang, J et al., 2002, Cancer Res, 62: 3743-3750;
29. Doronin, K et al., 2001, J Virol, 75: 3314-3324;
30. Mullen, J T et al. Annals of Surgery, in press;
31. Miyatake, S I et al., 1999, Gene Ther, 6: 564-572;
32. Hemminki, A et al., 2001, Cancer Res, 61: 6377-6381;
33. Dmitriev, I et al., 1998, J Virol, 72: 9706-9713;
34. van der Poel, H G et al., 2002, J Urol, 168: 266-272;
35. Shayakhmetov, D M et al., 2002, Cancer Res, 62: 1063-1068;
36. Gromeier, M. et al., 2000, Proc Natl Acad Sci, 97: 6803-6808;
37. Nevins, J R, 1981, Cell, 26: 213-220;
38. Steinwaerder, D S et al., 2000, Hum Gene Ther, 11: 1933-1948;
39. Steinwaerder, D S et al., 2001, Nat Med, 7: 240-243;
40. Grote, D et al., 2001, Blood, 97: 3746-3754;
41. Asada, T, 1974, Cancer, 34: 1907-1928;
42. Boviatsis, E J et al., 1994, Cancer Res, 54: 5745-5751;
43. Kramm, C M et al., 1996, Hum Gene Ther, 7: 1989-1994;
44. Kasuya, H et al., 1999, J Surg Oncol, 72: 136-141;
45. Kramm, C M et al., 1997, Hum Gene Ther, 8: 2057-2068;
46. Carroll, N M et al., 1997, J Surg Res, 69: 413-417;
47. Yoon, S S et al., 1998, Ann Surg, 228: 366-374;
48. Todo, T et al., 2000, Cancer Gene Ther, 7: 939-946;
49. Samoto, K et al., 2002, Neurosurgery, 50: 599-605; discussion 605-596;
50. Freytag, S O et al., 1998, Hum Gene Ther, 9: 1323-1333;
51. Fu, X. and Zhang, X., 2002, Cancer Res, 62: 2306-2312;
52. Zhang, J F et al., 1996, Proc Natl Acad Sci 93: 4513-4518;
53. Wildner, O et al., 1999, Cancer Res, 59: 410-413;
54. Morris, J C and Wildner, O, 2000, Mol Ther, 1: 56-62;
55. Nanda, D et al., 2001, Cancer Res, 61: 8743-8750;
56. Zager, J S et al., 2001, Mol Med, 7: 561-568;
57. Wong, R J et al., 2001, Hum Gene Ther, 12: 253-265;
58. Todo, T et al., 2001, Cancer Res, 61: 153-161;
59. Nakamura, H et al., 2001. Cancer Res, 61: 5447-5452;
60. McCart, J A, 2000, Gene Ther, 7: 1217-1223;
61. Andreansky, S et al., 1998, Gene Ther, 5: 121-130;
62. Parker, J N et al., 2000, Proc Natl Acad Sci 97: 2208-2213;
63. Pechan, P A et al., 1996, Hum Gene Ther, 7: 2003-2013; and
64. Meignier, B. et al., 1988, J Infect Dis, 158: 602-614, all incorporated by reference.

Generally, the virus may be purified to render it essentially free of undesirable contaminants, such as defective interfering viral particles or endotoxins and other pyrogens, so that it will not cause any undesired reactions in the cell, animal, or individual receiving the virus. A means of purifying the virus involves the use of buoyant density gradients, such as cesium chloride gradient centrifugation.

In one embodiment, the oncolytic virus, e.g. vaccinia virus, further contains foreign DNA, i.e., DNA which is not derived from said virus. This DNA may encode the antigen to which an antigen-specific immune response is desired.

In other embodiments, the foreign DNA may be a heterologous promoter region, a structural gene, or a promoter operatively linked to such a gene. Representative promoters include, but are not limited to, the CMV promoter, LacZ promoter, Egr promoter or known HSV promoters. In a one embodiment, the structural gene is selected from the group of a cytokine/chemokine, a suicide gene, a fusogenic protein or a marker gene. Cytokines/chemokines that may be used include, but are not limited to, IL-4, IL-12 and GM-CSF. Suicide genes that may be used include, but are not limited to, p450 and cytosine deaminase. A fusogenic protein is for example Gibbon ape leukemia virus envelope. Common marker genes are luciferase, GFP or one of its variants, and LacZ.

In a further embodiment the oncolytic virus is further modified to have an altered host cell specificity. Such mutants are for example known for HSV-1 from WO 2004/033639, incorporated by reference, US 2005271620 included by reference, Kamiyama et al. (2006) and Menotti et al. (2006). Here, glycoproteins of HSV-1 such as gD, gC are fused to a ligand, especially to single-chain antibodies, that specifically bind to target cells of choice. Further, to detarget such viruses from their natural receptors and heparin sulfate proteoglycan deletions and/or point mutations are made in gB, gC and/or gD (WO 2004/033639, incorporated by reference, Zhou and Roizman, 2006).

### Chemotherapeutic Drugs

Drugs may also further be administered to a mammal in accordance with the methods and compositions taught herein. Generally, any drug that reduces the growth of cells without significantly affecting the immune system may be used, or at least not suppressing the immune system to the extent of eliminating the positive effects of a DNA vaccine that is administered to the subject. In one embodiment, the drugs are chemotherapeutic drugs.

A wide variety of chemotherapeutic drugs may be used, provided that the drug stimulates the effect of a vaccine, e.g., DNA vaccine. In certain embodiments, a chemotherapeutic drug may be a drug that (a) induces apoptosis of cells, in particular, cancer cells, when contacted therewith; (b) reduces tumor burden; and/or (c) enhances CD8+ T cell-mediated antitumor immunity. In certain embodiments, the drug must also be one that does not inhibit the immune system, or at least not at certain concentrations.

In one embodiment, the chemotherapeutic drug is epigallocatechin-3-gallate (EGCG) or a chemical derivative or pharmaceutically acceptable salt thereof.

Epigallocatechin gallate (EGCG) is the major polyphenol component found in green tea. EGCG has demonstrated antitumor effects in various human and animal models, including cancers of the breast, prostate, stomach, esophagus, colon, pancreas, skin, lung, and other sites. EGCG has been shown to act on different pathways to regulate cancer cell growth, survival, angiogenesis and metastasis. For example, some studies suggest that EGCG protects against cancer by causing cell cycle arrest and inducing apoptosis. It is also reported that telomerase inhibition might be one of the major mechanisms underlying the anticancer effects of EGCG. In comparison with commonly-used antitumor agents, including retinoids and doxorubicin, EGCG has a relatively low toxicity and is convenient to administer due to its oral bioavailability. Thus, EGCG has been used in clinical trials and appears to be a potentially ideal antitumor agent.

Exemplary analogs or derivatives of EGCG include (−)-EGCG, (+)-EGCG, (−)-EGCG-amide, (−)-GCG, (+)-GCG, (+)-EGCG-amide, (−)-ECG, (−)-CG, genistein, GTP-1, GTP-2, GTP-3, GTP-4, GTP-5, Bn-(+)-epigallocatechin gallate (US 2004/0186167, incorporated by reference), and dideoxy-epigallocatechin gallate (Furuta, et al., Bioorg. Med. Chem. Letters, 2007, 11: 3095-3098), For additional examples, see US 2004/0186167 (incorporated by reference in its entirety); Waleh, et al., Anticancer Res., 2005, 25: 397-402; Wai, et al., Bioorg. Med. Chem., 2004, 12: 5587-5593; Smith, et al., Proteins: Struc. Func. & Bioinform., 2003, 54: 58-70; U.S. Pat. No. 7,109,236 (incorporated by reference in its entirety); Landis-Piwowar, et al., Int. J. Mol. Med., 2005, 15: 735-742; Landis-Piwowar, et al., J. Cell. Phys., 2007, 213: 252-260; Daniel, et al., Int. J. Mol. Med., 2006, 18: 625-632; Tanaka, et al., Ang. Chemie Int., 2007, 46: 5934-5937.

Another chemotherapeutic drug that may be used is (a) 5,6 di-methylxanthenone-4-acetic acid (DMXAA), or a chemical derivative or analog thereof or a pharmaceutically acceptable salt thereof. Exemplary analogs or derivatives include xanthenone-4-acetic acid, flavone-8-acetic acid, xanthen-9-one-4-acetic acid, methyl (2,2-dimethyl-6-oxo-1,2-dihydro-6H-3,11-dioxacyclopenta[α]anthracen-10-yl)acetate, methyl (2-methyl-6-oxo-1,2-dihydro-6H-3,11-dioxacyclopenta[α]anthracen-10-yl)acetate, methyl (3,3-dimethyl-7-oxo-3H,7H-4,12-dioxabenzo[α]anthracen-10-yl)acetate, methyl-6-alkyloxyxanthen-9-one-4-acetates (Gobbi, et al., 2002, J. Med. Chem., 45: 4931) or a. For additional examples, see WO 2007/023302 A1, WO 2007/023307 A1, US 2006/9505, WO 2004/39363 A1, WO 2003/80044 A1, AU 2003/217035 A1, and AU 2003/282215 A1, each incorporated by reference in their entirety.

A chemotherapeutic drug may also be cisplatin, or a chemical derivative or analog thereof or a pharmaceutically acceptable salt thereof. Exemplary analogs or derivatives include dichloro[4,4′-bis(4,4,4-trifluorobutyl)-2,2′-bipyridine]platinum (Kyler et al., Bioorganic & Medicinal Chemistry, 2006, 14: 8692-8700), cis-[Rh2(—O2CCH3)2(CH3CN)6]2+ (Lutterman et al., J. Am. Chem. Soc., 2006, 128: 738-739), (+)-cis-(1,1-Cyclobutanedicarboxylato)((2R)-2-methyl-1,4-butanediamine-N,N′)platinum (O'Brien et al., Cancer Res., 1992, 52: 4130-4134), cis-bisneodecanoato-trans-R,R-1,2-diaminocyclohexane platinum(II) (Lu et al., J. of Clin. Oncol., 2005, 23: 3495-3501), carboplatin (Woloschuk, Drug Intell. Clin. Pharm., 1988, 22: 843-849), sebriplatin (Kanazawa et al., Head & Neck, 2006, 14: 38-43), satraplatin (Amorino et al., Cancer Chemother. and Pharmacol., 2000, 46: 423-426), azane (dichloroplatinum) (CID: 11961987), azanide (CID: 6712951), platinol (CID: 5702198), lopac-P-4394 (CID: 5460033), MOLI001226 (CID: 450696), trichloroplatinum (CID: 420479), platinate(1−), amminetrichloro-, ammonium (CID: 160995), triammineplatinum (CID: 119232), biocisplatinum (CID: 84691), platiblastin (CID: 2767) and pharmaceutically acceptable salts thereof. For additional examples, see U.S. Pat. No. 5,922,689, U.S. Pat. No. 4,996,337, U.S. Pat. No. 4,937,358, U.S. Pat. No. 4,808,730, U.S. Pat. No. 6,130,245, U.S. Pat. No. 7,232,919, and U.S. Pat. No. 7,038,071, each incorporated by reference in their entirety.

Another chemotherapeutic drug that may be used is apigenin, or a chemical derivative or analog thereof or a pharmaceutically acceptable salt thereof. Exemplary analogs or derivatives include acacetin, chrysin, kampherol, luteolin, myricetin, naringenin, quercetin (Wang et al., Nutrition and Cancer, 2004, 48: 106-114), puerarin (US 2006/0276458, incorporated by reference in its entirety) and pharmaceutically acceptable salts thereof. For additional examples, see US 2006/189680 A1, incorporated by reference in its entirety).

Another chemotherapeutic drug that may be used is doxorubicin, or a chemical derivative or analog thereof or a pharmaceutically acceptable salt thereof. Exemplary analogs or derivatives include anthracyclines, 3′-deamino-3′-(3-cyano-4-morpholinyl)doxorubicin, WP744 (Faderl, et al., Cancer Res., 2001, 21: 3777-3784), annamycin (Zou, et al., Cancer Chemother. Pharmacol., 1993, 32:190-196), 5-imino-daunorubicin, 2-pyrrolinodoxorubicin, DA-125 (Lim, et al., Cancer Chemother. Pharmacol., 1997, 40: 23-30), 4-demethoxy-4′-O-methyldoxorubicin, PNU 152243 and pharmaceutically acceptable salts thereof (Yuan, et al., Anti-Cancer Drugs, 2004, 15: 641-646). For additional examples, see EP 1242438 B1, U.S. Pat. No. 6,630,579, AU 2001/29066 B2, U.S. Pat. No. 4,826,964, U.S. Pat. No. 4,672,057, U.S. Pat. No. 4,314,054, AU 2002/358298 A1, and U.S. Pat. No. 4,301,277, each incorporated by reference in their entirety);

Other chemotherapeutic drugs that may be used are anti-death receptor 5 antibodies and binding proteins, and their derivatives, including antibody fragments, single-chain antibodies (scFvs), Avimers, chimeric antibodies, humanized antibodies, human antibodies and peptides binding death receptor 5. For examples, see US 2007/31414 and US 2006/269554, each incorporated by reference in their entirety.

Another chemotherapeutic drug that may be used is bortezomib, or a chemical derivative or analog thereof or a pharmaceutically acceptable salt thereof. Exemplary analogs or derivatives include MLN-273 and pharmaceutically acceptable salts thereof (Witola, et al., Eukaryotic Cell, 2007, doi:10.1128/EC.00229-07). For additional possibilities, see Groll, et al., Structure, 14:451.

Another chemotherapeutic drug that may be used is 5-aza-2-deoxycytidine, or a chemical derivative or analog thereof or a pharmaceutically acceptable salt thereof. Exemplary analogs or derivatives include other deoxycytidine derivatives and other nucleotide derivatives, such as deoxyadenine derivatives, deoxyguanine derivatives, deoxythymidine derivatives and pharmaceutically acceptable salts thereof.

Another chemotherapeutic drug that may be used is genistein, or a chemical derivative or analog thereof or a pharmaceutically acceptable salt thereof. Exemplary analogs or derivatives include 7-O-modified genistein derivatives (Zhang, et al., Chem. & Biodiv., 2007, 4: 248-255), 4′,5,7-tri[3-(2-hydroxyethylthio)propoxy]isoflavone, genistein glycosides (Polkowski, Cancer Letters, 2004, 203: 59-69), other genistein derivatives (Li, et al., Chem & Biodiv., 2006, 4: 463-472; Sarkar, et al., Mini. Rev. Med. Chem., 2006, 6: 401-407) or pharmaceutically acceptable salts thereof. For additional examples, see U.S. Pat. No. 6,541,613, U.S. Pat. No. 6,958,156, and WO/2002/081491, each incorporated by reference in their entirety.

Another chemotherapeutic drug that may be used is celecoxib, or a chemical derivative or analog thereof or a pharmaceutically acceptable salt thereof. Exemplary analogs or derivatives include N-(2-aminoethyl)-4-[5-(4-tolyl)-3-(trifluoromethyl)-1H-pyrazol-1-yl]benzenesulfonamide, 4-[5-(4-aminophenyl)-3-(trifluoromethyl)-1H-pyrazol-1-yl]benzenesulfonamide, OSU03012 (Johnson, et al., Blood, 2005, 105: 2504-2509), OSU03013 (Tong, et. al, Lung Cancer, 2006, 52: 117-124), dimethyl celecoxib (Backhus, et al., J. Thorac. and Cardiovasc. Surg., 2005, 130: 1406-1412), and other derivatives or pharmaceutically acceptable salts thereof (Ding, et al., Int. J. Cancer, 2005, 113: 803-810; Zhu, et al., Cancer Res., 2004, 64: 4309-4318; Song, et al., J. Natl. Cancer Inst., 2002, 94: 585-591). For additional examples, see U.S. Pat. No. 7,026,346, incorporated by reference in its entirety.

One of skill in the art will readily recognize that other chemotherapeutics can be used with the methods and kits disclosed in the present invention, including proteasome inhibitors (in addition to bortezomib) and inhibitors of DNA methylation. Other drugs that may be used include Paclitaxel; selenium compounds; SN38, etoposide, 5-Fluorouracil; VP-16, cox-2 inhibitors, Vioxx, cyclooxygenase-2 inhibitors, curcumin, MPC-6827, tamoxifen or flutamide, etoposide, PG490, 2-methoxyestradiol, AEE-788, aglycon protopanaxadiol, aplidine, ARQ-501, arsenic trioxide, BMS-387032, canertinib dihydrochloride, canfosfamide hydrochloride, combretastatin A-4 prodrug, idronoxil, indisulam, INGN-201, mapatumumab, motexafin gadolinium, oblimersen sodium, OGX-011, patupilone, PXD-101, rubitecan, tipifarnib, trabectedin PXD-101, methotrexate, Zerumbone, camptothecin, MG-98, VX-680, Ceflatonin, Oblimersen sodium, motexafin gadolinium, 1D09C3, PCK-3145, ME-2 and apoptosis-inducing-ligand (TRAIL/Apo-2 ligand). Others are provided in a report entitled “competitive outlook on apoptosis in oncology, December 2006, published by Bioseeker, and available, e.g., at http://bizwiz.bioseeker.com/bw/Archives/Files/TOC_BSG0612193.pdf.

Generally, any drug that affects an apoptosis target may also be used. Apoptosis targets include the tumour-necrosis factor (TNF)-related apoptosis-inducing ligand (TRAIL) receptors, the BCL2 family of anti-apoptotic proteins (such as Bcl-2), inhibitor of apoptosis (IAP) proteins, MDM2, p53, TRAIL and caspases. Exemplary targets include B-cell CLL/lymphoma 2, Caspase 3, CD4 molecule, Cytosolic ovarian carcinoma antigen 1, Eukaryotic translation elongation factor 2, Farnesyltransferase, CAAX box, alpha; Fc fragment of IgE; Histone deacetylase 1;Histone deacetylase 2; Interleukin 13 receptor, alpha 1; Phosphodiesterase 2A, cGMP-stimulatedPhosphodiesterase 5A, cGMP-specific; Protein kinase C, beta 1;Steroid 5-alpha-reductase, alpha polypeptide 1; 8.1.15 Topoisomerase (DNA) I; Topoisomerase (DNA) II alpha; Tubulin, beta polypeptide; and p53 protein.

In certain embodiments, the compounds described herein, e.g., EGCG, are naturally-occurring and may, e.g., be isolated from nature. Accordingly, in certain embodiments, a compound is used in an isolated or purified form, i.e., it is not in a form in which it is naturally occurring. For example, an isolated compound may contain less than about 50%, 30%, 10%, 1%, 0.1% or 0.01% of a molecule that is associated with the compound in nature. A purified preparation of a compound may comprise at least about 50%, 70%, 80%, 90%, 95%, 97%, 98% or 99% of the compound, by molecule number or by weight. Compositions may comprise, consist essentially of consist of one or more compounds described herein. Some compounds that are naturally occurring may also be synthesized in a laboratory and may be referred to as “synthetic.” Yet other compounds described herein are non-naturally occurring.

In certain embodiments, the chemotherapeutic drug is in a preparation from a natural source, e.g., a preparation from green tea.

Pharmaceutical compositions comprising 1, 2, 3, 4, 5 or more chemotherapeutic drugs or pharmaceutically acceptable salts thereof are also provided herein. A pharmaceutical composition may comprise a pharmaceutically acceptable carrier. A composition, e.g., a pharmaceutical composition, may also comprise a vaccine, e.g., a DNA vaccine, and optionally 1, 2, 3, 4, 5 or more vectors, e.g., other DNA vaccines or other constructs, e.g., described herein.

Compounds may be provided with a pharmaceutically acceptable salt. The term “pharmaceutically acceptable salts” is art-recognized, and includes relatively non-toxic, inorganic and organic acid addition salts of compositions, including without limitation, therapeutic agents, excipients, other materials and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine; (trihydroxymethyl)aminoethane; and the like. See, for example, J. Pharm. Sci., 66:1-19 (1977).

Also provided herein are compositions and kits comprising one or more DNA vaccines and one or more chemotherapeutic drugs, and optionally one or more other constructs described herein.

Therapeutic Compositions and their Administration

A vaccine composition comprising a nucleic acid, a particle comprising the nucleic acid or a cell expressing this nucleic acid, may be administered to a mammalian subject. The vaccine composition may be administered in a pharmaceutically acceptable carrier in a biologically-effective and/or a therapeutically-effective amount.

Certain conditions as described herein are disclosed in the Examples. The composition may be given alone or in combination with another protein or peptide such as an immunostimulatory molecule. Treatment may include administration of an adjuvant, used in its broadest sense to include any nonspecific immune stimulating compound such as an interferon. Adjuvants contemplated herein include resorcinols, non-ionic surfactants such as polyoxyethylene oleyl ether and n-hexadecyl polyethylene ether.

A therapeutically effective amount is a dosage that, when given for an effective period of time, achieves the desired immunological or clinical effect.

A therapeutically active amount of a nucleic acid encoding the fusion polypeptide may vary according to factors such as the disease state, age, sex, and weight of the individual, and the ability of the peptide to elicit a desired response in the individual.

Dosage regimes may be adjusted to provide the optimum therapeutic response. For example, several divided doses may be administered daily or the dose may be proportionally reduced as indicated by the exigencies of the therapeutic situation. A therapeutically effective amount of the protein, in cell associated form may be stated in terms of the protein or cell equivalents.

Thus an effective amount of the vaccine may be between about 1 nanogram and about 1 gram per kilogram of body weight of the recipient, between about 0.1 pg/kg and about 10 mg/kg, between about 1 pg/kg and about 1 mg/kg. Dosage forms suitable for internal administration may contain (for the latter dose range) from about 0.1 μg to 100 μg of active ingredient per unit. The active ingredient may vary from 0.5 to 95% by weight based on the total weight of the composition. Alternatively, an effective dose of cells transfected with the DNA vaccine constructs of the present invention is between about 10⁴ and 10⁸ cells. Those skilled in the art of immunotherapy will be able to adjust these doses without undue experimentation.

In certain embodiments, the routes of administration of the DNA may include (a) intratumoral, peritumoral, and/or intradermal “gene gun” delivery wherein DNA-coated gold particles in an effective amount are delivered using a helium-driven gene gun (BioRad, Hercules, Calif.) with a discharge pressure set at a known level, e.g., of 400 p.s.i.; (b) intramuscular (i.m.) or intravaginal injection using a conventional syringe needle; and (c) use of a needle-free biojector such as the Biojector 2000 (Bioject Inc., Portland, Oreg.) which is an injection device consisting of an injector and a disposable syringe. The orifice size controls the depth of penetration. For example, 50 μg of DNA may be delivered using the Biojector with no. 2 syringe nozzle.

Other routes of administration include the following. The term “systemic administration” refers to administration of a composition or agent such as a DNA vaccine as described herein, in a manner that results in the introduction of the composition into the subject's circulatory system or otherwise permits its spread throughout the body. “Regional” administration refers to administration into a specific, and somewhat more limited, anatomical space, such as intraperitoneal, intrathecal, subdural, or to a specific organ. “Local administration” refers to administration of a composition or drug into a limited, or circumscribed, anatomic space, such as intratumoral injection into a tumor mass, subcutaneous injections, intradermal, intramuscular, or intravaginal injections. Those of skill in the art will understand that local administration or regional administration may also result in entry of a composition into the circulatory system i.e., rendering it systemic to one degree or another. Other routes of administration include oral, intranasal or rectal or any other route known in the art.

For accomplishing the objectives of the present invention, nucleic acid therapy may be accomplished by direct transfer of a functionally active DNA into mammalian somatic tissue or organ in vivo. DNA transfer can be achieved using a number of approaches described below. These systems can be tested for successful expression in vitro by use of a selectable marker (e.g., G418 resistance) to select transfected clones expressing the DNA, followed by detection of the presence of the antigen-containing expression product (after treatment with the inducer in the case of an inducible system) using an antibody to the product in an appropriate immunoassay.

The DNA molecules, e.g., encoding a fusion polypeptides, may also be packaged into retrovirus vectors using packaging cell lines that produce replication-defective retroviruses, as is well-known in the art (e.g., Cone, R. D. et al., Proc Natl Acad Sci USA 81:6349-53, 1984; Mann, R F et al., Cell 33:153-9, 1983; Miller, A D et al., Molec Cell Biol 5:431-7, 1985; Sorge, J, et al., Molec Cell Biol 4:1730-7, 1984; Hock, R A et al., Nature 320:257, 1986; Miller, A D et al., Molec Cell Biol 6:2895-2902 (1986). Newer packaging cell lines which are efficient an safe for gene transfer have also been described (Bank et al., U.S. Pat. No. 5,278,056, incorporated by reference).

The above approach can be utilized in a site specific manner to deliver the retroviral vector to the tissue or organ of choice. Thus, for example, a catheter delivery system can be used (Nabel, E G et al., Science 244:1342 (1989)). Such methods, using either a retroviral vector or a liposome vector, are particularly useful to deliver the nucleic acid to be expressed to a blood vessel wall, or into the blood circulation of a tumor.

Depending on the route of administration, the composition may be coated in a material to protect the compound from the action of enzymes, acids and other natural conditions which may inactivate the compound. Thus it may be necessary to coat the composition with, or co-administer the composition with, a material to prevent its inactivation. For example, an enzyme inhibitors of nucleases or proteases (e.g., pancreatic trypsin inhibitor, diisopropylfluorophosphate and trasylol) or in an appropriate carrier such as liposomes (including water-in-oil-in-water emulsions as well as conventional liposomes (Strejan et al., J. Neuroimmunol 7:27, 1984).

Other pharmaceutically acceptable carriers for the nucleic acid vaccine compositions according to the present invention are liposomes, pharmaceutical compositions in which the active protein is contained either dispersed or variously present in corpuscles consisting of aqueous concentric layers adherent to lipidic layers. The active protein may be present in the aqueous layer and in the lipidic layer, inside or outside, or, in any event, in the non-homogeneous system generally known as a liposomic suspension. The hydrophobic layer, or lipidic layer, generally, but not exclusively, comprises phospholipids such as lecithin and sphingomyelin, steroids such as cholesterol, more or less ionic surface active substances such as dicetylphosphate, stearylamine or phosphatidic acid, and/or other materials of a hydrophobic nature. Those skilled in the art will appreciate other suitable embodiments of the present liposomal formulations.

A chemotherapeutic drug may be administered in doses that are similar to the doses that the chemotherapeutic drug is used to be administered for cancer therapy. Alternatively, it may be possible to use lower doses, e.g., doses that are lower by 10%, 30%, 50%, or 2, 5, or 10 fold lower. Generally, the dose of chemotherapeutic agent is a dose that is effective to increase the effectiveness of a DNA vaccine, but less than a dose that results in significant immunosuppression or immunosuppression that essentially cancels out the effect of the DNA vaccine.

The route of administration of chemotherapeutic drugs may depend on the drug. For use in the methods described herein, a chemotherapeutic drug may be used as it is commonly used in known methods. Generally, the drugs will be administered orally or they may be injected. The regimen of administration of the drugs may be the same as it is commonly used in known methods. For example, certain drugs are administered one time, other drugs are administered every third day for a set period of time, yet other drugs are administered every other day or every third, fourth, fifth, sixth day or weekly. The Examples provide exemplary regimens for administrating the drugs, as well as DNA vaccines.

The compositions of the present invention, may be administered simultaneously or subsequently. When administered simultaneously, the different components may be administered as one composition. Accordingly, also provided herein are compositions, e.g., pharmaceutical compositions comprising one or more agents.

In one embodiment, a subject first receives one or more doses of chemotherapeutic drug and then one or more doses of DNA vaccine. In the case of DMXAA, it may be preferable to administer to the subject a dose of DNA vaccine first and then a dose of chemotherapeutic drug. One may administer 1, 2, 3, 4, 5 or more doses of DNA vaccine and 1, 2, 3, 4, 5 or more doses of chemotherapeutic agent.

A method may further comprise subjecting a subject to another cancer treatment, e.g., radiotherapy, an anti-angiogenesis agent and/or a hydrogel-based system.

As used herein “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in the therapeutic compositions is contemplated. Supplementary active compounds can also be incorporated into the compositions.

Pharmaceutically acceptable diluents include saline and aqueous buffer solutions. Pharmaceutical compositions suitable for injection include sterile aqueous solutions (where water soluble) or dispersions and sterile powders for the extemporaneous preparation of sterile injectable solutions or dispersion. Isotonic agents, for example, sugars, polyalcohols such as mannitol, sorbitol, sodium chloride may be included in the pharmaceutical composition. In all cases, the composition should be sterile and should be fluid. It should be stable under the conditions of manufacture and storage and must include preservatives that prevent contamination with microorganisms such as bacteria and fungi. Dispersions can also be prepared in glycerol, liquid polyethylene glycols, and mixtures thereof and in oils. Under ordinary conditions of storage and use, these preparations may contain a preservative to prevent the growth of microorganisms.

The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

Prevention of the action of microorganisms in the pharmaceutical composition can be achieved by various antibacterial and antifungal agents, for example, parabens, chlorobutanol, phenol, ascorbic acid, thimerosal, and the like.

Compositions may be formulated in dosage unit form for ease of administration and uniformity of dosage. Dosage unit form refers to physically discrete units suited as unitary dosages for a mammalian subject; each unit contains a predetermined quantity of active material (e.g., the nucleic acid vaccine) calculated to produce the desired therapeutic effect, in association with the required pharmaceutical carrier. The specification for the dosage unit forms of the invention are dictated by and directly dependent on (a) the unique characteristics of the active material and the particular therapeutic effect to be achieved, and (b) the limitations inherent in the art of compounding such an active compound for the treatment of, and sensitivity of, individual subjects.

For lung instillation, aerosolized solutions are used. In a sprayable aerosol preparations, the active protein may be in combination with a solid or liquid inert carrier material. This may also be packaged in a squeeze bottle or in admixture with a pressurized volatile, normally gaseous propellant. The aerosol preparations can contain solvents, buffers, surfactants, and antioxidants in addition to the protein of the invention.

Diseases that may be treated as described herein include hyper proliferative diseases, e.g., cancer, whether localized or having metastasized. Exemplary cancers include head and neck cancers and cervical cancer. Any cancer can be treated provided that there is a tumor associated antigen that is associated with the particular cancer. Other cancers include skin cancer, lung cancer, colon cancer, kidney cancer, breast cancer, prostate cancer, pancreatic cancer, bone cancer, brain cancer, as well as blood cancers, e.g., myeloma, leukemia and lymphoma. Generally, any cell growth can be treated provided that there is an antigen associated with the cell growth, which antigen or homolog thereof can be encoded by a DNA vaccine.

Treating a subject includes curing a subject or improving at least one symptom of the disease or preventing or reducing the likelihood of the disease to return. For example, treating a subject having cancer could be reducing the tumor mass of a subject, e.g., by about 10%, 30%, 50%, 75%, 90% or more, eliminating the tumor, preventing or reducing the likelihood of the tumor to return, or partial or complete remission.

All references cited herein are all incorporated by reference herein, in their entirety, whether specifically incorporated or not. All publications, patents, patent applications, GenBank sequences and ATCC deposits, cited herein are hereby expressly incorporated by reference for all purposes. In particular, all nucleotide sequences, amino acid sequences, nucleic constructs, DNA vaccines, methods of administration, particular orders of administration of DNA vaccines and agents that are described in the patents, patent applications and other publications referred to herein or authored by one or more of the inventors of this application are specifically incorporated by reference herein. In case of conflict, the definitions within the instant application govern.

Having now fully described this invention, it will be appreciated by those skilled in the art that the same can be performed within a wide range of equivalent parameters, concentrations, and conditions without departing from the spirit and scope of the invention and without undue experimentation.

The present description is further illustrated by the following examples, which should not be construed as limiting in any way.

### EXAMPLES

### Example 1

### Material and Methods For Examples 2-6

### A. Mice

Female C57BL/6 mice (H-2K^{b} and I-A^{b}), 5 to 6 weeks of age, were purchased from National Cancer Institute (Frederick, Md.). Transgenic mice, OT-1, that express TCR specific for ovalbumin peptide, SIINFEKL, were purchased from The Jackson Laboratory. All of the mice were maintained under specific pathogen-free conditions in the animal facility at Johns Hopkins Hospital (Baltimore, Md.). Animals were used in compliance with institutional animal health care regulations, and all animal experimental procedures were approved by the Johns Hopkins Institutional Animal Care and Use Committee.

### B. Cell Lines

The production and maintenance of TC-1 cells or TC-1-luciferase transduced (TC-1 luc) cells have been described previously (Lin et al., Cancer. Res., 56:21-6 (1996); Kim et al., Human Gene Ther., 18:575-88 (2007)). Mouse melanoma cell B16/F10 and thymoma cells EL4 (H-2^{b}) were purchased from ATCC (Rockville, Md., USA). For the generation of CTLs specific for H-2K^{b}-OVA, 1×10⁷EG7 cells (EL4 cells transfected with ovalbumin cDNA) were irradiated (10,000 rad) and cultured for 6 days in complete RPMI-1640 medium with 1×10⁷ spleen cells from OT-1 mice. All cell lines were grown in RPMI-1640, supplemented with 10% (v/v) fetal bovine serum, 50 Um′ penicillin/streptomycin, 2 mM L-glutamine, 1 mM sodium pyruvate, 2 mM nonessential amino acids, and 0.4 mg/ml G418 at 37° C. with 5% CO₂.

### C. Plasmid DNA Constructs

The generation of recombinant plasmid pcDNA3 encoding CRT/E7 (p-CRT/E7) or recombinant pcDNA3 encoding ovalbumin (p-OVA) has been described previously (Kim et al., J. Clin. Invest, 112:109-17 (2003); Peng et al., J. Biomed. Sci., 12:689-700 (2005)). The accuracy of the DNA construct was confirmed by DNA sequencing. For the gene gun-mediated intradermal vaccination, 2 μg/mouse of recombinant plasmid DNA were delivered to the shaved abdominal region of C57BL/6 mice using a helium-driven gene gun (BioRad, Hercules, Calif., USA) with a discharge pressure of 400 p.s.i., according to a previously described protocol (Chen et al., Cancer Res., 60:1035-42 (2000)).

### D. Recombinant Vaccinia Viruses

The wild type vaccinia virus (Vac-WT) was prepared as described previously (Wu et al., Proc. Natl. Acad. Sci. USA, 92:11671-5 (1995)). The luciferase-expressing vaccinia virus (Vac-luc) was generated using a previously described protocol. It contains two reporter genes (luc and lacZ) inserted into the thymidine kinase region of VV (tk−) as described (Chen et al., J. Immunotherapy, 24:46-57 (2001)). The vaccinia virus expressing the full-length chicken OVA (Vac-OVA) was generated using a previously described protocol (Norbury et al., J. Immunol., 166:4355-62 (2001)). The generation of recombinant vaccinia virus encoding calreticulin (CRT) linked to a model tumor antigen HPV-16 E7 (Vac-CRT/E7) was performed using a protocol similar to what has been described earlier (Earl et al., AIDS Res. Hum. Retroviruses, 9:589-94 (1993)). The generation of recombinant vaccinia virus expressing green fluorescent protein (Vac-GFP) was performed using a protocol similar to what has been described earlier (Ward et al., Methods Mol. Biol., 269:205-18 (2004)).

### E. In Vivo Bioluminescence Imaging

Viral replication levels were quantitatively compared within the tumor administered through different routes of injection. TC-1 tumor bearing mice (tumor size=8-10 mm) were administered by either intraperitoneal (i.p.) or intra-tumoral (i.t.) injection of 1×10⁷ pfu/mouse of vaccinia-luc in 200 μL phosphate-buffered saline. Bioluminescence imaging was conducted on days 1, 3, and 7 after virus injection on a cryogenically cooled IVIS system (Xenogen/Caliper Life Sciences). The region of interest (ROI) as manually drawn over tumor areas by using Living Image software 2.5 (Xenogen/Caliper Life Sciences).

### F. Characterization of CD31+ Cells Infected by Vaccinia

The frequency of CD31⁺ cells infected by vaccinia virus after TC-1 tumor bearing mice (tumor size=8-10 mm) were administered by either intraperitoneal (i.p.) or intra-tumoral (i.t.) injection of 1×10⁷ pfu vaccinia-GFP in 200 μL phosphate-buffered saline were characterized. Tumor cells were harvested 24 hours after viral injection, made into single cell suspensions, and subjected to CD31 staining.

In order to evaluate for killing of CD31⁺ cells, TC-1 tumors were grown in C57BL/6 mice and harvested as tumor size reached 8-10 mm. Tumors were dissociated into single cell suspensions and seeded (3×10⁵/well) into 24-well microtiter plate in complete medium. At 24 hours, Vac-WT or Vac-OVA at 0.5 MOI were added to each well, and at 48 hours, the complete medium was changed and activated OT-1 T cells were added to each well at an effector-to-target ratio of 1:1 (E/T=1:1). Cells were then harvested 4 hours later and stained with PE anti-mouse CD31 mAb and 7-AAD, and analyzed by flow cytometry using the FACSCalibur flow cytometer, and CellQuest software (Becton Dickinson, San Jose, Calif.). Data are presented as absolute numbers of CD31⁺ 7-AAD⁻ cells per 3×10⁵ cells.

### G. Heterologous Prime-Boost Immunization

Groups of mice (five per group) were inoculated with either B16/F10 cells or TC-1 cells (5×10⁴/mouse) at Day 0. Mice were then primed with 2 μg of either control pcDNA3, p-OVA or p-CRT/E7 DNA by gene-gun at day 5, and were boosted with i.t. injection (1×10⁷ pfu/mouse, in 200 uL PBS) of Vac-WT, Vac-OVA, or Vac-CRT/E7 at day 12.

### H. Evaluation of Frequency of E7-Specific CD8+ T Cells by Intracellular Cytokine Staining and Flow Cytometry Analysis

For characterization of E7-specific CD8⁺ T cells, both splenocytes and tumor xenografts were harvested 1 week after last immunization. Prior to intracellular cytokine staining, 2×10⁶ pooled splenocytes and pooled tumors from each treatment group were separately incubated for 16 hours with either an H-2K^{b}-restricted peptide (SIINFEKL; 1.0 μM) or an I-A^{b}-restricted peptide (LSQAVHAAHAEINEAGR; 1.0 μM). In addition, 2×10⁶ pooled splenocytes and pooled tumors from each treatment group were incubated for 16 hours with 1 μg/ml of E7 peptide (aa 49-57) containing an MHC class I epitope for detecting E7-specific CD8+ T cell precursors (Feltkamp et al., Eur. J Immunol., 23:2242-9 (1993)). Cells were then harvested, stained for CD8 and IFN-g using previously described standard protocols (Cheng et al., J Clin. Invest., 108:669-78 (2001)). Samples were analyzed on a FACSCalibur flow cytometer, using CellQuest software (Becton Dickinson, San Jose, Calif.). All of the analyses shown were carried out on a gated lymphocyte population.

### I. In Vitro Cytotoxicity Assay

Luciferase-expressing TC-1 tumor cells were added to 96-well plates at a dose of 2×10⁴/well. After 24 hours, Vac-WT or Vac-OVA (MOI=0.5) were added to each well. At 48 hours, the complete medium was changed and activated OT-1 T cells at an E:T ratio of 1:1 were added to each well. Bioluminescence imaging was performed 4 hours later. The degree of CTL-mediated killing of the tumor cells was indicated by the decrease of luminescence activity using the IVIS luminescence imaging system series 200. Bioluminescence signals were acquired for 10 seconds.

### J. Statistical Analysis

Statistical analysis was performed using Prism 3.0 software (GraphPad, San Diego, USA). All data are expressed as means±standard deviation (SD) and are representative of at least two independent experiments. Comparisons between individual data points were made using a Student's t-test or repeated measure ANOVA (analysis of variance) test, as appropriate. Tumor size was measured during the treatment twice a week by digital calipers and tumor volume (mm³) was calculated using the following equation: (tumor length×width×height)/2. Death of mouse was arbitrarily defined as tumor diameter greater than 2 cm. Differences in survival between experimental groups were analyzed using the Log rank test. A p-value of 0.05 was set for the significance of difference among groups.

### Example 2

### Tumor-Bearing Mice Primed with DNA Encoding a Foreign Antigen and Treated with Intratumoral Injection of Vaccinia Virus Encoding the Same Foreign Antigen LED to Significant Therapeutic Anti-Tumor Effects

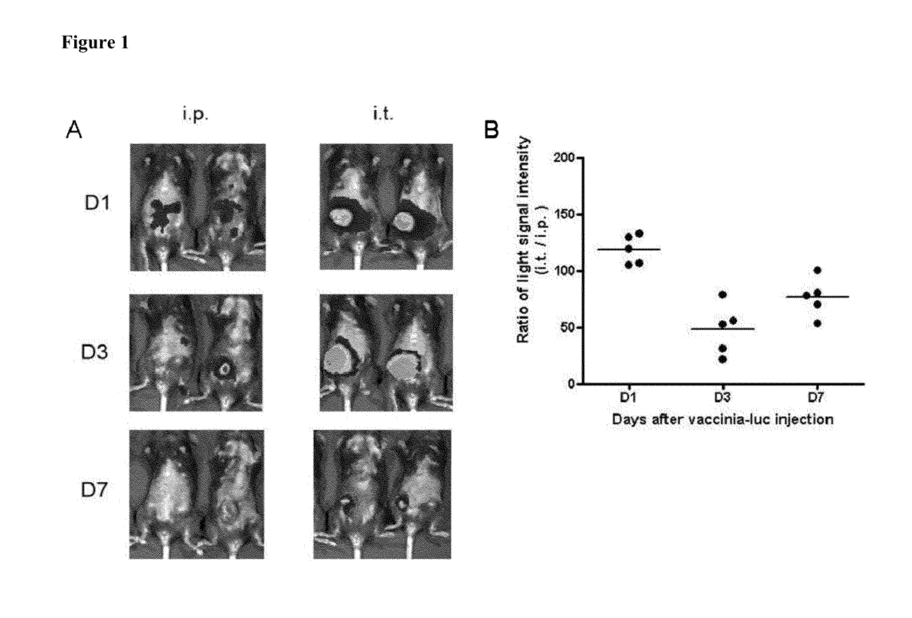

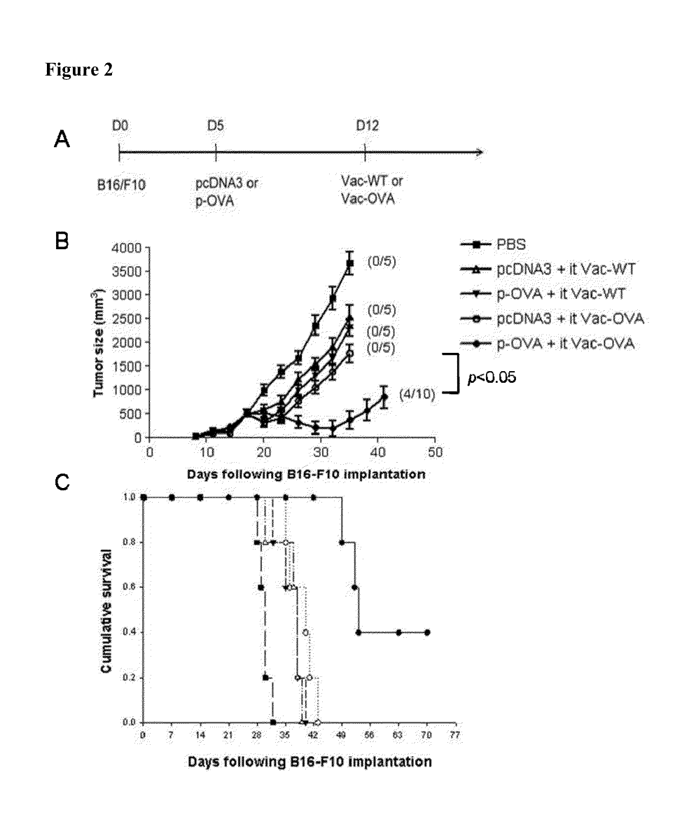

Intratumoral injection of vaccinia encoding a marker gene, such as luciferase, was recently demonstrated to result in significant expression of luciferase within the tumor, indicating that intratumoral injection of vaccinia can lead to significant viral infection of the tumor cells (FIG. 1). Thus, in order to determine the antitumor effects generated in tumor-bearing mice primed with DNA encoding a foreign antigen, such as OVA, and treated with intratumoral injection of vaccinia virus encoding the same foreign antigen, groups of C57BL/6 mice (5 per group) were first challenged with B16 tumor cells and then primed with control pcDNA3 alone or pcDNA3 encoding ovalbumin (p-OVA). One week later, mice were treated with intratumoral injections of either wild-type vaccinia (Vac-WT) or vaccinia encoding OVA (Vac-OVA). Tumor-bearing mice treated with 1×PBS were used as negative controls. A graphical representation of the treatment regimen is depicted in FIG. 2A. As shown in FIG. 2B, tumor-bearing mice primed with the p-OVA followed by intratumoral Vac-OVA injection showed the best therapeutic antitumor effects compared to treatment with the other prime-boost regimens. Furthermore, tumor-bearing mice primed with the p-OVA prime followed by intratumoral Vac-OVA injection showed improved survival compared to treatment with the other therapeutic regimens (p<0.01) (FIG. 2C). Thus, the data indicate that the treatment with p-OVA followed by intratumoral Vac-OVA injection produced significant therapeutic anti-tumor effects and long-term survival in B16 tumor-bearing mice.

The same therapeutic approach was further tested using another tumor model, TC-1. Groups of C57BL/6 mice (5 per group) were first challenged with TC-1 tumor cells and then primed them with control pcDNA3 or p-OVA. One week later, mice were treated with either Vac-WT or Vac-OVA by intratumoral injection. Tumor-bearing mice treated with PBS were used as negative controls. A graphical representation of the treatment regimen is depicted in FIG. 3A. As shown in FIG. 3B, tumor-bearing mice treated with the p-OVA followed by intratumoral Vac-OVA injection showed the best therapeutic antitumor effects compared to treatment with the other prime-boost regimens. Furthermore, tumor-bearing mice treated with the p-OVA followed by intratumoral Vac-OVA injection showed improved survival compared to treatment with the other therapeutic regimens (p<0.01; FIG. 3C). Thus, the data indicated that the treatment with p-OVA followed by intratumoral Vac-OVA injection produced significant therapeutic anti-tumor effects and long-term survival in TC-1 tumor-bearing mice.

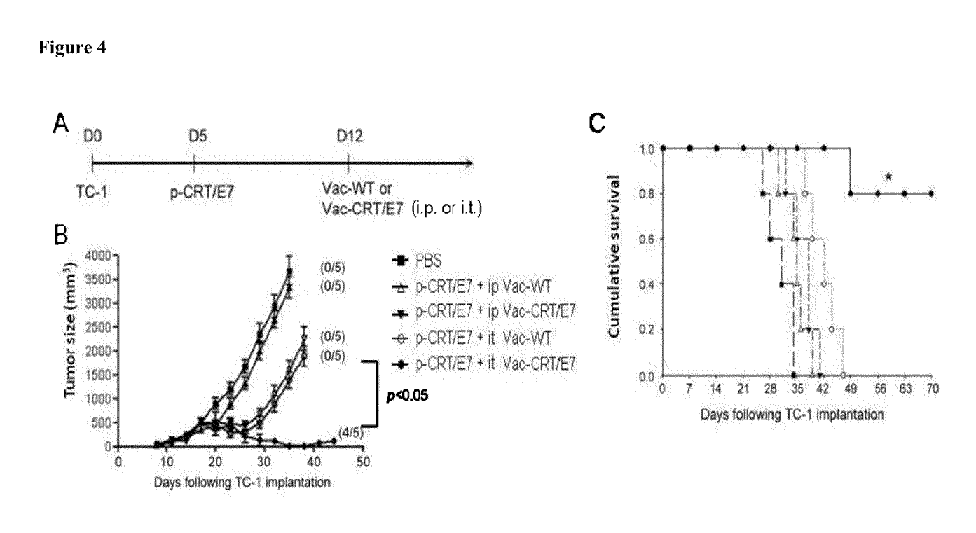

The therapeutic approach was further tested using an antigenic system specific to TC-1 tumor cells, specifically E7. It was found that vaccination with CRT/E7 DNA vaccine intradermally followed by intratumoral injection of vaccinia encoding CRT/E7 also generated significant therapeutic anti-tumor effects and long-term survival in TC-1 tumor-bearing mice (FIG. 4). Taken together, the data demonstrate that the treatment with a foreign antigen-specific DNA vaccine followed by intratumoral injection of vaccinia encoding the same foreign antigen produced significant therapeutic anti-tumor effects and long-term survival in tumor-bearing mice in two different tumor models.

### Example 3

### Tumor-Bearing Mice Primed with DNA Encoding Foreign Antigen and Treated with Intratumoral Injection of Vaccinia Encoding the Same Foreign Antigen Leads to Significant Number of Foreign Antigen-Specific CD8+ T Cells

In order to determine the antigen-specific CD8⁺ T cell immune response against OVA in tumor-bearing mice using the DNA prime and intratumoral viral boost model, groups of C57BL/6 mice (5 per group) were first challenged with B16 tumor cells and then treated with either pcDNA3 or p-OVA followed by intratumoral injection with either Vac-WT or Vac-OVA, as previously described in FIG. 2. Tumor-bearing mice treated with 1×PBS were used as negative controls. Cells were harvested from the spleens and tumors of vaccinated mice 7 days after vaccinia injection and were characterized for the presence of OVA-specific CD8⁺ T cells using intracellular cytokine staining for IFN-γ followed by flow cytometry analysis. As shown in FIG. 5, tumor-bearing mice that were treated with p-OVA followed by intratumoral Vac-OVA injection generated a significantly higher numbers/percentages of OVA-specific CD8⁺ T cells both in the spleens as well as tumors compared to tumor-bearing mice treated with the other regimens.

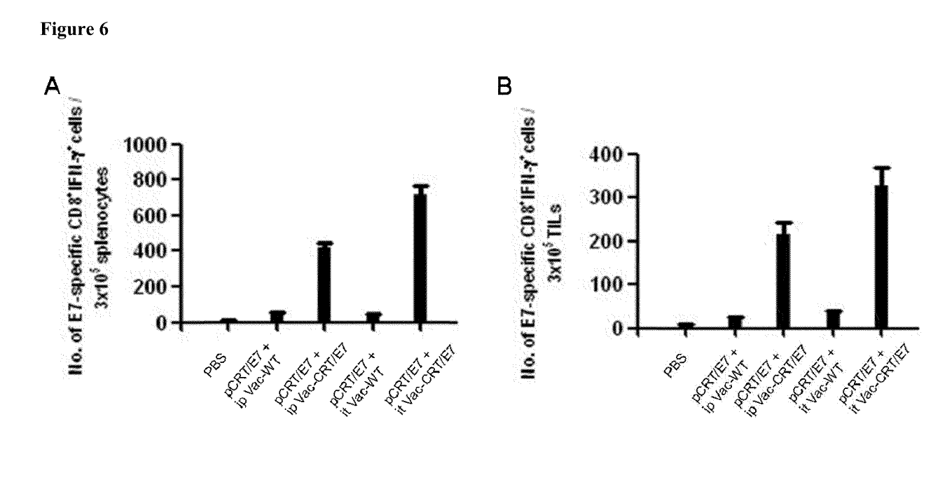

The antigen-specific immune responses elicited in another tumor model, TC-1, which uses a different antigenic system, E7, were also determined Groups of C57BL/6 mice (5 per group) were first challenged with TC-1 tumor cells and then primed them with either pcDNA3 or p-CRT/E7 DNA vaccine intradermally. One week later, mice were treated with either Vac-WT or Vac-CRT/E7 by either intraperitoneal or intratumoral injection. Tumor-bearing mice treated with PBS were used as negative controls. It was observed that tumor-bearing mice that were treated with p-CRT/E7 DNA followed by intratumoral Vac-CRT/E7 injection generated a significantly higher number of E7-specific CD8⁺ T cells both in the spleens as well as tumors compared to tumor-bearing mice treated with the other regimens (FIG. 6). Taken together, the data indicate that treatment of tumor-bearing mice with a foreign antigen-specific DNA vaccine followed by intratumoral injection of vaccinia encoding the same foreign antigen leads to the strongest antigen-specific CD8+ T cell immune responses in the spleens and tumors.

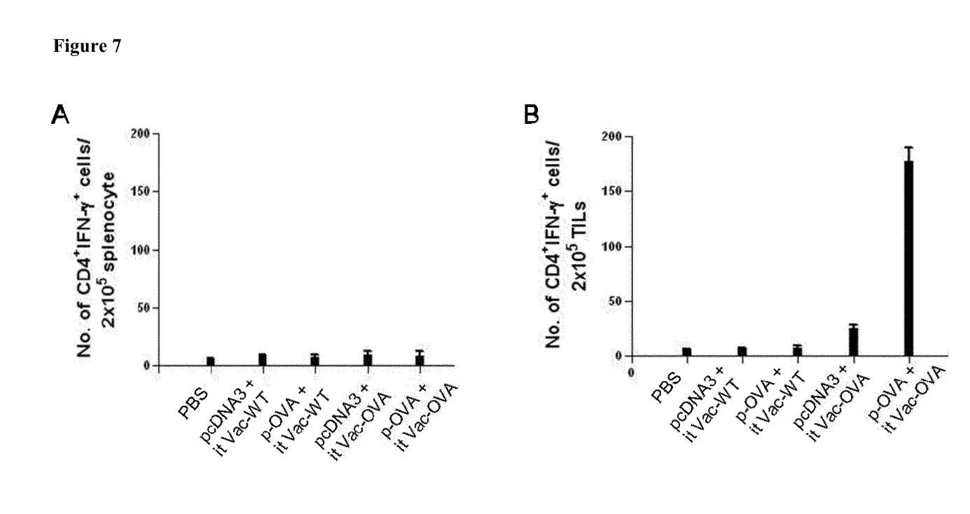

OVA-specific CD4⁺ T cell immune responses in tumor-bearing mice treated with p-OVA followed by intratumoral Vac-OVA injection were also determined. It was found that while the OVA-specific CD4⁺ T cell immune responses in the spleens of treated mice were not significantly different from those in tumor-bearing mice treated with the other regimens, the OVA-specific CD4⁺ T cell immune responses within the tumors of treated mice were significantly higher compared to those in tumor-bearing mice treated with the other regimens (FIG. 7). Thus, the data indicate that treatment with p-OVA followed by intratumoral Vac-OVA injection leads to increased OVA-specific CD4+ T cell immune responses in the tumors, but not in the spleens of tumor-bearing mice.

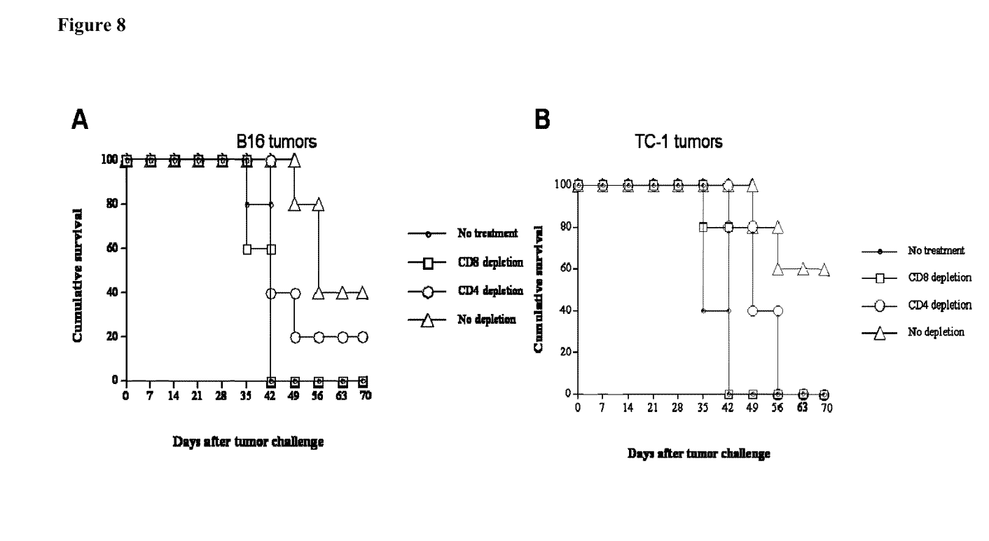

In order to determine the subset of immune cells that are important for the observed antitumor effects, in vivo antibody depletion experiments were performed in tumor-bearing mice treated with the p-OVA followed by intratumoral Vac-OVA injection. It was found that mice depleted of CD8⁺ T cells showed a significant reduction in survival compared to treated mice without depletion in both tumor models (FIG. 8). Furthermore, depletion of CD4⁺ T cells showed a slight reduction in survival, although not as significant as CD8⁺ T cell depletion. Taken together, the data indicate that CD8⁺ T cells, as well as CD4⁺ T cells, play an important role in the antitumor effects observed in mice treated with p-OVA followed by intratumoral Vac-OVA injection.

### Example 4

### Treatment with OVA Expressing Vaccinia not Only Kills the Tumor Cells Directly, but Also Renders Tumor Cells More Susceptible to Killing by OVA-Specific T Cells

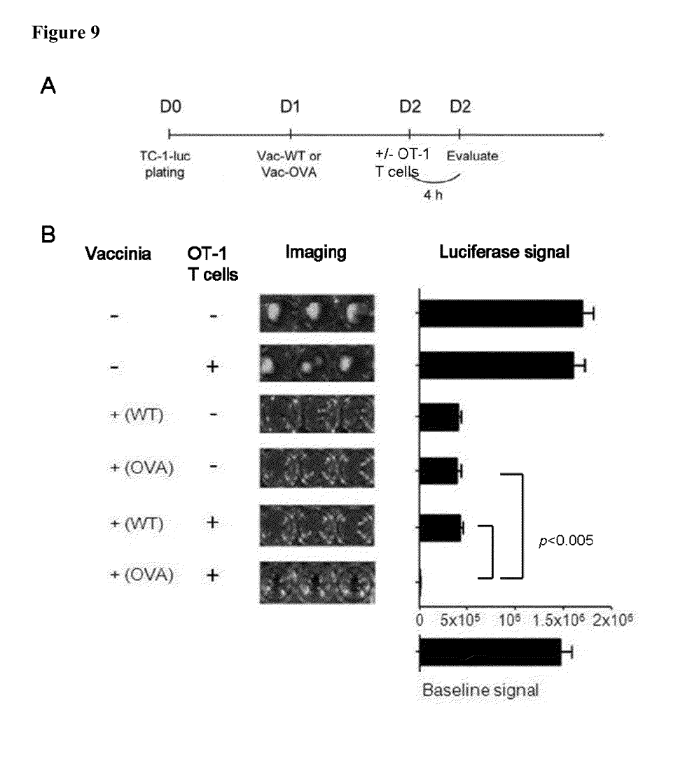

In order to determine if treatment of tumor cells with Vac-OVA renders tumor cells more susceptible to viral oncolysis as well as OVA-specific T cell-mediated killing, a cytotoxicity assay was performed using luciferase-expressing TC-1 tumor cells. TC-1/luc tumor cells were plated on Day 0 and treated with either Vac-OVA or Vac-WT on Day 1. The cells were then treated with or without OVA-specific CD8+ T cells (OT-1 T cells) on Day 2 as shown in FIG. 9A. Four hours later, the CTL-mediated killing of the TC-1 tumor cells in each well was monitored using bioluminescent imaging system. The degree of CTL-mediated killing of the tumor cells was indicated by the decrease of luminescence activity. As shown in FIG. 9B, it was observed that tumor cells incubated with Vac-WT or Vac-OVA alone demonstrated a significant reduction in luciferase activity, indicating that tumor killing was contributed by viral oncolysis. Furthermore, the lowest luciferase activity was observed in TC-1 cells treated with Vac-OVA in conjunction with OT-1 T cells, but not in cells treated with Vac-WT. The data indicates that the increased tumor lysis is contributed by OVA-specific cytotoxic T cell-mediated killing. Taken together, the data indicate that the treatment of tumor cells with Vac-OVA and OT-1 cells can lead to tumor lysis by a combination of viral oncolysis and OVA-specific cytotoxic T cell-mediated killing.

### Example 5

### Intratumoral Injection of Vaccinia Leads to Infection of CD31+ Non-Tumor Cells by Vaccinia

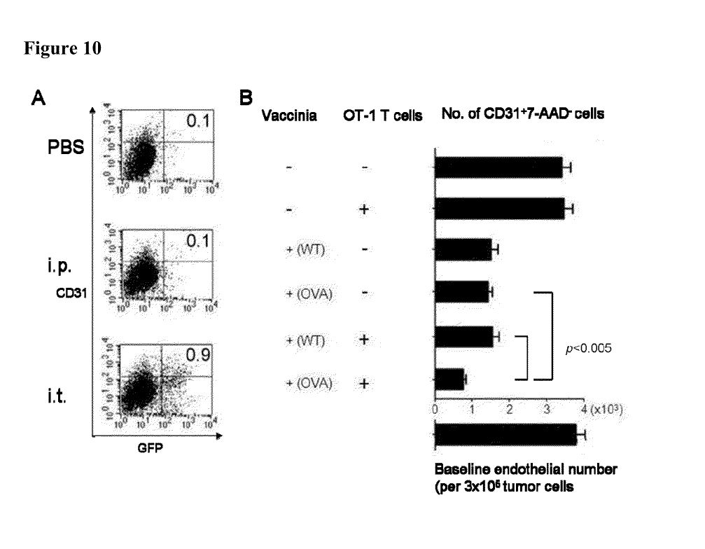

It was further investigated whether Vac-OVA treatment could exert cytotoxic effects on the surrounding non-tumor cells, including CD31⁺ endothelial and stromal cells. In order to determine the number of CD31⁺ non-tumor cells infected by vaccinia in tumor-bearing mice, groups of C57BL/6 mice (5 per group) were subcutaneously challenged with TC-1 tumor cells and treated with either intratumoral (i.t.) or intraperitoneal (i.p.) injection with Vac-GFP. Tumor cells were harvested 24 hours after vaccinia virus injection, stained for CD31 and characterized by flow cytometry analysis. As shown in FIG. 10A, the percentage of CD31⁺ non-tumor cells infected with Vac-GFP was significantly higher in tumor-bearing mice injected intratumorally with Vac-GFP compared to mice injected intraperitoneally or mice treated with PBS. Thus, the data indicate that intratumoral injection of vaccinia leads to increased infection of CD31+ non-tumor cells by vaccinia compared to intraperitoneal injection.

### Example 6

### Treatment with Vaccinia-OVA not Only Kills the Surrounding CD31+ Stromal Cells in the Tumor Microenvironment Directly but Also Renders them More Susceptible to Killing by OVA-Specific T Cells

It was further determined if treatment of explanted tumor injected with Vac-OVA would render the CD31⁺ non-tumor cells derived from the surrounding tumor stroma more susceptible to viral oncolysis and to OVA-specific CD8⁺ T cell-mediated killing. Therefore, explanted TC-1 tumor cells were plated in 96-well plates on day 0 and treated them with Vac-OVA or Vac-WT on day 1. The cells were then treated with or without OVA-specific CD8⁺ T cells (OT-1 T cells) on day 2. Four hours later, the cells were analyzed by flow cytometry analysis for expression of CD31 and 7-AAD. As shown in FIG. 10B, it was observed that CD31⁺ cells incubated with Vac-WT or Vac-OVA alone demonstrated a significant reduction in luciferase activity, indicating that killing was contributed by viral oncolysis. Furthermore, the lowest luciferase activity was observed in CD31⁺ cells treated with Vac-OVA and OT-1 T cells, but not in cells treated with Vac-WT, suggesting that the increased tumor lysis is contributed by OVA-specific cytotoxic T cell-mediated killing. Taken together, the data indicates that the treatment of CD31⁺ cells with Vac-OVA and OT-1 cells can lead to lysis by a combination of viral oncolysis and OVA-specific cytotoxic T cell-mediated killing.

### Example 7

### Material and Methods For Examples 8-14

### A. Mice

Female C57BL/6 mice, 6 to 8 weeks of age, were purchased from National Cancer Institute (Frederick, Md.).

### B. Cell Lines

TC-1 cells expressing the HPV16 E6-E7 proteins (Lin et al., Cancer. Res., 56:21-6 (1996)) and the TC-1 cells expressing the firefly luciferase gene (TC-1 luc) were developed in our laboratory and have been described previously (Huang et al., Vaccine, 25:7824-31 (2007)).

### C. Antibodies and Tetramer

Fluorochrome-conjugated anti-mouse monoclonal antibodies (Abs) CD8a-APC, CD103-APC, α4β7-APC were purchased from eBiosciences; CD8A-FITC and the 7-aminoactinomycin D (7-AAD) were purchased from BD Pharmingen; CCR9-FITC was purchased from BioLegend; H2D^{b} E-7 tetramer, which allows for the staining of cells that bind the E-7 peptide, was provided by National Institute of Allergy and Infectious Diseases tetramer core facility Ammonium chloride solution (ACK) was purchased from Quality Biological INC.

### D. Lymphocyte Preparation

Blood was obtained from the tail vessel of the mice and mixed with PBS. Mice were euthanized and organs were removed by dissection. Cervicovaginal (cervical and vaginal tissues) cell suspensions were obtained by enzymatic dispersion in RPMI 1640 digestion buffer for 1 hour at 37° C. while shaking. Cervicovaginal cells were passed through a 70-μM cell strainer (Becton Dickinson). Iliac lymph node and spleen cell suspensions were mechanically disrupted and filtered through a 70-μM cell strainer. Blood, cervicovaginal, spleen and lymph node cell suspensions were washed in RPMI/FBS 2% and freed from erythrocytes by treatment with ammonium chloride solution.

### E. Immunization Procedures

Mice were immunized by intracervicovaginal (lateral wall of the cervicovaginal tract) or intramuscular injection at day 0 (pNGVL4a-sig/E7(detox)/HSP70 DNA vaccine, 50 μg) and day 7 (TA-HPV 1×10⁷ PFU). The total volume injected was 50 μl in both routes. Mice were anesthetized before immunization.

pNGVL4a-sig/E7(detox)/HSP70 is a therapeutic HPV DNA vaccine encoding a chimeric protein consisting of a signal peptide (sig) linked to HPV-16 E7 antigen and heat shock protein 70 (HSP70) (Timble et al., Vaccine 21:4036-42 (2003)). pNGVL4a-sig/E7(detox)/HSP70 DNA vaccine has been used in a clinical trials in patients with high grade intraepithelial lesions and proven to be safe (Trimble et al., Am. Assoc. Cancer Research 15:361-67 (2009)). Currently, the DNA vaccine, pNGVL4a-sig/E7(detox)/HSP70, is being used in combination with a recombinant therapeutic HPV vaccine, TA-HPV, in the context of a DNA prime and vaccinia boost regimen in patients with grade 3 cervical intraepithelial neoplasia (ClinicalTrials.gov Identifier NCT00788164) (Sci. Transl. Med. 6, 221 ra 13 (2014).). TA-HPV is a recombinant vaccinia vaccine that encodes HPV-16/18 E6 and E7 proteins. TA-HPV has been used in several clinical trials and proved to be safe (Borysiewicz et al. Lancet 347:1523-27 (1996); Kaufmann et al., Am. Assoc. Cancer Res. 8:3637-85 (2002); Davidson et al., Cancer Res. 63:6032-41 (2003); Smyth et al., Am. Assoc. Cancer Res. 10:2954-61 (2004); Fiander et al., Gynecological Cancer Soc. 16:1075-81 (2006)). As such, pNGVL4a-sig/E7(detox)/HSP70 DNA vaccine and TA-HPV are favorable for use in a prime-boost regimen.

### F. Cell Surface Staining and Flow Cytometry Analysis

All staining was performed in flow tube in a final volume of 300 μl FACS buffer (PBS+2% FBS) for 1 hour at 4° C. To avoid nonspecific antibody binding through surface Fc receptor, all cells were pre-incubated with CD16/32 mouse BD Fc Block™ (Becton Dickinson pharmingen). Analyses were performed on a Becton-Dickinson FACScan with CELLQuest software (Becton Dickinson Immunocytometry System, Mountain View, Calif.).

### G. In Vivo Tumor Protection and Imaging Techniques

2×10⁴ TC-1 luc cells were injected into the submucosal area of the cervicovaginal tract wall of the mice. Mice were vaccinated on day 2 (pNGVL4a-sig/E7(detox)/HSP70 DNA) and day 7 (TA-HPV) either intramuscularly or intracervicovaginally after tumor challenge. Genital tumor growth was monitored by bioluminescence in a Xenogen imaging system once a week. Briefly, D-Luciferin was dissolved to 7.8 mg/mL in PBS, filter sterilized, and stored at −80° C. Mice were given D-Luciferin by i.p. injection (200 μl/mouse, 75 mg/kg) and anesthetized with isoflurane. In vivo bioluminescence imaging for luciferase was conducted on a cryogenically cooled IVIS system using Living Image acquisition and analysis software (Xenogen). Mice were then placed onto the warmed stage inside the light-tight camera box with continuous exposure to 1%-2% isoflurane. Images were acquired 10 min after D-luciferin administration and imaged for 2 min. The levels of light from the bioluminescent cells were detected by IVIS camera system, integrated, and digitized. Region of interest from displayed images was designated around the vagina and quantified as total photon counts using Living Image 2.50 software (Xenogen).

### H. Statistical Analyses

All data are expressed as mean±standard deviation (S.D.) and are representative of at least two separate experiments. Comparisons between individual data points were made using Student's t-test. The non-parametric Mann-Whitney test was used for comparing two different groups. All p values <0.05 were considered significant.

### Example 8

### Vaccination with pNGVL4a-Sig/E7(Detox)/HSP70 DNA IM Prime Followed by TA-HPV Boost Intraperitoneally Elicits Stronger E7-Specific CD8+ T Cell Response Compared to a Homologous DNA-DNA Prime-Boost Regimen

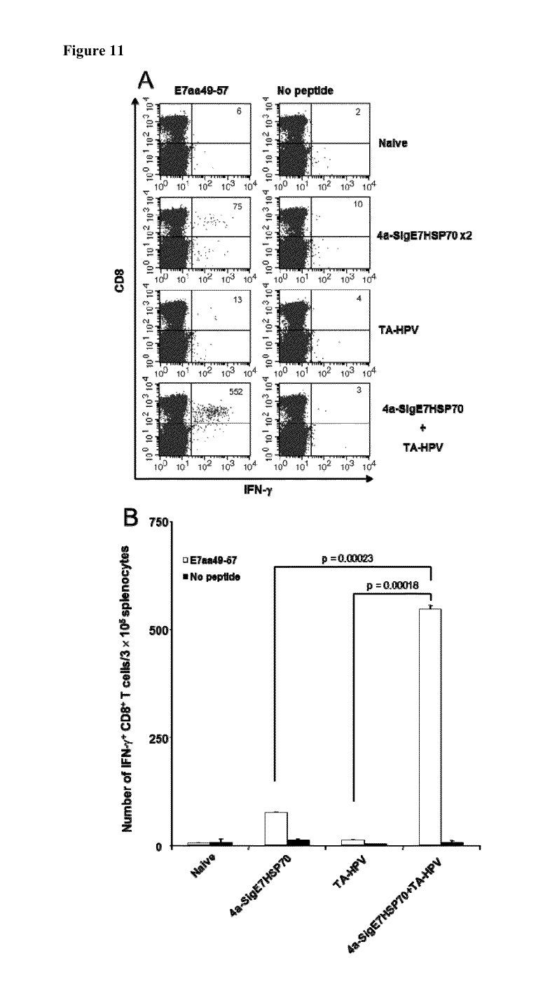

The optimal prime-boost vaccination regimen to generate antigen-specific CD8+ T cells was determined using various combinations of the pNGVL4a-sig/E7(detox)/HSP70 DNA and TA-HPV vaccines. C57BL/6 mice (five per group) were vaccinated either with heterologous prime-boost with pNGVL4a-sig/E7(detox)/HSP70 DNA intramuscularly (IM) followed by TA-HPV intraperitoneally, homologous prime-boost with pNGVL4a-sig/E7(detox)/HSP70 DNA IM, TA-HPV alone or no vaccination. As shown in FIG. 11, the heterologous prime-boost regimen generated the greatest number of IFN-γ secreting E7-specific CD8+ T cells among total splenocytes compared to homologous prime-boost vaccination or TA-HPV alone. This data suggests that DNA priming followed by vaccinia-based boosting is an effective prime-boost regimen to generate activated E7-specific CD8+ T cells.

### Example 9

### Vaccination with pNGVL4a-Sig/E7(Detox)/HSP70 DNA Prime Followed by TA-HPV Boost by Intracervicovaginal Delivery Generates a Greater Number of E7-Specific CD8+ T Cells in the Cervicovaginal Tract Compared to Vaccination Through Intramuscular Injection

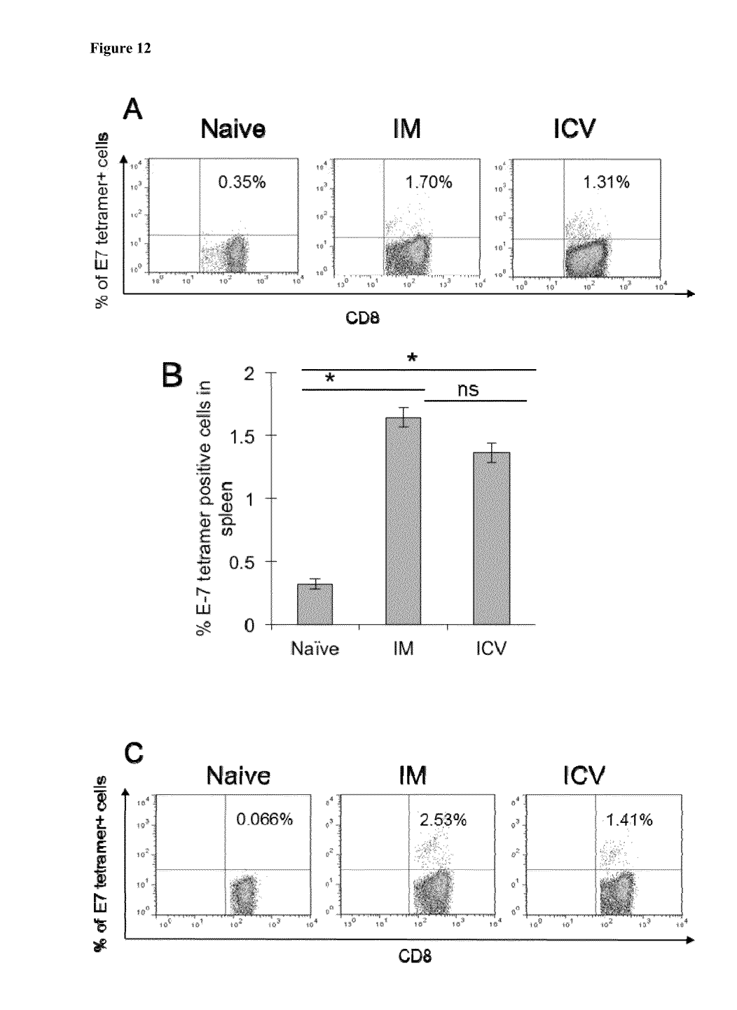

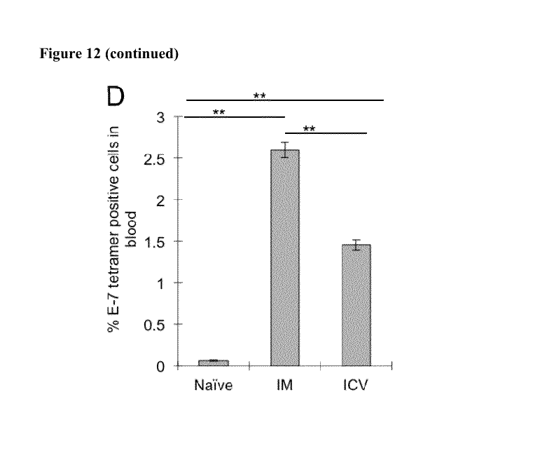

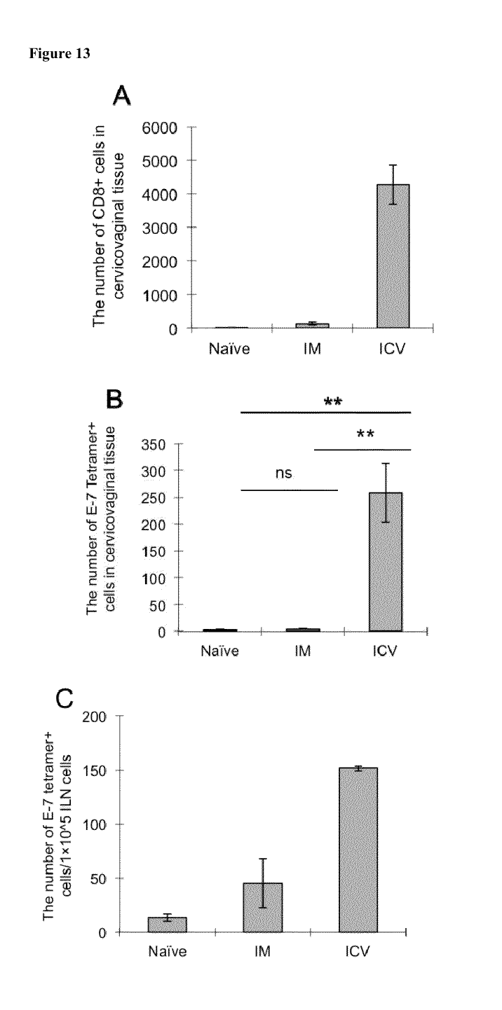

The effect of different administration routes on a vaccination regimen consisting of pNGVL4a-sig/E7(detox)/HSP70 DNA prime followed by TA-HPV boost on the generation of antigen-specific CD8+ T cells was examined. C57BL/6 mice were vaccinated with pNGVL4a-sig/E7(detox)/HSP70 DNA followed six days later by TA-HPV with the regimen administered either intracervicovaginally (ICV) or IM. One week after TA-HPV vaccination, mice were tested for E7-specific CD8+ T cells in various locations by flow cytometry analysis using E7 peptide-loaded H-2D^{b} tetramer staining As shown in FIGS. 12A and B, ICV and IM vaccination with pNGVL4a-sig/E7(detox)/HSP70 DNA followed by TA-HPV generated significantly higher percentages of E7-specific CD8+ T cells among splenocytes of mice compared to those of naïve mice. However, there appeared to be no significant difference between vaccination through the IM and ICV routes. FIGS. 12C and D shows that mice vaccinated with IM pNGVL4a-sig/E7(detox)/HSP70 DNA and TA-HPV generated significantly more E7-specific CD8+ T cells in the peripheral blood compared to mice that were ICV vaccinated. Furthermore, ICV vaccinated mice generated significantly more E7-specific CD8+ t cells than naïve mice. In contrast, ICV vaccination with pNGVL4a-sig/E7(detox)/HSP70 DNA and TA-HPV induced the highest percentage of E7-specific CD8+ T cells in the murine cervicovaginal tracts compared to IM vaccinated mice and naïve mice (FIGS. 13A and B). Taken together, this data indicates that vaccination through intracervicovaginal delivery represents a significantly more efficient method to generate a high number of E7-specific CD8+ T cells in the cervicovaginal tract compared to vaccination through intramuscular injection.

### Example 10

### Intracervicovaginal Vaccination with pNGVL4a-Sig/E7(Detox)/HSP70 DNA Prime Followed by TA-HPV Boost Generates a Significantly Higher Number of E7-Specific CD8+ T Cells in the Regional Lymph Nodes than Intramuscular Vaccination

Next, the effect of vaccine administration route on antigen-specific CD8+ T cells in the regional lymph nodes was studied. C57BL/6 mice were vaccinated with pNGVL4a-sig/E7(detox)/HSP70 DNA followed six days later by TA-HPV with the regimen administered either intracervicovaginally (ICV) or IM. One week after the last vaccination, the iliac lymph nodes (ILNs) were isolated and tested for E7-specific CD8+ T cells by flow cytometry analysis using E7 peptide-loaded H-2D^{b} tetramer staining. As shown in FIG. 13C, mice vaccinated ICV with pNGVL4a-sig/E7(detox)/HSP70 DNA and TA-HPV had the highest percentage of E7-specific CD8+ T cells in the ILNs compared to mice that were IM vaccinated and naïve mice. This indicates that ICV vaccination represents a more efficient way to induce a potent local E7-specific cell-mediated immune response compared to IM vaccination.

### Example 11

### Intracervicovaginal Vaccination with pNGVL4a-Sig/E7(Detox)/HSP70 DNA DNA Prime Followed by TA-HPV Boost Induces the Expression of α4β7 and CCR9 on E7-Specific CD8+ T Cells

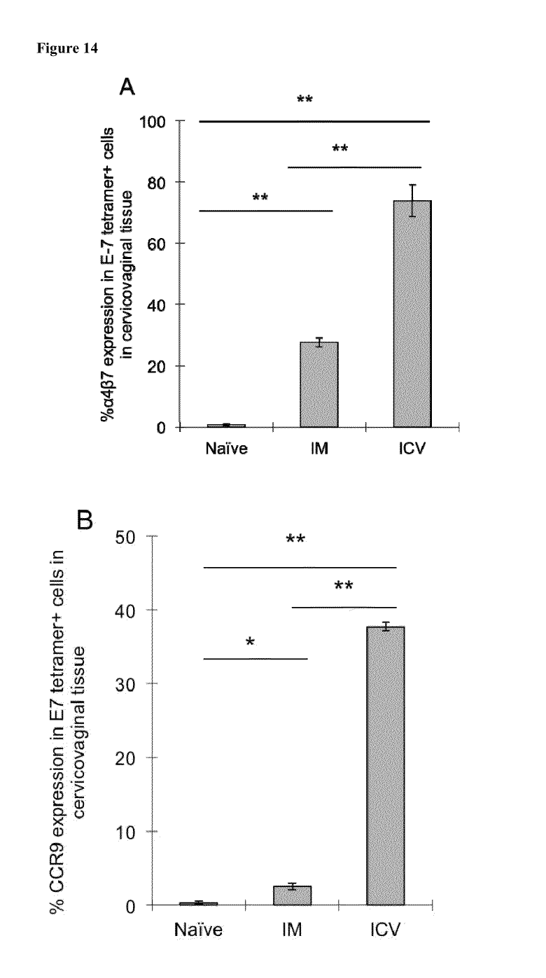

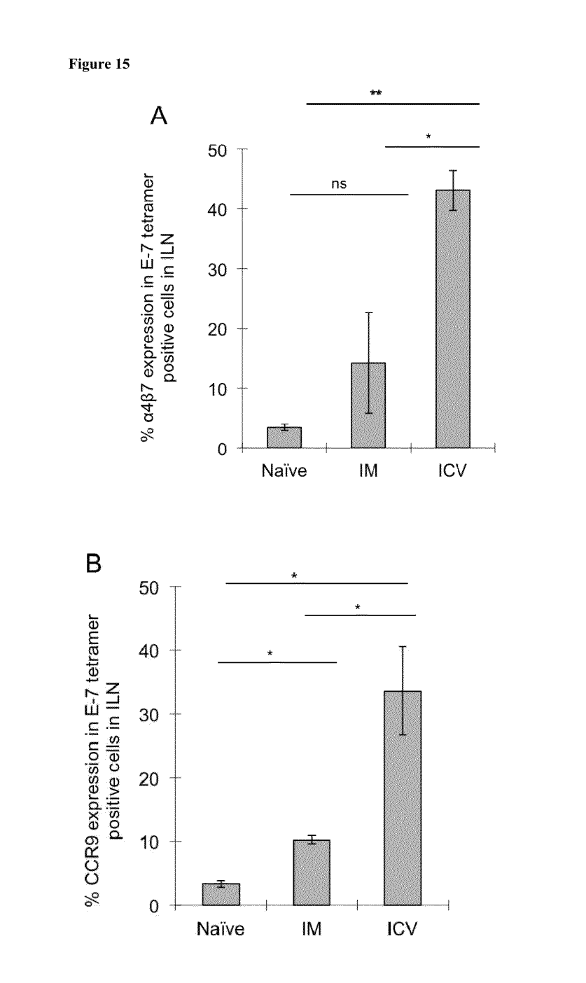

In order to determine whether the E7-specific CD8+ T cells induced by the prime-boost regimen were tissue-resident memory T cells, the expression of tissue-specific molecules α4β7 and CCR9 was evaluated. α4β7 is a mucosa-associated homing integrin that functions by interacting with Mucosal Addressin Cell Adhesion Molecule-1 (MAdCAM-1). Also involved in the homing and retention of lymphocytes in mucosal tissue is the chemokine receptor CCR9 whose ligand is CCL25, which is commonly expressed in the epithelium of respiratory, gastrointestinal and urogenital tissues. As shown in FIG. 14, mice treated with ICV pNGVL4a-sig/E7(detox)/HSP70 DNA and TA-HPV had the highest percentage of E7-specific CD8+ T cells expressing α4β7 or CCR9 among all E7 tetramer positive cells in the cervicovaginal tract. Furthermore, ICV vaccinated mice had the highest percentage of E7-specific CD8+ T cells expressing α4β7 or CCR9 in the regional ILN (FIG. 15). This data indicates that ICV vaccination is an effective method to generate antigen-specific CD8+ T cells that express α4β7 or CCR9.

### Example 12

### Intracervicovaginal Vaccination with pNGVL4a-Sig/E7(Detox)/HSP70 DNA Prime Followed by TA-HPV Boost Induces the Co-Expression of α4β7, CCR9 and CD103 on E7-Specific CD8+ T Cells

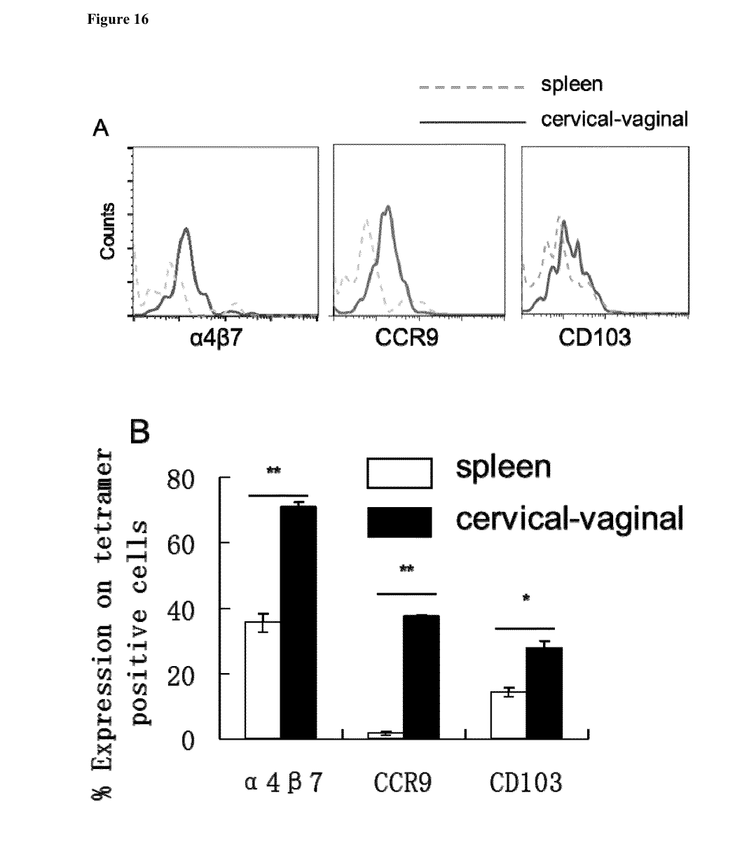

In order to determine the effect of the ICV prime-boost vaccination regimen on mucosal tissue-resident memory T cells locally and systemically, the expression of α4β7, CCR9 and CD103 was measured on E7-specific CD8+ T cells in the spleen and cervicovaginal tract of vaccinated mice. Mice were vaccinated ICV with pNGVL4a-sig/E7(detox)/HSP70 DNA followed by TA-HPV and their splenocytes and cervicovaginal tissues were analyzed by flow cytometry. As shown in FIG. 16, both α4β7 and CCR9 expression on E7-specific CD8+ T cells was significantly higher in the cervicovaginal tract compared to the spleen. This data suggests that ICV vaccination with our DNA-vaccinia prime-boost regimen increases the presence of E7-specific CD8+ T cells that have a surface phenotype consistent with that of mucosal tissue-resident memory T cells in the cervicovaginal tract.

### Example 13

### Intracervicovaginal Vaccination with pNGVL4a-Sig/E7(Detox)/HSP70 DNA Prime Followed by TA-HPV Boost Generates a Significantly Improved Therapeutic Antitumor Effect Compared to Intramuscular Vaccination

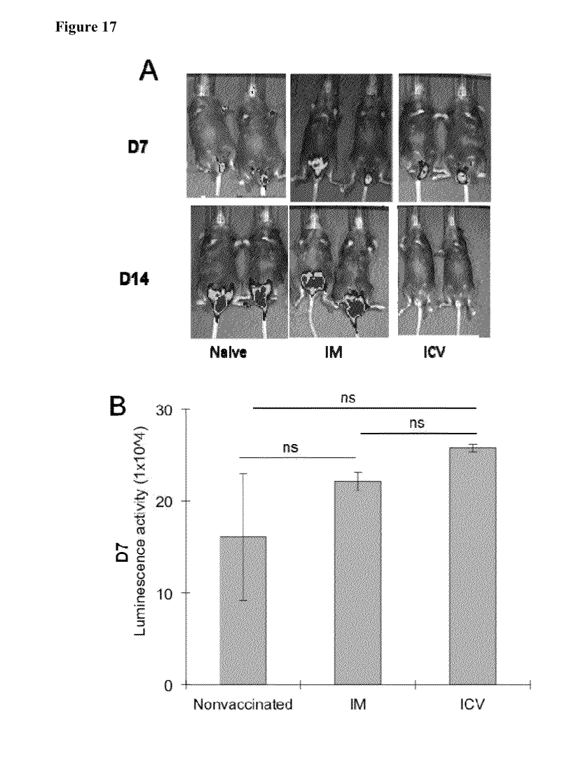

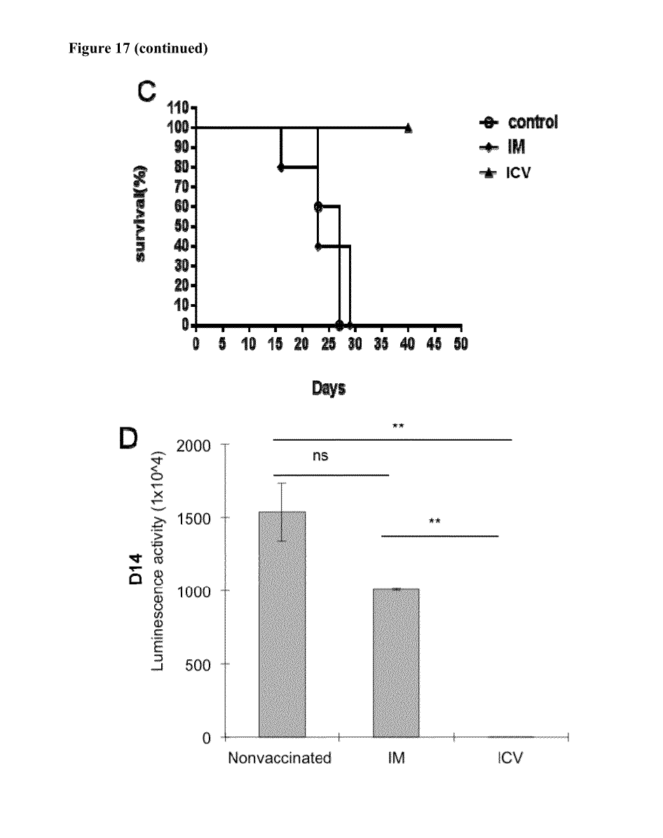

The therapeutic effect of the prime-boost regimen administered via different routes was assessed using a luciferase-expressing TC-1 tumor model. The level of luciferase activity represents the tumor load in mice. C57BL/6 mice were challenged subcutaneously with E7- and luciferase-expressing TC-1 tumor cells. One day later, mice were immunized with pNGVL4a-sig/E7(detox)/HSP70 DNA and 6 days later, mice were immunized with TA-HPV. Mice were monitored for tumor growth by luminescence imaging on day 7 and day 14 after tumor challenge. As shown in FIG. 17, mice receiving ICV vaccination with pNGVL4a-sig/E7(detox)/HSP70 DNA and TA-HPV experienced significantly greater antitumor effects, as measured by decreased luminescence, on day 14 compared to mice receiving IM vaccination or no vaccination. ICV vaccinated mice had no detectable luminescence on day 14, suggesting that they were eradicated of TC-1 tumor cells. Furthermore, all mice receiving the ICV vaccination regimen survived past 40 days, where as all mice receiving IM vaccination or no vaccination died before 30 days. This data indicates that vaccination through ICV is more efficient in generating a potent therapeutic antitumor effect and prolonging survival compared to vaccination through intramuscular injection.

### Example 14

### Intramuscular Vaccination with pNGVL4a-Sig/E7(Detox)/HSP70 DNA Prime Followed by Intracervicovaginal Vaccination with a TA-HPV Boost Generates the Highest Number of E7-Specific CD8+ T in Both the Spleen and the Cervicovaginal Tract

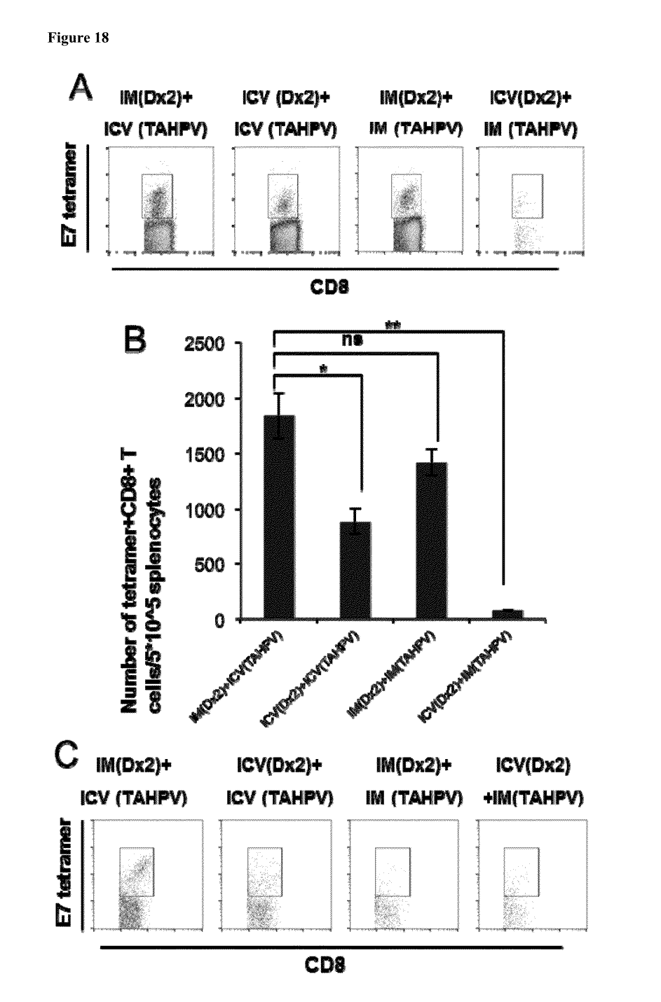

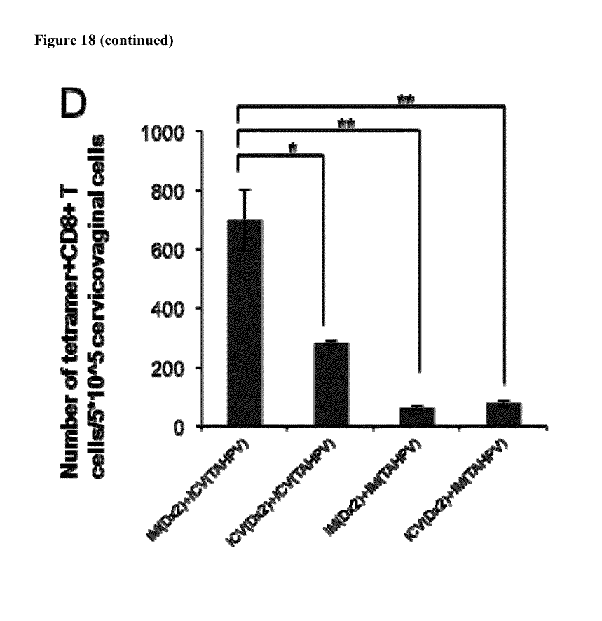

The systemic (spleen) and local (cervicovaginal tract) HPV E7 specific CD8+ T-cell mediated immune responses induced by different combinations of prime-boost delivery routes were assessed. C57BL/6 mice (5 per group) were vaccinated with pNGVL4a-sig/E7(detox)/HSP70 DNA (50 μg per mouse) intramuscularly or intracervicovaginally twice with a 7 day interval between vaccinations, followed by intramuscular or intracervicovaginal TA-HPV boost 7 days after the second DNA vaccination. Splenocytes and cervicovaginal cells were harvested and analyzed by flow cytometry 7 days after the last vaccination. FIG. 18 shows that IM DNA priming twice followed by ICV TA-HPV boost triggers the highest E7-specific CD8+ T cell production in both the cervicovaginal tract and in the spleen. Furthermore, ICV TA-HPV boost, regardless of the site of DNA priming, generates superior local production of E7-specific CD8+ T-cells (FIG. 18D). Interestingly, although IM DNA priming followed by IM TA-HPV boost is effective for the systemic production of CD8+ T cells but this combination is not effective for the generation of local HPV E7-specific CD8+ T cells in the cervicovaginal tract. Taken together, these results suggest that IM DNA prime followed by ICV TA-HPV boost is the most desirable combination to generate HPV E7 specific CD8+ T cells both the cervicovaginal tract and in the spleen.

1. A method of inducing or enhancing an antigen-specific immune response in a mammal, comprising the steps of:
(a) priming the mammal by administering to the mammal an effective amount of a nucleic acid composition encoding said antigen or a biologically active homolog thereof; and
(b) boosting the mammal by administering to the mammal an effective amount of a recombinant oncolytic virus comprising a nucleic acid encoding said antigen or the biologically active homolog thereof,
thereby inducing or enhancing the antigen-specific immune response.

2-3. (canceled)

4. The method of claim 1, wherein the antigen is a viral antigen.

5. The method of claim 1, wherein the antigen is selected from the group consisting of ovalbumin, HPV E6, and HPV E7.

6. The method of claim 5, wherein the antigen comprises an ovalbumin protein comprising an amino acid sequence at least 90% identical to an amino acid sequence of SEQ ID NO:139.

7. The method of claim 5, wherein the antigen comprises an HPV E7 protein comprising an amino acid sequence at least 90% identical to an amino acid sequence selected from the group consisting of LSRHFMHQKRTAMFQDPQERPRKLPQ (SEQ ID NO:140) and AMFQDPQERPRKLPQLCTELQTTIHDIILEC (SEQ ID NO:141).

8. The method of claim 5, wherein the antigen comprises an HPV E7 protein comprising an amino acid sequence at least 90% identical to an amino acid sequence selected from the group consisting of PTLHEYMLDLQPETTDLYCYEQ (SEQ ID NO:142), HEYMLDLQPET (SEQ ID NO:143), TLHEYMLDLQPETTD (SEQ ID NO:144), EYMLDLQPETTDLY (SEQ ID NO:145), DEIDGPAGQAEPDRAHY (SEQ ID NO:146) and GPAGQAEPDRAHYNI (SEQ ID NO:147).

9. The method of claim 1, wherein the nucleic acid composition is a DNA vaccine.

10. The method of claim 1, wherein the nucleic acid composition is administered from the group consisting of intradermally, subcutaneously, intraperitoneally, intramuscularly and intravenously.

11. The method of claim 1, wherein administering a nucleic acid composition comprises administering by a gene gun or in vivo electroporation.

12. The method of claim 1, wherein the nucleic acid composition comprises a transfection reagent, nanoparticle, or viral vector.

13. The method of claim 1, wherein the mammal is a human having a tumor and wherein the nucleic acid composition is administered intratumorally or peritumorally.

14. The method of claim 1, wherein the oncolytic virus is selected from the group consisting of vaccinia virus, adenovirus, herpes simplex virus, poxvirus, vesicular stomatitits virus, measles virus, Newcastle disease virus, influenza virus, and reovirus.

15. The method of claim 1, wherein the oncolytic virus is thymidine kinase negative.

16. (canceled)

17. The method of claim 1, wherein the oncolytic virus is administered into the anal cavity wall, oral cavity wall, or subcutaneously.

18. (canceled)

19. The method of claim 1, wherein the nucleic acid composition is present within an oncolytic virus.

20-28. (canceled)

29. The method of claim 1, wherein the mammal is a human.

30. The method of claim 1, wherein the mammal is afflicted with cancer.

31. The method of claim 1, wherein the mammal is afflicted with a persistent virus infection.

32. A method for treating or preventing advanced stage cancer in a mammal comprising (a) priming the mammal by administering to the mammal an effective amount of a nucleic acid composition encoding the antigen or a biologically active homolog thereof; and
(b) boosting the mammal by administering to the mammal an effective amount of an oncolytic virus comprising a nucleic acid encoding the antigen or the biologically active homolog thereof, thereby inducing or enhancing the antigen-specific immune response.

33. (canceled)

34. The method of claim 32, wherein the cancer is an HPV associated cancer.

35. A kit comprising a priming composition and a boosting composition, the kit comprising:
(a) a priming composition comprising DNA encoding an immunogenic foreign antigen and a pharmaceutically acceptable carrier; and
(a) a boosting composition comprising a virus encoding said foreign antigen and a pharmaceutically acceptable carrier.

36. The method of claim 1, wherein the nucleic acid composition or oncolytic virus is administered intracervicovaginally into the tract wall.

37. The method of claim 36, wherein the oncolytic virus is administered intracervicovaginally.

38. The method of claim 36, wherein the nucleic acid composition is administered intramuscularly and the oncolytic virus is administered intracervicovaginally.

39. The method of claim 36, wherein both the nucleic acid composition and the oncolytic virus are administered intracervicovaginally.

40. The method of claim 1, wherein the oncolytic virus is TA-HPV.
